(19) United States
(12) Patent Application Publication
(10) Pub. No.: US 2023/0282305 A1
(43) Pub. Date: Sep. 7, 2023

(54) Predictive Universal Signatures for Multiple Disease Indications

(71) Applicant: Vir Biotechnology, Inc., San Francisco, CA (US)

(72) Inventors: Amalio Telenti, San Francisco, CA (US); Julia Di Iulio, San Francisco, CA (US)

(21) Appl. No.: 18/019,905

(22) PCT Filed: Aug. 6, 2021
(86) PCT No.: PCT/US2021/044903

§ 371 Date: Feb. 6, 2023

Related U.S. Application Data
(60) Provisional application No. 63/062,665, filed on Aug. 7, 2020.
(60) Provisional application No. 63/129,931, filed on Dec. 23, 2020.
(60) Provisional application No. 63/192,461, filed on May 24, 2021.

(51) Int. Cl. G16B 20/00; G16B 40/20; G16H 50/70; G16H 50/20

(52) U.S. Cl. CPC G16B 20/00; G16B 40/20; G16H 50/70; G16H 50/20

(57) ABSTRACT

Universal signatures represent generalizable features that are informative for generating predictions for different disease activities across different diseases. More specifically, one or more universal signatures are learned from data pertaining to a first disease indication and then applied to generate predictions for a one or more additional disease indications. The implementation of one or more universal signatures is useful for generating predictions for disease indications, such as disease indications involving rare or novel diseases, where it may be infeasible to develop a model due to insufficient training data.

### CROSS REFERENCE TO RELATED APPLICATIONS

This application claims the benefit of and priority to U.S. Provisional Patent Application No. 63/062,665 filed Aug. 7, 2020, U.S. Provisional Patent Application No. 63/129,931 filed Dec. 23, 2020, and U.S. Provisional Patent Application No. 63/192,461 filed May 24, 2021, the entire disclosure of each of which is hereby incorporated by reference in its entirety for all purposes.

### BACKGROUND OF THE INVENTION

Significant effort has been expended towards developing state-of-the-art models that are trained and deployed on datasets for predicting disease activity in patients. For example, models are developed using a training dataset including data related to a disease and the models are subsequently deployed on a test dataset to generate predictions for the disease. These state-of-the art models require the development of disease-specific signatures that are only applicable for making predictions for that particular disease. Put another way, these trained models are only useful for generating predictions for the same disease for which the models were trained for.

There are significant limitations to this strategy. First, obtaining a training dataset that is sufficient for training a model can be difficult for certain diseases, such as a disease for which there are not enough real life data points. This can be the case for rare diseases or for novel diseases. Second, even if a sufficient training dataset is obtained, the process of training a model for multiple diseases is computationally expensive and often risks overfitting each model to the training dataset. As a result, the model suffers a significant loss in performance when applied to a test dataset or when the models are generalized to new sources of data (e.g., new sources of data with differences in geography and patient populations).

### SUMMARY

Disclosed herein are universal signatures that represent generalizable features that are informative for making predictions for different disease indications. In various embodiments, a machine learning approach is implemented to identify common elements in data sets and then these common elements are tested empirically to determine whether they are informative about a second data set from a disease or process distinct from the original data set. Sets of genes, hereafter referred to as universal signatures, are predictive across diverse datasets and/or species (e.g. rhesus to humans). These universal signatures are useful in different use cases, examples of which include the cases of progression of latent to active tuberculosis, and severity of COVID-19 and influenza A H1N1 infection. Therefore, universal signatures can be deployed in settings that lack disease-specific biomarkers. Thus, a small set of archetypal human immunophenotypes, captured by universal signatures, can explain a larger set of responses to diverse diseases.

Embodiments described herein are methods for developing one or more universal signatures according to data associated with a first disease indication. The one or more universal signatures are used to generate predictions for disease activity in a second (e.g., different) disease indication. Furthermore, described herein are embodiments directed to non-transitory computer readable mediums comprising instructions that, when executed by a processor, cause the processor to develop one or more universal signatures according to data associated with a first disease. Furthermore, such instructions can cause the processor to use the one or more universal signatures to generate predictions for disease activity in a second (e.g., different) disease.

Altogether, the development and implementation of the one or more universal signatures represents a form of transfer learning, where the one or more universal signatures learned from data relating to a first disease indication can be applied to solve a new problem, which in this case involves generating predictions for a second disease indication (e.g., a different disease or a disease in a different species). Thus, universal signatures can be informative across unrelated datasets pertaining to different diseases. The use of transfer learned universal signatures is useful for generating predictions for diseases where sufficient examples in training datasets are limited or difficult to obtain. For example, the learned universal signature of a first disease indication can be applied to generate predictions for disease activity of a rare or novel disease. Additionally, the use of transfer learned universal signatures avoids the problem of overfitted models. Universal signatures may sacrifice a level of sensitivity and/or specificity for any particular individual disease to ensure that the universal signatures are generally predictive for disease activities across multiple diseases. More generally, the work provides support to the concept of human immunophenotypes based on universal signatures.

Disclosed herein is a method for identifying one or more universal signatures useful for evaluating disease activity of two or more diseases, the method comprising: obtaining or having obtained expressions of a plurality of markers across individuals for a first disease indication; analyzing the expressions of the plurality of markers using a machine-learned analysis to identify one or more universal signatures from the first disease indication, wherein the one or more universal signatures are features that are predictive for a second disease indication, wherein each of the first disease indication and the second disease indication is characterized by a common condition.

Additionally disclosed herein is a method for generating a prediction of a second disease indication for a patient, the method comprising: obtaining or having obtained expressions of one or more universal signatures from the subject, the one or more universal signatures derived from a machine-learned analysis of a plurality of markers across individuals associated with a first disease indication, wherein each of the first disease indication and the second disease indication is characterized by a common condition; and based on the expressions for the one or more universal signatures, generating the prediction of the second disease indication.

In various embodiments, the one or more universal signatures comprise one or more of genes, nucleic acids, metabolites, or protein biomarkers. In various embodiments, the common condition is any one of a precursor to a disease, a sub phenotype of a disease, progression from latent to acute infection, progression from acute to chronic infection, response to an intervention, susceptibility to disease or infection, presence of acute inflammation, presence of chronic inflammation, a dysregulated pathway expression, a cellular phenotype, or a clinical phenotype. In various embodiments, the clinical phenotype is any one of high blood pressure, fever, loss of blood, loss of consciousness, increased heart rate, or need for mechanical ventilation. In various embodiments, the first disease indication describes a disease activity of a first disease, and wherein the second disease indication describes a disease activity of a second disease, and wherein the first disease indication differs from the second disease indication by any of a different disease activity of a disease, a disease activity of different diseases, different disease activity of different diseases.

In various embodiments, each of the first disease indication or second disease indication is any one of activity of an inflammatory disease, activity of a disease observed in an animal model, activity of a bacterial infectious disease, a progression from latent to acute infection, and wherein the disease activity of the second disease is any one of disease of a cancer, activity of a human disease that represents an equivalent phenotype of a disease in an animal, activity of an infectious disease from a non-bacterial infectious agent, protection after vaccination, estimated time to death due to disease, or a diseased condition. In various embodiments, the first disease is an inflammatory disease and the second disease is a cancer. In various embodiments, the first disease is observed in an animal model and wherein the second disease is an equivalent disease phenotype in humans. In various embodiments, the first disease is a bacterial infectious disease and wherein the second disease is a disease from a non-bacterial infectious agent. In various embodiments, the disease activity of the first disease is a progression from latent to acute infection and wherein the disease activity of the second disease is protection after vaccination.

In various embodiments, the machine-learned analysis is random forest or gradient boosting for identifying the one or more universal signatures. In various embodiments, the intervention is any one of a small molecule therapeutic, a biologic, a vaccine, or a gene therapy. In various embodiments, individuals with the second disease have encountered or are likely to encounter the common condition.

In various embodiments, generating a prediction of the second disease indication for the patient comprises performing an unsupervised clustering of the expressions of the one or more universal signatures to classify the patient. In various embodiments, generating the prediction of the second disease indication for a patient comprises performing a dimensionality reduction analysis of the expressions of the one or more universal signatures.

In various embodiments, the method further comprises: determining whether to include the subject in a clinical trial study according to the predicted disease activity of the disease in the subject.

In various embodiments, the one or more universal signatures comprise one or more genes selected from NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, BCAP31, ATG4B, CSRP1, RDH11, GCLM, CDC7, GLOD5, IDH2, FMR1, PPARA, CCNE1, DDB1, BMP1, EHD4, VAV3, MPG, SPAG4, PSMD3, BCKDHA, GRAMD1B, and SEC61A1. In various embodiments, the one or more universal signatures comprise one or more genes selected from CRB3, BCAP31, GMPPB, CD4, STARD3, CALR, CSRP1, CPT1A, LDLRAP1, RRAS, HMGCR, RASGRP2, PTS, SORDSLC26A6, VAT1, GPAA1, CXCR3, NAMPT, EPHX1, SEPT9, GMPPA, B4GALT7, AAAS, TP53INP1, GYS1, FASN, NOC4L, RRP9, MXIL TP53, SLC7A11, FOXP3, DNASE1L1, MGAT1, SEC61A1, FYCO1, S100A10, LSS, IFRD1, DCP2, EDC4, ANKZFL IDUA, IGFBP2, DDX39A, UCHL1, NR4A1, PDIA5, and ENGASE. In various embodiments, the one or more universal signatures comprise one or more genes selected from NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, ITGA2, MPZL2, FEZ1, JAK2, BAZ1A, ICAM4, DAPP1, RIPK1, RNF144B, LAP3, C1QA, TYMP, GCH1, C1QB, CREM, ETV7, FOSB, MRPL15, PSEN1, MXI1, and TRAFD1.

In various embodiments, the one or more universal signatures comprise one or more genes selected from DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, RTP4, CTSD, HIST1H2BG, IL27, TNFRSF1B, SORBS1, NOP2, TNFSF13B, HLA-DRB5, RHOG, PSMB9, HSPA6, CD63, SLC2A8, IFITM1, CKB, ALDOA, MSRB1, OSMR, DRAP1, and PLA2G4A. In various embodiments, the one or more universal signatures comprise one or more genes selected from LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, KIAA1324, CENPN, IKBKE, SERPINF2, FAM162A, SNX2, SERPING1, CLCA2, DPEP3, TNFAIP2, FSTL4, CTSD, BCAR1, MKX, RGS2, SAMD9, GCLM, BST1, IRS2, RNASE6, and ELOVL3. In various embodiments, the one or more universal signatures comprise one or more genes selected from GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, TNFRSF21, PSMB9, GNAI1, TSPAN13, ATP6V0B, SLC4A4, ILF2, AKAP12, HLA-DRB5, PGR, AGTRAP, P3H1, CDADC1, TRIM5, PTGER3, ADCY6, ERBB2, NFYA, STATE, MMD, and RPL10A.

In various embodiments, the one or more universal signatures comprise one or more genes selected from MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, IFNGR2, B3GNT5, KYNU, LPGAT1, SLC9A3R1, HP, PADI4, PSME1, MGST2, NR4A1, SPP1, DEFA3, ME1, RBP7, DUSP6, and MCRS1. In various embodiments, the one or more universal signatures comprise one or more genes selected from POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, RFC2, CAMK1G, CBX8, HM13, PSMB10, GCLM, SLC25A3, MYD88, IL33, ITGAM, PPIA, SEC22B, CXCR3, SCRN1, RXRA, SDHA, GLDC, FGF6, PRKG2, TFPI, and IMMT. In various embodiments, the one or more universal signatures comprise one or more genes selected from CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, PICALM, KRT6A, FMR1, PCSK9, DNASE1L3, ENDOG, TPD52L1, PEX6, MPO, CHRNA7, SLFN5, TNFRSF1A, CD24, CASC1, LLGL2, DLG5, MYO5C, PGR, PFKFB2, AK2, and COL19A1.

In various embodiments, the one or more universal signatures comprise one or more genes selected from HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, ROCK1, CD83, AK7, MSR1, LCN2, SPN, ASS1, HDGF, CXCL16, POLR3D, GK, OLFM4, STK3, RCBTB1, FOLR3, FBXO32, TMEM98, PRDX2, CKB, UHRF1BP1L and CTSG. In various embodiments, the one or more universal signatures comprise one or more genes selected from AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMPI, HSD11B2, SLC25A25, RAB32, CXCL9, KCNE2, FCAR, CFP, IGF1, PEX16, RNF214, PIM1, JUNB, MDM2, PFKFB4, SIAH2, EGR2, KCNK10, EHMT2, FPR1, CD27, CETN2, and TGM1.

In various embodiments, the one or more universal signatures comprise one or more genes selected from SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, MT2A, COL4A2, PLCB4, GYS1, PRKCG, RXFP2, PLA2G4C, ALDH1A2, IL1A, IBTK, SPARC, OAS3, EPHA4, HLA-B, MICB, CCL18, SLC39A6, GLCE, TUBB2B, FBXO8, and SNX6. In various embodiments, the one or more universal signatures comprise one or more genes selected from NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, SLC25A19, MAP7, XCL1, ACSL6, TFRC, CAT, NKD1, CNBP, ALDH1L1, CCL7, SLC20A1, KRAS, CSF1, CASP2, HDAC11, KIR2DS4, CEACAM19, CFH, CAB39L, DEPDC1, and PSMAL In various embodiments, the one or more universal signatures comprise one or more genes selected from CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, CENPJ, ARL8A, CTLA4, GFRA1, WASF1, RIPK1, ENO3, KRT19, PLVAP, RAD18, ACHE, FBLN5, MGST2, ANAPC5, RFX5, CASP7, STC1, NCK2, IFI27, APOA4, and MSRB2.

Additionally disclosed herein is a non-transitory computer-readable medium for identifying one or more universal signatures useful for evaluating two or more disease indications, the computer-readable medium comprising instructions that, when executed by a processor, cause the processor to perform the steps comprising: obtaining or having obtained expressions of a plurality of markers across individuals for a first disease indication; analyzing the expressions of the plurality of markers using a machine-learned analysis to identify one or more universal signatures from the first disease indication, wherein the one or more universal signatures are features that are predictive for a second disease indication, wherein each of the first disease indication and the second disease indication is characterized by a common condition.

Additionally disclosed herein is a non-transitory computer-readable medium for generating a prediction of a second disease indication for a patient, the computer-readable medium comprising instructions that, when executed by a processor, cause the processor to perform the steps comprising: obtaining or having obtained expressions of one or more universal signatures from the subject, the one or more universal signatures derived from a machine-learned analysis of a plurality of markers across individuals associated with a first disease indication, wherein each of the first disease indication and the second disease indication is characterized by a common condition; and based on the expressions for the one or more universal signatures, generating the prediction of the second disease indication.

In various embodiments, the one or more universal signatures comprise one or more of genes, nucleic acids, metabolites, or protein biomarkers. In various embodiments, the common condition is any one of a precursor to a disease, a sub phenotype of a disease, progression from latent to acute infection, progression from acute to chronic infection, response to an intervention, susceptibility to disease or infection, presence of acute inflammation, presence of chronic inflammation, a dysregulated pathway expression, a cellular phenotype, or a clinical phenotype (e.g., high blood pressure, fever, loss of blood, loss of consciousness, or increased heart rate). In various embodiments, the clinical phenotype is any one of high blood pressure, fever, loss of blood, loss of consciousness, increased heart rate, or need for mechanical ventilation.

In various embodiments, the first disease indication describes a disease activity of a first disease, and wherein the second disease indication describes a disease activity of a second disease, and wherein the first disease indication differs from the second disease indication by any of a different disease activity of a disease, a disease activity of different diseases, different disease activity of different diseases. In various embodiments, each of the first disease indication or second disease indication is any one of activity of an inflammatory disease, activity of a disease observed in an animal model, activity of a bacterial infectious disease, a progression from latent to acute infection, a dysregulated blood cell population makeup, or a dysregulated pathway expression, and wherein the disease activity of the second disease is any one of disease of a cancer, activity of a human disease that represents an equivalent phenotype of a disease in an animal, activity of an infectious disease from a non-bacterial infectious agent, protection after vaccination, estimated time to death due to disease, or a diseased condition. In various embodiments, the first disease is an inflammatory disease and the second disease is a cancer. In various embodiments, the first disease is observed in an animal model and wherein the second disease is an equivalent disease phenotype in humans. In various embodiments, the first disease is a bacterial infectious disease and wherein the second disease is a disease from a non-bacterial infectious agent. In various embodiments, the disease activity of the first disease is a progression from latent to acute infection and wherein the disease activity of the second disease is protection after vaccination.

In various embodiments, the machine-learned analysis is random forest or gradient boosting for identifying the one or more universal signatures. In various embodiments, the intervention is any one of a small molecule therapeutic, a biologic, a vaccine, or a gene therapy. In various embodiments, individuals with the second disease have encountered or are likely to encounter the common condition.

In various embodiments, generating the prediction of the second disease indication for the patient comprises performing an unsupervised clustering of the expressions of the one or more universal signatures to classify the subject. In various embodiments, generating the prediction of the second disease indication for the patient comprises performing a dimensionality reduction analysis of the expressions of the one or more universal signatures. In various embodiments, the non-transitory computer-readable medium further comprises instructions that, when executed by the processor, cause the processor to perform the steps comprising: determining whether to include the subject in a clinical trial study according to the prediction of the disease indication for the patient.

In various embodiments, the one or more universal signatures comprise one or more genes selected from NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, BCAP31, ATG4B, CSRP1, RDH11, GCLM, CDC7, GLOD5, IDH2, FMR1, PPARA, CCNE1, DDB1, BMP1, EHD4, VAV3, MPG, SPAG4, PSMD3, BCKDHA, GRAMD1B, and SEC61A1. In various embodiments, the one or more universal signatures comprise one or more genes selected from CRB3, BCAP31, GMPPB, CD4, STARD3, CALR, CSRP1, CPT1A, LDLRAP1, RRAS, HMGCR, RASGRP2, PTS, SORDSLC26A6, VAT1, GPAA1, CXCR3, NAMPT, EPHX1, SEPT9, GMPPA, B4GALT7, AAAS, TP53INP1, GYS1, FASN, NOC4L, RRP9, MXIL TP53, SLC7A11, FOXP3, DNASE1L1, MGAT1, SEC61A1, FYCO1, S100A10, LSS, IFRD1, DCP2, EDC4, ANKZFL IDUA, IGFBP2, DDX39A, UCHL1, NR4A1, PDIA5, and ENGASE. In various embodiments, the one or more universal signatures comprise one or more genes selected from NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, ITGA2, MPZL2, FEZ1, JAK2, BAZ1A, ICAM4, DAPP1, RIPK1, RNF144B, LAP3, C1QA, TYMP, GCH1, C1QB, CREM, ETV7, FOSB, MRPL15, PSEN1, MXIL and TRAFD1.

In various embodiments, the one or more universal signatures comprise one or more genes selected from DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, RTP4, CTSD, HIST1H2BG, IL27, TNFRSF1B, SORBS1, NOP2, TNFSF13B, HLA-DRB5, RHOG, PSMB9, HSPA6, CD63, SLC2A8, IFITM1, CKB, ALDOA, MSRB1, OSMR, DRAP1, and PLA2G4A. In various embodiments, the one or more universal signatures comprise one or more genes selected from LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, KIAA1324, CENPN, IKBKE, SERPINF2, FAM162A, SNX2, SERPING1, CLCA2, DPEP3, TNFAIP2, FSTL4, CTSD, BCAR1, MKX, RGS2, SAMD9, GCLM, BST1, IRS2, RNASE6, and ELOVL3. In various embodiments, the one or more universal signatures comprise one or more genes selected from GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, TNFRSF21, PSMB9, GNAI1, TSPAN13, ATP6V0B, SLC4A4, ILF2, AKAP12, HLA-DRB5, PGR, AGTRAP, P3H1, CDADC1, TRIM5, PTGER3, ADCY6, ERBB2, NFYA, STATE, MMD, and RPL10A.

In various embodiments, the one or more universal signatures comprise one or more genes selected from MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, IFNGR2, B3GNT5, KYNU, LPGAT1, SLC9A3R1, HP, PADI4, PSME1, MGST2, NR4A1, SPP1, DEFA3, ME1, RBP7, DUSP6, and MCRS1. In various embodiments, the one or more universal signatures comprise one or more genes selected from POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, RFC2, CAMK1G, CBX8, HM13, PSMB10, GCLM, SLC25A3, MYD88, IL33, ITGAM, PPIA, SEC22B, CXCR3, SCRN1, RXRA, SDHA, GLDC, FGF6, PRKG2, TFPI, and IMMT. In various embodiments, the one or more universal signatures comprise one or more genes selected from CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, PICALM, KRT6A, FMR1, PCSK9, DNASE1L3, ENDOG, TPD52L1, PEX6, MPO, CHRNA7, SLFN5, TNFRSF1A, CD24, CASC1, LLGL2, DLG5, MYO5C, PGR, PFKFB2, AK2, and COL19A1. In various embodiments, the one or more universal signatures comprise one or more genes selected from HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, ROCK1, CD83, AK7, MSR1, LCN2, SPN, ASS1, HDGF, CXCL16, POLR3D, GK, OLFM4, STK3, RCBTB1, FOLR3, FBXO32, TMEM98, PRDX2, CKB, UHRF1BP1L and CTSG. In various embodiments, the one or more universal signatures comprise one or more genes selected from AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMPI, HSD11B2, SLC25A25, RAB32, CXCL9, KCNE2, FCAR, CFP, IGF1, PEX16, RNF214, PIM1, JUNB, MDM2, PFKFB4, SIAH2, EGR2, KCNK10, EHMT2, FPR1, CD27, CETN2, and TGM1.

In various embodiments, the one or more universal signatures comprise one or more genes selected from SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, MT2A, COL4A2, PLCB4, GYS1, PRKCG, RXFP2, PLA2G4C, ALDH1A2, ILIA, IBTK, SPARC, OAS3, EPHA4, HLA-B, MICB, CCL18, SLC39A6, GLCE, TUBB2B, FBXO8, and SNX6. In various embodiments, the one or more universal signatures comprise one or more genes selected from NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, SLC25A19, MAP7, XCL1, ACSL6, TFRC, CAT, NKD1, CNBP, ALDH1L1, CCL7, SLC20A1, KRAS, CSF1, CASP2, HDAC11, KIR2DS4, CEACAM19, CFH, CAB39L, DEPDC1, and PSMA1. In various embodiments, the one or more universal signatures comprise one or more genes selected from CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, CENPJ, ARL8A, CTLA4, GFRA1, WASF1, RIPK1, ENO3, KRT19, PLVAP, RAD18, ACHE, FBLN5, MGST2, ANAPC5, RFX5, CASP7, STC1, NCK2, IFI27, APOA4, and MSRB2.

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “third party entity 330A,” indicates that the text refers specifically to the element having that particular reference numeral. A reference numeral in the text without a following letter, such as “third party entity 330,” refers to any or all of the elements in the figures bearing that reference numeral (e.g. “third party entity 330” in the text refers to reference numerals “third party entity 330A” and/or “third party entity 330B” in the figures).

These and other features, aspects, and advantages of the present invention will become better understood with regard to the following description, and accompanying drawings, where:

Figure (FIG. 1 depicts a high-level block diagram process for generating universal signatures from a first disease indication and applying the universal signatures for generating predictions for a second disease indication, in accordance with an embodiment.

FIG. 2A depicts a flow process for generating universal signatures using data associated with a first disease indication, in accordance with an embodiment.

FIG. 2B depicts a flow process for generating a prediction for a second disease indication using the universal signature, in accordance with an embodiment.

FIG. 3 depicts an overall system environment for generating and using universal signatures, in accordance with an embodiment.

FIG. 4 illustrates an example computer for implementing the methods described in FIGS. 1 and 2A/2B and the entities shown in FIG. 3.

FIG. 5A depicts an example diagram of generating universal signatures from a training set and their implementation in a test set.

FIG. 5B depicts the performance of the universal signatures on their target datasets.

FIG. 5C depicts an example study design including signatures, training datasets, and test datasets.

FIG. 5D depicts performance of different signatures, supporting the notion that published signatures contain valuable information that can be used to train predictive models and classifiers.

FIG. 5E depicts top performing signatures across the various training datasets.

FIG. 6A depicts receiver operating curves for validating signatures extracted from Rhesus or human datasets against a Rhesus dataset.

FIG. 6B depicts a receiver operating curve for validating universal signatures extracted from Rhesus and human datasets against a Rhesus dataset.

FIG. 6C depicts receiver operating curves for validating signatures extracted from Rhesus or human datasets against a human dataset.

FIG. 6D depicts a receiver operating curve for validating universal signatures extracted from Rhesus and human datasets against a human dataset.

FIG. 7A depicts results following a dimensionality reduction analysis and unsupervised clustering of human data using universal signatures learned from Rhesus Macaque datasets.

FIG. 7B depicts the performance in a tuberculosis progression use case using different sizes of universal signatures

FIG. 7C depicts a comparison of universal signatures obtained from different signature groups in a tuberculosis progression use case.

FIG. 8 depicts results of a dimensionality reduction analysis of a human glioma dataset using universal signatures learned using hallmark pathways signatures trained on a tuberculosis dataset.

FIG. 9A depicts results of a dimensionality reduction analysis and unsupervised clustering of a human SARS-CoV-2 infection dataset and a human H1N1 infection dataset using universal signatures learned from a human Dengue virus infection dataset.

FIG. 9B depicts the performance in a severe viral disease use case using different sizes of universal signatures.

FIG. 9C depicts a comparison of universal signatures obtained from different signature groups in a severe viral disease use case.

FIG. 10 depicts performance of universal signatures as compared to single signatures.

FIG. 11 depicts the performance of universal signatures of varying sizes.

FIG. 12 depicts the number of literature signatures at differing thresholds (70, 80 and 90 percentile).

### DETAILED DESCRIPTION OF THE INVENTION

### Definitions

Terms used in the claims and specification are defined as set forth below unless otherwise specified.

The term “subject,” “individual,” or “patient” are used interchangeably and encompass a cell, tissue, organism, human or non-human, mammal or non-mammal, male or female, whether in vivo, ex vivo, or in vitro. In various embodiments, different subjects can be human or non-human, and as such, the generation and use of universal signatures, as described herein, can be generated and/or deployed for both human and non-human subjects.

The terms “marker,” “markers,” “biomarker,” and “biomarkers” are used interchangeably and encompass, without limitation, lipids, lipoproteins, proteins, cytokines, chemokines, growth factors, peptides, nucleic acids, genes, oligonucleotides, metabolites, mutations, variants, polymorphisms, modifications, fragments, subunits, degradation products, elements, and other analytes or sample-derived measures. A marker can also include mutated proteins, mutated nucleic acids, structural variants including copy number variations, inversions, and/or transcript variants.

The term “expression of markers” refers to a quantity or state of a marker. For example, expression of a peptide can refer to a quantitative amount of the peptide e.g., quantity of the peptide in a sample. As another example, expression of a nucleic acid can refer to a quantitative amount of the nucleic acid e.g., quantity of the nucleic acid in a sample. As another example, expression of a gene can refer to the quantitative amount of gene product (e.g., a transcript such as RNA nucleic acid transcribed from the gene, or a protein translated from the mRNA of the gene). As another example, expression of a gene can refer to a state of the gene, such as an active state or a silenced state. As another example, expression of a marker refers to quantities of metabolites or metabolic patterns from metabolomics.

The terms “universal signature,” “transfer signature,” or “shared signature” are used interchangeably and refers to one or more markers that are predictive for two or more disease indications. In various embodiments, a universal signature includes one marker, such as a gene marker. In various embodiments, a universal signature includes two or more markers, such as two or more gene markers. Generally, a universal signature, as disclosed herein, is identified by analyzing data related to a first disease indication. Such a universal signature can then be applied for generating predictions for additional disease indications. In various embodiments, a universal signature is associated with a common condition of the first disease indication and the second disease indication. For example, the universal signature can play a role in the underlying biology of the common condition of the first disease indication and the second disease indication. This enables the universal signature to be predictive of the first disease indication and the second disease indication.

The term “disease indication” refers to disease activity or state of a disease. The term “different disease indication” refers to any of 1) different disease activity of a disease, 2) a disease activity of different diseases, or 3) different disease activity of different diseases. Generally, a first disease indication and a second disease indication differ either by the disease activity, the disease, or both. For example, a first disease indication can be vaccine protection in tuberculosis, where the disease activity refers to vaccine protection and the disease is tuberculosis. A second disease indication can be progression of tuberculosis, where disease activity refers to progression and the disease is tuberculosis. As another example, a first disease indication can be chronic infection in infectious diseases, where the disease activity refers to chronic infection and the diseases are infectious diseases. A second disease indication can refer to the same disease activity (e.g., chronic infection) in a different disease (e.g., glioma). The phrase “different disease” also encompasses a disease in different species. For example, tuberculosis in a human and tuberculosis in a non-human (e.g., Rhesus Macaque) are considered different diseases.

The phrase “disease activity of a disease” refers to any one of activity of an inflammatory disease, activity of a cancer, activity of a disease observed in an animal model, activity of a bacterial infectious disease, activity of a viral infectious disease, a progression from latent to acute infection, disease of a cancer, activity of a human disease that represents an equivalent phenotype of a disease in an animal, activity of an infectious disease from a non-bacterial infectious agent, protection after vaccination, antibody response to vaccination, estimated time to death due to disease, or a diseased condition.

The term “sample” or “test sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art.

The term “obtaining data” or “obtaining a dataset” encompasses obtaining a set of data determined from at least one sample. Obtaining a dataset encompasses obtaining a sample and processing the sample to experimentally determine the data. The phrase also encompasses creating a dataset. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

The phrase “common condition” refers to any one of a precursor to a disease, a sub phenotype of a disease, progression from latent to acute infection, progression from acute to chronic infection, response to an intervention, susceptibility to disease or infection, presence of acute inflammation, presence of chronic inflammation, a dysregulated pathway expression, a cellular phenotype, or a clinical phenotype (e.g., high blood pressure, fever, loss of blood, loss of consciousness, or increased heart rate). In various embodiments, a first disease and a second disease share a common condition (e.g., share a common precursor or common sub phenotype).

Therefore, one or more universal signatures developed from a first disease indication can be predictive for disease activity for a second disease indication due to the sharing of the common condition between the first and second diseases.

It must be noted that, as used in the specification and the appended claims, the singular forms “a,” “an” and “the” include plural referents unless the context clearly dictates otherwise.

Overview

FIG. 1 depicts a high-level block diagram process 100 for generating one or more universal signatures from data associated with a first disease indication and applying the one or more universal signatures for generating predictions for a second disease indication, in accordance with an embodiment. In particular, FIG. 1 depicts two different processes: 1) a development process 150 for identifying one or more universal signatures from data of a first disease indication and 2) a deployment process 160 for applying the one or more universal signatures to generate a prediction for a second disease indication (e.g., predict disease activity of a second disease).

Data associated with a first disease indication 110 is obtained. In various embodiments, data associated with a first disease indication 110 comprises data that are derived from individuals. Such individuals can be known to have the first disease indication (e.g., disease activity of a first disease). For example, the individuals may have been clinically diagnosed with the first disease. Data associated with a first disease indication 110 can include expressions of markers of the individuals who are known to exhibit disease activity of the first disease.

As shown in FIG. 1, a feature extraction 115 process is performed on the data associated with a first disease indication 110 to identify one or more universal signatures 120. In various embodiments, the feature extraction 115 process involves implementing machine-learned methods to identify one or more universal signatures 120. These one or more universal signatures 120 can be informative for generating predictions for the first disease indication, given that the one or more universal signatures 120 were extracted from data associated with a first disease indication 110. Additionally, the one or more universal signatures 120 are also informative for generating predictions for a second disease indication. Thus, these one or more universal signatures 120 represents signatures that are useful for generating predictions for multiple disease indications.

Referring now to the deployment process 160, the one or more universal signatures 120 identified during the development process 150 are used to generate a prediction for a second disease indication. In various embodiments, a common condition 125 guides the selection of the one or more universal signatures that are to be used for generating a prediction for a second disease indication. For example, the first disease indication and second disease indication may share a common condition 125 that characterize, at least in part, each of the first and second disease indications. Examples of a common condition 125 include a precursor to a disease, a sub phenotype of a disease, progression from latent to acute infection, progression from acute to chronic infection, response to an intervention, susceptibility to disease or infection, presence of acute inflammation, presence of chronic inflammation, a dysregulated pathway expression, a cellular phenotype, or a clinical phenotype (e.g., high blood pressure, fever, loss of blood, loss of consciousness, or increased heart rate). The common condition 125 indicates likely commonality in the underlying biology of the first and second disease indications such that the one or more universal signatures developed for the first disease indication can be predictive for the second disease indication.

As shown in FIG. 1, the deployment process 160 involves generating predictions for a set of patients 130 associated with a second disease of the second disease indication. In various embodiments, the patients have experienced the common condition 125. In various embodiments, the patients need not have experienced the common condition 125 but are likely to experience the common condition. The one or more universal signatures 120 are therefore predictive of disease activity of the second disease in the patients 130. In various embodiments, the patients 130 may be subjects who are to be enrolled in a clinical trial. In this scenario, the implementation of the one or more universal signatures 120 enables the screening of patients 130 who are eligible or ineligible for enrollment.

Although FIG. 1 explicitly depicts patients 130 as a part of the deployment process 160, in various embodiments, patients 130 need not be explicitly involved during the deployment process 160. For example, during the deployment process 160, data derived from the patients 130 can be used for analysis. Such data can be obtained as a dataset from a third party who performed the assays to obtain the data derived from the patients 130.

The deployment process 160 involves analyzing 135 the expressions of markers (e.g., genes) the one or more universal signatures from the patients 130. The analysis of the expressions of markers of the one or more universal signatures yields a prediction for the second disease indication 140. In one embodiment, the analysis of the expressions of the markers of the one or more universal signatures involves the application of a machine learning model that is trained to predict disease activity of the second disease using the one or more universal signatures. In other words, the machine learning model can be previously trained using a training dataset with expressions of markers of the universal signatures and the corresponding disease activity of the second disease. In one embodiment, the analysis of the expressions of markers of the universal signatures involves an unsupervised clustering process for classifying the patients 130 into a category. The prediction for the second disease indication 140 can be used for various purposes, such as determining whether patients 130 are eligible or ineligible for enrollment in a clinical trial. In various embodiments, the prediction for the second disease indication 140 can be used to guide the care that is provided to a patient 130 (e.g., selection of an intervention that is provided to a patient 130).

Although FIG. 1 depicts a single iteration of each of the development process 150 and the deployment process 160, in various embodiments, the development process 150 and the deployment process 160 can be performed multiple times for different disease indications. For example, the development process 150 can be performed multiple times to develop universal signatures 120 from different data associated with different disease indications. The development process 150 can also be performed multiple times using different universal signatures to generate predictions for different disease indications. In various embodiments, the development process 150 is performed multiple times to generate different sets of universal signatures. Then, during the deployment process 160, a set of universal signatures are selected for use in generating a prediction for a second disease indication. As described above, the set of universal signatures is selected based on the common condition 125 between the first and second disease indication.

Additionally, in various embodiments, a universal signature identified from a development process 150 can be applied more than once across different deployment processes 160 for different disease indications. For example, a universal signature determined from data associated with a first disease indication can be applied to generate predictions for additional disease indications that share a common condition 125 with the first disease indication. In various embodiments, the multiple disease indications can be two disease indications, three disease indications, four disease indications, five disease indications, six disease indications, seven disease indications, eight disease indications, nine disease indications, or ten disease indications. In various embodiments, the multiple disease indications can be eleven or more disease indications.

Methods for Developing Universal Signatures

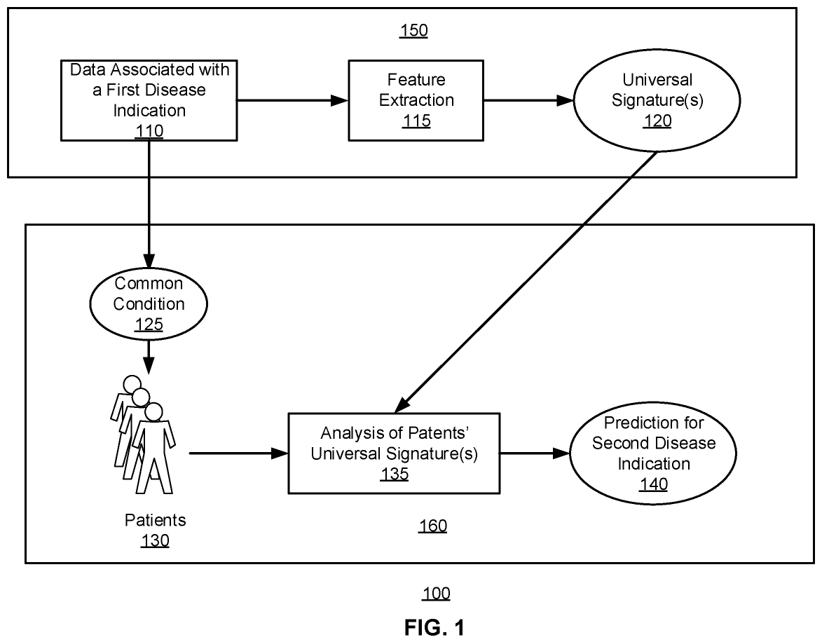

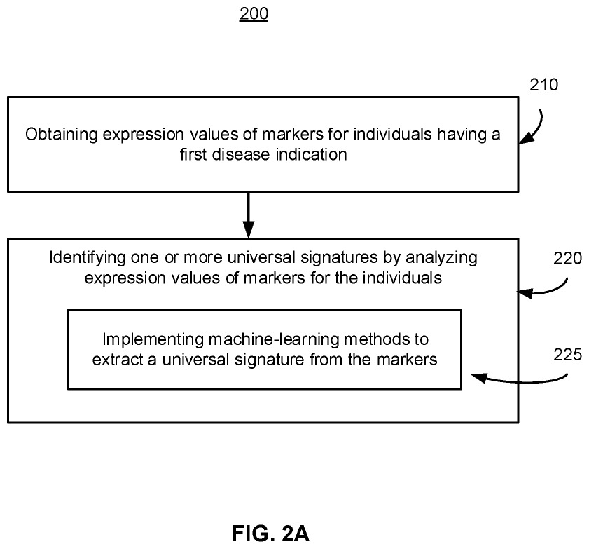

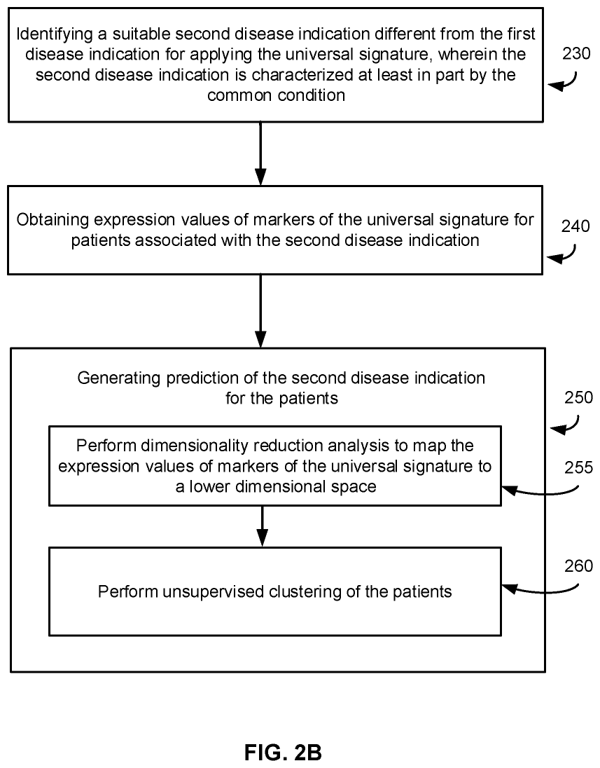

Reference is now made to FIG. 2A, which depicts a flow process 200 for generating one or more universal signatures using data associated with a first disease indication, in accordance with an embodiment. Specifically, FIG. 2A describes in further detail the development process 150 (described above in reference to FIG. 1).

Step 210 involves obtaining data associated with a first disease indication, such as expressions of markers for individuals associated with the first disease indication. In various embodiments, the individuals have been clinically diagnosed and exhibit disease activity of the first disease. In some embodiments, the individuals have not been clinically diagnosed with the first disease and do not exhibit disease activity of the first disease. For example, such individuals may be healthy individuals. In various embodiments, these individuals have encountered a condition (e.g., a common condition as is described in further detail below) of the first disease. In some embodiments, the individuals need not have encountered the condition but may be likely to encounter the condition of the first disease in the future.

In various embodiments, the expressions of markers for individuals associated with the first disease indication is in response to a perturbation or stimuli. Put another way, the expression of markers for individuals may have been determined from the individuals at a timepoint relative to a perturbation or stimuli. Examples of a perturbation or stimulus include an infection (e.g., bacterial infection or viral infection) or a treatment (e.g., drug treatment, medication, or a vaccination). As a specific example, the perturbation is a vaccine, and therefore the expression of markers for individuals can be determined from individuals at any of the different timepoints of 1) pre-vaccination, 2) pre-challenge, or 3) post-challenge.

Therefore, in some embodiments, the expressions of markers obtained at step 210 represent the response to the perturbation or stimulus.

In various embodiments, data associated with a first disease indication can include data from different studies. Thus, the data from the different studies can be aggregated to generate an aggregated dataset. As an example, a first study can include data from a human clinical trial. A second study can include data from a non-human study. Such a non-human study can be a pre-clinical trial study that involves a non-human subject (e.g., a study involving mammalian subjects, such as Rhesus Macaques). Thus, the aggregated dataset includes data from two or more studies and in such embodiments, the identification of one or more universal signatures, as described in further detail below, involves analyzing data from different sources (e.g., from human and non-human subjects). In various embodiments, when identifying one or more universal signatures from multiple sources, the top performing N markers from each source is included as a universal signature. In various embodiments, the top performing N markers across all sources are selected as a universal signature.

In one embodiment, obtaining the expressions of markers encompasses obtaining samples from the individuals and performing one or more assays on the samples to obtain the expressions of markers. Example assays for obtaining expressions of biomarkers include quantitating biomarkers using antibodies or performing gene expression profiling with microarrays or RNAseq. These examples are described herein in further detail. In various embodiments, obtaining the expressions of markers of universal signatures encompasses receiving, from a third party, a dataset including the expressions of markers of universal signatures of the individuals. In such embodiments, the third party may have performed the assay on samples obtained from the individuals to generate the dataset including expressions of markers. In various embodiments, data associated with the first disease indication 110 is curated from datasets. For example, such datasets can be curated from publicly available databases that include expressions of markers in patients who were previously known to have disease activity of the first disease. Examples of publicly available databases include the NCBI Gene Expression Omnibus (GEO) database (e.g., Accession numbers GSE79362, GSE102440, GSE110480, GSE17924, GSE21802, GSE111368, GSE145926, GSE48023, GSE48018) and the NIH Genomic Data Commons Data Portal. In such embodiments, datasets from different databases are aggregated to generate a single dataset for which subsequent analysis can be performed.

Generally, the dataset includes expressions of a plurality of markers for a plurality of individuals. In various embodiments, the dataset includes expressions of tens, hundreds, thousands, tens of thousands, or hundreds of thousands of markers. In some embodiments, the dataset includes expressions of at least 10, at least 100, at least 200, at least 300, at least 400, at least 500, at least 600, at least 700, at least 800, at least 900, or at least 1000 markers. In some embodiments, the dataset includes expressions of at least 2000, at least 3000, at least 4000, at least 5000, at least 6000, at least 7000, at least 8000, at least 9000, or at least 10,000 markers. In various embodiments, the dataset includes expressions of a plurality of markers for tens, hundreds, thousands, tens of thousands, or hundreds of thousands of individuals. In some embodiments, the dataset includes expressions of a plurality of markers for at least 100, at least 200, at least 300, at least 400, at least 500, at least 600, at least 700, at least 800, at least 900, or at least 1000 individuals. In some embodiments, the dataset includes expressions of a plurality of markers for at least 2000, at least 3000, at least 4000, at least 5000, at least 6000, at least 7000, at least 8000, at least 9000, or at least 10,000 individuals.

In various embodiments, the dataset includes additional information pertaining to each individual. As an example, the additional information can include a reference ground truth that are useful for implementing machine-learning methods for extracting a universal signature. A reference ground truth can indicate the presence or absence of disease activity in the individual. For example, if the individual is a healthy individual who has not exhibited disease activity, a reference ground truth value can be assigned to the training example involving the healthy individual. A different individual who is exhibiting disease activity can be assigned a different reference ground truth value. For example, assuming that the disease activity is a progression from latent to acute infection, the reference ground truth for the individual identifies whether or not the individual progressed from a latent infection to an acute infection. As another example, assuming that the disease activity is protection after receiving vaccination, the reference ground truth for the individual indicates whether or not the individual exhibits immunity to the first disease due to the vaccination. In various embodiments, a reference ground truth value of “1” can be assigned to indicate that the individual exhibits disease activity of the disease whereas a reference ground truth value of “0” can be assigned to indicate that the individual does not exhibit disease activity (e.g., the individual is healthy).

At step 220, one or more universal signatures are identified by analyzing the expressions of markers in the dataset. The identified universal signatures include markers that represent a subset of the biomarkers in the dataset. Generally, a universal signature can contain markers that represent features that are informative for predicting disease activity in the first disease, given that the universal signature is identified from a training dataset associated with the first disease indication. However, as described further below, the universal signature can additionally be informative for predicting disease activity in one or more additional diseases.

In one embodiment, a universal signature is identified through univariate feature selection methods. For example, the expression of each marker in the dataset can be analyzed to determine the correlation between the expression of the marker and the reference ground truth (e.g., a reference ground truth indicating presence or absence of disease activity in an individual). The correlation between the biomarker and the reference ground truth can be represented as a coefficient, an example of which is the Pearson correlation coefficient. Depending on the coefficient, the univariate analysis can reveal whether a biomarker is positively correlated (e.g., Pearson correlation coefficient equal to or close to 1), negatively correlated (e.g., Pearson correlation coefficient equal to or close to −1), or limitedly correlated (e.g., Pearson correlation coefficient equal to or close to 0) to the reference ground truth. In various embodiments, positively or negatively correlated biomarkers can be useful when included in the universal signature. For example, the top N biomarkers that are most positively or negatively correlated with reference ground truth values can be selected for the universal signature. Other univariate feature selection methods involve performing a statistical significance test (e.g., a t-test p-value ranking) to identify biomarkers that most correlate with the disease activity of the first disease.

In one embodiment, identifying one or more universal signatures involves, at step 225, implementing machine-learning methods, including deep learning, to extract one or more universal signatures from the biomarkers of the dataset. Example machine-learning methods include random forest, gradient boosting (XGBoost), neural networks, and support vector machines (SVMs).

In one embodiment, a universal signature includes a set of markers that had the highest weights in the random forest models, the highest weights indicating that the set of markers best discriminate between control (e.g., non-diseased) and disease state of the first disease indication. In other words, the markers that have the highest predictive power on the training dataset are combined be used as the universal signature. As one example, for random forest feature selection, a method of mean decrease impurity can be implemented to identify the set of markers that are the most influential for the disease activity of the first disease. A node in the decision tree contains a measure, also referred to as an impurity. Therefore, as model is trained, the impact of each feature can be determined according to how much the feature changes the impurity in the tree. Heavily influential features are selected and combined as a universal signature. In various embodiments, to account for the differences of the markers (e.g., different gene numbers), the feature importance are first standardized before being combined. The markers with the highest standardized feature importance are selected as the universal signature.

As another example, for random forest feature selection, a method of mean decrease accuracy can be implemented. The goal for this method is to determine the impact of each feature on the performance of the model by shuffling the values of features such that the performance of the model is reduced. The shuffling of values for features that are predictive for the disease activity will likely negatively impact the performance of the model whereas less important features, when their values are shuffled, will impact the performance of the model limitedly.

In various embodiments, step 220 involves identifying at least one universal signature, at least two universal signatures, at least three universal signatures, at least four universal signatures, at least five universal signatures, at least six universal signatures, at least seven universal signatures, at least eight universal signatures, at least nine universal signatures, at least ten universal signatures, at least eleven universal signatures, at least twelve universal signatures, at least thirteen universal signatures, at least fourteen universal signatures, at least fifteen universal signatures, at least sixteen universal signatures, at least seventeen universal signatures, at least eighteen universal signatures, at least nineteen universal signatures, at least twenty universal signatures, at least twenty one universal signatures, at least twenty two universal signatures, at least twenty three universal signatures, at least twenty four universal signatures, at least twenty five universal signatures, at least twenty six universal signatures, at least twenty seven universal signatures, at least twenty eight universal signatures, at least twenty nine universal signatures, at least thirty universal signatures, at least thirty one universal signatures, at least thirty two universal signatures, at least thirty three universal signatures, at least thirty four universal signatures, at least thirty five universal signatures, at least thirty six universal signatures, at least thirty seven universal signatures, at least thirty eight universal signatures, at least thirty nine universal signatures, at least forty universal signatures, at least forty one universal signatures, at least forty two universal signatures, at least forty three universal signatures, at least forty four universal signatures, at least forty five universal signatures, at least forty six universal signatures, at least forty seven universal signatures, at least forty eight universal signatures, at least forty nine universal signatures, or at least fifty universal signatures. In various embodiments, step 220 involves identifying at least sixty, at least seventy, at least eighty, at least ninety, or at least one hundred universal signatures.

Example Universal Signature

In various embodiments, a universal signature includes one marker, such as a gene marker. In various embodiments, a universal signature includes at least two markers, at least three markers, at least four markers, at least five markers, at least six markers, at least seven markers, at least eight markers, at least nine markers, at least ten markers, at least eleven markers, at least twelve markers, at least thirteen markers, at least fourteen markers, at least fifteen markers, at least sixteen markers, at least seventeen markers, at least eighteen markers, at least nineteen markers, at least twenty markers, at least twenty one markers, at least twenty two markers, at least twenty three markers, at least twenty four markers, at least twenty five markers, at least twenty six markers, at least twenty seven markers, at least twenty eight markers, at least twenty nine markers, at least thirty markers, at least thirty one markers, at least thirty two markers, at least thirty three markers, at least thirty four markers, at least thirty five markers, at least thirty six markers, at least thirty seven markers, at least thirty eight markers, at least thirty nine markers, at least forty markers, at least forty one markers, at least forty two markers, at least forty three markers, at least forty four markers, at least forty five markers, at least forty six markers, at least forty seven markers, at least forty eight markers, at least forty nine markers, or at least fifty markers. In various embodiments, a universal signature includes at least sixty markers, at least seventy markers, at least eighty markers, at least ninety markers, or at least one hundred markers.

Table 5 documents example sets of universal signatures generated from different datasets. In the examples shown in Table 5, each set of universal signatures includes 50 markers. In some embodiments, fewer or additional universal signatures may be included in a set of universal signatures. For example, as shown in Table 5, the markers in a set of universal signatures are ranked from 1-50. In some embodiments, the markers are ranked based on standardized feature importance

A universal signature can comprise the top 5, 10, 15, 20, 25, 30, 35, 40, 45, or 50 markers from the ranked set of markers shown in Table 5. In various embodiments, the universal signature comprises five markers selected from: (a) NUP93, PPM1G, C6orf62, PJA1, and MEST; (b) CRB3, BCAP31, GMPPB, CD4, and STARD3; (c) NUB1, CASP1, WARS, TRIM21, and STAT1; (d) DNAAF1, UQCRC2, XPNPEP1, ACSM1, and DDX60; (e) LRRC28, E2F4, MRPL15, CCL22, and OTUD1; (f) GSTM3, GYG1, CCL22, MOCS2, and LY6E; (g) MAFB, LGALS3, VCAN, PDK4, and CD81; (h) POLH, PTGER3, RUNX1, CASP6, and CHPT1; (i) CPEB4, CDKN3, TRIM14, ANXA9, and CRYAB; (j) HUWE1, KCNK5, STX11, MORC3, and NETO2; (k) AKR1A1, NDST1, RNF144B, HDAC9, and PSMB3; (l) SPOCK3, PVR, CHTF8, SLC20A1, and PARP8; (m) NLRC5, CACNB2, CELSR1, PARP8, and ECT2; or (n) CCK, SESN2, NACAD, PCSK9, and CIR.

In various embodiments, the universal signature comprises ten markers selected from: (a) NUP93, PPM1G, C6orf62, PJA1, MEST, NDUFS2, DDOST, DHRS7B, NOLC1, and POLA2; (b) CRB3, BCAP31, GMPPB, CD4, STARD3, CALR, CSRP1, CPT1A, LDLRAP1, and RRAS; (c) NUB1, CASP1, WARS, TRIM21, STAT1, MOCOS, BCL2L14, ATF3, KIF2A, and PDCD1LG2; (d) DNAAF1, UQCRC2, XPNPEP1, ACSM1, DDX60, TPI1, EFNA3, ZDHHC19, DDIT3, and DNAJC12; (e) LRRC28, E2F4, MRPL15, CCL22, OTUD1, NSUN7, CHEK1, ADGRA2, ZFPM2, and GYS2; (f) GSTM3, GYG1, CCL22, MOCS2, LY6E, CD151, S100A12, HEBP2, EIF3B, and BAAT; (g) MAFB, LGALS3, VCAN, PDK4, CD81, OLFM4, MMP8, CD1D, KLF4, and CSTA; (h) POLH, PTGER3, RUNX1, CASP6, CHPT1, APOBEC3F, USP14, PEX16, HLA-DQA1, and IRF4; (i) CPEB4, CDKN3, TRIM14, ANXA9, CRYAB, CHST11, ANAPC11, RNASE3, FN1, and ARNTL2; (j) HUWE1, KCNK5, STX11, MORC3, NETO2, BATF2, CCL3L1, SAMD9, CCL2, and PPFIA4; (k) AKR1A1, NDST1, RNF144B, HDAC9, PSMB3, PFKP, MB, MYC, PEX14, and TAF13; (l) SPOCK3, PVR, CHTF8, SLC20A1, PARP8, FGG, ZFAND2A, CCL25, CALR, and TM7SF2; (m) NLRC5, CACNB2, CELSR1, PARP8, ECT2, HTATIP2, NRP1, NCK2, TMEM100, and CLCA2; or (n) CCK, SESN2, NACAD, PCSK9, C1R, SLC7A1, ECM1, XCL1, ARG2, and SPSB1.

In various embodiments, the universal signature comprises fifteen markers selected from: (a) NUP93, PPM1G, C6orf62, PJA1, MEST, NDUFS2, DDOST, DHRS7B, NOLC1, POLA2, PRSS23, SHMT1, RIPK1, AKR1A1, and PRPF3; (b) CRB3, BCAP31, GMPPB, CD4, STARD3, CALR, CSRP1, CPT1A, LDLRAP1, RRAS, HMGCR, RASGRP2, PTS, SORD, and SLC26A6; (c) NUB1, CASP1, WARS, TRIM21, STAT1, MOCOS, BCL2L14, ATF3, KIF2A, PDCD1LG2, SNX10, SEC24D, UBE2L6, LDHC, and FAS; (d) DNAAF1, UQCRC2, PNPEP1, ACSM1, DDX60, TPI1, EFNA3, ZDHHC19, DDIT3, DNAJC12, RET, IL20RB, TNFSF10, DLG4, and CKAP4; (e) LRRC28, E2F4, MRPL15, CCL22, OTUD1, NSUN7, CHEK1, ADGRA2, ZFPM2, GYS2, CD151, RAD51C, ARHGEF2, PFN1, and AP4B1; (f) GSTM3, GYG1, CCL22, MOCS2, LY6E, CD151, S100A12, HEBP2, EIF3B, BAAT, MRPL11, OAS1, RFX5, PSMD7, and ALDH2; (g) MAFB, LGALS3, VCAN, PDK4, CD81, OLFM4, MMP8, CD1D, KLF4, CSTA, IDH1, ITPRIPL2, HMOX1, VSIG4, and FRMD5; (h) POLH, PTGER3, RUNX1, CASP6, CHPT1, APOBEC3F, USP14, PEX16, HLA-DQA1, IRF4, TNNC2, RIT1, ALG1, PDCD4, and CYP2E1; (i) CPEB4, CDKN3, TRIM14, ANXA9, CRYAB, CHST11, ANAPC11, RNASE3, FN1, ARNTL2, KRT82, PRIM2, MOCS2, IL21R, and MAPK8; (j) HUWE1, KCNK5, STX11, MORC3, NETO2, BATF2, CCL3L1, SAMD9, CCL2, PPFIA4, RPH3A, CXCL11, ERMAP, GBP2, and CASP1; (k) AKR1A1, NDST1, RNF144B, HDAC9, PSMB3, PFKP, MB, MYC, PEX14, TAF13, BMX, PRKAA2, PTGER3, C3, and SPTAN1; (l) SPOCK3, PVR, CHTF8, SLC20A1, PARP8, FGG, ZFAND2A, CCL25, CALR, TM7SF2, FUS, DDAH2, SPAG4, FBXL14, and LGALS8; (m) NLRC5, CACNB2, CELSR1, PARP8, ECT2, HTATIP2, NRP1, NCK2, TMEM100, CLCA2, BAALC, PTPN14, IRF9, SAA2, and HR; (n) CCK, SESN2, NACAD, PCSK9, C1R, SLC7A1, ECM1, XCL1, ARG2, SPSB1, DNAH17, TNNC1, CPN1, SYNGR2, and CPA4.

In various embodiments, the universal signature comprises twenty markers selected from: (a) NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, and CHI3L2; (b) CRB3, BCAP31, GMPPB, CD4, STARD3, CALR, CSRP1, CPT1A, LDLRAP1, RRAS, HMGCR, RASGRP2, PTS, SORD, SLC26A6, VAT1, GPAA1, CXCR3, NAMPT, and EPHX1; (c) NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, and PSME2; (d) DNAAF1, UQCRC2, PNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, and MDH2; (e) LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, and BEST3; (f) GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, and PSMA4; (g) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, and S100A12; (h) POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, and TLR8; (i) CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, and ANKRD34B; (j) HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, and BAZ1A; (k) AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, and THOP1; (l) SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, and POLK; (m) NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, and MT1H; (n) CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, and BCAP31.

In various embodiments, the universal signature comprises twenty five markers selected from: (a) NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, and AIFM1; (b) CRB3, BCAP31, GMPPB, CD4, STARD3, CALR, CSRP1, CPT1A, LDLRAP1, RRAS, HMGCR, RASGRP2, PTS, SORD, SLC26A6, VAT1, GPAA1, CXCR3, NAMPT, EPHX1, SEPT9, GMPPA, B4GALT7, AAAS, and TP53INP1; (c) NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, and PLA2G4C; (d) DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, and LTB4R; (e) LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, and F2RL1; (f) GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, and EDF1; (g) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, and COL17A1; (h) POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, and SLCO2A1; (i) CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, and MSH2; (j) HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, and TRO; (k) AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, and FECH; (l) SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, and AGGF1; (m) NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, and NKX3-1; (n) CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, and HSPA1B.

In various embodiments, the universal signature comprises thirty markers selected from: (a) NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, and BCAP31; (b) CRB3, BCAP31, GMPPB, CD4, STARD3, CALR, CSRP1, CPT1A, LDLRAP1, RRAS, HMGCR, RASGRP2, PTS, SORD, SLC26A6, VAT1, GPAA1, CXCR3, NAMPT, EPHX1, SEPT9, GMPPA, B4GALT7, AAAS, TP53INP1, GYS1, FASN, NOC4L, RRP9, and MXI1; (c) NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, and ITGA2; (d) DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, and RTP4; (e) LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, and KIAA1324; (f) GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, and TNFRSF21; (g) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, and MYOF; (h) POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, and RFC2; (i) CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, and PICALM; (j) HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, and ROCK1; (k) AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMP7, HSD11B2, and SLC25A25; (l) SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, and MT2A; (m) NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, and SLC25A19; (n) CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, and CENPJ.

In various embodiments, the universal signature comprises thirty five markers selected from: (a) NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, BCAP31, ATG4B, CSRP1, RDH11, GCLM, and CDC7; (b) CRB3, BCAP31, GMPPB, CD4, STARD3, CALR, CSRP1, CPT1A, LDLRAP1, RRAS, HMGCR, RASGRP2, PTS, SORD, SLC26A6, VAT1, GPAA1, CXCR3, NAMPT, EPHX1, SEPT9, GMPPA, B4GALT7, AAAS, TP531NP1, GYS1, FASN, NOC4L, RRP9, MXI1, TP53, SLC7A11, FOXP3, DNASE1L1, and MGAT1; (c) NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, ITGA2, MPZL2, FEZ1, JAK2, BAZ1A, and ICAM4; (d) DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, RTP4, CTSD, HIST1H2BG, IL27, TNFRSF1B, and SORBS1; (e) LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, KIAA1324, CENPN, IKBKE, SERPINF2, FAM162A, and SNX2; (f) GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, TNFRSF21, PSMB9, GNAI1, TSPAN13, ATP6V0B, and SLC4A4; (g) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, and IFNGR2; (h) POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, RFC2, CAMK1G, CBX8, HM13, PSMB10, and GCLM; (i) CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, PICALM, KRT6A, FMR1, PCSK9, DNASE1L3, and ENDOG; (j) HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, ROCK1, CD83, AK7, MSR1, LCN2, and SPN; (k) AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMPI, HSD11B2, SLC25A25, RAB32, CXCL9, KCNE2, FCAR, and CFP; (l) SPOCK3, PVR, CHTF8, SLC20A1, PARP8, FGG, ZFAND2A, CCL25, CALK, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, MT2A, COL4A2, PLCB4, GYS1, PRKCG, and RXFP2; (m) NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, SLC25A19, MAP7, XCL1, ACSL6, TFRC, and CAT; (n) CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, CENPJ, ARL8A, CTLA4, GFRA1, WASF1, and RIPK1.

In various embodiments, the universal signature comprises forty markers selected from: (a) NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, BCAP31, ATG4B, CSRP1, RDH11, GCLM, CDC7, GLOD5, IDH2, FMR1, PPARA, and CCNE1; (b) CRB3, BCAP31, GMPPB, CD4, STARD3, CALK, CSRP1, CPT1A, LDLRAP1, RRAS, HMGCR, RASGRP2, PTS, SORD, SLC26A6, VAT1, GPAA1, CXCR3, NAMPT, EPHX1, SEPT9, GMPPA, B4GALT7, AAAS, TP53INP1, GYS1, FASN, NOC4L, RRP9, MXI1, TP53, SLC7A11, FOXP3, DNASE1L1, MGAT1, SEC61A1, FYCO1, S100A10, LSS, and IFRD1; (c) NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, ITGA2, MPZL2, FEZ1, JAK2, BAZ1A, ICAM4, DAPP1, RIPK1, RNF144B, LAP3, and C1QA; (d) DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, RTP4, CTSD, HIST1H2BG, IL27, TNFRSF1B, SORBS1, NOP2, TNFSF13B, HLA-DRB5, RHOG, and PSMB9; (e) LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, KIAA1324, CENPN, IKBKE, SERPINF2, FAM162A, SNX2, SERPING1, CLCA2, DPEP3, TNFAIP2, and FSTL4; (f) GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, TNFRSF21, PSMB9, GNAI1, TSPAN13, ATP6V0B, SLC4A4, ILF2, AKAP12, HLA-DRB5, PGR, and AGTRAP; (g) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, IFNGR2, B3GNT5, KYNU, LPGAT1, SLC9A3R1, and HP; (h) POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, RFC2, CAMK1G, CBX8, HM13, PSMB10, GCLM, SLC25A3, MYD88, IL33, ITGAM, and PPIA; (i) CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, PICALM, KRT6A, FMR1, PCSK9, DNASE1L3, ENDOG, TPD52L1, PEX6, MPO, CHRNA7, and SLFN5; (j) HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, ROCK1, CD83, AK7, MSR1, LCN2, SPN, ASS1, HDGF, CXCL16, POLR3D, and GK; (k) AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMPI, HSD11B2, SLC25A25, RAB32, CXCL9, KCNE2, FCAR, CFP, IGF1, PEX16, RNF214, PIM1, and JUNB; (l) SPOCK3, PVR, CHTF8, SLC20A1, PARP8, FGG, ZFAND2A, CCL25, CALK, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, MT2A, COL4A2, PLCB4, GYS1, PRKCG, RXFP2, PLA2G4C, ALDH1A2, ILIA, IBTK, and SPARC; (m) NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, SLC25A19, MAP7, XCL1, ACSL6, TFRC, CAT, NKD1, CNBP, ALDH1L1, CCL7, and SLC20A1; (n) CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, CENPJ, ARL8A, CTLA4, GFRA1, WASF1, RIPK1, ENO3, KRT19, PLVAP, RAD18, and ACHE.

In various embodiments, the universal signature comprises forty five markers selected from: (a) NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, BCAP31, ATG4B, CSRP1, RDH11, GCLM, CDC7, GLOD5, IDH2, FMR1, PPARA, CCNE1, DDB1, BMP1, EHD4, VAV3, and MPG; (b) CRB3, BCAP31, GMPPB, CD4, STARD3, CALK, CSRP1, CPT1A, LDLRAP1, RRAS, HMGCR, RASGRP2, PTS, SORD, SLC26A6, VAT1, GPAA1, CXCR3, NAMPT, EPHX1, SEPT9, GMPPA, B4GALT7, AAAS, TP53INP1, GYS1, FASN, NOC4L, RRP9, MXI1, TP53, SLC7A11, FOXP3, DNASE1L1, MGAT1, SEC61A1, FYCO1, S100A10, LSS, IFRD1, DCP2, EDC4, ANKZF1, IDUA, and IGFBP2; (c) NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, ITGA2, MPZL2, FEZ1, JAK2, BAZ1A, ICAM4, DAPP1, RIPK1, RNF144B, LAP3, C1QA, TYMP, GCH1, C1QB, CREM, and ETV7; (d) DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, RTP4, CTSD, HIST1H2BG, IL27, TNFRSF1B, SORBS1, NOP2, TNFSF13B, HLA-DRB5, RHOG, PSMB9, HSPA6, CD63, SLC2A8, IFITM1, and CKB; (e) LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, KIAA1324, CENPN, IKBKE, SERPINF2, FAM162A, SNX2, SERPING1, CLCA2, DPEP3, TNFAIP2, FSTL4, CTSD, BCAR1, MKX, RGS2, and SAMD9; (f) GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, TNFRSF21, PSMB9, GNAI1, TSPAN13, ATP6V0B, SLC4A4, ILF2, AKAP12, HLA-DRB5, PGR, AGTRAP, P3H1, CDADC1, TRIM5, PTGER3, and ADCY6; (g) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, IFNGR2, B3GNT5, KYNU, LPGAT1, SLC9A3R1, HP, PADI4, PSME1, MGST2, NR4A1, and SPP1; (h) POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, RFC2, CAMK1G, CBX8, HM13, PSMB10, GCLM, SLC25A3, MYD88, IL33, ITGAM, PPIA, SEC22B, CXCR3, SCRN1, RXRA, and SDHA; (i) CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, PICALM, KRT6A, FMR1, PCSK9, DNASE1L3, ENDOG, TPD52L1, PEX6, MPO, CHRNA7, SLFN5, TNFRSF1A, CD24, CASC1, LLGL2, and DLG5; (j) HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, ROCK1, CD83, AK7, MSR1, LCN2, SPN, ASS1, HDGF, CXCL16, POLR3D, GK, OLFM4, STK3, RCBTB1, FOLR3, and FBXO32; (k) AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMPI, HSD11B2, SLC25A25, RAB32, CXCL9, KCNE2, FCAR, CFP, IGF1, PEX16, RNF214, PIM1, JUNB, MDM2, PFKFB4, SIAH2, EGR2, and KCNK10; (l) SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, MT2A, COL4A2, PLCB4, GYS1, PRKCG, RXFP2, PLA2G4C, ALDH1A2, ILIA, IBTK, SPARC, OAS3, EPHA4, HLA-B, MICB, and CCL18; (m) NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, SLC25A19, MAP7, XCL1, ACSL6, TFRC, CAT, NKD1, CNBP, ALDH1L1, CCL7, SLC20A1, KRAS, CSF1, CASP2, HDAC11, and KIR2DS4; (n) CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, CENPJ, ARL8A, CTLA4, GFRA1, WASF1, RIPK1, ENO3, KRT19, PLVAP, RAD18, ACHE, FBLN5, MGST2, ANAPC5, RFX5, and CASP7.

In various embodiments, the universal signature comprises fifty markers selected from: (a) NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, BCAP31, ATG4B, CSRP1, RDH11, GCLM, CDC7, GLOD5, IDH2, FMR1, PPARA, CCNE1, DDB1, BMP1, EHD4, VAV3, MPG, SPAG4, PSMD3, BCKDHA, GRAMD1B, and SEC61A1; (b) CRB3, BCAP31, GMPPB, CD4, STARD3, CALR, CSRP1, CPT1A, LDLRAP1, RRAS, HMGCR, RASGRP2, PTS, SORD, SLC26A6, VAT1, GPAA1, CXCR3, NAMPT, EPHX1, SEPT9, GMPPA, B4GALT7, AAAS, TP53INP1, GYS1, FASN, NOC4L, RRP9, MXI1, TP53, SLC7A11, FOXP3, DNASE1L1, MGAT1, SEC61A1, FYCO1, S100A10, LSS, IFRD1, DCP2, EDC4, ANKZF1, IDUA, IGFBP2, DDX39A, UCHL1, NR4A1, PDIA5, and ENGASE; (c) NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, ITGA2, MPZL2, FEZ1, JAK2, BAZ1A, ICAM4, DAPP1, RIPK1, RNF144B, LAP3, C1QA, TYMP, GCH1, C1QB, CREM, ETV7, FOSB, MRPL15, PSEN1, MXI1, and TRAFD1; (d) DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, RTP4, CTSD, HIST1H2BG, IL27, TNFRSF1B, SORBS1, NOP2, TNFSF13B, HLA-DRB5, RHOG, PSMB9, HSPA6, CD63, SLC2A8, IFITM1, CKB, ALDOA, MSRB1, OSMR, DRAP1, and PLA2G4A; (e) LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, KIAA1324, CENPN, IKBKE, SERPINF2, FAM162A, SNX2, SERPING1, CLCA2, DPEP3, TNFAIP2, FSTL4, CTSD, BCAR1, MKX, RGS2, SAMD9, GCLM, BST1, IRS2, RNASE6, and ELOVL3; (f) GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, TNFRSF21, PSMB9, GNAI1, TSPAN13, ATP6V0B, SLC4A4, ILF2, AKAP12, HLA-DRB5, PGR, AGTRAP, P3H1, CDADC1, TRIM5, PTGER3, ADCY6, ERBB2, NFYA, STATE, MMD, and RPL10A; (g) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, IFNGR2, B3GNT5, KYNU, LPGAT1, SLC9A3R1, HP, PADI4, PSME1, MGST2, NR4A1, SPP1, DEFA3, ME1, RBP7, DUSP6, and MCRS1; (h) POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, RFC2, CAMK1G, CBX8, HM13, PSMB10, GCLM, SLC25A3, MYD88, IL33, ITGAM, PPIA, SEC22B, CXCR3, SCRN1, RXRA, SDHA, GLDC, FGF6, PRKG2, TFPI, and IMMT; (i) CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, PICALM, KRT6A, FMR1, PCSK9, DNASE1L3, ENDOG, TPD52L1, PEX6, MPO, CHRNA7, SLFN5, TNFRSF1A, CD24, CASC1, LLGL2, DLG5, MYO5C, PGR, PFKFB2, AK2, and COL19A1; (j) HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, ROCK1, CD83, AK7, MSR1, LCN2, SPN, ASS1, HDGF, CXCL16, POLR3D, GK, OLFM4, STK3, RCBTB1, FOLR3, FBXO32, TMEM98, PRDX2, CKB, UHRF1BP1L, and CTSG; (k) AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMPI, HSD11B2, SLC25A25, RAB32, CXCL9, KCNE2, FCAR, CFP, IGF1, PEX16, RNF214, PIM1, JUNB, MDM2, PFKFB4, SIAH2, EGR2, KCNK10, EHMT2, FPR1, CD27, CETN2, and TGM1; (l) SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, MT2A, COL4A2, PLCB4, GYS1, PRKCG, RXFP2, PLA2G4C, ALDH1A2, IL1A, IBTK, SPARC, OAS3, EPHA4, HLA-B, MICB, CCL18, SLC39A6, GLCE, TUBB2B, FBXO8, and SNX6; (m) NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, SLC25A19, MAP7, XCL1, ACSL6, TFRC, CAT, NKD1, CNBP, ALDH1L1, CCL7, SLC20A1, KRAS, CSF1, CASP2, HDAC11, KIR2DS4, CEACAM19, CFH, CAB39L, DEPDC1, and PSMA1; (n) CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, CENPJ, ARL8A, CTLA4, GFRA1, WASF1, RIPK1, ENO3, KRT19, PLVAP, RAD18, ACHE, FBLN5, MGST2, ANAPC5, RFX5, CASP7, STC1, NCK2, IFI27, APOA4, and MSRB2.

In various embodiments, a universal signature can be used to predict progression of tuberculosis in an individual. In various embodiments, the progression of tuberculosis can be the progression of latent tuberculosis to active tuberculosis. In various embodiments, the progression of tuberculosis occurs within one year. In various embodiments, a universal signature can be used to predict progression of a glioma in an individual In various embodiments, the progression of a glioma can be a severe progression of glioma such that the patient is likely to expire within a year. In various embodiments, a universal signature can be used to predict either the progression of tuberculosis or the progression of glioma in an individual. In such embodiments, the universal signature comprises markers selected from: (a) NUP93, PPM1G, C6orf62, PJA1, and MEST; (b) CRB3, BCAP31, GMPPB, CD4, and STARD3; (c) NUB1, CASP1, WARS, TRIM21, and STAT1; (d) NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, and AIFM1; (e) CRB3, BCAP31, GMPPB, CD4, STARD3, CALR, CSRP1, CPT1A, LDLRAP1, RRAS, HMGCR, RASGRP2, PTS, SORD, SLC26A6, VAT1, GPAA1, CXCR3, NAMPT, EPHX1, SEPT9, GMPPA, B4GALT7, AAAS, and TP53INP1; (f) NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, and PLA2G4C; (g) NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, BCAP31, ATG4B, CSRP1, RDH11, GCLM, CDC7, GLOD5, IDH2, FMR1, PPARA, CCNE1, DDB1, BMP1, EHD4, VAV3, MPG, SPAG4, PSMD3, BCKDHA, GRAMD1B, and SEC61A1; (h) CRB3, BCAP31, GMPPB, CD4, STARD3, CALR, CSRP1, CPT1A, LDLRAP1, RRAS, HMGCR, RASGRP2, PTS, SORD, SLC26A6, VAT1, GPAA1, CXCR3, NAMPT, EPHX1, SEPT9, GMPPA, B4GALT7, AAAS, TP53INP1, GYS1, FASN, NOC4L, RRP9, MXI1, TP53, SLC7A11, FOXP3, DNASE1L1, MGAT1, SEC61A1, FYCO1, S100A10, LSS, IFRD1, DCP2, EDC4, ANKZF1, IDUA, IGFBP2, DDX39A, UCHL1, NR4A1, PDIA5, and ENGASE; or (i) NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, ITGA2, MPZL2, FEZ1, JAK2, BAZ1A, ICAM4, DAPP1, RIPK1, RNF144B, LAP3, C1QA, TYMP, GCH1, C1QB, CREM, ETV7, FOSB, MRPL15, PSEN1, MXI1, and TRAFD1.

In various embodiments, a universal signature can be used to predict presence of an infection, severity of an infection, progression of an infection, or a patient response to a vaccine against an infection. In various embodiments, the infection is a viral infection. In various embodiments, the infection can be any one of a SARS CoV-2 infection, a HBV infection, H1N1 infection, or influenza infection. In various embodiments, the severity of an infection can be classified as one of severe or not severe. In various embodiments, the severity of the symptoms of an individual with a viral infection can be the severity of the symptoms after one year. In some embodiments, the universal signature useful for predicting presence of an infection, severity of an infection, progression of an infection, or patient response to a vaccine against an infection comprises markers selected from: (a) DNAAF1, UQCRC2, XPNPEP1, ACSM1, and DDX60; (b) LRRC28, E2F4, MRPL15, CCL22, and OTUD1; (c) GSTM3, GYG1, CCL22, MOCS2, and LY6E; (d) MAFB, LGALS3, VCAN, PDK4, and CD81; (e) POLH, PTGER3, RUNX1, CASP6, and CHPT1; (f) CPEB4, CDKN3, TRIM14, ANXA9, and CRYAB; (g) HUWE1, KCNK5, STX11, MORC3, and NETO2; (h) AKR1A1, NDST1, RNF144B, HDAC9, and PSMB3; (i) SPOCK3, PVR, CHTF8, SLC20A1, and PARP8; (j) NLRC5, CACNB2, CELSR1, PARP8, and ECT2; or (k) CCK, SESN2, NACAD, PCSK9, and C1R. In some embodiments, the universal signature useful for predicting presence of an infection, severity of an infection, progression of an infection, or patient response to a vaccine against an infection comprises markers selected from: (a) DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, and LTB4R; (b) LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, and F2RL1; (c) GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, and EDF1; (d) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, and COL17A1; (e) POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, and SLCO2A1; (f) CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, and MSH2; (g) HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, and TRO; (h) AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, and FECH; (i) SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, and AGGF1; (j) NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, and NKX3-1; (k) CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, and HSPA1B. In some embodiments, the universal signature useful for predicting presence of an infection, severity of an infection, progression of an infection, or patient response to a vaccine against an infection comprises markers selected from: (a) DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, RTP4, CTSD, HIST1H2BG, IL27, TNFRSF1B, SORBS1, NOP2, TNFSF13B, HLA-DRB5, RHOG, PSMB9, HSPA6, CD63, SLC2A8, IFITM1, CKB, ALDOA, MSRB1, OSMR, DRAP1, and PLA2G4A; (b) LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, KIAA1324, CENPN, IKBKE, SERPINF2, FAM162A, SNX2, SERPING1, CLCA2, DPEP3, TNFAIP2, FSTL4, CTSD, BCAR1, MKX, RGS2, SAMD9, GCLM, BST1, IRS2, RNASE6, and ELOVL3; (c) GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, TNFRSF21, PSMB9, GNAI1, TSPAN13, ATP6V0B, SLC4A4, ILF2, AKAP12, HLA-DRB5, PGR, AGTRAP, P3H1, CDADC1, TRIM5, PTGER3, ADCY6, ERBB2, NFYA, STATE, MMD, and RPL10A; (d) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, IFNGR2, B3GNT5, KYNU, LPGAT1, SLC9A3R1, HP, PADI4, PSME1, MGST2, NR4A1, SPP1, DEFA3, ME1, RBP7, DUSP6, and MCRS1; (e) POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, RFC2, CAMK1G, CBX8, HM13, PSMB10, GCLM, SLC25A3, MYD88, IL33, ITGAM, PPIA, SEC22B, CXCR3, SCRN1, RXRA, SDHA, GLDC, FGF6, PRKG2, TFPI, and IMMT; (f) CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, PICALM, KRT6A, FMR1, PCSK9, DNASE1L3, ENDOG, TPD52L1, PEX6, MPO, CHRNA7, SLFN5, TNFRSF1A, CD24, CASC1, LLGL2, DLG5, MYO5C, PGR, PFKFB2, AK2, and COL19A1; (g) HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, ROCK1, CD83, AK7, MSR1, LCN2, SPN, ASS1, HDGF, CXCL16, POLR3D, GK, OLFM4, STK3, RCBTB1, FOLR3, FBXO32, TMEM98, PRDX2, CKB, UHRF1BP1L, and CTSG; (h) AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMPI, HSD11B2, SLC25A25, RAB32, CXCL9, KCNE2, FCAR, CFP, IGF1, PEX16, RNF214, PIM1, JUNB, MDM2, PFKFB4, SIAH2, EGR2, KCNK10, EHMT2, FPR1, CD27, CETN2, and TGM1; (i) SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, MT2A, COL4A2, PLCB4, GYS1, PRKCG, RXFP2, PLA2G4C, ALDH1A2, ILIA, IBTK, SPARC, OAS3, EPHA4, HLA-B, MICB, CCL18, SLC39A6, GLCE, TUBB2B, FBXO8, and SNX6; (j) NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, SLC25A19, MAP7, XCL1, ACSL6, TFRC, CAT, NKD1, CNBP, ALDH1L1, CCL7, SLC20A1, KRAS, CSF1, CASP2, HDAC11, KIR2DS4, CEACAM19, CFH, CAB39L, DEPDC1, and PSMA1; (k) CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, CENPJ, ARL8A, CTLA4, GFRA1, WASF1, RIPK1, ENO3, KRT19, PLVAP, RAD18, ACHE, FBLN5, MGST2, ANAPC5, RFX5, CASP7, STC1, NCK2, IFI27, APOA4, and MSRB2.

In particular embodiments, the universal signature useful for predicting presence of an infection, severity of an infection, progression of an infection, or patient response to a vaccine against an infection comprises markers selected from: (a) MAFB, LGALS3, VCAN, PDK4, and CD81; (b) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, and COL17A1; or (c) MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, IFNGR2, B3GNT5, KYNU, LPGAT1, SLC9A3R1, HP, PADI4, PSME1, MGST2, NR4A1, SPP1, DEFA3, ME1, RBP7, DUSP6, and MCRS1. In particular embodiments, the infection is a viral infection selected from SARS-CoV-2 or H1N1.

Applying Universal Signatures to a Second Disease Indication

FIG. 2B depicts a flow process for generating a prediction for a second disease indication using the universal signature, in accordance with an embodiment. Specifically, FIG. 2B describes in further detail the deployment process 160 (described above in reference to FIG. 1). The goal of this process shown in FIG. 2B is to apply the universal signature on a suitable second disease indication to predict disease activity for the second disease.

Step 230 involves identifying a suitable second disease indication that is different from the first disease indication used to identify the universal signature. A suitable second disease indication is a disease indication in which the universal signature can be applied for predicting disease activity of the suitable second disease indication.

In various embodiments, the process of identifying a second disease indication involves comparing a condition that characterizes the second disease indication with a condition that characterizes the first disease indication. A condition of the first or second disease indication refers to any one of a precursor to a disease, a phenotype or sub-phenotype of a disease, progression from latent to acute infection, progression from acute to chronic infection, response to an intervention, susceptibility to disease or infection, presence of acute inflammation, presence of chronic inflammation, a clinical phenotype, or a clinical condition (e.g., high blood pressure, fever, loss of blood, loss of consciousness, or increased heart rate). In one embodiment, if the condition of the first disease indication and the condition of the second disease indication are the same, the condition is a common condition of the first and second disease indications. Given the common condition that characterizes both the first and second disease indications, the second disease indication can be selected for applying the universal signature which was previously developed from data of the first disease indication.

As an example, a first disease indication may refer to progression in infectious diseases. A second disease indication may refer to patient survival time after diagnosis with a brain tumor (e.g., glioma). Here, both infectious diseases and brain tumors are characterized by at least a common condition of chronic infection. Therefore, in comparing the conditions of infectious diseases and brain tumors, the common condition of chronic infection is identified. The second disease indication involving the disease of brain tumors is a suitable disease indication for applying the universal signature determined from data describing progression in infectious diseases.

As another example, a first disease indication and a second disease indication may share a common condition of a clinical phenotype. As a specific example, a first disease indication can involve H1N1 and a clinical phenotype of the disease is the need for mechanical ventilation. Therefore, a second disease indication can be identified that similarly shares the clinical phenotype of a need for mechanical ventilation. An example of an identified second disease indication involves SARS-CoV-2, as patients with SARS-CoV-2 often encounter the need for mechanical ventilation. Thus, the universal signature determined from data of H1N1 can be applied to generate predictions for SARS-CoV-2 patients. As another specific example, a first disease indication may involve H1N1 and a clinical phenotype of the disease is a response to a vaccination, as measured by antibody titers. A second disease indication, such as HBV, can be identified that shares the clinical phenotype of a response to a vaccination as measured by antibody titers. Thus, universal the signature determined from data of vaccine-administered H1N1 patients can be used to generate predictions for vaccine-administered HBV patients.

As another example, a first disease indication and a second disease indication may share a common condition of a cellular phenotype. A first disease indication can involve a cellular phenotype including a dysregulated cell population. A dysregulated cell population can be a cell population with aberrant behavior (e.g., dysregulated gene expression, biomarker expression, or protein synthesis). A second disease indication can be identified that shares the cellular phenotype of a dysregulated cell population (e.g., dysregulated gene expression, biomarker expression, or protein synthesis). Therefore, the universal signature determined from data of the first disease indication can be used to generate predictions for the second disease indication.

As another example, a first disease indication and a second disease indication may share a common condition of a dysregulated pathway expression. A dysregulated pathway expression refers to one or more aberrant pathways where markers of the pathway are differentially expressed in comparison to their expressions in a healthy state. As such, an aberrant pathway may be associated with and/or be the cause of multiple diseases (e.g., diseases of the first disease indication and second disease indication). In various embodiments, a dysregulated pathway expression refers to aberrant expression of one, two, three, four, five, six, seven, eight, nine, or ten markers of the pathway. In various embodiments, a dysregulated pathway expression refers to aberrant expression of at least ten markers of the pathway.

In various embodiments, each of the first disease indication and the second disease indication may be characterized by multiple conditions. Here, the process of identifying a second disease indication as suitable for applying the universal signature can involve determining whether there are a threshold number of common conditions between the first disease indication and the second disease indication. If the first disease indication and the second disease indication share at least a threshold number of common conditions, then the second disease indication is suitable for applying the universal signature developed using data for the first disease indication. In various embodiments, the threshold number of common conditions is one common condition, two common conditions, three common conditions, four common conditions, five common conditions, six common conditions, seven common conditions, eight common conditions, nine common conditions, or ten common conditions.

Step 240 involves obtaining expressions of markers of the universal signature expressed by patients, such as patients 130 described above in FIG. 1, associated with the second disease of the second disease indication. In various embodiments, the patients may have been clinically diagnosed with the second disease of the second disease indication. In such embodiments, the universal signature can be used to predict disease activity in these patients. In various embodiments, the patients may not yet be clinically diagnosed with the second disease but are suspected to have the second disease. Thus, the universal signature can be used to predict disease activity (e.g., presence or absence of a disease) for these patients. In various embodiments, the patients have encountered the common condition that characterizes the second disease indication. However, in other embodiments, the patients have not yet encountered the common condition that characterizes the second disease indication.

In one embodiment, obtaining the expressions of markers of the universal signature encompasses obtaining samples from the patients associated with or having the second disease of the second disease indication and performing one or more assays on the samples to obtain the expressions of the markers of the universal signature. Example assays for obtaining expressions of the markers of the universal signature include quantitating biomarkers using antibodies or performing gene expression profiling with microarrays or RNAseq. In various embodiments, obtaining the expressions of the markers of the universal signature encompasses receiving, from a third party, a dataset including the expressions of the markers of the universal signature. In such embodiments, the third party may have performed the assay on samples obtained from patients associated with or having the second disease of the second disease indication to generate the expressions of markers of the universal signature.

Step 250 involves generating a prediction of the second disease indication for the patients by analyzing the expressions of markers of the universal signature of the patients. Step 250 describes, in further detail, step 135 in FIG. 1. In one embodiment, the prediction represents a classification of the disease activity for the patients. For example, the prediction can be a classification that the second disease of the patient is likely to progress from a latent form (e.g., latent TB) to an active form (e.g., active TB). As another example, the prediction can be a classification that the survival time for the patient with the second disease is above or below a certain threshold hold time (e.g., 6 months, 1 year, 2 years, 3 years, 4 years, 5 years, 10 years, or 20 years).

In one embodiment, analyzing the expressions of the markers of the universal signature involves applying a machine learning model that generates predictions for a second disease indication (e.g., disease activity of a second disease). In this scenario, the markers of the universal signature serve as features for the machine learning model, which outputs the prediction of disease activity of the second disease indication 140. The machine learning model can be trained using a dataset including training examples that include expression of at least markers of the universal signature. In various embodiments, the training examples can further include a reference ground truth, which is an indication of the disease activity of the second disease. Here, the machine learning model can be trained using supervised learning such that the machine learning model can more accurately predict disease activity of the second disease based on the universal signature.

In various embodiments, the machine learning model can be trained using a machine learning implemented method such as any one of a linear regression algorithm, logistic regression algorithm, decision tree algorithm, support vector machine classification, Naïve Bayes classification, K-Nearest Neighbor classification, random forest algorithm, deep learning algorithm, or gradient boosting algorithm. In various embodiments, the machine learning model is trained using supervised learning algorithms, unsupervised learning algorithms, or semi-supervised learning algorithms (e.g., partial supervision).

In various embodiments, the process of training the machine learning model occurs subsequent to the development process (e.g., development process 150 described in FIG. 1) which involves the identification of the universal signature from the first disease indication. Thus, the universal signature learned from data of the first disease indication are transferred to train the machine learning model that is predictive for a second disease indication.

In various embodiments, a non-machine learning method is implemented to analyze the expression of the universal signature. For example, analyzing the expression of the markers of the universal signature involves performing an unsupervised cluster analysis of the patients 130 according to their expressions of the markers of the universal signature. The individual clusters are labeled and therefore, the patients in a cluster are classified according to the label. Therefore, the predicted disease activity of the second disease for a patient is based upon the cluster in which the patient is grouped into.

In various embodiments, the individual clusters are labeled by using patient data from the first disease indication. In various embodiments, patients of the first disease indication, whose disease activity is known, are overlaid on the reduced dimensionality. Therefore, the known disease activity of the patients of the first disease indication can be used to label the individual clusters. For example, patients of the first disease indication can be known as either responding to or not responding to a vaccination. Therefore, when overlaid on the reduced dimensionality, the clusters can be labeled as likely responders or non-responders according to the allocation of patients of the first disease indication. For example, if a majority of patients (e.g., greater than 50% of patients) of the first disease indication, who are identified as responders to a vaccine, are located more proximal or are overlapping with a first cluster in comparison to a second cluster, then the first cluster can be labeled as responders to the vaccine. As another example, if a majority of patients (e.g., greater than 50% of patients) of the first disease indication, who are identified as non-responders to a vaccine, are located more proximal or are overlapping with a first cluster in comparison to a second cluster, then the first cluster can be labeled as non-responders to the vaccine.

In various embodiments, the individual clusters are labeled by using patient data from the first disease indication. In various embodiments, gene expression of patients of the first disease indication, whose disease activity is known are used. Specifically, the expression data between training and test sets were not directly compared, as the range of expression is most likely more different across datasets than across phenotypes within a dataset. Thus, the direction of the signal is used rather than the amplitude: for each marker present in the universal signature, the median expression in each cluster was compared and the direction of the signal was recorded in each cluster (high, low or intermediate—in the presence of more than 2 clusters). The same analysis was performed in the training dataset where the universal signature was obtained from, using the true labels (case/control) instead of clusters to group the samples. Clusters in the test dataset were assessed for to determine the highest proportion of genes that matched the label of interest in the training dataset (in terms of signal direction) and defined it as “case cluster”, while the other cluster(s) were defined as control cluster.

Examples of unsupervised cluster analysis include hierarchical clustering, k-means clustering, clustering using mixture models, density based spatial clustering of applications with noise (DBSCAN), ordering points to identify the clustering structure (OPTICS), or combinations thereof. In preferred embodiments, unsupervised cluster analysis includes hierarchical density based spatial clustering of applications with noise (HDBSCAN).

In various embodiments, analyzing the expressions of markers of the universal signature involves performing dimensionality reduction analysis. For example, in scenarios in which multiple genes of a universal signature are used for generating a prediction for a second disease indication, dimensionality reduction analysis is useful for mapping the expressions of the markers of the universal signature into a lower dimensional space. Thus, predictions of the second disease indication can be made for patients according to expressions of the markers of the universal signature that have been mapped onto a lower dimensional space. Examples of dimensionality reduction analysis include principal component analysis (PCA), kernel PCA, graph-based kernel PCA, linear discriminant analysis, generalized discriminant analysis, autoencoder, non-negative matrix factorization, T-distributed stochastic neighbor embedding (t-SNE), or uniform manifold approximation and projection (UMAP) and dens-UMAP. Additional details of performing UMAP is described in Narayan, A. et al, “Density-Preserving Data Visualization Unveils Dynamic Patterns Of Single-Cell Transcriptomic Variability.” bioRxiv 2020.05.12.077776, which is hereby incorporated by reference in its entirety.

In various embodiments, combinations of the aforementioned methods (e.g., application of machine learning model, unsupervised clustering, and dimensionality reduction analysis) can be performed to generate a prediction of the second disease indication. As one example, in the embodiment shown in FIG. 2B, step 250 involves step 255 of performing a dimensionality reduction analysis to map the expressions of markers of the universal signature to a lower dimensional space. This method can avoid the effects of the curse of dimensionality. Next, step 260 involves performing unsupervised clustering of the patients. Here, the unsupervised clustering can be performed on the expressions of the markers of the universal signature that have been mapped to the lower dimensional space. As another example, a dimensionality reduction analysis can be first performed to map the expressions of markers of the universal signatures to a lower dimensional space, which can then serve as inputs to the trained machine learning model. Thus, the machine learning model can output a prediction of the second disease indication according to the expressions of the markers of the universal signature that are organized in the lower dimensional space.

In various embodiments, the prediction of the second disease indication for the patients can be useful for guiding the care that is provided to a patient. For example, given the prediction of the second disease indication that indicates that the patient is likely to undergo a progression of disease, the patient can be provided an intervention to slow or combat the progression of the disease.

In various embodiments, the prediction of the second disease indication for the patients can be useful for evaluating whether patients are eligible or ineligible for enrollment in clinical trials. For example, the prediction of the second disease indication can be evaluated against an eligibility criterion such that patients that meet the eligibility criterion can be enrolled in the clinical trial whereas patients that fail to meet the eligibility criterion are not enrolled. This is useful for particular clinical trials that enroll large numbers of patients in hopes of obtaining a sufficient number of patients that satisfy a particular criterion. Here, at the time of enrollment, it is not known whether the patients are likely to satisfy the criterion or not. For example, classic trials typically enroll a large number of patients with the hopes that a sufficient number of those enrolled patients meet the criterion after the fact. A large number of enrolled patients in a classic trial are subsequently eliminated for not meeting the criterion at a later timepoint.

For example, a control group for a clinical trial involving tuberculosis patients may require a sufficient number of patients to progress to active tuberculosis within a certain time frame (e.g., 6 months or 1 year). Thus, enrolled patients that do not progress within the time frame are eliminated from the trial.

Using the universal signature, the prediction of the second disease indication enables the prospective identification of patients with tuberculosis that would likely meet this criterion and therefore, can be enrolled in the clinical trial. Altogether, the use of the universal signature for generating predictions for a second disease indication for purposes of enrolling patients in clinical trials represents an enrichment strategy such that fewer patients need to be enrolled. This can be highly beneficial for clinical trials in which a limited numbers of patients are available e.g., in rare or novel diseases. For example, fewer enrolled patients in a clinical trial will result in substantial economic benefits.

System Environment

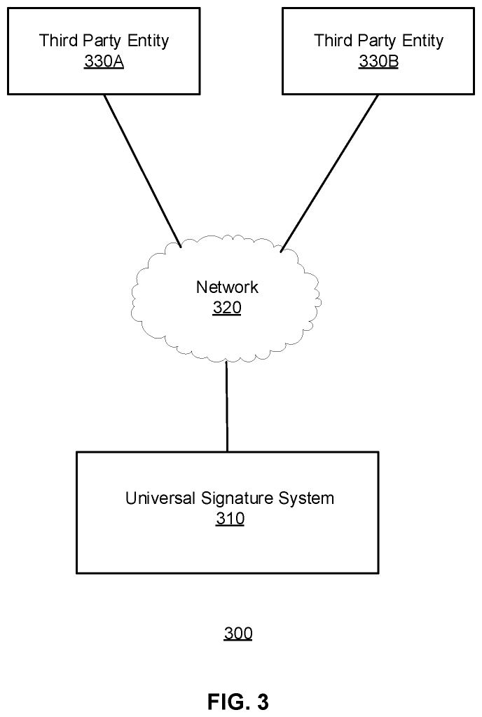

FIG. 3 depicts an overall system environment 300 for generating and using one or more universal signatures, in accordance with an embodiment. The overall system environment 300 includes a universal signature system 310 and one or more third party entities 330A and 330B in communication with one another through a network 320. FIG. 3 depicts one embodiment of the overall system environment 300. In other embodiments, additional or fewer third party entities 330 in communication with the universal signature system 310 can be included.

In various embodiments, the universal signature system 310 performs the methods described above in reference to FIGS. 1, 2A, and 2B (e.g., methods for identifying one or more universal signatures relevant for a first disease indication and applying one or more universal signatures to generate a prediction for a second disease indication). The universal signature system 310 can provide the predictions regarding patients associated with the second disease indication to third party entities 330A and 330B.

In various embodiments, the universal signature system 310 performs a subset of the methods described in FIGS. 1, 2A, and 2B and third party entities 330 can perform another subset of the methods. In one embodiment, the universal signature system 310 performs the steps of identifying one or more universal signatures from a first disease indication and one or more of the third party entities 330 perform the steps of applying the one or more universal signatures to generate predictions for a second disease indication. In this embodiment, the universal signature system 310 may provide the identified one or more universal signatures to a third party entity 330 such that the third party entity 330 can use the one or more universal signatures to generate predictions for patients associated with the second disease indication.

Third Party Entity

In various embodiments, the third party entity 330 represents a partner entity of the universal signature system 310. The third party entity 330 can operate either upstream or downstream of the universal signature system 310. As one example, the third party entity 330 operates upstream of the universal signature system 310 and provide information to the universal signature system 310 that enables the universal signature system 310 to perform the methods for identifying universal signatures. Here, the universal signature system 310 receives data, such as expressions of markers, of patients associated with a first disease indication from the third party entity 330. Thus, the universal signature system 310 analyzes the received data to identify one or more universal signatures.

As another example, the third party entity 330 operates downstream of the universal signature system 310. In this scenario, the universal signature system 310 uses the one or more universal signatures to generate a prediction for a second disease indication provides the prediction to the third party entity 330. The third party entity 330 can subsequently use the prediction for their purposes. For example, the third party entity 330 may be a healthcare provider. Therefore, the third party entity 330 can provide appropriate medical attention (e.g., medical advice, a treatment, an intervention, or the like) to a patient based on the prediction.

Network

This disclosure contemplates any suitable network 320 that enables connection between the universal signature system 310 and other third party entities 330A and 330B. The network 320 may comprise any combination of local area and/or wide area networks, using both wired and/or wireless communication systems. In one embodiment, the network 320 uses standard communications technologies and/or protocols. For example, the network 320 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 320 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 320 may be represented using any suitable format, such as hypertext markup language (HTML) or extensible markup language (XML). In some embodiments, all or some of the communication links of the network 704 may be encrypted using any suitable technique or techniques.

Non-Transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for implementing a machine learning model for the purposes of predicting a clinical phenotype.

Computing Device

The methods described above, including the methods of developing and applying one or more universal signatures, are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

In various embodiments, the different methods described above in relation to FIGS. 1, 2A, 2B such as the methods for identifying and applying one or more universal signatures, as well as the entities shown in FIG. 3, may be implemented using one or more computing devices. For example, the universal signature system 310, third party entity 330A, and third party entity 330B may each employ one or more computing devices 400.

The methods for developing and applying one or more universal signatures can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results e.g., a prediction of disease activity of a second disease. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

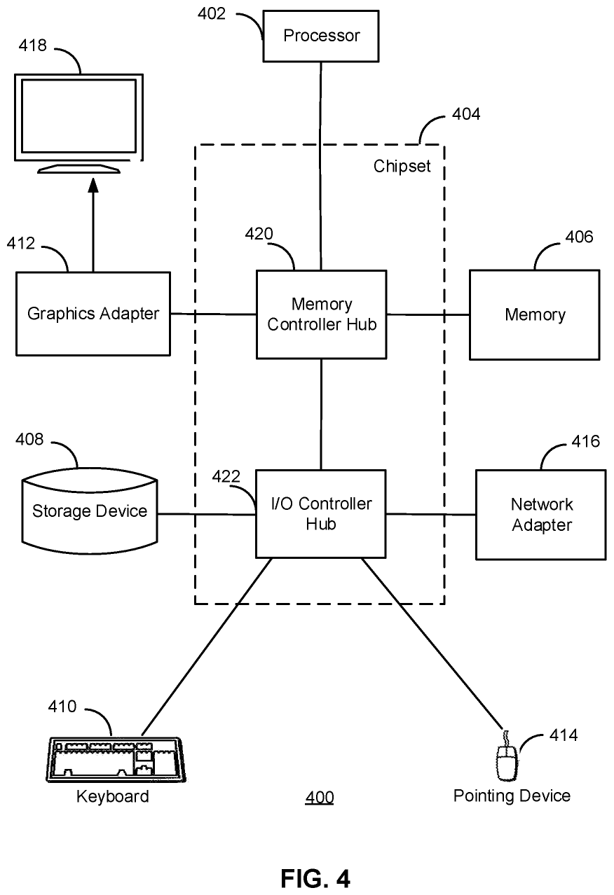

FIG. 4 illustrates an example computing device 400 for implementing methods described in FIGS. 1, 2A, and 2B and the entities shown in FIG. 3. In some embodiments, the computing device 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input interface 414, and network adapter 416 are coupled to the I/O controller hub 422. Other embodiments of the computing device 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of input interface, a keyboard, or some combination thereof, and is used to input data into the computing device 400. In some embodiments, the computing device 400 may be configured to receive input (e.g., commands) from the input interface 414 via gestures from the user. The graphics adapter 412 displays images and other information on the display 418. For example, the display 418 can show a prediction of disease activity, such as a prediction of disease activity of a second disease 140 described above in FIG. 1. The network adapter 416 couples the computing device 400 to one or more computer networks.

The computing device 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computing devices 400 can vary from the embodiments described herein. For example, the computing device 400 can lack some of the components described above, such as graphics adapters 412, input interface 414, and displays 418. In some embodiments, a computing device 400 can include a processor 402 for executing instructions stored on a memory 406.

Example Assays for Obtaining Expressions of Markers

In one embodiment, obtaining the expressions of markers encompasses obtaining samples from the individuals and performing one or more assays on the samples to obtain the quantities (e.g., expression values) of markers.

One approach for measuring expression levels is to perform identification with the use of antibodies. As used herein, the term “antibody” is intended to refer broadly to any immunologic binding agent such as IgG, IgM, IgA, IgD and IgE. Generally, IgG and/or IgM are the most common antibodies in the physiological situation and are most easily made in a laboratory setting. The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. In various embodiments, immunodetection methods can be employed to detect levels of expression. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle and Ben-Zeev O, 1999; Gulbis and Galand, 1993; De Jager et al., 1993; and Nakamura et al., 1987, each incorporated herein by reference.

Another approach for measuring expression levels is to perform gene expression profiling with microarrays. Microarrays comprise a plurality of polymeric molecules spatially distributed over, and stably associated with, the surface of a substantially planar substrate, e.g., biochips. In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information. One example of a microarray is a single nucleotide polymorphism (SNP)—Chip array, which is a DNA microarray that enables detection of polymorphisms in DNA.

Another approach for measuring expression levels is to perform gene expression profiling with high throughput sequencing (RNAseq). RNA-seq (RNA Sequencing), one example of which is Whole Transcriptome Shotgun Sequencing (WTSS), is a technology that utilizes the capabilities of next-generation sequencing to reveal a snapshot of RNA presence and quantity from a genome at a given moment in time. An example of a RNA-seq technique is Perturb-seq. The transcriptome of a cell is dynamic; it continually changes as opposed to a static genome. The recent developments of Next-Generation Sequencing (NGS) allow for increased base coverage of a DNA sequence, as well as higher sample throughput. This facilitates sequencing of the RNA transcripts in a cell, providing the ability to look at alternative gene spliced transcripts, post-transcriptional changes, gene fusion, mutations/SNPs and changes in gene expression. In addition to mRNA transcripts, RNA-Seq can look at different populations of RNA to include total RNA, nascent RNA, small RNA, such as miRNA, tRNA, and ribosomal profiling. RNA-Seq can also be used to determine exon/intron boundaries and verify or amend previously annotated 5′ and 3′ gene boundaries, Ongoing RNA-Seq research includes observing cellular pathway alterations that arise (e.g., for a particular disease indication), and gene expression level changes (e.g., for particular disease indications).

### EXAMPLES

### Example 1: Example Diseases, Common Conditions, and Universal Signatures

Further disclosed herein are particular combinations of 1) a first disease indication, 2) second disease indication, and 3) common condition shared between the first disease indication and second disease indication. Example combinations of first disease indication, second disease indication, and common condition are shown below.

| First Disease | Second Disease | |
|---|---|---|
| Indication | Indication | Common Condition |
| Progression to active | Glioma | Cancer/chronic |
| Tuberculosis | | infection |
| Rhesus macaque | Progression from | TB infection |
| protection to | latent to acute TB | |
| Tuberculosis (TB) | infection in humans | |
| after vaccination | | |
| Dengue infection in | H1N1 infection in | Severe infection |
| humans | humans | phenotype |
| Dengue infection in | SARS-CoV-2 | Severe infection |
| humans | infection in humans | phenotype |
| H1N1 infection in | SARS-CoV-2 | Severe infection |
| humans | infection in humans | phenotype |

### Example 2: Overview of Methods for Generating and Using Signatures

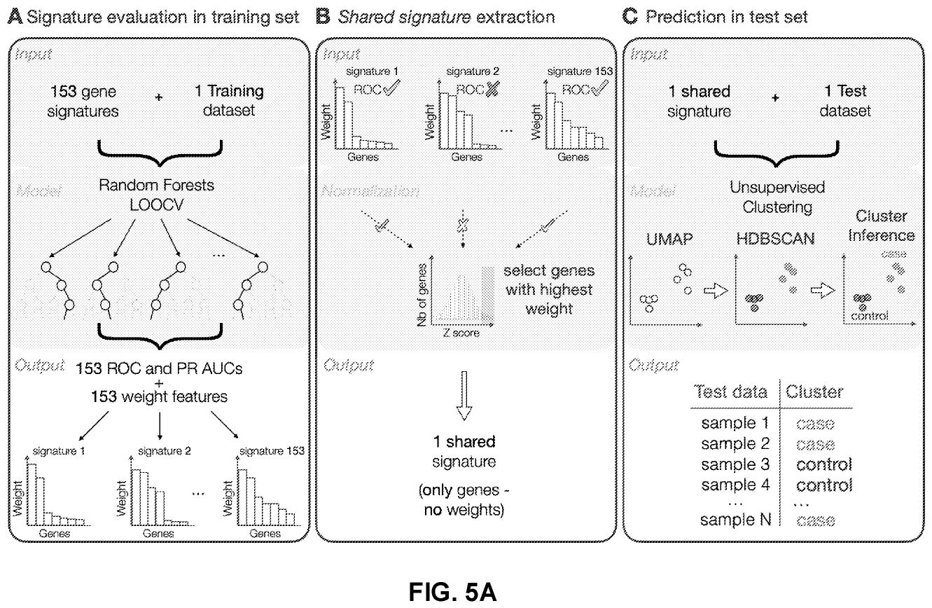

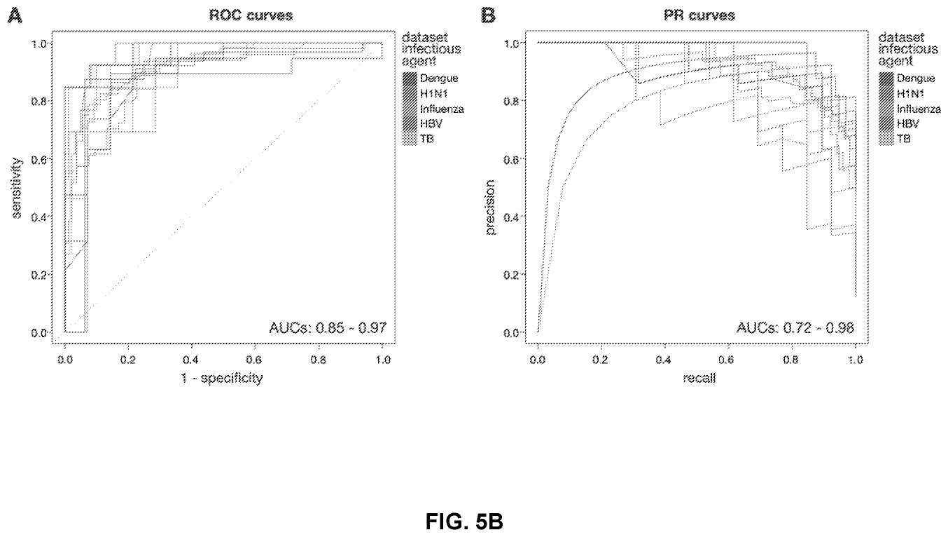

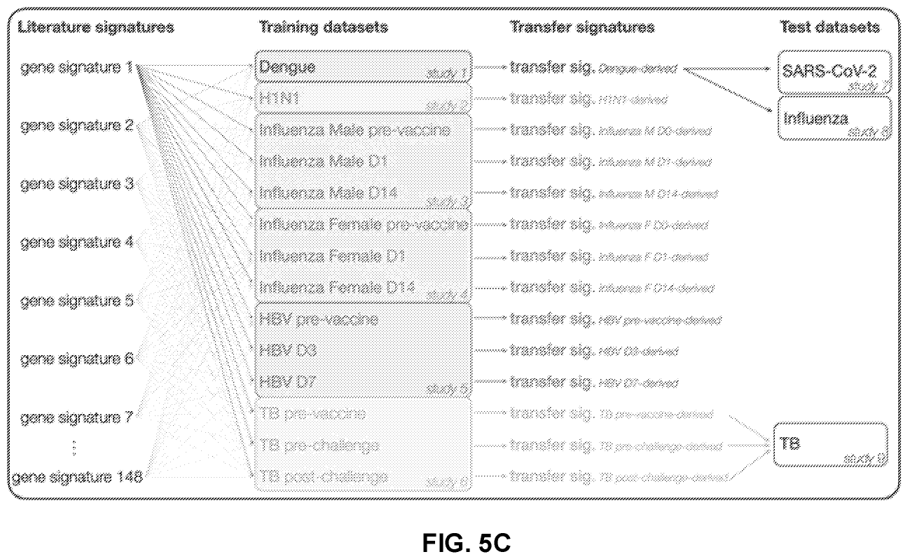

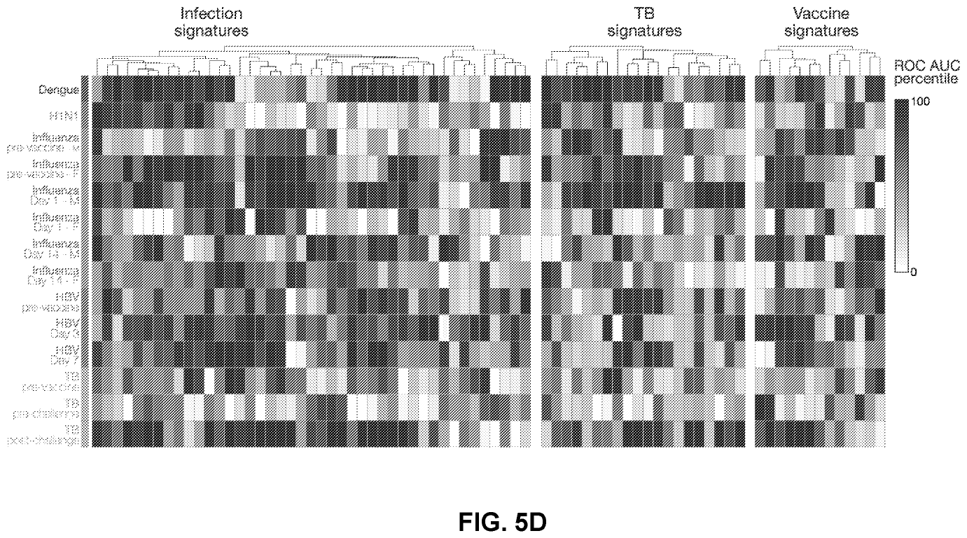

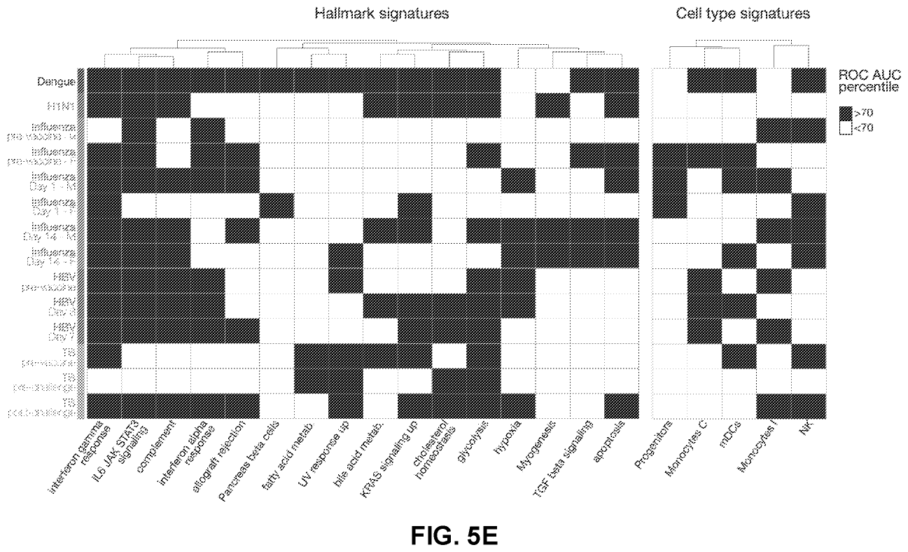

FIG. 5A depicts an example study design of generating a universal signature from a training set and their implementation in a test set. The study design uses random forest models to evaluate the collection of signatures on each training transcriptome datasets, followed by the extraction of a common set of predictive genes (referred to as a universal signature or a shared signature) from each training dataset and finally using the universal signature obtained from one training dataset to predict the outcome in an unseen, unrelated test datasets using unsupervised methods to exclude overfitting.

FIG. 5A shows three steps to progress from literature signatures (left panel) to universal signatures (middle panel) to prediction in unseen datasets (right panel). For example, a study aims at predicting (i) SARS-CoV2 and Influenza severe disease using a universal signature extracted from a Dengue infection dataset and (ii) tuberculosis progression in humans using transfer signatures extracted from a Rhesus tuberculosis vaccine dataset. The study includes other biologically related training datasets, and other biologically related or unrelated test datasets to evaluate the performance of transfer signatures.

Generally, in the first step, performance of 153 signatures on each training data set was characterized. Training datasets were from six studies covering responses to dengue infection, influenza H1N1 infection, and to vaccination to influenza, hepatitis B virus, and one study on tuberculosis in rhesus macaques. Machine learning models were trained and evaluated with the feature set restricted to the genes contained in the signature. Effectively, for any training dataset, for example on dengue infection, 153 models were obtained, from which ROC values and the individual importance of the genes in the original signature were extracted. The ROC AUCs were computed using the label prediction of each sample left out with the leave-one-out cross-validation strategy. As the different datasets do not contain the same fraction of cases and controls, it is not possible to directly compare ROC AUCs; for this reason, the results are expressed in percentiles rather than raw ROC AUC values.

ROC AUCs percentiles were obtained by comparing the literature signature to random list of genes of the same size. A large proportion of signatures performed well across training datasets, supporting the notion that published signatures contain valuable information that can be used to train predictive models and classifiers

To establish a universal signature for each training dataset, signatures were selected that had a ROC AUC higher than the 70th percentile compared to random list of genes of the same size. For the purpose of defining a universal signature, the cognate signature was excluded for this step in order to focus on genes that were also relevant in at least one external study.

Signatures that had a ROC AUC percentile above a given threshold were used at this step. Percentiles were determined as follows: for each signature—training dataset pair, 100 random genes signatures of the same size were used to compare the performance of the literature signature. Percentiles were used to be able to compare the numbers across datasets that did not have the same case/control distributions. The thresholds of 70, 80 and 90 were empirically tested and the 70^{th} percentile was chosen, as the two latter were too stringent (in terms of number of signatures that passed the threshold) when the signatures were split by group. In order to be able to compare the gene importance feature across signatures for a given training dataset, each gene signature importance feature was standardized to obtain a mean of 0 and a standard deviation of 1 (z-scores). The z-scores were then aggregated, and the top unique genes were selected as representing the universal signature.

The first 50 genes with the highest standardized importance feature score were selected. As expected, universal signatures performed well on their target datasets (datasets they were trained on). FIG. 5B depicts the performance of the universal signatures on their target datasets. AUC ROC varied between 0.85 and 0.97 and PR AUC of 0.72 to 0.98 for the various training datasets. In all but one training dataset (TB pre-vaccine), they matched or improved the performance, in terms of ROC AUC, of the best performing literature signature, including the cognate signature. Each line depicts the curve obtained for a given training dataset. The lines are colored based on the infectious agent studied in the training dataset.

Because universal signatures include genes specifically selected because they had the highest weight in the random forest models, the approach leads to optimized signatures for a given training study dataset. Fitting an overly expressive model will limit the generalizability of signatures to new datasets. Therefore, moving forward, the universal signatures will include a list of genes and there are no weights attached to the genes. Thus, the next step of dimensionality reduction involved the use of the universal signatures without any weights, followed by unsupervised clustering and a hyperparameter-less decision boundary to explore the generalization ability of gene signature-based prediction on a new test dataset.

FIG. 5C depicts an example study design including signatures, training datasets, and test datasets. This schema highlights the pairing of literature signatures and datasets used for training to generate the universal signatures (referred to as “transfer signatures in FIG. 5C) and finally the pairing of universal signatures and test datasets. This figure complements the study design depicted above in FIG. 5A. From left to right: each literature signature (N=148) is used with each training dataset (N=14) as an input to train a random forest model (see FIG. 5A). In other words, there are 148 random forest models per training dataset. The gene importance feature and ROC AUC from all random forest models obtained for a given training dataset is used as input to generate one “universal signature” per training dataset. In other words, a single universal signature is obtained by combining the information obtained from a set of literature gene signatures (here, start with all literature signatures, except the cognate signature—signature coming from the same paper than the dataset—for a given training dataset). Finally, the universal signature derived from each training dataset can be used as an input for unsupervised clustering of a new test dataset. The pairings between universal signatures and test datasets used in this study are depicted by the arrows. Example literature signatures are described in Table 4, example training datasets are described in Table 2, and example test datasets are described in Table 3. Abbreviations used in FIG. 5C are as follows: D0, Day 0 is equivalent to pre-vaccine. D1, Day 1. D3, Day 3. D7, Day 7. D14, Day 14. F, Female. M, Male.

Literature signatures: Five categories of signatures from publications were derived, hereafter referred to as “literature signatures”: (i) curated sets of gene lists—referred as hallmark signatures (N=50, https://www.gsea-msigdb.org/gsea/msigdb/collections.jsp) (1), (ii) gene signatures associated with cell composition in PBMC—referred as cell type signatures (N=22) (2), (iii) vaccine protection and response signatures—referred as vaccine signatures (N=13), (iv) progression from latent to active TB infection signatures—referred as TB signatures (N=20) and (v) viral and bacterial infection signatures—referred as infection signatures (N=43). Of note, due to gene nomenclature conversion issues, some signatures may be missing some genes identified in the parent paper.

Training datasets: 14 different training datasets were used from six studies: one study on dengue infection (4) (Table 2—study 1), one study on influenza H1N1 infection (5) (Table 2—study 2), one study on trivalent Influenza vaccination comprising two cohorts, one with males (Table 2—study 3) and one with females (6) (Table 2—study 4)—each comprising 3 datasets obtained at different timepoints (pre-vaccination, day 1 and day 14 post-vaccination), one study on hepatitis B virus (HBV) vaccination (7) (Table 2—study 5)—comprising 3 datasets obtained at different timepoints (pre-vaccination, day 3 and day 7 post-vaccination) and one study on tuberculosis (TB) vaccination in rhesus macaques (8) (Table 2—study 6)—comprising 3 datasets obtained at different timepoints (pre-vaccination, pre-challenge with TB and 28 days post-challenge with TB). Of note, several studies contained multiple non-independent datasets (or timepoints). This design is expected to help understand the biology of shared transcriptome signature and enables to monitor what are the earliest time points with predictive power.

Test datasets: 3 test datasets from three studies were used: one study on bronchoalveolar lavage in SARS-CoV-2 infection (9) (Table 3—study 7), one study on influenza infection (10) (Table 3—study 8) and one longitudinal study on TB progression in latently infected individuals (11) (Table 3—study 9). Of note, all test datasets were independent from each other and from any training datasets.

Phenotypes used: Multiple phenotypes in the training and test datasets were explored; the phenotype can be categorized in four groups, namely (i) severity of symptoms during viral infection (for dengue, influenza and SARS-CoV-2 infection studies), (ii) vaccine response (for both HBV and influenza vaccination studies), (iii) disease state—for TB vaccination study in rhesus macaque, and (iv) time to disease in the longitudinal study TB progression. Further description and the number of individuals in each phenotype category per study is provided in Tables 2 and 3. Of note, the phenotype extracted from the publicly available datasets is not necessarily the one used in the original study. As an example, categorical/binary phenotypes were used even when the original study used numerical phenotype in order to be consistent across datasets and to better mimic future potential practical use cases.

The successful implementation of universal signatures described above leaves open the question of how to choose the universal signature to be applied in a new dataset. Specifically, training and test data sets were selected for diseases that were likely related due to underlying disease pathogenesis. For example, TB vaccination efficacy may relate to prevention of progression of TB, and the severity of viral disease caused by Dengue, SARS-CoV-2 and influenza may be considered to be related. To challenge this biological-understanding-biased decision, the performance of transfer signatures and test data sets from biological processes that were less clearly related were also evaluated. To this end the transfer signatures described above and additional transfer signatures from influenza and hepatitis B vaccination were used to predict the severity of inflammatory and autoimmune diseases (rheumatoid arthritis and asthma) and to predict survival from malignancy as measured in datasets from cancer.

“Related pairs” were defined as training-test pairs from diseases with apparent biological relationships. “Unrelated pairs” were defined as training-test pairs from unrelated diseases. All possible pairs of training (n=14) and test datasets (n=3 “related pairs”, n=34 “unrelated pairs”) were evaluated. Tables 7A (“related pairs”) and 7B (“unrelated pairs”) provide the F1 score obtained when comparing the inferred case cluster versus the inferred control cluster. The highest score is also provided for each test dataset.

As hypothesized, the original training-test pairs from diseases with more apparent biological relationships (dengue and SARS-CoV-2 and influenza; tuberculosis in an animal model and in humans) were appropriate choices (“related pairs”, Tables 7A and 7C showing F1 score and log 2 enrichment scores respectively). Additionally, good performance was observed for severe respiratory viral infection transfer signatures in rheumatoid arthritis, which reinforces the concept of shared immunophenotypes, and suggests that diseases with less apparent relationships clinically nevertheless have underlying similarities in biology that are identified by the machine learning-based approach described herein. In addition, some transfer signatures were occasionally predictors of outcome for certain cancer types (“unrelated pairs”, Table 7B and 7D showing F1 score and log 2 enrichment scores respectively). These observations extend the interest of exploring transfer signatures from infectious diseases to unrelated fields such as auto-immunity and in cancer.

### Example 3: Example Methods of Predictive Universal Signatures

FIG. 5D depicts performance of different signatures, supporting the notion that published signatures contain valuable information that can be used to train predictive models and classifiers. Specifically, FIG. 5D depicts a heatmap of the AUROCs obtained through random forest models. Each column represents a signature from the literature, grouped by signature group. Each row represents a training dataset. In order to be able to compare the AUROC across the datasets (which do not have the same case/control distribution), the AUROC are depicted in percentiles. The percentiles are obtained by comparing the performance of the literature signature to 100 random gene lists of the same size. The same cutoff as used for the signature retention in the model was used (70^{th} percentile). Missing data is depicted in grey. The color annotation next indicates the infectious agent datasets. Influenza refers here to a tri-valent vaccine consisting of H1N1, H3N2 and IBV.

Additionally, FIG. 5E depicts top performing signatures across the various training datasets. In particular, FIG. 5E depicts a cutoff of AUC of 0.70, where signatures exhibiting an AUC greater than 0.70 are shown in blue and signatures exhibiting an AUC less than 0.70 are shown in white. Specifically, FIG. 5E displays the best performing hallmark and cell type signatures. Each row represents a training dataset (in the same order as in panel A). Columns represent the signatures—hallmark (left subpanel) and cell type (right panel)—that reached the 70^{th} percentile in at least one training dataset. For visual simplicity, the coloring here is binary as depicted in the legend.

As more specific examples, universal signatures for disease were generated by analyzing Rhesus Macaque or human datasets that included expressions of markers. These universal signatures were then applied to Rhesus Macaque (RM) or human data pertaining to a second disease indication. This experiment demonstrates the ability to develop universal signatures from data pertaining to a first disease indication that are then predictive for a second disease indication. In one scenario, the first disease indication and second disease indication differ according to the animal species in which the disease manifests (e.g., first disease in a RM and second disease in a human). Thus, the universal signatures are applicable across different disease indications, which in this scenario refers to diseases in different organisms.

Rhesus Macaque and human datasets were obtained from the following NCBI Gene Expression Omnibus databases: Accession number 79362, 102440, 110480, 17924, 21802, 111368, 145926, 48023, and 48018. To generate universal signatures, a feature selection process is performed on a dataset pertaining to a first disease indication. As used in the subsequent examples below, a feature selection process is performed on any of: a RM dataset including data pertaining to TB vaccine protection, a human dataset including data pertaining to progression of TB (e.g., progression of latent TB to active TB), an infectious disease database including human data pertaining to infectious diseases, or a human dataset including data pertaining to presence of TB, or an aggregation of two datasets (e.g., a RM dataset including data pertaining to TB vaccine protection and a human dataset including data pertaining to progression of TB). These datasets include expression data for genes and/or gene products such as gene transcripts (e.g., mRNA) and biomarkers/proteins.

Generally, a supervised feature selection process using random forest was performed on the dataset to identify signatures that are informative for the first disease indication. For example, a supervised feature selection process using random forest was performed on the RM dataset to identify RM signatures that are informative for distinguishing between RMs that exhibit TB vaccine protection and RMs that do not exhibit TB vaccine protection. A Random Forest model is run on each “gene signature-training dataset” pair. In the model, normalized gene expression of the subset of genes is used to classify the phenotype of interest. The models are trained using leave-one-out cross validation (LOOCV). The LOOCV strategy results in one RF model trained per sample per “gene signature-training dataset” pair. To obtain the combined gene importance feature, the feature importance scores are averaged across all models from a given “gene signature-training dataset” pair, resulting in one score of “importance” per gene per “gene signature-training dataset” pair, where the importance measure reflect the mean decrease in node impurity. The receiving operating characteristic (ROC) area under the curve (AUC) are computed using the predictions of the single left-out sample per trained model. In order to be able to compare the gene importance feature across signatures for a given training dataset, each gene signature importance feature is standardized to obtain a mean of 0 and a standard deviation of 1. The standardized scores are then aggregated, and the top unique genes are selected to be included in the universal signature.

Given the universal signature obtained from analysis of the first disease indication, the universal signature is applied to generate a prediction for a second disease indication. For example, a second dataset includes expressions of markers, a subset of which are included in the universal signature learned from data of a first disease indication. Thus, analyzing the expression of markers of the universal signature from the second dataset generates predictions for any of: vaccine protection in RM data, progression of TB in human data, or outlook (e.g., survival time) of human patients with brain cancer (e.g., glioma).

In this example, generating a prediction for the second disease indication involves performing a dimensionality reduction analysis on the quantities of the second dataset according to the signatures learned from the first dataset. Here, a uniform manifold approximation and projection (UMAP) analysis was conducted to map the expressions of the universal signature in the second dataset to a lower dimensional space. The dimension reduction was performed using dens-UMAP (http://cb.csail.mit.edu/cb/densvis/), that enable to maintain the local density of datapoint in the initial data space (Narayan, A. et al, “Density-Preserving Data Visualization Unveils Dynamic Patterns Of Single-Cell Transcriptomic Variability.” bioRxiv 2020.05.12.077776), Next, an unsupervised clustering analysis, specifically hierarchical density based spatial clustering (HDBScan), was performed on the expressions in the lower dimensional space to cluster and classify the patients. HDBSCAN can cluster data of varying shape and density, where the only parameter required to be provided is the minimal number of samples per cluster. The minimal number of samples was tested empirically for each unsupervised clustering, by identifying the number of samples per cluster that resulted in the lowest number of outliers and samples with low probability (<0.05) of cluster assignment. Thus, patients that fall within a particular cluster are predicted to have a particular disease activity (e.g., active or latent TB progression, better patient outlook or worse patient outlook, etc.).

More specifically, once clusters were identified, the inference of cluster attribution (case or control) was estimated based on the expression of the genes in the signature. Specifically, the direction of the signal rather than the amplitude was used for cluster attribution: for each gene present in the universal signature, the median expression in each cluster was compared and the direction of the signal in each cluster was recorded (high, low or intermediate—in the presence of more than 2 clusters). The same analysis was conducted in the training dataset where the universal signature was obtained from, using the true labels (case/control) instead of clusters to group the samples. Next, clusters in the test dataset were assessed according to the highest proportion of genes that matched the label of interest in the training dataset (in terms of signal direction), thereby defining clusters as either “case cluster” or control cluster. In the rare case where two clusters had the same proportion of matches, the sum of the absolute difference (in median expression) of the genes that matched the direction of the signal in the training dataset was compared. Of note, biological understanding can be used to decide which phenotype label in the training dataset would resemble the phenotype of interest (“case”) in the test dataset. For example, in the tuberculosis use case where the universal signature was obtained with the post-challenge timepoint, it was expected that the rhesus macaques that were not protected by the vaccine at the end of the study, were the most likely to resemble the individuals that were going to develop acute TB within in a year, as the rhesus macaques were already in a disease state at that time point and the unprotected animals were expected to have a much higher level of immune gene expression in the disease state. On the contrary, when the universal signatures obtained from the pre-vaccine or pre-challenge datasets were used, it was expected that the “case” phenotype to the be rhesus macaques that were protected by the vaccine at the end of the study, as the animals with higher basal level of immune gene expression (such as interferon stimulated genes) are expected to have a higher likelihood of vaccine protection.

### Example 4: Example Machine Learning Methods for Generating Predictive Universal Signatures from Datasets

Gene Signature evaluation in training datasets: A random forest model was run on each “literature signature-training dataset” pair (hereafter referred as S-D pair). In order to prevent overfitting the model to a specific pair and given the downstream goal of identifying genes that were common biomarkers across experiments and conditions, rather than specific to a single study or pair, hyperparameters were not tuned and were used as follow: number of trees (N=1,000); all other hyperparameters were the default in randomForest function from the R package “randomForest”. In the model, normalized gene expression of the subset of genes present in the signature was used to classify the phenotype of interest. For RNAseq input datasets, the normalization consisted in log 10 (reads per million mapped read+1e-7) and genes with initially less than 20 reads in every samples in the dataset were removed. For microarray input datasets, the normalized data from the GEO repository was retrieved, the normalized signal of all probes were averaged per gene and the log 10 (average normalized signal per gene+1e-7) was used as input for the model. The code used for running the random forest modeling was adapted from https://github.com/jasonzhao0307/R_lib_jason/blob/master/RF_output.R

Given the small sample size of most datasets and limited availability of datasets, the models were trained using leave-one-out cross validation (LOOCV), where for each sample of a dataset, all other samples from the same dataset are used to train the RF model, and the resulting model is used to predict the label or phenotype of the remaining sample. The LOOCV strategy results in one RF model trained per sample per S-D pair. To obtain the combined gene importance feature for a specific S-D pair, the gene importance scores were averaged across all models from a given S-D pair, resulting in one score of “importance” per gene per S-D pair, where the importance measure reflects the mean decrease in node impurity. The receiving operating characteristic (ROC) and precision recall (PR) area under the curve (AUC) are computed using the scores of the single left-out sample per trained model.

Extraction of universal signatures: Only literature signatures that had a ROC AUC percentile above a given threshold were used at this step. Percentiles were determined as follows: for each S-D pair, 100 random gene lists of the same size were used to compare the performance of the literature signature. Percentiles were used to be able to compare the numbers across datasets that did not have the same case/control distributions. The thresholds of 70, 80 and 90 were empirically tested and the 70^{th} percentile was chosen, as the two latter were too stringent (in terms of number of literature signatures that passed the threshold) when the signatures were split by group. In order to be able to compare the gene importance feature across literature signatures for a given training dataset, each gene literature signature importance feature was standardized to obtain a mean of 0 and a standard deviation of 1 (z-scores). The z-scores were then aggregated, and the top unique genes were selected as representing the universal signature.

The number of genes (N=10, 20 and 50) were empirically tested. The size of 50 genes was chosen for further analyses, with the rationale that (i) 50 genes appeared to provide the best performance in the datasets for which the signature length appeared to play the largest impact and (ii) the larger the signature length the more likely the signature will generalize to other datasets under different conditions. The gene lists of universal signatures derived from all contributing literature signatures are provided in Table 5.

Gene set overrepresentation was performed on the Biological Process GO ontology. Significance was judged by Benjamini-Hochberg correct p-value cutoff of 0.01. The top 10 significant GO sets are laid out in a plane by placing sets of higher overlap closer to each other. Specifically the ‘enrichplot’ and ‘clusterProfiler’ R packages have been used. Gene enrichment for Tuberculosis (e.g., TB, TB Pre-vaccine, TB pre-challenge, and TB post-challenge) and Dengue universal signatures are provided in Tables 8-13.

Additionally, the performance of literature signatures is shown in Table 6. The classifying performance of the predicted phenotypes obtained from the random forest models (with leave-one-out cross validation) using the literature signatures was assessed for each training dataset. The columns in Table 6 represent the training datasets and the rows the literature signatures. In order to be able to compare the performance across datasets (which do not have the same case/control distribution), the ROC AUCs were evaluated in terms of percentiles. The percentiles are obtained by comparing the literature signature performance to 100 random gene lists of the same size. The higher the percentile the better the performance of the signature. Missing data—due to gene conversion issues or no expression in the training datasets—are entered as “NA”.

### Example 5: Example Universal Signatures from Rhesus Macaque or Human Datasets

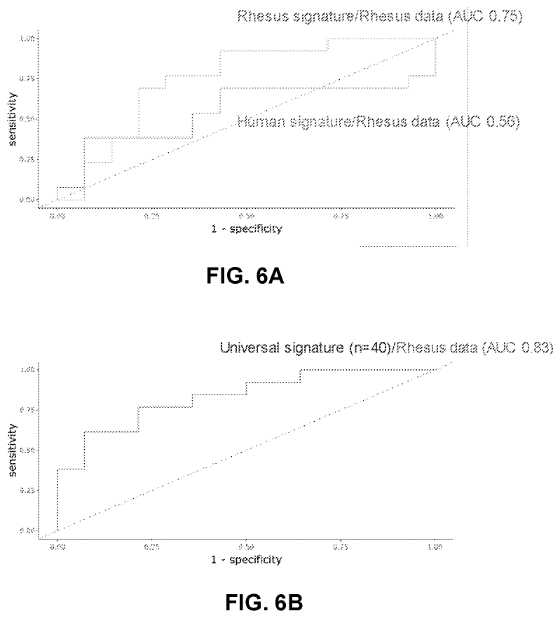

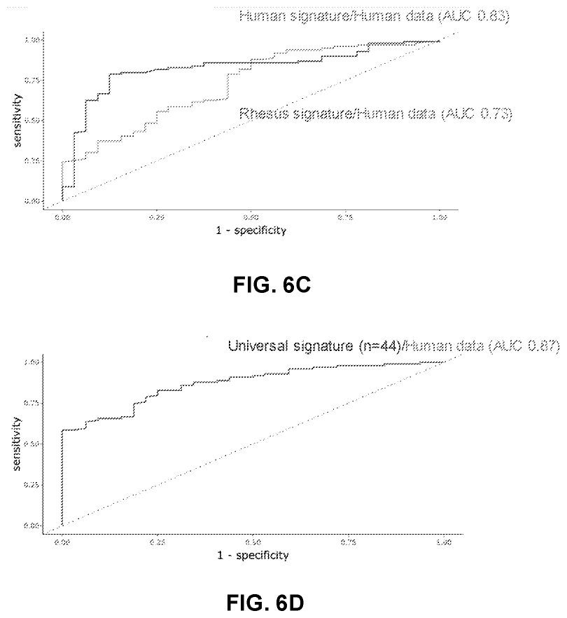

FIG. 6A depicts receiver operating curves for classifying RM data using signatures derived from RM or human datasets. Here, RM signatures were extracted from RM datasets including data describing tuberculosis vaccine protection in RMs. The human signatures were extracted from human datasets including data describing progression of latent TB to active TB in humans. A feature selection process using random forest, as described above in Example 1, was implemented to extract signatures from their respective datasets. Therefore, the extracted RM signatures represent features that are informative for differentiating between a RM that is likely to exhibit TB vaccine protection and a RM that is unlikely to exhibit TB vaccine protection. Additionally, the extracted human signatures represent features that are informative for differentiating between a human who is likely to progress from latent TB to active TB and a human who is unlikely to progress from latent TB to active TB.

As shown in FIG. 6A, the RM signatures and human signatures were validated against the RM data. The application of the RM signatures to RM data, hereafter referred to as the cognate analysis, represents a method of predicting a disease indication for the RM data using signatures that were selected to be predictive of that same disease indication (e.g., TB vaccine protection). In contrast, the application of the human signatures to the RM data is a cross-species analysis. Here, the cognate analysis resulted in an AUC=0.75 and the cross-species analysis was less predictive (AUC=0.56).

In comparison, FIG. 6B depicts a receiver operating curve for predicting disease activity of RM data using a universal signature. Here, the universal signature was obtained from the datasets by combining the top performing genes from both human and RM and rerunning a RF with leave one out cross-validation (LOOCV). The AUC value of 0.87 demonstrates the performance of the universal signature on the 1 left out set. Of note, the universal signature achieve a higher performance (AUC=0.87) in comparison to the RM or human signatures described in FIG. 6A. This demonstrates that combining signatures from different sources (e.g., signatures from data pertaining to RM and human) enables the identification of a universal signature that is more predictive than signatures that are derived from either RM or human datasets alone.

Similarly, FIG. 6C depicts receiver operating curves for classifying human data using signatures extracted from RM or human datasets. Similar to the methods described above in reference to FIG. 6A, human signatures and RM signatures were extracted from human datasets (describing progression of TB) and RM datasets (describing TB vaccine protection). These human signatures and RM signatures were then validated against 1 left out set of human data to predict progression of latent TB to active TB in humans. The application of human signatures to human data represents a cognate analysis as it involves a method of predicting a disease indication using signatures that were selected to be predictive of that same disease indication (e.g., progression of TB). In contrast, the application of the RM signatures to the human data is a cross-species analysis. Here, the cognate analysis resulted in an AUC=0.83. The cross-species analysis was less predictive (AUC=0.73).

FIG. 6D depicts a receiver operating curve for classifying human data using a universal signature derived from both RM and human datasets. As described above, the universal signature was trained on diverse sets of data derived from infectious disease databases by performing a random forest feature selection process. Therefore, the extracted universal signature represents features that are informative for differentiating between disease activity of patients associated with infectious diseases. The universal signature was applied to human data to predict progression of TB (latent to active) in humans. Here, this application of the universal signature to human data represents a cross-disease analysis and implements the aforementioned transfer learning approach where the universal signature learned from one disease indication (e.g., infectious diseases) is useful for a prediction of a second disease indication (TB progression). Here, the cross-disease analysis yielded an AUC=0.87. Of note, the AUC of this cross-disease analysis (AUC=0.87) was an improvement on the AUC of the cognate analysis (AUC=0.83) described above in reference to FIG. 6C. This further demonstrates the applicability of using a universal signature learned from multiple sources that are more predictive than signatures learned from either RM or human datasets alone.

### Example 6: Example Methods for Implementing Predictive Universal Signatures

Universal signatures were used in an unsupervised analysis to cluster samples from new test datasets, that originated from independent studies (notably new condition, new organism or new infectious agent). The dimension reduction was performed using Uniform Manifold Approximation and Projection (UMAP), followed by Hierarchical Density-Based Spatial Clustering of Application with Noise (HDBSCAN) which can cluster data of varying shape and density. In this approach, the only parameter required is the minimal number of samples per cluster. For this purpose, the minimal number was tested empirically by identifying the number of samples per cluster that resulted in the lowest number of outliers multiplied by a penalty score equivalent to the square of the number of clusters. This approach limits the creation of excessive numbers of clusters, which could make interpretation difficult. The minimum number of samples per cluster was set to contain at least 7% of the total population. HDBSCAN was run using the hdbscan command from the R package “dbscan” (https://github.com/mhahsler/dbscan). The samples considered as outliers by HDBSCAN, were attributed to the closest cluster label using the 3 nearest neighbors with the knn command from the R package “dbscan” (https://github.com/mhahsler/dbscan). The code used for running the dimensionality reduction and unsupervised clustering was adapted from https://github.com/NikolayOskolkov/ClusteringHighDimensions/blob/master/easy_scrnaseq_tsn e_cluster.R

Once the clusters were identified, the inference of cluster attribution (case or control) was estimated based on the expression of the genes in the signature. Specifically, the direction of the signal rather than the absolute value was used. For each gene present in the universal signature, the median expression in each cluster was compared and the direction of the signal in each cluster (high, low or intermediate—in the presence of more than 2 clusters) was recorded. The same analysis was conducted in the training dataset where the universal signature was obtained from, using the true labels (case/control) instead of clusters to group the samples. Next, the cluster in the test dataset that had the highest proportion of genes that matched the label of interest in the training dataset (in terms of signal direction) was identified and defined as “case cluster”, while the other cluster(s) were defined as control cluster. In the rare case where two clusters had the same proportion of matches, the sum of the absolute difference (in median expression) of the genes that matched the direction of the signal in the training dataset was compared. Of note, biological understanding was used to decide which phenotype label in the training dataset would resemble the most the phenotype of interest (“case”) in the test dataset, if not the clusters will be inverted. For example, in the tuberculosis use case, when the universal signature obtained with the post-challenge timepoint was used, it was expected that rhesus macaques that were not protected by the vaccine at the end of the study, were the most likely to resemble the individuals that were going to develop acute TB within in a year, as the rhesus macaques were already in a disease state at that time point and the unprotected animals were expected to have a much higher level of immune gene expression in the disease state. While on the opposite, when the universal signatures obtained from the pre-vaccine or pre-challenge datasets were used, it was reasoned that the “case” phenotype to the be rhesus macaques that were protected by the vaccine at the end of the study, as the animals with higher basal level of immune gene expression (such as interferon stimulated genes) are expected to have a higher likelihood of vaccine protection.

### Example 7: Universal Signatures from Rhesus Macaques Distinguish Human Patient Clusters with Differing Tuberculosis Progression

Universal signatures were evaluated to assess the challenge of enriching a clinical trial with individuals that are likely to reach a given endpoint. The scenario is the use of a pharmacological or vaccine intervention to prevent progression from latent tuberculosis to active disease. Progression to active tuberculosis is a rare event (estimated as 0.084 cases per 100 person-years); therefore, it would be important to be able to recruit individuals that are the most likely to develop active infection within one year. Indeed, in the presence of a limited numbers of individuals that may reach a study endpoint the study may lack power to detect differences between the placebo and vaccine or treatment group.

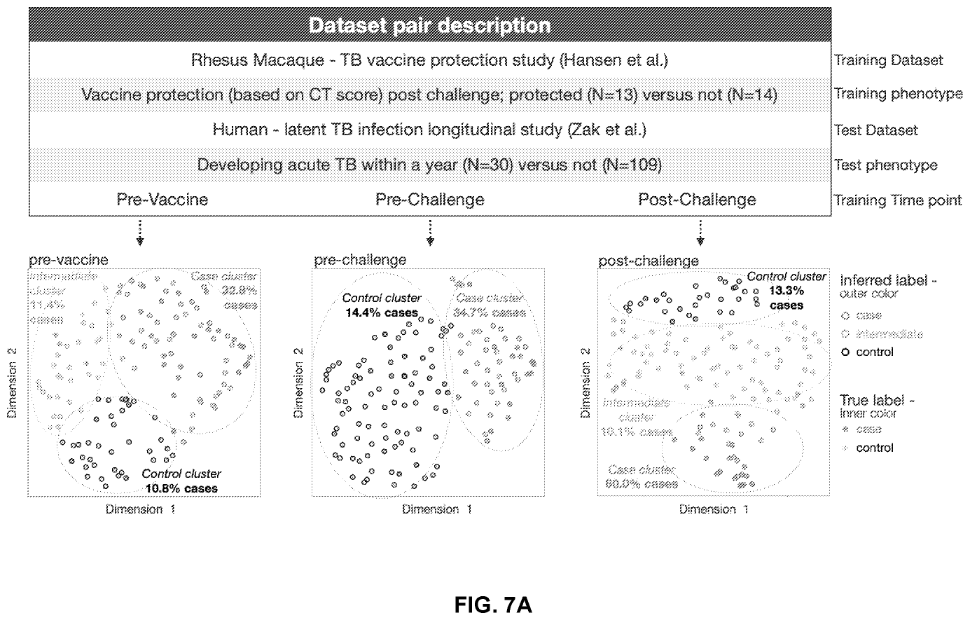

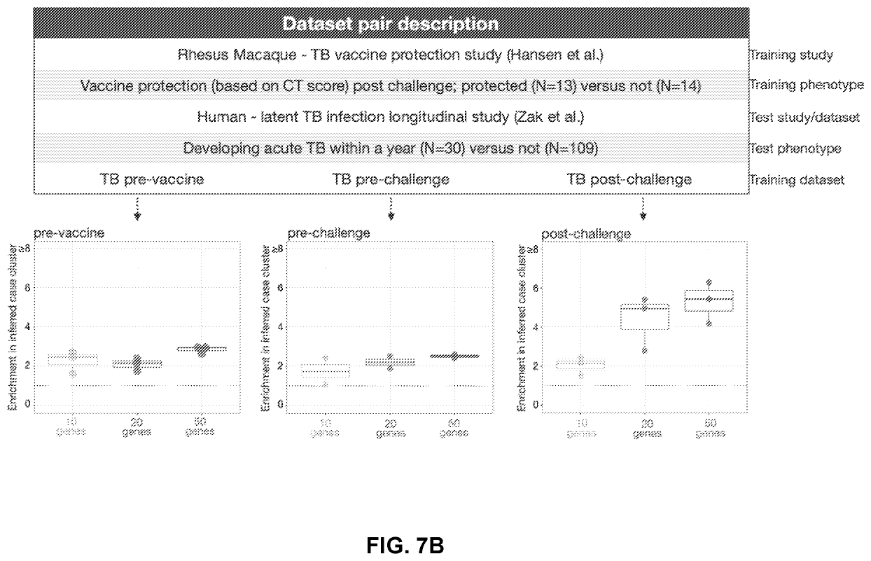

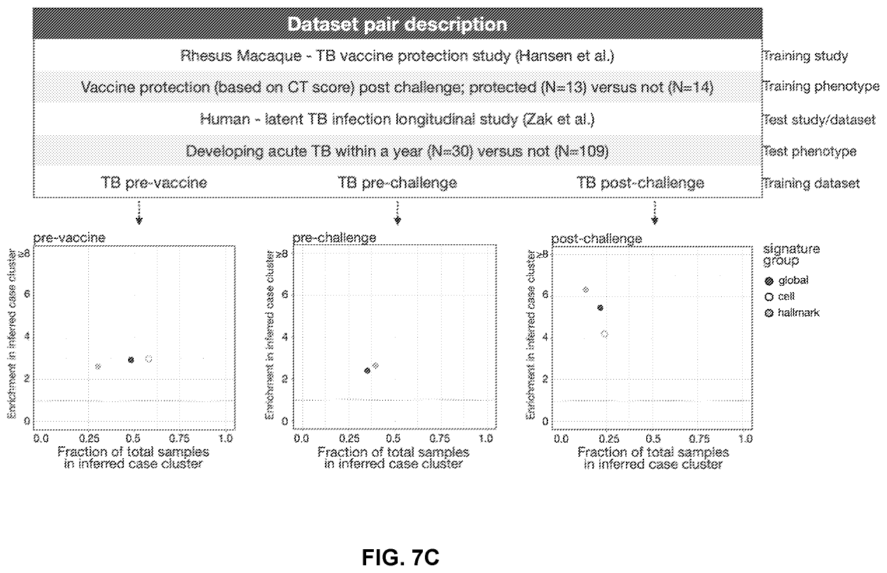

Here, universal signatures obtained with the datasets from the Hansen et al. study were evaluated (Hansen, S. G., et al. Prevention of tuberculosis in rhesus macaques by a cytomegalovirus-based vaccine. Nat Med 24, 130-143 (2018)). This study assessed the efficacy of a TB vaccine on Rhesus macaques, with longitudinal samples from 27 Rhesus macaques collected pre-vaccine, after vaccination but before TB challenge and four weeks post challenge. The phenotype used for training the random forest models was protection from TB (vaccine efficacy), defined as a computed tomography score of <10 (protected, N=13) at any time point post challenge versus not (not protected, N=14). Here, the target dataset was the data from Zak, D. E., et al. A blood RNA signature for tuberculosis disease risk: a prospective cohort study. Lancet 387, 2312-2322 (2016)., a longitudinal study assessing progression from latent to active TB. Cases were defined as individuals that developed TB within a year (N=30) and controls as individuals that did not develop TB within a year after entry in the study (N=109). The results of the unsupervised clustering are shown in FIG. 7A, which depicts results following a dimensionality reduction analysis and unsupervised clustering of human tuberculosis data using universal signatures learned from Rhesus Macaque tuberculosis vaccine protection datasets.

Here, a universal signature was extracted (e.g., using the feature extraction process described above) from RM datasets include data describing tuberculosis vaccine protection in RMs. Three different timepoints of data were analyzed to extract universal signatures: 1) pre-vaccine, 2) pre-challenge, and 3) post-challenge.

The universal signature was applied to human data to predict TB progression (latent TB to Active TB). This application of the universal signature to human data represents a cross-disease and cross-species analysis where the universal signature learned from one disease indication (e.g., TB vaccine protection in RMs) is useful for a prediction of a second disease indication (e.g., TB progression in humans).

The human data was analyzed by performing a dimensional reduction analysis on the universal signature, specifically a uniform manifold approximation and projection (UMAP) analysis. As shown in FIG. 7A, the top panel displays the study design and the bottom panel displays the UMAP projection of the test dataset using the 50 top genes from the commonality signature obtained from the training dataset—trained with samples obtained at 3 different timepoints: pre-vaccine, pre-challenge and post-challenge. Each sample of the test dataset is represented by a dot. The outer dot color indicates the inferred label (from the unsupervised clustering based solely on genes present in commonality signature obtained from training dataset) and the inner dot color indicates the true label. The percentage of true cases in the different clusters is displayed next to each cluster. The colored circles surrounding the clusters are approximate and used solely for visual guidance.

As shown in FIG. 7A, subjects were classified into at least two categories. For example, for the pre-vaccine and post-challenge training timepoints, the implementation of the universal signatures enabled the classification of subjects into 1) control cluster (e.g., will not develop acute TB within a year), 2) an intermediate cluster (e.g., a possibility of developing acute TB within a year), and 3) a case cluster (e.g., a high possibility of developing acute TB within a year). For the pre-challenge training timepoint, the implementation of the universal signatures enabled the classification of subjects into 1) control cluster (e.g., will not develop acute TB within a year) and 2) a case cluster (e.g., a high possibility of developing acute TB within a year).

With the universal signature defined on the pre-vaccine rhesus macaque samples, 32.8% of the predicted cases were correct, i.e., developed active TB within a year, while the samples outside of this cluster contained only 11.1% of true cases. Here, the unsupervised clustering lead to a 3.0-fold enrichment and a 73.3% recall. In a similar setting, but with the universal signature derived from pre-challenge samples, a 2.0-fold enrichment (34.7% versus 14.4%) and a 56.7% recall was obtained, while with the signature derived from post-challenge samples, a 5.5-fold enrichment (60.0% versus 11.0%) and 60.0% recall was obtained.

Altogether, this example demonstrates that universal signatures learned from one disease indication (e.g., TB vaccine protection in RM) can be transfer learned and applied for predicting progressors or non-progressors of TB in a human dataset. Additionally, the use of the universal signatures would allow the prospective recruitment of individuals into clinical trials with a greater likelihood of reaching adequate power.

FIG. 7B depicts the performance in a tuberculosis progression use case using different sizes of universal signatures (e.g., 10 genes, 20 genes, or 50 genes). The top panel shows the study design as also displayed in FIG. 7A. The bottom panel displays the enrichment of cases in the inferred case cluster compared to the other cluster(s)—y axis—using universal signatures of differing size—x axis. The three plots represent the results obtained with universal signatures trained with samples obtained at 3 different timepoints shown in the top panel: pre-vaccine, pre-infectious challenge and post-challenge. The results are depicted as boxplot with the individual data overlaid, where each dot represents the result obtained with a universal signature derived from a different group of literature signatures (global, cell type and hallmark). The enrichment per universal signature group is further detailed for the 50-gene-long universal signatures in FIG. 7C.

FIG. 7C depicts a comparison of universal signatures obtained from different signature groups in a tuberculosis progression use case. The bottom panel displays the enrichment of cases in the inferred case cluster compared to the other cluster(s) using 50-gene-long universal signatures—y axis—versus the fraction of samples present in the inferred case cluster—x axis. The three plots represent the results obtained with universal signatures trained with samples obtained at 3 different timepoints shown in the top panel: pre-vaccine, pre-infectious challenge and post-challenge. Each dot represents the result obtained with a universal signature derived from a different group of literature signatures (global, cell type and hallmark), where ‘global’ encompasses all signatures. The missing dot for the cell type universal signature trained on the TB pre-challenge dataset indicates that there were not enough (<50) genes present in the signatures that passed the initial 70^{th} percentile threshold used to extract the universal signature.

### Example 8: Universal Signatures from Hallmark Pathways in Tuberculosis Distinguish Human Glioma Patient Clusters with Differing Survival Times

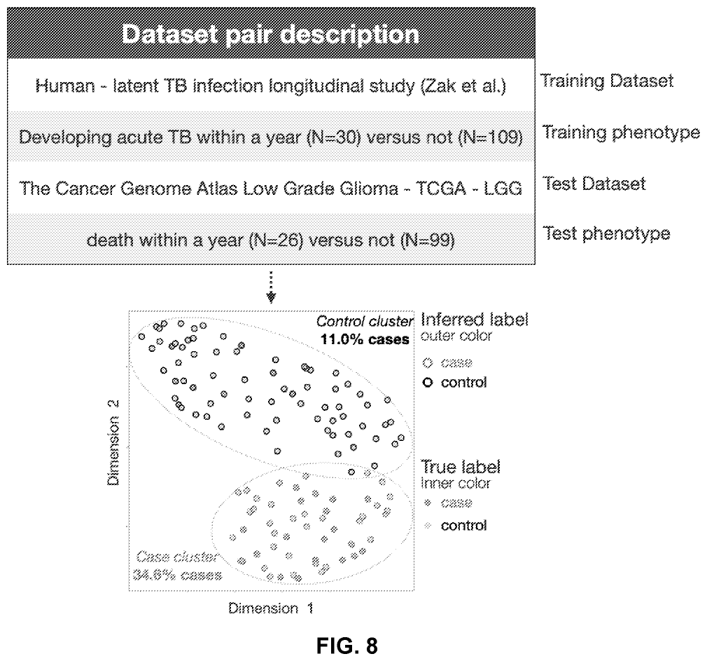

FIG. 8 depicts results of a dimensionality reduction analysis and unsupervised clustering of a human glioma dataset using a universal signature learned from hallmark pathways in tuberculosis. The diseases of TB and human glioma share a common condition of chronic infection.

Here, the universal signature was extracted (e.g., using the feature extraction process described in Example 1) from human datasets include data describing presence of tuberculosis in human individuals. The universal signature was applied to human data, specifically on a human glioma dataset obtained from the Cancer Genome Atlas (TCGA), to classify patient outlook with glioma. Patient outlook refers to the patient survival time.

As shown in FIG. 8, the top panel displays the study design and the bottom panel displays the UMAP projection of the test dataset using the 50 top genes from the commonality signature obtained from the training dataset. Each sample of the test dataset is represented by a dot. The outer dot color indicates the inferred label (from the unsupervised clustering based solely on genes present in commonality signature obtained from training dataset) and the inner dot color indicates the true label. The percentage of true cases in the different clusters is displayed next to each cluster. The colored circles surrounding the clusters are approximate and used solely for visual guidance.

As evident in FIG. 8, the UMAP analysis is able to generally organize data points of the patients in the lower dimensional space according to their patient outlook. Thus, clustering the data points on the lower dimensional space e.g., by using HDBScan, enables the classification of individuals according to their patient outlook. Specifically, subjects were classified into two categories: 1) control cluster (e.g., subject is unlikely to die within 1 year) and 2) case cluster (e.g., subject is likely to die within 1 year).

Again, these results establish that universal signatures learned from one disease indication (e.g., TB infection) can be transfer learned and applied for a second disease (e.g., patient outlook for glioma patients).

### Example 9: Universal Signatures from Dengue Viral Infection Distinguish Severity of Infection in Other Diseases

Universal signatures were assessed for their use in the setting of viral infection to predict or classify the severity of the symptoms of individuals that are hospitalized. Here, universal signatures were extracted from the dataset from the Devignot et al. study, consisting of children with acute dengue infection, with blood samples collected within 3 to 7 days after onset of fever (Devignot, S., et al. Genome-wide expression profiling deciphers host responses altered during dengue shock syndrome and reveals the role of innate immunity in severe dengue. PLoS One 5, e11671 (2010)). For the purpose of this analysis, children with severe manifestations of disease (shock syndrome and hemorrhagic fever; N=32) were considered as cases, while children that had uncomplicated dengue fever were considered controls (N=16). Data from Liao, M., et al. Single-cell landscape of bronchoalveolar immune cells in patients with COVID-19. Nat Med 26, 842-844 (2020) and Dunning, J., et al. Progression of whole-blood transcriptional signatures from interferon-induced to neutrophil-associated patterns in severe influenza. Nat Immunol 19, 625-635 (2018) were used as two different target datasets.

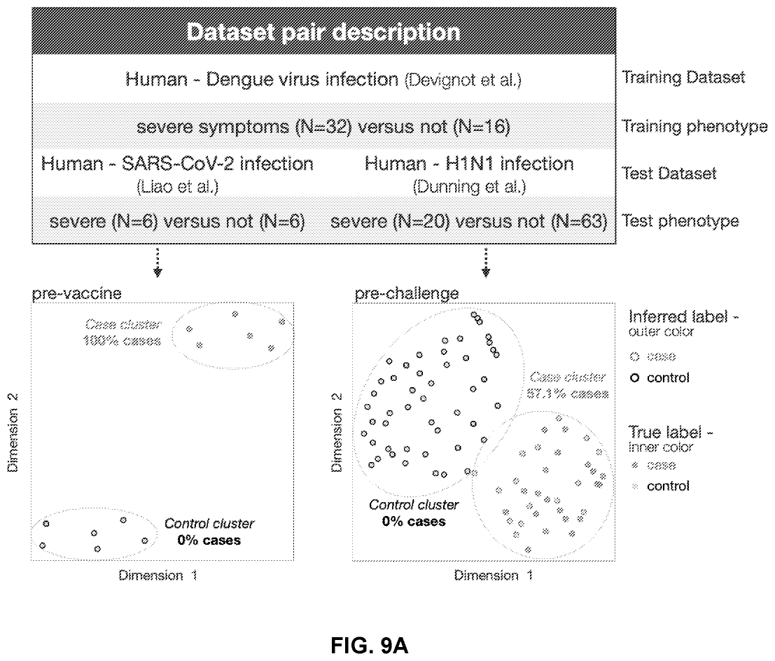

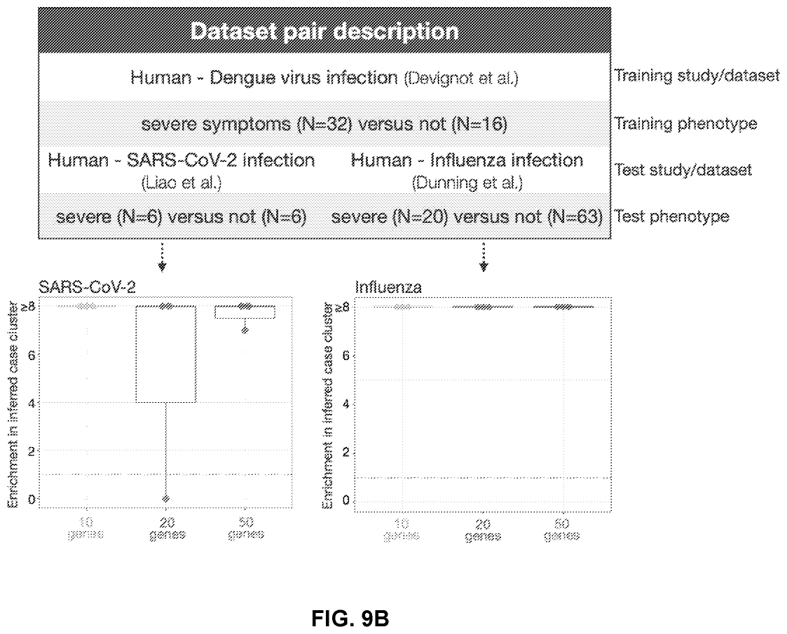

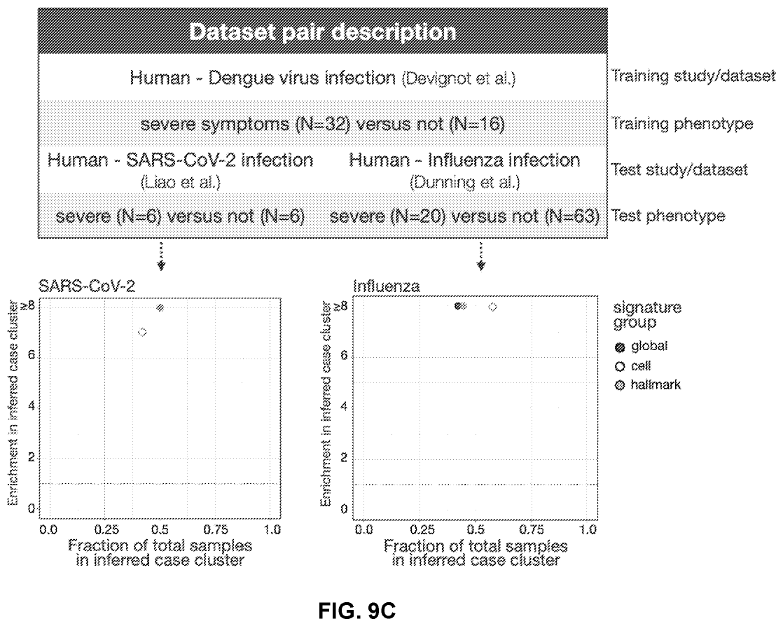

FIG. 9A depicts results of a dimensionality reduction analysis and unsupervised clustering of a human SARS-CoV-2 infection dataset and a human H1N1 infection dataset using universal signatures learned from a human Dengue virus infection dataset. The diseases of human Dengue virus infection, SARS-CoV-2, and H1N1 share a common condition of severe infection phenotype. FIG. 9A summarizes the biological content of the transfer signatures (TS) by displaying the gene set overrepresentation performed on the Biological Process GO ontology (e.g., Dengue TS). Dots represent term enrichment with color coding: red indicates high enrichment, blue indicates low enrichment. The sizes of the dots represent the percentage of contributing genes in a GO term. Significance was judged by Benjamini-Hochberg correct p-value cutoff of 0.01.

The study of Liao et al characterized bronchoalveolar lavage fluid immune cells from patients infected with SARS-CoV-2. For the purpose of this analysis, cases were the individuals that were described as having severe disease (N=6), while individuals with moderate disease (N=3) or not infected (N=3) were considered as controls (total N=6). The RNA samples were obtained 4-10 days after the phenotypes were established. All true cases of severe SARS-CoV-2 study were correctly classified in unsupervised clustering.

The study of Dunning et al characterized blood samples from individuals hospitalized with influenza. For the purpose of this analysis, cases were considered as the individuals that required mechanical ventilation (N=20), while individuals that did not require respiratory support were considered as controls (N=63). Given that the phenotypes were established at the same time or before the RNA samples were obtained in both studies, the unsupervised clustering results therefore reflect the performance of universal signatures as classifiers rather than predictors. The inferred case cluster included 57.1% true cases (individuals that required mechanical ventilation), while none of the samples in the inferred control cluster were true cases. Both the SARS-CoV-2 and the influenza study achieved a 100% recall, thus supporting the transportability of signatures across different viral infections as represented by the capacity to classify and predict disease severity. Analysis of the content of the Dengue universal signature confirmed the enrichment of genes of the immune response (Table 8 and FIG. 7A).

As shown in FIG. 9A, the top panel displays the study design and the bottom panel displays the UMAP projection of the test dataset using the 50 top genes from the commonality signature obtained from the training dataset. Each sample of the test dataset is represented by a dot. The outer dot color indicates the inferred label (from the unsupervised clustering based solely on genes present in commonality signature obtained from training dataset) and the inner dot color indicates the true label. The percentage of true cases in the different clusters is displayed next to each cluster. The colored circles surrounding the clusters are approximate and used solely for visual guidance.

Using the universal signature, classification of infection severity for SARS-CoV-2 subjects was successful in differentiating between a case cluster (e.g., severe infection) and a control cluster (e.g., not severe infection). Additionally, using the universal signature, classification of infection severity for H1N1 subjects was successful in differentiating between a case cluster (e.g., severe infection) and a control cluster (e.g., not severe infection).

Again, these results establish that universal signatures learned from one disease indication (e.g., Dengue virus infection) can be transfer learned and applied for multiple second diseases (e.g., SARS CoV-2 infection and H1N1 infection).

FIG. 9B depicts the performance in a severe viral disease use case using different sizes of universal signatures. The top panel shows the study design as displayed in FIG. 9A. The bottom panel displays the enrichment of cases in the inferred case cluster compared to the other cluster(s)—y axis—using universal signatures of differing size—x axis. The results are depicted as boxplot with the individual data overlaid, where each dot represents the result obtained with a universal signature derived from a different group of literature signatures (global, cell type and hallmark). The enrichment per universal signature group is further detailed for the 50-gene-long universal signatures in FIG. 9C.

FIG. 9C depicts a comparison of universal signatures obtained from different signature groups in a severe viral disease use case. The bottom panel displays the enrichment of cases in the inferred case cluster compared to the other cluster(s) using 50 gene commonality signatures—y axis—versus the fraction of samples present in the inferred case cluster—x axis. Each dot represents the result obtained with a universal signature derived from a different group of literature signatures (global, cell type and hallmark), where ‘global’ encompasses all signatures. The color code is provided in the legend. In the SARS-CoV-2 example, due to the small sample size, multiple universal signatures obtained from different groups of signatures (global and hallmark) generated the same clustering, yielding to the same results in terms of enrichment and fraction and are therefore overlaid and non-visible individually. Here, enrichments depicted as >8 indicate that all cases were correctly labeled/present in the inferred case cluster, as seen in FIG. 9A.

### Example 10: Comparing Performance of Universal Signatures

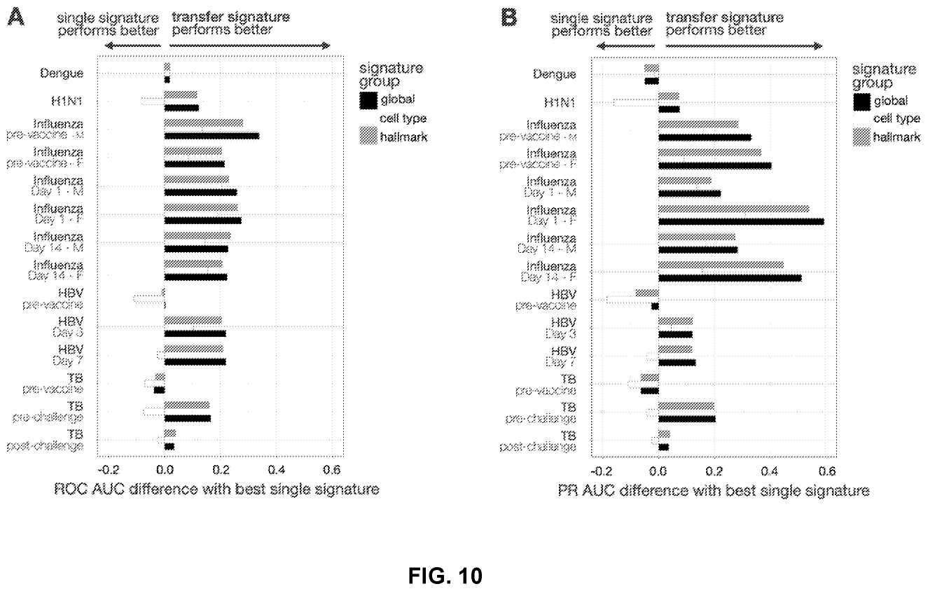

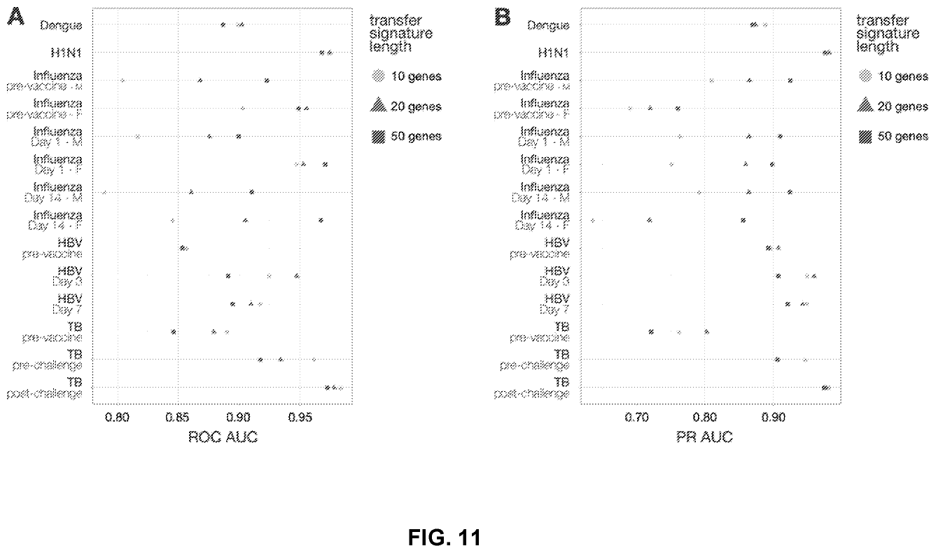

FIG. 10 depicts performance of universal signatures as compared to single signatures. The classifying performance of the predicted phenotypes obtained from the random forest models (with leave-one-out cross validation) using the transfer or single literature signatures was assessed for each training dataset. Both panels display the difference in performance (as measured in ROC AUC—Panel A—or PR AUC— Panel B) between the universal signature and the best single performing literature signature (including the cognate signature for the dataset). The universal signatures that outperformed the best single literature signature have a positive difference and inversely the ones that did not perform as well have a negative difference. For the purpose of this analysis, we developed not only one universal signature per training dataset (that was obtained when starting with all literature signatures), but also one universal signature for the cell type and hallmark group of signatures, per training dataset. In other words, we started with different subset of literature signatures to compute the universal signature and the results are depicted for those three groups of signatures, where ‘global’ encompasses all signatures. In most instances, the universal signature outperforms the best performing single signature, with the advantage of increasing the likelihood of generalization in new datasets as universal signatures are obtained from multiple literature signatures, reducing the risk of extracting condition/study specific markers.

### Example 11: Example Performance of Varying Numbers of Universal Signatures

FIG. 11 depicts the performance of universal signatures of varying sizes. The classifying performance of the predicted phenotypes obtained from the random forest models (with leave-one-out cross validation) using universal signatures of varying sizes was assessed for each respective training dataset. Three lengths of universal signatures are depicted in different color and shape. The color code is provided in the legend. Panel A displays the ROC AUC obtained for each training dataset. Panel B displays the PR AUC obtained for each training dataset. The size of 50 genes was chosen for further analyses, with the rationale that (i) 50 genes appeared to provide the best performance in the datasets for which the universal signature length appeared to play the largest impact and (ii) the larger the signature length the more likely the signature will generalize to other datasets with different conditions.

Of note, the results described above for the various use cases used a 50-gene-long transfer signature; however, similar results were obtained when selecting only the top 20 genes, while the performance dropped with some of the 10-gene transfer signatures (FIG. 7B, FIG. 9B, FIG. 11). Similar results were obtained when using transfer signatures derived with only hallmark signatures compared to transfer signatures based on all literature signatures (FIG. 7C and FIG. 9C). Overall, both the SARS-CoV-2 and the influenza studies support the value of transfer of signatures, as defined by our approach, across different viral infections to classify disease severity.

### Example 12: Establishing Threshold for Extracting Universal Signatures

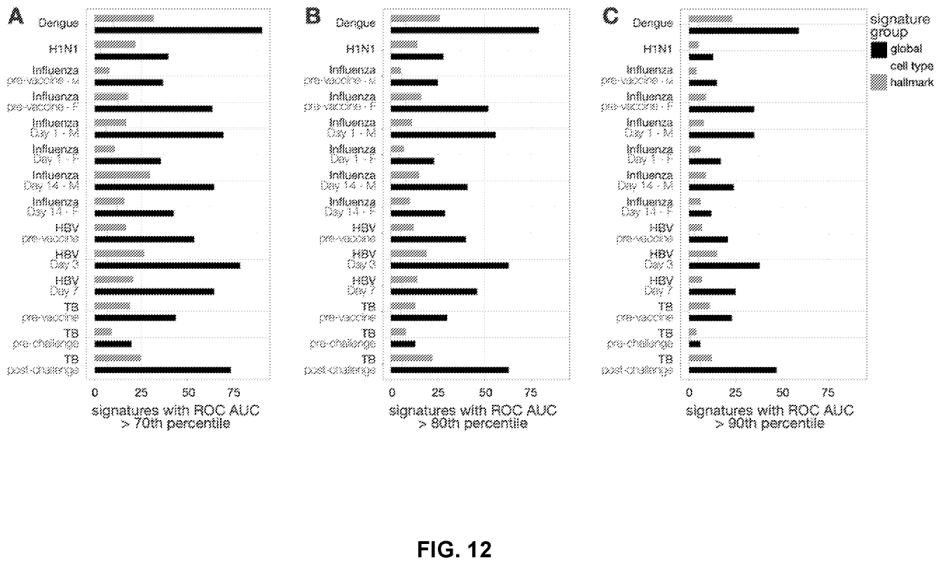

FIG. 12 depicts the number of literature signatures at differing thresholds (70, 80 and 90 percentile). Specifically, the thresholds of 70, 80 and 90 were empirically tested and the 70^{th} percentile was chosen for generating universal signatures, as the two latter were too stringent (in terms of number of literature signatures that passed the threshold) when the signatures were split by group. The barplots display, for the three groups of signatures used to generate universal signatures (global, cell type and hallmark), the number of signatures with ROC AUC higher than the 70^{th} percentile (Panel A), 80^{th} percentile (Panel B) and 90^{th} percentile (Panel C) for each signature group. The classifying performance of the predicted phenotypes are obtained from the random forest models (with leave-one-out cross validation) using the literature signatures was assessed for each training dataset. The percentiles are obtained by comparing the literature signature performance to 100 random gene lists of the same size. The higher the percentile, the better the performance of the signature.

Tables

TABLE 1
Example combinations of first disease indication,
second disease indication, and common condition.

| First Disease | Second Disease | |
|---|---|---|
| Indication | Indication | Common Condition |
| Progression to active | Glioma | Cancer |
| Tuberculosis | | |
| Rhesus macaque | Progression from | TB infection |
| protection to | latent to acute TB | |
| Tuberculosis (TB) | infection in humans | |
| after vaccination | | |
| Dengue infection in | H1N1 infection in | Severe infection |
| humans | humans | phenotype |
| Dengue infection in | SARS-CoV-2 | Severe infection |
| humans | infection in humans | phenotype |
| H1N1 infection in | SARS-CoV-2 | Severe infection |
| humans | infection in humans | phenotype |

TABLE 2
Example training datasets used from six different studies for generating universal signatures

| | Training | | | | | |
|---|---|---|---|---|---|---|
| Training | sub dataset | Evaluation | Binary phenotypes used | | Number of | |
| Study | Name | metric | for training | Labels | samples | Source GEO |
| 1 | Dengue | Severity of | Fever | control | 16 | https://www.ncbi.nlm.nih.gov/geo/query/ |
| | | symptoms | Hemorragic fever or shock | case | 32 | acc.cgi?acc=GSE17924 |
| | | | syndrome | | | |
| 2 | H1N1 | Severity of | Mechanical ventilation | case | 13 | https://www.ncbi.nlm.nih.gov/geo/query/ |
| | | symptoms | No mechanical ventilation | control | 12 | acc.cgi?acc=GSE21802 |
| 3 | Influenza | Trivalent | Seroconverter for all 3 | case | 56 | https://www.ncbi.nlm.nih.gov/geo/query/ |
| | pre- | vaccine | strains (H1N1, H3N2, FluB) | | | acc.cgi?acc=GSE48018 |
| | vaccine M | response at | Not Seroconverter for all 3 | control | 54 | |
| | | Day 28 | strains (H1N1, H3N2, FluB) | | | |
| | Influenza | Trivalent | Seroconverter for all 3 | case | 54 | |
| | Day 1 M | vaccine | strains (H1N1, H3N2, FluB) | | | |
| | | response at | Not Seroconverter for all 3 | control | 53 | |
| | | Day 28 | strains (H1N1, H3N2, FluB) | | | |
| | Influenza | Trivalent | Seroconverter for all 3 | case | 51 | |
| | Day 14 M | vaccine | strains (H1N1, H3N2, FluB) | | | |
| | | response at | Not Seroconverter for all 3 | control | 54 | |
| | | Day 28 | strains (H1N1, H3N2, FluB) | | | |
| 4 | Influenza | Trivalent | Seroconverter for all 3 | case | 13 | https://www.ncbi.nlm.nih.gov/geo/query/ |
| | pre- | vaccine | strains (H1N1, H3N2, FluB) | | | acc.cgi?acc=GSE48023 |
| | vaccine F | response at | Not Seroconverter for all 3 | control | 94 | |
| | | Day 28 | strains (H1N1, H3N2, FluB) | | | |
| | Influenza | Trivalent | Seroconverter for all 3 | case | 13 | |
| | Day 1 F | vaccine | strains (H1N1, H3N2, FluB) | | | |
| | | response at | Not Seroconverter for all 3 | control | 91 | |
| | | Day 28 | strains (H1N1, H3N2, FluB) | | | |
| | Influenza | Trivalent | Seroconverter for all 3 | case | 13 | |
| | Day 14 F | vaccine | strains (H1N1, H3N2, FluB) | | | |
| | | response at | Not Seroconverter for all 3 | control | 82 | |
| | | Day 28 | strains (H1N1, H3N2, FluB) | | | |
| 5 | HBV pre- | Vaccine | Responder | case | 19 | https://www.ncbi.nlm.nih.gov/geo/query/ |
| | vaccine | response | Non responder | control | 14 | acc.cgi?acc=GSE110480 |
| | HBV Day 3 | Vaccine | Responder | case | 19 | |
| | | response | Non responder | control | 14 | |
| | HBV Day 7 | Vaccine | Responder | case | 19 | |
| | | response | Non responder | control | 14 | |
| 6 | TB pre- | Disease state | Max CT score >10 after | control | 14 | https://www.ncbi.nlm.nih.gov/geo/query/ |
| | vaccine | post challenge | vaccination and challenge | | | acc.cgi?acc=GSE102440 |
| | | | Max CT score <10 after | case | 13 | |
| | | | vaccination and challenge | | | |
| | TB pre- | Disease state | Max CT score >10 after | control | 14 | |
| | challenge | post challenge | vaccination and challenge | | | |
| | | | Max CT score <10 after | case | 13 | |
| | | | vaccination and challenge | | | |
| | TB post- | Disease state | Max CT score >10 after | case | 14 | |
| | challenge | post challenge | vaccination and challenge | | | |
| | | | Max CT score <10 after | control | 13 | |
| | | | vaccination and challenge | | | |

TABLE 3
Example test datasets from three studies for evaluating universal signatures

| | | | binary | | | |
|---|---|---|---|---|---|---|
| | Test | | phenotypes | | Number | |
| Test | dataset | Evaluation | used for | | of | |
| Study | Name | metric | evaluation | Label | samples | Source |
| 7 | SARS-CoV- | Severity of | Not severe | Control | 6 | https://www.ncbi.nlm.nih.gov/geo/query/acc.cgi?acc=GSE145926 |
| | 2 | symptoms | Severe | Case | 6 | |
| 8 | Influenza | Severity of | Mechanical | Case | 20 | https://www.ncbi.nlm.nih.gov/geo/query/acc.cgi?acc=GSE111368 |
| | | symptoms | ventilation | | | |
| | | | No | Control | 63 | |
| | | | Mechanical | | | |
| | | | ventilation | | | |
| 9 | TB | Time to | Active | Case | 30 | https://www.ncbi.nlm.nih.gov/geo/query/acc.cgi?acc=GSE79362 |
| | | active TB | tuberculosis | | | |
| | | | within 1 year | | | |
| | | | Latent | Control | 109 | |
| | | | tuberculosis | | | |
| | | | for more | | | |
| | | | than 1 year | | | |
| 10 | Rheumatoid | Rheumatoid | patient | case | 18 | https://www.ncbi.nlm.nih.gov/geo/query/acc.cgi?acc=GSE15573 |
| | Arthritis | Arthritis | healthy | control | 15 | |
| | | status | | | | |
| 11 | Rheumatoid | Response | no response | case | 22 | https://www.ncbi.nlm.nih.gov/geo/query/acc.cgi?acc=GSE15258 |
| | Arthritis | to treatment | response | control | 53 | |
| | | | (high or | | | |
| | | | medium) | | | |
| 12 | Asthma | Loss of | asthma | case | 25 | https://www.ncbi.nlm.nih.gov/geo/query/acc.cgi?acc=GSE19301 |
| | Adults | asthma | exacerbation | | | |
| | | control | no asthma | control | 93 | |
| | | | exacerbation | | | |
| 13 | Asthma | Loss of | asthma | case | 39 | https://www.ncbi.nlm.nih.gov/geo/query/acc.cgi?acc=GSE115823 |
| | Children | asthma | exacerbation | | | |
| | | control | no asthma | control | 63 | |
| | | | exacerbation | | | |
| 14 | TARGET | Time to | death within | case | 10 | https://portal.gdc.cancer.gov/repository |
| | ALLP2 | death | 1 year | | | |
| | | | death in | control | 96 | |
| | | | more than 1 | | | |
| | | | year | | | |
| | TARGET | Time to | death within | case | 14 | |
| | ALLP3 | death | 1 year | | | |
| | | | death in | control | 20 | |
| | | | more than 1 | | | |
| | | | year | | | |
| | TARGET | Time to | death within | case | 18 | |
| | AML | death | 1 year | | | |
| | | | death in | control | 58 | |
| | | | more than 1 | | | |
| | | | year | | | |
| | TARGET | Time to | death within | case | 6 | |
| | OS | death | 1 year | | | |
| | | | death in | control | 23 | |
| | | | more than 1 | | | |
| | | | year | | | |
| | TARGET | Time to | death within | case | 14 | |
| | WT | death | 1 year | | | |
| | | | death in | control | 36 | |
| | | | more than 1 | | | |
| | | | year | | | |
| 15 | TCGA | Time to | death within | case | 76 | |
| | BLCA | death | 1 year | | | |
| | | | death in | control | 102 | |
| | | | more than 1 | | | |
| | | | year | | | |
| | TCGA | Time to | death within | case | 20 | |
| | BRCA | death | 1 year | | | |
| | | | death in | control | 131 | |
| | | | more than 1 | | | |
| | | | year | | | |
| | TCGA | Time to | death within | case | 20 | |
| | CESC | death | 1 year | | | |
| | | | death in | control | 52 | |
| | | | more than 1 | | | |
| | | | year | | | |
| | TCGA | Time to | death within | case | 7 | |
| | CHOL | death | 1 year | | | |
| | | | death in | control | 11 | |
| | | | more than 1 | | | |
| | | | year | | | |
| | TCGA | Time to | death within | case | 50 | |
| | COAD | death | 1 year | | | |
| | | | death in | control | 52 | |
| | | | more than 1 | | | |
| | | | year | | | |
| | TCGA | Time to | death within | case | 30 | |
| | ESCA | death | 1 year | | | |
| | | | death in | control | 37 | |
| | | | more than 1 | | | |
| | | | year | | | |
| | TCGA | Time to | death within | case | 58 | |
| | GBM | death | 1 year | | | |
| | | | death in | control | 71 | |
| | | | more than 1 | | | |
| | | | year | | | |
| | TCGA | Time to | death within | case | 84 | |
| | HNSC | death | 1 year | | | |
| | | | death in | control | 133 | |
| | | | more than 1 | | | |
| | | | year | | | |
| | TCGA | Time to | death within | case | 51 | |
| | KIRC | death | 1 year | | | |
| | | | death in | control | 122 | |
| | | | more than 1 | | | |
| | | | year | | | |
| | TCGA | Time to | death within | case | 12 | |
| | KIRP | death | 1 year | | | |
| | | | death in | control | 32 | |
| | | | more than 1 | | | |
| | | | year | | | |
| | TCGA | Time to | death within | case | 56 | |
| | LAML | death | 1 year | | | |
| | | | death in | control | 31 | |
| | | | more than 1 | | | |
| | | | year | | | |
| | TCGA | Time to | death within | case | 26 | |
| | LGG | death | 1 year | | | |
| | | | death in | control | 99 | |
| | | | more than 1 | | | |
| | | | year | | | |
| | TCGA | Time to | death within | case | 57 | |
| | LIHC | death | 1 year | | | |
| | | | death in | control | 73 | |
| | | | more than 1 | | | |
| | | | year | | | |
| | TCGA | Time to | death within | case | 58 | |
| | LUAD | death | 1 year | | | |
| | | | death in | control | 125 | |
| | | | more than 1 | | | |
| | | | year | | | |
| | TCGA | Time to | death within | case | 74 | |
| | LUSC | death | 1 year | | | |
| | | | death in | control | 138 | |
| | | | more than 1 | | | |
| | | | year | | | |
| | TCGA | Time to | death within | case | 25 | |
| | MESO | death | 1 year | | | |
| | | | death in | control | 47 | |
| | | | more than 1 | | | |
| | | | year | | | |
| | TCGA | Time to | death within | case | 29 | |
| | OV | death | 1 year | | | |
| | | | death in | control | 200 | |
| | | | more than 1 | | | |
| | | | year | | | |
| | TCGA | Time to | death within | case | 40 | |
| | PAAD | death | 1 year | | | |
| | | | death in | control | 52 | |
| | | | more than 1 | | | |
| | | | year | | | |
| | TCGA | Time to | death within | case | 8 | |
| | READ | death | 1 year | | | |
| | | | death in | control | 19 | |
| | | | more than 1 | | | |
| | | | year | | | |
| | TCGA | Time to | death within | case | 27 | |
| | SARC | death | 1 year | | | |
| | | | death in | control | 71 | |
| | | | more than 1 | | | |
| | | | year | | | |
| | TCGA | Time to | death within | case | 26 | |
| | SKCM | death | 1 year | | | |
| | | | death in | control | 194 | |
| | | | more than 1 | | | |
| | | | year | | | |
| | TCGA | Time to | death within | case | 75 | |
| | STAD | death | 1 year | | | |
| | | | death in | control | 71 | |
| | | | more than 1 | | | |
| | | | year | | | |
| | TCGA | Time to | death within | case | 23 | |
| | UCEC | death | 1 year | | | |
| | | | death in | control | 68 | |
| | | | more than 1 | | | |
| | | | year | | | |
| | TCGA | Time to | death within | case | 11 | |
| | UCS | death | 1 year | | | |
| | | | death in | control | 23 | |
| | | | more than 1 | | | |
| | | | year | | | |
| | TCGA | Time to | death within | case | 5 | |
| | UVM | death | 1 year | | | |
| | | | death in | control | 18 | |
| | | | more than 1 | | | |
| | | | year | | | |

TABLE 4
Example literature signatures and corresponding references from which literature signatures are derived

| | | | Number | | |
|---|---|---|---|---|---|
| | | | of | | |
| | | | mapped | | |
| | | | ENSG | | |
| | | | genes in | | |
| Signature | | Study | the | | |
| category | Signature Name | Phenotype | signature | Organism | Reference |
| cell type | Monaco CellRep 2019 | PBMC | 4 | Homo | Monaco, G. et al “RNA-Seq Signatures |
| | B Ex signature | deconvolution | | Sapiens | Normalized by mRNA Abundance Allow |
| cell type | Monaco CellRep 2019 | PBMC | 19 | Homo | Absolute Deconvolution of Human Immune |
| | B NSM signature | deconvolution | | Sapiens | Cell Types.” Cell Reports, 2019, 26(6), |
| cell type | Monaco CellRep 2019 | PBMC | 42 | Homo | 1627-1640. |
| | B Naive signature | deconvolution | | Sapiens | |
| cell type | Monaco CellRep 2019 | PBMC | 21 | Homo | |
| | B SM signature | deconvolution | | Sapiens | |
| cell type | Monaco CellRep 2019 | PBMC | 227 | Homo | |
| | Basophils LD signature | deconvolution | | Sapiens | |
| cell type | Monaco CellRep 2019 | PBMC | 27 | Homo | |
| | MAIT signature | deconvolution | | Sapiens | |
| cell type | Monaco CellRep 2019 | PBMC | 64 | Homo | |
| | Monocytes C signature | deconvolution | | Sapiens | |
| cell type | Monaco CellRep 2019 | PBMC | 17 | Homo | |
| | Monocytes I signature | deconvolution | | Sapiens | |
| cell type | Monaco CellRep 2019 | PBMC | 49 | Homo | |
| | Monocytes NC signature | deconvolution | | Sapiens | |
| cell type | Monaco CellRep 2019 | PBMC | 56 | Homo | |
| | NK signature | deconvolution | | Sapiens | |
| cell type | Monaco CellRep 2019 | PBMC | 262 | Homo | |
| | Neutrophils signature | deconvolution | | Sapiens | |
| cell type | Monaco CellRep 2019 | PBMC | 181 | Homo | |
| | Plasmablasts signature | deconvolution | | Sapiens | |
| cell type | Monaco CellRep 2019 | PBMC | 255 | Homo | |
| | Progenitors signature | deconvolution | | Sapiens | |
| cell type | Monaco CellRep 2019 | PBMC | 7 | Homo | |
| | T CD4 Naive signature | deconvolution | | Sapiens | |
| cell type | Monaco CellRep 2019 | PBMC | 3 | Homo | |
| | T CD8 EM signature | deconvolution | | Sapiens | |
| cell type | Monaco CellRep 2019 | PBMC | 11 | Homo | |
| | T CD8 Naive signature | deconvolution | | Sapiens | |
| cell type | Monaco CellRep 2019 | PBMC | 6 | Homo | |
| | T CD8 TE signature | deconvolution | | Sapiens | |
| cell type | Monaco CellRep 2019 | PBMC | 4 | Homo | |
| | Th17 signature | deconvolution | | Sapiens | |
| cell type | Monaco CellRep 2019 | PBMC | 11 | Homo | |
| | Th2 signature | deconvolution | | Sapiens | |
| cell type | Monaco CellRep 2019 | PBMC | 10 | Homo | |
| | Tregs signature | deconvolution | | Sapiens | |
| cell type | Monaco CellRep 2019 | PBMC | 36 | Homo | |
| | mDCs signature | deconvolution | | Sapiens | |
| cell type | Monaco CellRep 2019 | PBMC | 156 | Homo | |
| | pDCs signature | deconvolution | | Sapiens | |
| hallmark | MSigDB hallmark tnfa | Broad pathway | 201 | Homo | |
| | signaling via nfkb | curation | | Sapiens | |
| hallmark | MSigDB hallmark | Broad pathway | 200 | Homo | |
| | hypoxia | curation | | Sapiens | |
| hallmark | MSigDB hallmark | Broad pathway | 74 | Homo | GSEA Systematic Name: M5892 |
| | cholesterol homeostasis | curation | | Sapiens | |
| hallmark | MSigDB hallmark | Broad pathway | 199 | Homo | GSEA Systematic Name: M5893 |
| | mitotic spindle | curation | | Sapiens | |
| hallmark | MSigDB hallmark wnt | Broad pathway | 42 | Homo | GSEA Systematic Name: M5895 |
| | beta catenin signaling | curation | | Sapiens | |
| hallmark | MSigDB hallmark tgf | Broad pathway | 53 | Homo | GSEA Systematic Name: M5896 |
| | beta signaling | curation | | Sapiens | |
| hallmark | MSigDB hallmark il6 jak | Broad pathway | 86 | Homo | GSEA Systematic Name: M5897 |
| | stat3 signaling | curation | | Sapiens | |
| hallmark | MSigDB hallmark dna | Broad pathway | 150 | Homo | GSEA Systematic Name: M5898 |
| | repair | curation | | Sapiens | |
| hallmark | MSigDB hallmark g2m | Broad pathway | 198 | Homo | GSEA Systematic Name: M5901 |
| | checkpoint | curation | | Sapiens | |
| hallmark | MSigDB hallmark | Broad pathway | 163 | Homo | GSEA Systematic Name: M5902 |
| | apoptosis | curation | | Sapiens | |
| hallmark | MSigDB hallmark notch | Broad pathway | 32 | Homo | GSEA Systematic Name: M5903 |
| | signaling | curation | | Sapiens | |
| hallmark | MSigDB hallmark | Broad pathway | 200 | Homo | GSEA Systematic Name: M5905 |
| | adipogenesis | curation | | Sapiens | |
| hallmark | MSigDB hallmark | Broad pathway | 199 | Homo | GSEA Systematic Name: M5906 |
| | estrogen response | curation | | Sapiens | |
| | early | | | | |
| hallmark | MSigDB hallmark | Broad pathway | 199 | Homo | GSEA Systematic Name: M5907 |
| | estrogen response late | curation | | Sapiens | |
| hallmark | MSigDB hallmark | Broad pathway | 100 | Homo | GSEA Systematic Name: M5908 |
| | androgen response | curation | | Sapiens | |
| hallmark | MSigDB hallmark | Broad pathway | 199 | Homo | GSEA Systematic Name: M5909 |
| | myogenesis | curation | | Sapiens | |
| hallmark | MSigDB hallmark | Broad pathway | 96 | Homo | GSEA Systematic Name: M5910 |
| | protein secretion | curation | | Sapiens | |
| hallmark | MSigDB hallmark | Broad pathway | 97 | Homo | GSEA Systematic Name: M5911 |
| | interferon alpha | curation | | Sapiens | |
| | response | | | | |
| hallmark | MSigDB hallmark | Broad pathway | 201 | Homo | GSEA Systematic Name: M5913 |
| | interferon gamma | curation | | Sapiens | |
| | response | | | | |
| hallmark | MSigDB hallmark | Broad pathway | 200 | Homo | GSEA Systematic Name: M5915 |
| | apical junction | curation | | Sapiens | |
| hallmark | MSigDB hallmark | Broad pathway | 44 | Homo | GSEA Systematic Name: M5916 |
| | apical surface | curation | | Sapiens | |
| hallmark | MSigDB hallmark | Broad pathway | 36 | Homo | GSEA Systematic Name: M5919 |
| | hedgehog signaling | curation | | Sapiens | |
| hallmark | MSigDB hallmark | Broad pathway | 200 | Homo | GSEA Systematic Name: M5921 |
| | complement | curation | | Sapiens | |
| hallmark | MSigDB hallmark | Broad pathway | 113 | Homo | GSEA Systematic Name: M5922 |
| | unfolded protein | curation | | Sapiens | |
| | response | | | | |
| hallmark | MSigDB hallmark pi3k | Broad pathway | 105 | Homo | GSEA Systematic Name: M5923 |
| | akt mtor signaling | curation | | Sapiens | |
| hallmark | MSigDB hallmark | Broad pathway | 200 | Homo | GSEA Systematic Name: M5924 |
| | mtorc1 signaling | curation | | Sapiens | |
| hallmark | MSigDB hallmark e2f | Broad pathway | 200 | Homo | GSEA Systematic Name: M5925 |
| | targets | curation | | Sapiens | |
| hallmark | MSigDB hallmark myc | Broad pathway | 199 | Homo | GSEA Systematic Name: M5926 |
| | targets v1 | curation | | Sapiens | |
| hallmark | MSigDB hallmark myc | Broad pathway | 58 | Homo | GSEA Systematic Name: M5928 |
| | targets v2 | curation | | Sapiens | |
| hallmark | MSigDB hallmark | Broad pathway | 199 | Homo | GSEA Systematic Name: M5930 |
| | epithelial mesenchymal | curation | | Sapiens | |
| | transition | | | | |
| hallmark | MSigDB hallmark | Broad pathway | 200 | Homo | GSEA Systematic Name: M5932 |
| | inflammatory response | curation | | Sapiens | |
| hallmark | MSigDB hallmark | Broad pathway | 200 | Homo | GSEA Systematic Name: M5934 |
| | xenobiotic metabolism | curation | | Sapiens | |
| hallmark | MSigDB hallmark fatty | Broad pathway | 158 | Homo | GSEA Systematic Name: M5935 |
| | acid metabolism | curation | | Sapiens | |
| hallmark | MSigDB hallmark | Broad pathway | 200 | Homo | GSEA Systematic Name: M5936 |
| | oxidative | curation | | Sapiens | |
| | phosphorylation | | | | |
| hallmark | MSigDB hallmark | Broad pathway | 200 | Homo | GSEA Systematic Name: M5937 |
| | glycolysis | curation | | Sapiens | |
| hallmark | MSigDB hallmark | Broad pathway | 50 | Homo | GSEA Systematic Name: M5938 |
| | reactive oxygen | curation | | Sapiens | |
| | species pathway | | | | |
| hallmark | MSigDB hallmark p53 | Broad pathway | 199 | Homo | GSEA Systematic Name: M5939 |
| | pathway | curation | | Sapiens | |
| hallmark | MSigDB hallmark uv | Broad pathway | 159 | Homo | GSEA Systematic Name: M5941 |
| | response up | curation | | Sapiens | |
| hallmark | MSigDB hallmark uv | Broad pathway | 144 | Homo | GSEA Systematic Name: M5942 |
| | response dn | curation | | Sapiens | |
| hallmark | MSigDB hallmark | Broad pathway | 36 | Homo | GSEA Systematic Name: M5944 |
| | angiogenesis | curation | | Sapiens | |
| hallmark | MSigDB hallmark heme | Broad pathway | 200 | Homo | GSEA Systematic Name: M5945 |
| | metabolism | curation | | Sapiens | |
| hallmark | MSigDB hallmark | Broad pathway | 138 | Homo | GSEA Systematic Name: M5946 |
| | coagulation | curation | | Sapiens | |
| hallmark | MSigDB hallmark il2 | Broad pathway | 200 | Homo | GSEA Systematic Name: M5947 |
| | stat5 signaling | curation | | Sapiens | |
| hallmark | MSigDB hallmark bile | Broad pathway | 112 | Homo | GSEA Systematic Name: M5948 |
| | acid metabolism | curation | | Sapiens | |
| hallmark | MSigDB hallmark | Broad pathway | 105 | Homo | GSEA Systematic Name: M5949 |
| | peroxisome | curation | | Sapiens | |
| hallmark | MSigDB hallmark | Broad pathway | 199 | Homo | GSEA Systematic Name: M5950 |
| | allograft rejection | curation | | Sapiens | |
| hallmark | MSigDB hallmark | Broad pathway | 135 | Homo | GSEA Systematic Name: M5951 |
| | spermatogenesis | curation | | Sapiens | |
| hallmark | MSigDB hallmark kras | Broad pathway | 201 | Homo | GSEA Systematic Name: M5953 |
| | signaling up | curation | | Sapiens | |
| hallmark | MSigDB hallmark kras | Broad pathway | 199 | Homo | GSEA Systematic Name: M5956 |
| | signaling dn | curation | | Sapiens | |
| hallmark | MSigDB hallmark | Broad pathway | 40 | Homo | GSEA Systematic Name: M5957 |
| | pancreas beta cells | curation | | Sapiens | |
| TB | Anderson NEJM 2014 a | ATB versus LTBI | 31 | Homo | Anderson, S. et al, “Diagnosis of Childhood |
| | | | | Sapiens | Tuberculosis and Host RNA Expression in |
| TB | Anderson NEJM 2014 b | ATB versus | 37 | Homo | Africa.” N Engl J Med 2014; 370:1712-1723 |
| | | OtherDiseases | | Sapiens | |
| TB | Berry Nature 2010 a | ATB versus LTBI or | 262 | Homo | Berry, M. et al, “An interferon-inducible |
| | | HealthyControls | | Sapiens | neutrophil-driven blood transcriptional |
| | | | | | signature in human tuberculosis.” Nature |
| TB | Berry Nature 2010 b | ATB versus | 65 | Homo | 466, 973-977 (2010) |
| | | OtherDiseases | | Sapiens | |
| TB | Bloom PLoSone 2013 | ATB versus | 103 | Homo | Bloom. C., et al (2013) “Transcriptional |
| | | OtherDiseases or | | Sapiens | Blood Signatures Distinguish Pulmonary |
| | | HealthyControls | | | Tuberculosis, Pulmonary Sarcoidosis, |
| | | | | | Pneumonias and Lung Cancers.” PLoS |
| | | | | | ONE 8(8): e70630. |
| TB | Jacobsen JMolMed | ATB versus LTBI or | 3 | Homo | Jacobsen, M., Repsilber, D., Gutschmidt, A. |
| | 2007 | HealthyControls | | Sapiens | et al. Candidate biomarkers for |
| | | | | | discrimination between infection and |
| | | | | | disease caused by Mycobacterium |
| | | | | | tuberculosis . J Mol Med 85, 613-621 |
| | | | | | (2007). |
| TB | Kaforou PLoSMed | ATB versus LTBI | 22 | Homo | Kaforou M, Wright V J, Oni T, French N, |
| | 2013 a | | | Sapiens | Anderson S T, Bangani N, et al. (2013) |
| TB | Kaforou PLoSMed | ATB versus LTBI | 42 | Homo | Detection of Tuberculosis in HIV-Infected |
| | 2013 b | or OtherDiseases | | Sapiens | and -Uninfected African Adults Using Whole |
| TB | Kaforou PLoSMed | ATB versus | 31 | Homo | Blood RNA Expression Signatures: A Case- |
| | 2013 c | OtherDiseases | | Sapiens | Control Study. PLoS Med 10(10): |
| TB | Leong Tuberculosis | ATB versus LTBI | 24 | Homo | Leong. S., et al “Existing blood |
| | 2018 a | | | Sapiens | transcriptional classifiers accurately |
| TB | Leong Tuberculosis | ATB versus LTBI | 76 | Homo | discriminate active tuberculosis from latent |
| | 2018 b | | | Sapiens | infection in individuals from south India.” |
| | | | | | Tuberculosis (2018), 109, 41-51. |
| TB | Maertzdorf | ATB versus LTBI or | 12 | Homo | Maertzdorf, J. et al “Concise gene signature |
| | EMBOMolMed 2016 a | HealthyControls | | Sapiens | for point-of-care classification of |
| TB | Maertzdorf | ATB versus LTBI or | 4 | Homo | tuberculosis.” EMBO Mol Med (2016) 8: 86- |
| | EMBOMolMed 2016 b | HealthyControls | | Sapiens | 95. |
| TB | Sambarey | ATB versus LTBI or | 10 | Homo | Samberey, A. et al “Unbiased Identification |
| | EBioMedicine 2017 | HealthyControls or | | Sapiens | of Blood-based Biomarkers for Pulmonary |
| | | OtherDiseases | | | Tuberculosis by Modeling and Mining |
| | | | | | Molecular Interaction Networks.” |
| | | | | | EBioMedicine, 2017, 15, 112-126. |
| TB | Suliman | progression risk | 4 | Homo | Suliman, S. et al “Four-Gene Pan-African |
| | AmJRespCritCareMed | | | Sapiens | Blood Signature Predicts Progression to |
| | 2018 a | | | | Tuberculosis.” Am. Journal of Respiratory |
| TB | Suliman | progression risk | 47 | Homo | and Critical Care Medicine, 2018, 197(9), |
| | AmJRespCritCareMed | | | Sapiens | 1198-1208. |
| | 2018 b | | | | |
| TB | Sweeney | ATB versus LTBI or | 3 | Homo | Sweeney, T. et al “Genome-wide |
| | LancetRespMed 2018 | HealthyControls or | | Sapiens | expression for diagnosis of pulmonary |
| | | OtherDiseases | | | tuberculosis: a multicohort analysis.” Lancet |
| | | | | | Respiratory Medicine, (2016), 4(3), 213- |
| | | | | | 224. |
| TB | Verhagen | ATB versus LTBI or | 10 | Homo | Verhagen, L. M., Zomer, A., Maes, M. et al. |
| | BMCGenomics 2013 | HealthyControls | | Sapiens | A predictive signature gene set for |
| | | | | | discriminating active from latent |
| | | | | | tuberculosis in Warao Amerindian children. |
| | | | | | BMC Genomics 14, 74 (2013). |
| TB | Zak Lancet 2016 | progression risk | 16 | Homo | Zak, D. et al “A blood RNA signature for |
| | | | | Sapiens | tuberculosis disease risk: a prospective |
| | | | | | cohort study.” The Lancet (2016), |
| | | | | | 387(10035), 2312-2322. |
| TB | daCosta Tuberculosis | ATB versus | 3 | Homo | da Costa, L. et al “A real-time PCR |
| | 2015 | OtherDiseases | | Sapiens | signature to discriminate between |
| | | | | | tuberculosis and other pulmonary |
| | | | | | diseases.” Tuberculosis (2015), 95(4), 421- |
| | | | | | 425. |
| vaccine | Ehrenberg | SIV vaccine | 53 | Rhesus | Ehrenberg, P., et al “A vaccine-induced |
| | SciTransMed 2019 | protection | | Macaque | gene expression signature correlates with |
| | | | | | protection against SIV and HIV in multiple |
| | | | | | trials.” Science Translational Medicine |
| | | | | | (2019), 11(507). |
| vaccine | Hansen NatMed 2018 a | post challenge | 209 | Rhesus | Hansen, S., Zak, D., Xu, G. et al. |
| | | expression versus | | Macaque | Prevention of tuberculosis in rhesus |
| | | vaccine response - | | | macaques by a cytomegalovirus-based |
| | | disease | | | vaccine. Nat Med 24, 130-143 (2018). |
| | | signature | | | |
| vaccine | Hansen NatMed 2018 b | pre challenge | 248 | Rhesus | |
| | | expression versus | | Macaque | |
| | | vaccine response - | | | |
| | | protection | | | |
| | | signature | | | |
| vaccine | Hansen NatMed 2018 c | pre vaccine | 77 | Rhesus | |
| | | expression versus | | Macaque | |
| | | vaccine response - | | | |
| | | baseline | | | |
| | | signature | | | |
| vaccine | Bartholomeus Vaccine | HBV vaccine | 22 | Homo | Bartholomeus, E. et al “Transcriptome |
| | 2018 | response | | Sapiens | profiling in blood before and after hepatitis B |
| | | | | | vaccination shows significant differences in |
| | | | | | gene expression between responders and |
| | | | | | non-responders.” Vaccine (2018), 36(42), |
| | | | | | 6282-6289. |
| vaccine | Franco eLife 2013 a | trivalent influenza | 226 | Homo | Franco, L. et al “Integrative genomic |
| | | vaccine response | | Sapiens | analysis of the human immune response to |
| vaccine | Franco eLife 2013 b | trivalent influenza | 20 | Homo | influenza vaccination.” eLife. 2013; |
| | | vaccine immune | | Sapiens | 2:e00299. |
| | | response | | | |
| | | strongest genetic | | | |
| | | association | | | |
| vaccine | Franco eLife 2013 c | trivalent influenza | 28 | Homo | |
| | | vaccine response | | Sapiens | |
| | | Day 0 | | | |
| vaccine | Franco eLife 2013 d | trivalent influenza | 140 | Homo | |
| | | vaccine response | | Sapiens | |
| | | Day 1 | | | |
| vaccine | Franco eLife 2013 e | trivalent influenza | 18 | Homo | |
| | | vaccine response | | Sapiens | |
| | | Day 3 | | | |
| vaccine | Franco eLife 2013 f | trivalent influenza | 41 | Homo | |
| | | vaccine response | | Sapiens | |
| | | Day 14 | | | |
| vaccine | Tsang Cell 2014 a | Day 0 predictive | 61 | Homo | Tsang, J., et al “Global Analyses of Human |
| | | cell subset | | Sapiens | Immune Variation Reveal Baseline |
| | | signature | | | Predictors of Postvaccination Responses.” |
| vaccine | Tsang Cell 2014 b | Day 7 predictive | 100 | Homo | Cell (2014), 157(2), 499-513. |
| | | signature for | | Sapiens | |
| | | vaccine response | | | |
| infection | BermejoMartin | mechanical | 143 | Homo | Bermejo-Martin, J. F., Martin-Loeches, I., |
| | CriticCare 2010 | ventilation after | | Sapiens | Rello, J. et al. Host adaptive immunity |
| | | H1N1 infection | | | deficiency in severe pandemic influenza. |
| infection | Cameron JVirol 2007 a | SARS crisis | 31 | Homo | Crit Care 14, R167 (2010). |
| | | | | Sapiens | https://doi.org/10.1186/cc9259 |
| infection | Cameron JVirol 2007 b | SARS disease | 37 | Homo | Muramoto, Y. et al “Disease Severity Is |
| | | course | | Sapiens | Associated with Differential Gene |
| infection | Cameron JVirol 2007 c | SARS union crisis | 54 | Homo | Expression at the Early and Late Phases of |
| | | and disease | | Sapiens | Infection in Nonhuman Primates Infected |
| | | | | | with Different H5N1 Highly Pathogenic |
| | | | | | Avian Influenza Viruses.” Journal of |
| | | | | | Virology Jul 2014, 88 (16) 8981-8997. |
| infection | Muramoto JVirol 2014 | H5N1 | 159 | Cynomolgus | Cameron, M. et al “Interferon-Mediated |
| | a | pathogenicity ISG | | Macaque | Immunopathological Events Are Associated |
| | | subset | | | with Atypical Innate and Adaptive Immune |
| infection | Muramoto JVirol 2014 | H5N1 | 218 | Cynomolgus | Responses in Patients with Severe Acute |
| | b | pathogenicity | | Macaque | Respiratory Syndrome.” Journal of Virology |
| | | | | | Jul 2007, 81 (16) 8692-8706. |
| infection | Devignot PLoSone | Dengue | 257 | Homo | Devignot S, Sapet C, Duong V, Bergon A, |
| | 2010 | associated Shock | | Sapiens | Rihet P, Ong S, et al. (2010) Genome-Wide |
| | | Syndrome | | | Expression Profiling Deciphers Host |
| | | | | | Responses Altered during Dengue Shock |
| | | | | | Syndrome and Reveals the Role of Innate |
| | | | | | Immunity in Severe Dengue. PLoS ONE |
| | | | | | 5(7): e11671. |
| infection | Zilliox ClinVaccIm 2007 | Measles pre and | 171 | Homo | Zilliox, M. et al “Gene Expression Changes |
| | | post infection | | Sapiens | in Peripheral Blood Mononuclear Cells |
| | | DEG | | | during Measles Virus Infection.” Clinical and |
| | | | | | Vaccine Immunology Jul 2007, 14 (7) 918- |
| | | | | | 923. |
| infection | Islam Preprint 2020 | SARSCov2 post | 298 | Homo | Islam, M. R.; Fischer, A. A Transcriptome |
| | | mortem DEG | | Sapiens | Analysis Identifies Potential Preventive and |
| infection | Islam Preprint 2020 a | inflammatory | 391 | Human Cell | Therapeutic Approaches Towards COVID- |
| | | signal from | | Lines | 19. Preprints 2020, 2020040399 |
| | | lightcyan module | | | |
| | | associated with | | | |
| | | multiple viruses | | | |
| infection | Islam Preprint 2020 b | inflammatory | 403 | Human Cell | |
| | | signal from | | Lines | |
| | | midnightblue | | | |
| | | module | | | |
| | | associated with | | | |
| | | multiple viruses | | | |
| infection | Wen CellDiscovery | AntibodySecreting | 21 | Homo | Wen, W. Su, W. Tang, H. et al. Immune |
| | 2020 a | Cells DEG in | | Sapiens | cell profiling of COVID-19 patients in the |
| | | SARS-CoV-2 | | | recovery stage by single-cell sequencing. |
| | | infection | | | Cell Discov 6, 31 (2020). |
| infection | Wen CellDiscovery | B cells DEG in | 59 | Homo | |
| | 2020 b | SARS-CoV-2 | | Sapiens | |
| | | infection | | | |
| infection | Wen CellDiscovery | CD14 monocytes | 43 | Homo | |
| | 2020 c | DEG in SARS- | | Sapiens | |
| | | CoV-2 infection | | | |
| infection | Wen CellDiscovery | CD4 Tcells DEG | 35 | Homo | |
| | 2020 d | in SARS-CoV-2 | | Sapiens | |
| | | infection | | | |
| infection | Wen CellDiscovery | Dentritic Cells | 46 | Homo | |
| | 2020 e | DEG in SARS- | | Sapiens | |
| | | CoV-2 infection | | | |
| infection | Wen CellDiscovery | Myeloid Cells | 87 | Homo | |
| | 2020 f | DEG in SARS- | | Sapiens | |
| | | CoV-2 infection | | | |
| infection | Wen CellDiscovery | NK and Tcell | 60 | Homo | |
| | 2020 g | DEG in SARS- | | Sapiens | |
| | | CoV-2 infection | | | |
| infection | Wen CellDiscovery | union DEG in | 178 | Homo | |
| | 2020 h | SARS-CoV-2 | | Sapiens | |
| | | infection | | | |
| infection | Hubel NatIm 2019 | ISGs | 103 | Homo | Hubel, P. Urban, C., Bergant, V. et al. A |
| | | | | Sapiens | protein-interaction network of interferon- |
| | | | | | stimulated genes extends the innate |
| | | | | | immune system landscape. Nat Immunol |
| | | | | | 20, 493-502 (2019). |
| infection | Mayhew NatComm | infection | 29 | Homo | Mayhew, M. B., Buturovic, L., Luethy, R. et |
| | 2020 | | | Sapiens | al. A generalizable 29-mRNA neural- |
| | | | | | network classifier for acute bacterial and |
| | | | | | viral infections. Nat Commun 11, 1177 |
| | | | | | (2020). |
| infection | Dunning NatImm 2018 | healthy control | 22 | Homo | Dunning, J., Blankley, S., Hoang, L. T. et al. |
| | a | versus influenza | | Sapiens | Progression of whole-blood transcriptional |
| infection | Dunning NatImm 2018 | influenza (H1N1 | 37 | Homo | signatures from interferon-induced to |
| | b | or H3N2) severity - | | Sapiens | neutrophil-associated patterns in severe |
| | | GO viral | | | influenza. Nat Immunol 19, 625-635 (2018). |
| | | response | | | |
| infection | Dunning NatImm 2018 | influenza (H1N1 | 78 | Homo | |
| | C | or H3N2) severity - | | Sapiens | |
| | | GO bacteria | | | |
| | | response | | | |
| infection | Liao NatMed 2020 a | SARSCoV2 BALF | 27 | Homo | Liao, M., Liu, Y., Yuan, J. et al. Single-cell |
| | | DEGs | | Sapiens | landscape of bronchoalveolar immune cells |
| | | macrophage | | | in patients with COVID-19. Nat Med 26, |
| | | group 1 | | | 842-844 (2020). |
| infection | Liao NatMed 2020 b | SARSCoV2 BALF | 53 | Homo | |
| | | DEGs | | Sapiens | |
| | | macrophage | | | |
| | | group 2 | | | |
| infection | Liao NatMed 2020 c | SARSCoV2 BALF | 40 | Homo | |
| | | DEGs | | Sapiens | |
| | | macrophage | | | |
| | | group 3 | | | |
| infection | Liao NatMed 2020 d | SARSCoV2 BALF | 21 | Homo | |
| | | DEGs | | Sapiens | |
| | | macrophage | | | |
| | | group 4 | | | |
| infection | Liao NatMed 2020 e | SARSCoV2 BALF | 38 | Homo | |
| | | DEGs CCR7 T | | Sapiens | |
| | | cells | | | |
| infection | Liao NatMed 2020 f | SARSCoV2 BALF | 24 | Homo | |
| | | DEGs CD8 T cells | | Sapiens | |
| infection | Liao NatMed 2020 g | SARSCoV2 BALF | 34 | Homo | |
| | | DEGs NK cells | | Sapiens | |
| infection | Liao NatMed 2020 h | SARSCoV2 BALF | 28 | Homo | |
| | | DEGs prolif T | | Sapiens | |
| | | cells | | | |
| infection | Liao NatMed 2020 i | SARSCoV2 BALF | 23 | Homo | |
| | | DEGs Treg | | Sapiens | |
| infection | Liao NatMed 2020 j | SARSCoV2 BALF | 30 | Homo | |
| | | DEGs innate T | | Sapiens | |
| | | cells | | | |
| infection | BlancoMelo Cell 2020 a | DEG IAV in A549 | 94 | Homo | Blanco-Melo, D. et al “Imbalanced Host |
| | | cells | | Sapiens | Response to SARS-CoV-2 Drivers |
| infection | BlancoMelo Cell 2020 b | DEG MERSCoV | 92 | Homo | Development of COVID-19.” Cell (2020), |
| | | in MRC5 cells | | Sapiens | 181(5), 1036-1045. |
| infection | BlancoMelo Cell 2020 c | DEG RSVin A549 | 101 | Homo | |
| | | cells | | Sapiens | |
| infection | BlancoMelo Cell 2020 d | DEG SARSCoV1 | 97 | Homo | |
| | | in MRC5 cells | | Sapiens | |
| infection | BlancoMelo Cell 2020 e | DEG SARSCoV2 | 95 | Homo | |
| | | in A549-ACE2 | | Sapiens | |
| | | cells | | | |
| infection | BlancoMelo Cell 2020 f | DEG SARSCoV2 | 216 | Homo | |
| | | in BALF | | Sapiens | |
| infection | BlancoMelo Cell 2020 g | DEG NHBE cells | 118 | Homo | |
| | | | | Sapiens | |
| infection | Xiong EmergMicrobInf | DEG in | 100 | Homo | Xiong, Y. et al “Transcriptomic |
| | 2020 a | SARSCoV2 BALF | | Sapiens | characteristics of bronchoalveolar lavage |
| | | | | | fluid and peripheral blood mononuclear cells |
| | | | | | in COVID-19 patients.” Emerging Microbes |
| | | | | | and Infections (2020), 9(1), 761-770. |
| infection | Xiong EmergMicrobInf | DEG in | 205 | Homo | Monaco, G. et al “RNA-Seq Signatures |
| | 2020 b | SARSCoV2 | | Sapiens | Normalized by mRNA Abundance Allow |
| | | PBMC | | | Absolute Deconvolution of Human Immune |
| | | | | | Cell Types.” Cell Reports, 2019, 26(6), |
| | | | | | 1627-1640. |

TABLE 5
Example sets of universal/transfer signatures. Here,
a set of universal signatures includes 50 genes.

| Gene | | | Training subdataset |
|---|---|---|---|
| Rank | ENSG | Gene name | Name |
| 1 | ENSG00000102900 | NUP93 | TB pre-vaccine |
| 2 | ENSG00000115241 | PPM1G | TB pre-vaccine |
| 3 | ENSG00000112308 | C6orf62 | TB pre-vaccine |
| 4 | ENSG00000181191 | PJA1 | TB pre-vaccine |
| 5 | ENSG00000106484 | MEST | TB pre-vaccine |
| 6 | ENSG00000158864 | NDUFS2 | TB pre-vaccine |
| 7 | ENSG00000244038 | DDOST | TB pre-vaccine |
| 8 | ENSG00000109016 | DHRS7B | TB pre-vaccine |
| 9 | ENSG00000166197 | NOLC1 | TB pre-vaccine |
| 10 | ENSG00000014138 | POLA2 | TB pre-vaccine |
| 11 | ENSG00000150687 | PRSS23 | TB pre-vaccine |
| 12 | ENSG00000176974 | SHMT1 | TB pre-vaccine |
| 13 | ENSG00000137275 | RIPK1 | TB pre-vaccine |
| 14 | ENSG00000117448 | AKR1A1 | TB pre-vaccine |
| 15 | ENSG00000117360 | PRPF3 | TB pre-vaccine |
| 16 | ENSG00000134954 | ETS1 | TB pre-vaccine |
| 17 | ENSG00000111261 | MANSC1 | TB pre-vaccine |
| 18 | ENSG00000131828 | PDHA1 | TB pre-vaccine |
| 19 | ENSG00000131473 | ACLY | TB pre-vaccine |
| 20 | ENSG00000064886 | CHI3L2 | TB pre-vaccine |
| 21 | ENSG00000166508 | MCM7 | TB pre-vaccine |
| 22 | ENSG00000170464 | DNAJC18 | TB pre-vaccine |
| 23 | ENSG00000115850 | LCT | TB pre-vaccine |
| 24 | ENSG00000196449 | YRDC | TB pre-vaccine |
| 25 | ENSG00000156709 | AIFM1 | TB pre-vaccine |
| 26 | ENSG00000175793 | SFN | TB pre-vaccine |
| 27 | ENSG00000166147 | FBN1 | TB pre-vaccine |
| 28 | ENSG00000106682 | EIF4H | TB pre-vaccine |
| 29 | ENSG00000111729 | CLEC4A | TB pre-vaccine |
| 30 | ENSG00000185825 | BCAP31 | TB pre-vaccine |
| 31 | ENSG00000168397 | ATG4B | TB pre-vaccine |
| 32 | ENSG00000159176 | CSRP1 | TB pre-vaccine |
| 33 | ENSG00000072042 | RDH11 | TB pre-vaccine |
| 34 | ENSG00000023909 | GCLM | TB pre-vaccine |
| 35 | ENSG00000097046 | CDC7 | TB pre-vaccine |
| 36 | ENSG00000171433 | GLOD5 | TB pre-vaccine |
| 37 | ENSG00000182054 | IDH2 | TB pre-vaccine |
| 38 | ENSG00000102081 | FMR1 | TB pre-vaccine |
| 39 | ENSG00000186951 | PPARA | TB pre-vaccine |
| 40 | ENSG00000105173 | CCNE1 | TB pre-vaccine |
| 41 | ENSG00000167986 | DDB1 | TB pre-vaccine |
| 42 | ENSG00000168487 | BMP1 | TB pre-vaccine |
| 43 | ENSG00000103966 | EHD4 | TB pre-vaccine |
| 44 | ENSG00000134215 | VAV3 | TB pre-vaccine |
| 45 | ENSG00000103152 | MPG | TB pre-vaccine |
| 46 | ENSG00000061656 | SPAG4 | TB pre-vaccine |
| 47 | ENSG00000108344 | PSMD3 | TB pre-vaccine |
| 48 | ENSG00000248098 | BCKDHA | TB pre-vaccine |
| 49 | ENSG00000023171 | GRAMD1B | TB pre-vaccine |
| 50 | ENSG00000058262 | SEC61A1 | TB pre-vaccine |
| 1 | ENSG00000130545 | CRB3 | TB pre-challenge |
| 2 | ENSG00000185825 | BCAP31 | TB pre-challenge |
| 3 | ENSG00000173540 | GMPPB | TB pre-challenge |
| 4 | ENSG00000010610 | CD4 | TB pre-challenge |
| 5 | ENSG00000131748 | STARD3 | TB pre-challenge |
| 6 | ENSG00000179218 | CALR | TB pre-challenge |
| 7 | ENSG00000159176 | CSRP1 | TB pre-challenge |
| 8 | ENSG00000110090 | CPT1A | TB pre-challenge |
| 9 | ENSG00000157978 | LDLRAP1 | TB pre-challenge |
| 10 | ENSG00000126458 | RRAS | TB pre-challenge |
| 11 | ENSG00000113161 | HMGCR | TB pre-challenge |
| 12 | ENSG00000068831 | RASGRP2 | TB pre-challenge |
| 13 | ENSG00000150787 | PTS | TB pre-challenge |
| 14 | ENSG00000140263 | SORD | TB pre-challenge |
| 15 | ENSG00000225697 | SLC26A6 | TB pre-challenge |
| 16 | ENSG00000108828 | VAT1 | TB pre-challenge |
| 17 | ENSG00000197858 | GPAA1 | TB pre-challenge |
| 18 | ENSG00000186810 | CXCR3 | TB pre-challenge |
| 19 | ENSG00000105835 | NAMPT | TB pre-challenge |
| 20 | ENSG00000143819 | EPHX1 | TB pre-challenge |
| 21 | ENSG00000184640 | SEPT9 | TB pre-challenge |
| 22 | ENSG00000144591 | GMPPA | TB pre-challenge |
| 23 | ENSG00000027847 | B4GALT7 | TB pre-challenge |
| 24 | ENSG00000094914 | AAAS | TB pre-challenge |
| 25 | ENSG00000164938 | TP53INP1 | TB pre-challenge |
| 26 | ENSG00000104812 | GYS1 | TB pre-challenge |
| 27 | ENSG00000169710 | FASN | TB pre-challenge |
| 28 | ENSG00000184967 | NOC4L | TB pre-challenge |
| 29 | ENSG00000114767 | RRP9 | TB pre-challenge |
| 30 | ENSG00000119950 | MXI1 | TB pre-challenge |
| 31 | ENSG00000141510 | TP53 | TB pre-challenge |
| 32 | ENSG00000151012 | SLC7A11 | TB pre-challenge |
| 33 | ENSG00000049768 | FOXP3 | TB pre-challenge |
| 34 | ENSG00000013563 | DNASE1L1 | TB pre-challenge |
| 35 | ENSG00000131446 | MGAT1 | TB pre-challenge |
| 36 | ENSG00000058262 | SEC61A1 | TB pre-challenge |
| 37 | ENSG00000163820 | FYCO1 | TB pre-challenge |
| 38 | ENSG00000197747 | S100A10 | TB pre-challenge |
| 39 | ENSG00000160285 | LSS | TB pre-challenge |
| 40 | ENSG00000006652 | IFRD1 | TB pre-challenge |
| 41 | ENSG00000172795 | DCP2 | TB pre-challenge |
| 42 | ENSG00000038358 | EDC4 | TB pre-challenge |
| 43 | ENSG00000163516 | ANKZF1 | TB pre-challenge |
| 44 | ENSG00000127415 | IDUA | TB pre-challenge |
| 45 | ENSG00000115457 | IGFBP2 | TB pre-challenge |
| 46 | ENSG00000123136 | DDX39A | TB pre-challenge |
| 47 | ENSG00000154277 | UCHL1 | TB pre-challenge |
| 48 | ENSG00000123358 | NR4A1 | TB pre-challenge |
| 49 | ENSG00000065485 | PDIA5 | TB pre-challenge |
| 50 | ENSG00000167280 | ENGASE | TB pre-challenge |
| 1 | ENSG00000013374 | NUB1 | TB post-challenge |
| 2 | ENSG00000137752 | CASP1 | TB post-challenge |
| 3 | ENSG00000140105 | WARS | TB post-challenge |
| 4 | ENSG00000132109 | TRIM21 | TB post-challenge |
| 5 | ENSG00000115415 | STAT1 | TB post-challenge |
| 6 | ENSG00000075643 | MOCOS | TB post-challenge |
| 7 | ENSG00000121380 | BCL2L14 | TB post-challenge |
| 8 | ENSG00000162772 | ATF3 | TB post-challenge |
| 9 | ENSG00000068796 | KIF2A | TB post-challenge |
| 10 | ENSG00000197646 | PDCD1LG2 | TB post-challenge |
| 11 | ENSG00000086300 | SNX10 | TB post-challenge |
| 12 | ENSG00000150961 | SEC24D | TB post-challenge |
| 13 | ENSG00000156587 | UBE2L6 | TB post-challenge |
| 14 | ENSG00000166796 | LDHC | TB post-challenge |
| 15 | ENSG00000026103 | FAS | TB post-challenge |
| 16 | ENSG00000169245 | CXCL10 | TB post-challenge |
| 17 | ENSG00000170581 | STAT2 | TB post-challenge |
| 18 | ENSG00000185507 | IRF7 | TB post-challenge |
| 19 | ENSG00000120217 | CD274 | TB post-challenge |
| 20 | ENSG00000100911 | PSME2 | TB post-challenge |
| 21 | ENSG00000087253 | LPCAT2 | TB post-challenge |
| 22 | ENSG00000204264 | PSMB8 | TB post-challenge |
| 23 | ENSG00000116663 | FBX06 | TB post-challenge |
| 24 | ENSG00000143507 | DUSP10 | TB post-challenge |
| 25 | ENSG00000105499 | PLA2G4C | TB post-challenge |
| 26 | ENSG00000175334 | BANF1 | TB post-challenge |
| 27 | ENSG00000187266 | EPOR | TB post-challenge |
| 28 | ENSG00000156113 | KCNMA1 | TB post-challenge |
| 29 | ENSG00000143387 | CTSK | TB post-challenge |
| 30 | ENSG00000164171 | ITGA2 | TB post-challenge |
| 31 | ENSG00000149573 | MPZL2 | TB post-challenge |
| 32 | ENSG00000149557 | FEZ1 | TB post-challenge |
| 33 | ENSG00000096968 | JAK2 | TB post-challenge |
| 34 | ENSG00000198604 | BAZ1A | TB post-challenge |
| 35 | ENSG00000105371 | ICAM4 | TB post-challenge |
| 36 | ENSG00000070190 | DAPP1 | TB post-challenge |
| 37 | ENSG00000137275 | RIPK1 | TB post-challenge |
| 38 | ENSG00000137393 | RNF144B | TB post-challenge |
| 39 | ENSG00000002549 | LAP3 | TB post-challenge |
| 40 | ENSG00000173372 | C1QA | TB post-challenge |
| 41 | ENSG00000025708 | TYMP | TB post-challenge |
| 42 | ENSG00000131979 | GCH1 | TB post-challenge |
| 43 | ENSG00000173369 | C1QB | TB post-challenge |
| 44 | ENSG00000095794 | CREM | TB post-challenge |
| 45 | ENSG00000010030 | ETV7 | TB post-challenge |
| 46 | ENSG00000125740 | FOSB | TB post-challenge |
| 47 | ENSG00000137547 | MRPL15 | TB post-challenge |
| 48 | ENSG00000080815 | PSEN1 | TB post-challenge |
| 49 | ENSG00000119950 | MXI1 | TB post-challenge |
| 50 | ENSG00000135148 | TRAFD1 | TB post-challenge |
| 1 | ENSG00000154099 | DNAAF1 | HBV pre-vaccine |
| 2 | ENSG00000140740 | UQCRC2 | HBV pre-vaccine |
| 3 | ENSG00000108039 | XPNPEP1 | HBV pre-vaccine |
| 4 | ENSG00000166743 | ACSM1 | HBV pre-vaccine |
| 5 | ENSG00000137628 | DDX60 | HBV pre-vaccine |
| 6 | ENSG00000111669 | TPI1 | HBV pre-vaccine |
| 7 | ENSG00000143590 | EFNA3 | HBV pre-vaccine |
| 8 | ENSG00000163958 | ZDHHC19 | HBV pre-vaccine |
| 9 | ENSG00000175197 | DDIT3 | HBV pre-vaccine |
| 10 | ENSG00000108176 | DNAJC12 | HBV pre-vaccine |
| 11 | ENSG00000165731 | RET | HBV pre-vaccine |
| 12 | ENSG00000174564 | IL20RB | HBV pre-vaccine |
| 13 | ENSG00000121858 | TNFSF10 | HBV pre-vaccine |
| 14 | ENSG00000132535 | DLG4 | HBV pre-vaccine |
| 15 | ENSG00000136026 | CKAP4 | HBV pre-vaccine |
| 16 | ENSG00000070614 | NDST1 | HBV pre-vaccine |
| 17 | ENSG00000111640 | GAPDH | HBV pre-vaccine |
| 18 | ENSG00000138175 | ARL3 | HBV pre-vaccine |
| 19 | ENSG00000122194 | PLG | HBV pre-vaccine |
| 20 | ENSG00000146701 | MDH2 | HBV pre-vaccine |
| 21 | ENSG00000084207 | GSTP1 | HBV pre-vaccine |
| 22 | ENSG00000163220 | S100A9 | HBV pre-vaccine |
| 23 | ENSG00000027847 | B4GALT7 | HBV pre-vaccine |
| 24 | ENSG00000246705 | H2AFJ | HBV pre-vaccine |
| 25 | ENSG00000213903 | LTB4R | HBV pre-vaccine |
| 26 | ENSG00000158710 | TAGLN2 | HBV pre-vaccine |
| 27 | ENSG00000185507 | IRF7 | HBV pre-vaccine |
| 28 | ENSG00000167792 | NDUFV1 | HBV pre-vaccine |
| 29 | ENSG00000178789 | CD300LB | HBV pre-vaccine |
| 30 | ENSG00000136514 | RTP4 | HBV pre-vaccine |
| 31 | ENSG00000117984 | CTSD | HBV pre-vaccine |
| 32 | ENSG00000273802 | HIST1H2BG | HBV pre-vaccine |
| 33 | ENSG00000197272 | IL27 | HBV pre-vaccine |
| 34 | ENSG00000028137 | TNFRSF1B | HBV pre-vaccine |
| 35 | ENSG00000095637 | SORBS1 | HBV pre-vaccine |
| 36 | ENSG00000111641 | NOP2 | HBV pre-vaccine |
| 37 | ENSG00000102524 | TNFSF13B | HBV pre-vaccine |
| 38 | ENSG00000198502 | HLA-DRB5 | HBV pre-vaccine |
| 39 | ENSG00000177105 | RHOG | HBV pre-vaccine |
| 40 | ENSG00000240065 | PSMB9 | HBV pre-vaccine |
| 41 | ENSG00000173110 | HSPA6 | HBV pre-vaccine |
| 42 | ENSG00000135404 | CD63 | HBV pre-vaccine |
| 43 | ENSG00000136856 | SLC2A8 | HBV pre-vaccine |
| 44 | ENSG00000185885 | IFITM1 | HBV pre-vaccine |
| 45 | ENSG00000166165 | CKB | HBV pre-vaccine |
| 46 | ENSG00000149925 | ALDOA | HBV pre-vaccine |
| 47 | ENSG00000198736 | MSRB1 | HBV pre-vaccine |
| 48 | ENSG00000145623 | OSMR | HBV pre-vaccine |
| 49 | ENSG00000175550 | DRAP1 | HBV pre-vaccine |
| 50 | ENSG00000116711 | PLA2G4A | HBV pre-vaccine |
| 1 | ENSG00000168904 | LRRC28 | HBV Day 3 |
| 2 | ENSG00000205250 | E2F4 | HBV Day 3 |
| 3 | ENSG00000137547 | MRPL15 | HBV Day 3 |
| 4 | ENSG00000102962 | CCL22 | HBV Day 3 |
| 5 | ENSG00000165312 | OTUD1 | HBV Day 3 |
| 6 | ENSG00000179299 | NSUN7 | HBV Day 3 |
| 7 | ENSG00000149554 | CHEK1 | HBV Day 3 |
| 8 | ENSG00000020181 | ADGRA2 | HBV Day 3 |
| 9 | ENSG00000169946 | ZFPM2 | HBV Day 3 |
| 10 | ENSG00000111713 | GYS2 | HBV Day 3 |
| 11 | ENSG00000177697 | CD151 | HBV Day 3 |
| 12 | ENSG00000108384 | RAD51C | HBV Day 3 |
| 13 | ENSG00000116584 | ARHGEF2 | HBV Day 3 |
| 14 | ENSG00000108518 | PFN1 | HBV Day 3 |
| 15 | ENSG00000134262 | AP4B1 | HBV Day 3 |
| 16 | ENSG00000141753 | IGFBP4 | HBV Day 3 |
| 17 | ENSG00000135114 | OASL | HBV Day 3 |
| 18 | ENSG00000145431 | PDGFC | HBV Day 3 |
| 19 | ENSG00000141741 | MIEN1 | HBV Day 3 |
| 20 | ENSG00000127325 | BEST3 | HBV Day 3 |
| 21 | ENSG00000154447 | SH3RF1 | HBV Day 3 |
| 22 | ENSG00000161800 | RACGAP1 | HBV Day 3 |
| 23 | ENSG00000007933 | FMO3 | HBV Day 3 |
| 24 | ENSG00000122566 | HNRNPA2B1 | HBV Day 3 |
| 25 | ENSG00000164251 | F2RL1 | HBV Day 3 |
| 26 | ENSG00000110931 | CAMKK2 | HBV Day 3 |
| 27 | ENSG00000082781 | ITGB5 | HBV Day 3 |
| 28 | ENSG00000119686 | FLVCR2 | HBV Day 3 |
| 29 | ENSG00000148143 | ZNF462 | HBV Day 3 |
| 30 | ENSG00000116299 | KIAA1324 | HBV Day 3 |
| 31 | ENSG00000166451 | CENPN | HBV Day 3 |
| 32 | ENSG00000263528 | IKBKE | HBV Day 3 |
| 33 | ENSG00000167711 | SERPINF2 | HBV Day 3 |
| 34 | ENSG00000114023 | FAM162A | HBV Day 3 |
| 35 | ENSG00000205302 | SNX2 | HBV Day 3 |
| 36 | ENSG00000149131 | SERPING1 | HBV Day 3 |
| 37 | ENSG00000137975 | CLCA2 | HBV Day 3 |
| 38 | ENSG00000141096 | DPEP3 | HBV Day 3 |
| 39 | ENSG00000185215 | TNFAIP2 | HBV Day 3 |
| 40 | ENSG00000053108 | FSTL4 | HBV Day 3 |
| 41 | ENSG00000117984 | CTSD | HBV Day 3 |
| 42 | ENSG00000050820 | BCAR1 | HBV Day 3 |
| 43 | ENSG00000150051 | MKX | HBV Day 3 |
| 44 | ENSG00000116741 | RGS2 | HBV Day 3 |
| 45 | ENSG00000205413 | SAMD9 | HBV Day 3 |
| 46 | ENSG00000023909 | GCLM | HBV Day 3 |
| 47 | ENSG00000109743 | BST1 | HBV Day 3 |
| 48 | ENSG00000185950 | IRS2 | HBV Day 3 |
| 49 | ENSG00000169413 | RNASE6 | HBV Day 3 |
| 50 | ENSG00000119915 | ELOVL3 | HBV Day 3 |
| 1 | ENSG00000134202 | GSTM3 | HBV Day 7 |
| 2 | ENSG00000163754 | GYG1 | HBV Day 7 |
| 3 | ENSG00000102962 | CCL22 | HBV Day 7 |
| 4 | ENSG00000164172 | MOCS2 | HBV Day 7 |
| 5 | ENSG00000160932 | LY6E | HBV Day 7 |
| 6 | ENSG00000177697 | CD151 | HBV Day 7 |
| 7 | ENSG00000163221 | S100A12 | HBV Day 7 |
| 8 | ENSG00000051620 | HEBP2 | HBV Day 7 |
| 9 | ENSG00000106263 | EIF3B | HBV Day 7 |
| 10 | ENSG00000136881 | BAAT | HBV Day 7 |
| 11 | ENSG00000174547 | MRPL11 | HBV Day 7 |
| 12 | ENSG00000089127 | OAS1 | HBV Day 7 |
| 13 | ENSG00000143390 | RFX5 | HBV Day 7 |
| 14 | ENSG00000103035 | PSMD7 | HBV Day 7 |
| 15 | ENSG00000111275 | ALDH2 | HBV Day 7 |
| 16 | ENSG00000035720 | STAP1 | HBV Day 7 |
| 17 | ENSG00000111713 | GYS2 | HBV Day 7 |
| 18 | ENSG00000197045 | GMFB | HBV Day 7 |
| 19 | ENSG00000277632 | CCL3 | HBV Day 7 |
| 20 | ENSG00000041357 | PSMA4 | HBV Day 7 |
| 21 | ENSG00000164932 | CTHRC1 | HBV Day 7 |
| 22 | ENSG00000140932 | CMTM2 | HBV Day 7 |
| 23 | ENSG00000135218 | CD36 | HBV Day 7 |
| 24 | ENSG00000117411 | B4GALT2 | HBV Day 7 |
| 25 | ENSG00000107223 | EDF1 | HBV Day 7 |
| 26 | ENSG00000176749 | CDK5R1 | HBV Day 7 |
| 27 | ENSG00000184106 | TREML3P | HBV Day 7 |
| 28 | ENSG00000140464 | PML | HBV Day 7 |
| 29 | ENSG00000181333 | HEPHL1 | HBV Day 7 |
| 30 | ENSG00000146072 | TNFRSF21 | HBV Day 7 |
| 31 | ENSG00000240065 | PSMB9 | HBV Day 7 |
| 32 | ENSG00000127955 | GNAI1 | HBV Day 7 |
| 33 | ENSG00000106537 | TSPAN13 | HBV Day 7 |
| 34 | ENSG00000117410 | ATP6VOB | HBV Day 7 |
| 35 | ENSG00000080493 | SLC4A4 | HBV Day 7 |
| 36 | ENSG00000143621 | ILF2 | HBV Day 7 |
| 37 | ENSG00000131016 | AKAP12 | HBV Day 7 |
| 38 | ENSG00000198502 | HLA-DRB5 | HBV Day 7 |
| 39 | ENSG00000082175 | PGR | HBV Day 7 |
| 40 | ENSG00000177674 | AGTRAP | HBV Day 7 |
| 41 | ENSG00000117385 | P3H1 | HBV Day 7 |
| 42 | ENSG00000102543 | CDADC1 | HBV Day 7 |
| 43 | ENSG00000132256 | TRIM5 | HBV Day 7 |
| 44 | ENSG00000050628 | PTGER3 | HBV Day 7 |
| 45 | ENSG00000174233 | ADCY6 | HBV Day 7 |
| 46 | ENSG00000141736 | ERBB2 | HBV Day 7 |
| 47 | ENSG00000001167 | NFYA | HBV Day 7 |
| 48 | ENSG00000166888 | STAT6 | HBV Day 7 |
| 49 | ENSG00000108960 | MMD | HBV Day 7 |
| 50 | ENSG00000198755 | RPL10A | HBV Day 7 |
| 1 | ENSG00000204103 | MAFB | Dengue |
| 2 | ENSG00000131981 | LGALS3 | Dengue |
| 3 | ENSG00000038427 | VCAN | Dengue |
| 4 | ENSG00000004799 | PDK4 | Dengue |
| 5 | ENSG00000110651 | CD81 | Dengue |
| 6 | ENSG00000102837 | OLFM4 | Dengue |
| 7 | ENSG00000118113 | MMP8 | Dengue |
| 8 | ENSG00000158473 | CD1D | Dengue |
| 9 | ENSG00000136826 | KLF4 | Dengue |
| 10 | ENSG00000121552 | CSTA | Dengue |
| 11 | ENSG00000138413 | IDH1 | Dengue |
| 12 | ENSG00000205730 | ITPRIPL2 | Dengue |
| 13 | ENSG00000100292 | HMOX1 | Dengue |
| 14 | ENSG00000155659 | VSIG4 | Dengue |
| 15 | ENSG00000171877 | FRMD5 | Dengue |
| 16 | ENSG00000122641 | INHBA | Dengue |
| 17 | ENSG00000111275 | ALDH2 | Dengue |
| 18 | ENSG00000198682 | PAPSS2 | Dengue |
| 19 | ENSG00000012223 | LTF | Dengue |
| 20 | ENSG00000163221 | S100A12 | Dengue |
| 21 | ENSG00000110077 | MS4A6A | Dengue |
| 22 | ENSG00000197448 | GSTK1 | Dengue |
| 23 | ENSG00000092098 | RNF31 | Dengue |
| 24 | ENSG00000204301 | NOTCH4 | Dengue |
| 25 | ENSG00000065618 | COL17A1 | Dengue |
| 26 | ENSG00000143546 | S100A8 | Dengue |
| 27 | ENSG00000100448 | CTSG | Dengue |
| 28 | ENSG00000135604 | STX11 | Dengue |
| 29 | ENSG00000163661 | PTX3 | Dengue |
| 30 | ENSG00000138119 | MYOF | Dengue |
| 31 | ENSG00000111144 | LTA4H | Dengue |
| 32 | ENSG00000234127 | TRIM26 | Dengue |
| 33 | ENSG00000138061 | CYP1B1 | Dengue |
| 34 | ENSG00000118520 | ARG1 | Dengue |
| 35 | ENSG00000159128 | IFNGR2 | Dengue |
| 36 | ENSG00000176597 | B3GNT5 | Dengue |
| 37 | ENSG00000115919 | KYNU | Dengue |
| 38 | ENSG00000123684 | LPGAT1 | Dengue |
| 39 | ENSG00000109062 | SLC9A3R1 | Dengue |
| 40 | ENSG00000257017 | HP | Dengue |
| 41 | ENSG00000159339 | PADI4 | Dengue |
| 42 | ENSG00000092010 | PSME1 | Dengue |
| 43 | ENSG00000085871 | MGST2 | Dengue |
| 44 | ENSG00000123358 | NR4A1 | Dengue |
| 45 | ENSG00000118785 | SPP1 | Dengue |
| 46 | ENSG00000239839 | DEFA3 | Dengue |
| 47 | ENSG00000065833 | ME1 | Dengue |
| 48 | ENSG00000162444 | RBP7 | Dengue |
| 49 | ENSG00000139318 | DUSP6 | Dengue |
| 50 | ENSG00000187778 | MCRS1 | Dengue |
| 1 | ENSG00000170734 | POLH | H1N1 |
| 2 | ENSG00000050628 | PTGER3 | H1N1 |
| 3 | ENSG00000159216 | RUNX1 | H1N1 |
| 4 | ENSG00000138794 | CASP6 | H1N1 |
| 5 | ENSG00000111666 | CHPT1 | H1N1 |
| 6 | ENSG00000128394 | APOBEC3F | H1N1 |
| 7 | ENSG00000101557 | USP14 | H1N1 |
| 8 | ENSG00000121680 | PEX16 | H1N1 |
| 9 | ENSG00000196735 | HLA-DQA1 | H1N1 |
| 10 | ENSG00000137265 | IRF4 | H1N1 |
| 11 | ENSG00000101470 | TNNC2 | H1N1 |
| 12 | ENSG00000143622 | RIT1 | H1N1 |
| 13 | ENSG00000033011 | ALG1 | H1N1 |
| 14 | ENSG00000150593 | PDCD4 | H1N1 |
| 15 | ENSG00000130649 | CYP2E1 | H1N1 |
| 16 | ENSG00000034713 | GABARAPL2 | H1N1 |
| 17 | ENSG00000027847 | B4GALT7 | H1N1 |
| 18 | ENSG00000142166 | IFNAR1 | H1N1 |
| 19 | ENSG00000081189 | MEF2C | H1N1 |
| 20 | ENSG00000101916 | TLR8 | H1N1 |
| 21 | ENSG00000184205 | TSPYL2 | H1N1 |
| 22 | ENSG00000003056 | M6PR | H1N1 |
| 23 | ENSG00000185811 | IKZF1 | H1N1 |
| 24 | ENSG00000133313 | CNDP2 | H1N1 |
| 25 | ENSG00000174640 | SLCO2A1 | H1N1 |
| 26 | ENSG00000173933 | RBM4 | H1N1 |
| 27 | ENSG00000091483 | FH | H1N1 |
| 28 | ENSG00000053372 | MRTO4 | H1N1 |
| 29 | ENSG00000110042 | DTX4 | H1N1 |
| 30 | ENSG00000049541 | RFC2 | H1N1 |
| 31 | ENSG00000008118 | CAMK1G | H1N1 |
| 32 | ENSG00000141570 | CBX8 | H1N1 |
| 33 | ENSG00000101294 | HM13 | H1N1 |
| 34 | ENSG00000205220 | PSMB10 | H1N1 |
| 35 | ENSG00000023909 | GCLM | H1N1 |
| 36 | ENSG00000075415 | SLC25A3 | H1N1 |
| 37 | ENSG00000172936 | MYD88 | H1N1 |
| 38 | ENSG00000137033 | IL33 | H1N1 |
| 39 | ENSG00000169896 | ITGAM | H1N1 |
| 40 | ENSG00000196262 | PPIA | H1N1 |
| 41 | ENSG00000265808 | SEC22B | H1N1 |
| 42 | ENSG00000186810 | CXCR3 | H1N1 |
| 43 | ENSG00000136193 | SCRN1 | H1N1 |
| 44 | ENSG00000186350 | RXRA | H1N1 |
| 45 | ENSG00000073578 | SDHA | H1N1 |
| 46 | ENSG00000178445 | GLDC | H1N1 |
| 47 | ENSG00000111241 | FGF6 | H1N1 |
| 48 | ENSG00000138669 | PRKG2 | H1N1 |
| 49 | ENSG00000003436 | TFPI | H1N1 |
| 50 | ENSG00000132305 | IMMT | H1N1 |
| 1 | ENSG00000113742 | CPEB4 | Influenza pre-vaccine M |
| 2 | ENSG00000100526 | CDKN3 | Influenza pre-vaccine M |
| 3 | ENSG00000106785 | TRIM14 | Influenza pre-vaccine M |
| 4 | ENSG00000143412 | ANXA9 | Influenza pre-vaccine M |
| 5 | ENSG00000109846 | CRYAB | Influenza pre-vaccine M |
| 6 | ENSG00000171310 | CHST11 | Influenza pre-vaccine M |
| 7 | ENSG00000141552 | ANAPC11 | Influenza pre-vaccine M |
| 8 | ENSG00000169397 | RNASE3 | Influenza pre-vaccine M |
| 9 | ENSG00000115414 | FN1 | Influenza pre-vaccine M |
| 0 | ENSG00000029153 | ARNTL2 | Influenza pre-vaccine M |
| 11 | ENSG00000161850 | KRT82 | Influenza pre-vaccine M |
| 12 | ENSG00000146143 | PRIM2 | Influenza pre-vaccine M |
| 13 | ENSG00000164172 | MOCS2 | Influenza pre-vaccine M |
| 14 | ENSG00000103522 | IL21R | Influenza pre-vaccine M |
| 15 | ENSG00000107643 | MAPK8 | Influenza pre-vaccine M |
| 16 | ENSG00000173614 | NMNAT1 | Influenza pre-vaccine M |
| 17 | ENSG00000196247 | ZNF107 | Influenza pre-vaccine M |
| 18 | ENSG00000100448 | CTSG | Influenza pre-vaccine M |
| 19 | ENSG00000104432 | IL7 | Influenza pre-vaccine M |
| 20 | ENSG00000189127 | ANKRD34B | Influenza pre-vaccine M |
| 21 | ENSG00000144747 | TMF1 | Influenza pre-vaccine M |
| 22 | ENSG00000163755 | HPS3 | Influenza pre-vaccine M |
| 23 | ENSG00000122966 | CIT | Influenza pre-vaccine M |
| 24 | ENSG00000126602 | TRAP1 | Influenza pre-vaccine M |
| 25 | ENSG00000095002 | MSH2 | Influenza pre-vaccine M |
| 26 | ENSG00000145431 | PDGFC | Influenza pre-vaccine M |
| 27 | ENSG00000185973 | TMLHE | Influenza pre-vaccine M |
| 28 | ENSG00000013364 | MVP | Influenza pre-vaccine M |
| 29 | ENSG00000073861 | TBX21 | Influenza pre-vaccine M |
| 30 | ENSG00000073921 | PICALM | Influenza pre-vaccine M |
| 31 | ENSG00000205420 | KRT6A | Influenza pre-vaccine M |
| 32 | ENSG00000102081 | FMR1 | Influenza pre-vaccine M |
| 33 | ENSG00000169174 | PCSK9 | Influenza pre-vaccine M |
| 34 | ENSG00000163687 | DNASE1L3 | Influenza pre-vaccine M |
| 35 | ENSG00000167136 | ENDOG | Influenza pre-vaccine M |
| 36 | ENSG00000111907 | TPD52L1 | Influenza pre-vaccine M |
| 37 | ENSG00000124587 | PEX6 | Influenza pre-vaccine M |
| 38 | ENSG00000005381 | MPO | Influenza pre-vaccine M |
| 39 | ENSG00000175344 | CHRNA7 | Influenza pre-vaccine M |
| 40 | ENSG00000166750 | SLFN5 | Influenza pre-vaccine M |
| 41 | ENSG00000067182 | TNFRSF1A | Influenza pre-vaccine M |
| 42 | ENSG00000272398 | CD24 | Influenza pre-vaccine M |
| 43 | ENSG00000118307 | CASC1 | Influenza pre-vaccine M |
| 44 | ENSG00000073350 | LLGL2 | Influenza pre-vaccine M |
| 45 | ENSG00000151208 | DLG5 | Influenza pre-vaccine M |
| 46 | ENSG00000128833 | MYO5C | Influenza pre-vaccine M |
| 47 | ENSG00000082175 | PGR | Influenza pre-vaccine M |
| 48 | ENSG00000123836 | PFKFB2 | Influenza pre-vaccine M |
| 49 | ENSG00000004455 | AK2 | Influenza pre-vaccine M |
| 50 | ENSG00000082293 | COL19A1 | Influenza pre-vaccine M |
| 1 | ENSG00000086758 | HUWE1 | Influenza Day 1 M |
| 2 | ENSG00000164626 | KCNK5 | Influenza Day 1 M |
| 3 | ENSG00000135604 | STX11 | Influenza Day 1 M |
| 4 | ENSG00000159256 | MORC3 | Influenza Day 1 M |
| 5 | ENSG00000171208 | NETO2 | Influenza Day 1 M |
| 6 | ENSG00000168062 | BATF2 | Influenza Day 1 M |
| 7 | ENSG00000276085 | CCL3L1 | Influenza Day 1 M |
| 8 | ENSG00000205413 | SAMD9 | Influenza Day 1 M |
| 9 | ENSG00000108691 | CCL2 | Influenza Day 1 M |
| 10 | ENSG00000143847 | PPFIA4 | Influenza Day 1 M |
| 11 | ENSG00000089169 | RPH3A | Influenza Day 1 M |
| 12 | ENSG00000169248 | CXCL11 | Influenza Day 1 M |
| 13 | ENSG00000164010 | ERMAP | Influenza Day 1 M |
| 14 | ENSG00000162645 | GBP2 | Influenza Day 1 M |
| 15 | ENSG00000137752 | CASP1 | Influenza Day 1 M |
| 16 | ENSG00000196664 | TLR7 | Influenza Day 1 M |
| 17 | ENSG00000121053 | EPX | Influenza Day 1 M |
| 18 | ENSG00000154122 | ANKH | Influenza Day 1 M |
| 19 | ENSG00000242247 | ARFGAP3 | Influenza Day 1 M |
| 20 | ENSG00000198604 | BAZ1A | Influenza Day 1 M |
| 21 | ENSG00000130635 | COL5A1 | Influenza Day 1 M |
| 22 | ENSG00000143207 | COP1 | Influenza Day 1 M |
| 23 | ENSG00000110330 | BIRC2 | Influenza Day 1 M |
| 24 | ENSG00000103257 | SLC7A5 | Influenza Day 1 M |
| 25 | ENSG00000067445 | TRO | Influenza Day 1 M |
| 26 | ENSG00000124875 | CXCL6 | Influenza Day 1 M |
| 27 | ENSG00000121858 | TNFSF10 | Influenza Day 1 M |
| 28 | ENSG00000197465 | GYPE | Influenza Day 1 M |
| 29 | ENSG00000065618 | COL17A1 | Influenza Day 1 M |
| 30 | ENSG00000067900 | ROCK1 | Influenza Day 1 M |
| 31 | ENSG00000112149 | CD83 | Influenza Day 1 M |
| 32 | ENSG00000140057 | AK7 | Influenza Day 1 M |
| 33 | ENSG00000038945 | MSR1 | Influenza Day 1 M |
| 34 | ENSG00000148346 | LCN2 | Influenza Day 1 M |
| 35 | ENSG00000197471 | SPN | Influenza Day 1 M |
| 36 | ENSG00000130707 | ASS1 | Influenza Day 1 M |
| 37 | ENSG00000143321 | HDGF | Influenza Day 1 M |
| 38 | ENSG00000161921 | CXCL16 | Influenza Day 1 M |
| 39 | ENSG00000168495 | POLR3D | Influenza Day 1 M |
| 40 | ENSG00000198814 | GK | Influenza Day 1 M |
| 41 | ENSG00000102837 | OLFM4 | Influenza Day 1 M |
| 42 | ENSG00000104375 | STK3 | Influenza Day 1 M |
| 43 | ENSG00000136144 | RCBTB1 | Influenza Day 1 M |
| 44 | ENSG00000110203 | FOLR3 | Influenza Day 1 M |
| 45 | ENSG00000156804 | FBXO32 | Influenza Day 1 M |
| 46 | ENSG00000006042 | TMEM98 | Influenza Day 1 M |
| 47 | ENSG00000167815 | PRDX2 | Influenza Day 1 M |
| 48 | ENSG00000166165 | CKB | Influenza Day 1 M |
| 49 | ENSG00000111647 | UHRF1BP1L | Influenza Day 1 M |
| 50 | ENSG00000100448 | CTSG | Influenza Day 1 M |
| 1 | ENSG00000117448 | AKR1A1 | Influenza Day 14 M |
| 2 | ENSG00000070614 | NDST1 | Influenza Day 14 M |
| 3 | ENSG00000137393 | RNF144B | Influenza Day 14 M |
| 4 | ENSG00000048052 | HDAC9 | Influenza Day 14 M |
| 5 | ENSG00000277791 | PSMB3 | Influenza Day 14 M |
| 6 | ENSG00000067057 | PFKP | Influenza Day 14 M |
| 7 | ENSG00000198125 | MB | Influenza Day 14 M |
| 8 | ENSG00000136997 | MYC | Influenza Day 14 M |
| 9 | ENSG00000142655 | PEX14 | Influenza Day 14 M |
| 10 | ENSG00000197780 | TAF13 | Influenza Day 14 M |
| 11 | ENSG00000102010 | BMX | Influenza Day 14 M |
| 12 | ENSG00000162409 | PRKAA2 | Influenza Day 14 M |
| 13 | ENSG00000050628 | PTGER3 | Influenza Day 14 M |
| 14 | ENSG00000125730 | C3 | Influenza Day 14 M |
| 15 | ENSG00000197694 | SPTAN1 | Influenza Day 14 M |
| 16 | ENSG00000101000 | PROCR | Influenza Day 14 M |
| 17 | ENSG00000124608 | AARS2 | Influenza Day 14 M |
| 18 | ENSG00000140983 | RHOT2 | Influenza Day 14 M |
| 19 | ENSG00000102174 | PHEX | Influenza Day 14 M |
| 20 | ENSG00000172009 | THOP1 | Influenza Day 14 M |
| 21 | ENSG00000134809 | TIMM10 | Influenza Day 14 M |
| 22 | ENSG00000101849 | TBL1X | Influenza Day 14 M |
| 23 | ENSG00000101076 | HNF4A | Influenza Day 14 M |
| 24 | ENSG00000196517 | SLC6A9 | Influenza Day 14 M |
| 25 | ENSG00000066926 | FECH | Influenza Day 14 M |
| 26 | ENSG00000109572 | CLCN3 | Influenza Day 14 M |
| 27 | ENSG00000105352 | CEACAM4 | Influenza Day 14 M |
| 28 | ENSG00000137673 | MMP7 | Influenza Day 14 M |
| 29 | ENSG00000176387 | HSD11B2 | Influenza Day 14 M |
| 30 | ENSG00000148339 | SLC25A25 | Influenza Day 14 M |
| 31 | ENSG00000118508 | RAB32 | Influenza Day 14 M |
| 32 | ENSG00000138755 | CXCL9 | Influenza Day 14 M |
| 33 | ENSG00000159197 | KCNE2 | Influenza Day 14 M |
| 34 | ENSG00000186431 | FCAR | Influenza Day 14 M |
| 35 | ENSG00000126759 | CFP | Influenza Day 14 M |
| 36 | ENSG00000017427 | IGF1 | Influenza Day 14 M |
| 37 | ENSG00000121680 | PEX16 | Influenza Day 14 M |
| 38 | ENSG00000167257 | RNF214 | Influenza Day 14 M |
| 39 | ENSG00000137193 | PIM1 | Influenza Day 14 M |
| 40 | ENSG00000171223 | JUNB | Influenza Day 14 M |
| 41 | ENSG00000135679 | MDM2 | Influenza Day 14 M |
| 42 | ENSG00000114268 | PFKFB4 | Influenza Day 14 M |
| 43 | ENSG00000181788 | SIAH2 | Influenza Day 14 M |
| 44 | ENSG00000122877 | EGR2 | Influenza Day 14 M |
| 45 | ENSG00000100433 | KCNK10 | Influenza Day 14 M |
| 46 | ENSG00000204371 | EHMT2 | Influenza Day 14 M |
| 47 | ENSG00000171051 | FPR1 | Influenza Day 14 M |
| 48 | ENSG00000139193 | CD27 | Influenza Day 14 M |
| 49 | ENSG00000147400 | CETN2 | Influenza Day 14 M |
| 50 | ENSG00000092295 | TGM1 | Influenza Day 14 M |
| 1 | ENSG00000196104 | SPOCK3 | Influenza pre-vaccine F |
| 2 | ENSG00000073008 | PVR | Influenza pre-vaccine F |
| 3 | ENSG00000168802 | CHTF8 | Influenza pre-vaccine F |
| 4 | ENSG00000144136 | SLC20A1 | Influenza pre-vaccine F |
| 5 | ENSG00000151883 | PARP8 | Influenza pre-vaccine F |
| 6 | ENSG00000171557 | FGG | Influenza pre-vaccine F |
| 7 | ENSG00000178381 | ZFAND2A | Influenza pre-vaccine F |
| 8 | ENSG00000131142 | CCL25 | Influenza pre-vaccine F |
| 9 | ENSG00000179218 | CALR | Influenza pre-vaccine F |
| 10 | ENSG00000149809 | TM7SF2 | Influenza pre-vaccine F |
| 11 | ENSG00000089280 | FUS | Influenza pre-vaccine F |
| 12 | ENSG00000213722 | DDAH2 | Influenza pre-vaccine F |
| 13 | ENSG00000061656 | SPAG4 | Influenza pre-vaccine F |
| 14 | ENSG00000171823 | FBXL14 | Influenza pre-vaccine F |
| 15 | ENSG00000116977 | LGALS8 | Influenza pre-vaccine F |
| 16 | ENSG00000159921 | GNE | Influenza pre-vaccine F |
| 17 | ENSG00000170961 | HAS2 | Influenza pre-vaccine F |
| 18 | ENSG00000140749 | IGSF6 | Influenza pre-vaccine F |
| 19 | ENSG00000086062 | B4GALT1 | Influenza pre-vaccine F |
| 20 | ENSG00000122008 | POLK | Influenza pre-vaccine F |
| 21 | ENSG00000142731 | PLK4 | Influenza pre-vaccine F |
| 22 | ENSG00000065518 | NDUFB4 | Influenza pre-vaccine F |
| 23 | ENSG00000167414 | GNG8 | Influenza pre-vaccine F |
| 24 | ENSG00000185499 | MUC1 | Influenza pre-vaccine F |
| 25 | ENSG00000164252 | AGGF1 | Influenza pre-vaccine F |
| 26 | ENSG00000166794 | PPIB | Influenza pre-vaccine F |
| 27 | ENSG00000115902 | SLC1A4 | Influenza pre-vaccine F |
| 28 | ENSG00000179344 | HLA-DQB1 | Influenza pre-vaccine F |
| 29 | ENSG00000095539 | SEMA4G | Influenza pre-vaccine F |
| 30 | ENSG00000125148 | MT2A | Influenza pre-vaccine F |
| 31 | ENSG00000134871 | COL4A2 | Influenza pre-vaccine F |
| 32 | ENSG00000101333 | PLCB4 | Influenza pre-vaccine F |
| 33 | ENSG00000104812 | GYS1 | Influenza pre-vaccine F |
| 34 | ENSG00000126583 | PRKCG | Influenza pre-vaccine F |
| 35 | ENSG00000133105 | RXFP2 | Influenza pre-vaccine F |
| 36 | ENSG00000105499 | PLA2G4C | Influenza pre-vaccine F |
| 37 | ENSG00000128918 | ALDH1A2 | Influenza pre-vaccine F |
| 38 | ENSG00000115008 | IL1A | Influenza pre-vaccine F |
| 39 | ENSG00000005700 | IBTK | Influenza pre-vaccine F |
| 40 | ENSG00000113140 | SPARC | Influenza pre-vaccine F |
| 41 | ENSG00000111331 | OAS3 | Influenza pre-vaccine F |
| 42 | ENSG00000116106 | EPHA4 | Influenza pre-vaccine F |
| 43 | ENSG00000234745 | HLA-B | Influenza pre-vaccine F |
| 44 | ENSG00000204516 | MICB | Influenza pre-vaccine F |
| 45 | ENSG00000275385 | CCL18 | Influenza pre-vaccine F |
| 46 | ENSG00000141424 | SLC39A6 | Influenza pre-vaccine F |
| 47 | ENSG00000138604 | GLCE | Influenza pre-vaccine F |
| 48 | ENSG00000137285 | TUBB2B | Influenza pre-vaccine F |
| 49 | ENSG00000164117 | FBXO8 | Influenza pre-vaccine F |
| 50 | ENSG00000129515 | SNX6 | Influenza pre-vaccine F |
| 1 | ENSG00000140853 | NLRC5 | Influenza Day 1 F |
| 2 | ENSG00000165995 | CACNB2 | Influenza Day 1 F |
| 3 | ENSG00000075275 | CELSR1 | Influenza Day 1 F |
| 4 | ENSG00000151883 | PARP8 | Influenza Day 1 F |
| 5 | ENSG00000114346 | ECT2 | Influenza Day 1 F |
| 6 | ENSG00000109854 | HTATIP2 | Influenza Day 1 F |
| 7 | ENSG00000099250 | NRP1 | Influenza Day 1 F |
| 8 | ENSG00000071051 | NCK2 | Influenza Day 1 F |
| 9 | ENSG00000166292 | TMEM100 | Influenza Day 1 F |
| 10 | ENSG00000137975 | CLCA2 | Influenza Day 1 F |
| 11 | ENSG00000164929 | BAALC | Influenza Day 1 F |
| 12 | ENSG00000152104 | PTPN14 | Influenza Day 1 F |
| 13 | ENSG00000213928 | IRF9 | Influenza Day 1 F |
| 14 | ENSG00000134339 | SAA2 | Influenza Day 1 F |
| 15 | ENSG00000168453 | HR | Influenza Day 1 F |
| 16 | ENSG00000167378 | IRGQ | Influenza Day 1 F |
| 17 | ENSG00000117020 | AKT3 | Influenza Day 1 F |
| 18 | ENSG00000100321 | SYNGR1 | Influenza Day 1 F |
| 19 | ENSG00000125820 | NKX2-2 | Influenza Day 1 F |
| 20 | ENSG00000205358 | MT1H | Influenza Day 1 F |
| 21 | ENSG00000170099 | SERPINA6 | Influenza Day 1 F |
| 22 | ENSG00000162545 | CAMK2N1 | Influenza Day 1 F |
| 23 | ENSG00000132141 | CCT6B | Influenza Day 1 F |
| 24 | ENSG00000198554 | WDHD1 | Influenza Day 1 F |
| 25 | ENSG00000167034 | NKX3-1 | Influenza Day 1 F |
| 26 | ENSG00000166796 | LDHC | Influenza Day 1 F |
| 27 | ENSG00000172175 | MALT1 | Influenza Day 1 F |
| 28 | ENSG00000010278 | CD9 | Influenza Day 1 F |
| 29 | ENSG00000153132 | CLGN | Influenza Day 1 F |
| 30 | ENSG00000125454 | SLC25A19 | Influenza Day 1 F |
| 31 | ENSG00000135525 | MAP7 | Influenza Day 1 F |
| 32 | ENSG00000143184 | XCL1 | Influenza Day 1 F |
| 33 | ENSG00000164398 | ACSL6 | Influenza Day 1 F |
| 34 | ENSG00000072274 | TFRC | Influenza Day 1 F |
| 35 | ENSG00000121691 | CAT | Influenza Day 1 F |
| 36 | ENSG00000140807 | NKD1 | Influenza Day 1 F |
| 37 | ENSG00000169714 | CNBP | Influenza Day 1 F |
| 38 | ENSG00000144908 | ALDH1L1 | Influenza Day 1 F |
| 39 | ENSG00000108688 | CCL7 | Influenza Day 1 F |
| 40 | ENSG00000144136 | SLC20A1 | Influenza Day 1 F |
| 41 | ENSG00000133703 | KRAS | Influenza Day 1 F |
| 42 | ENSG00000184371 | CSF1 | Influenza Day 1 F |
| 43 | ENSG00000106144 | CASP2 | Influenza Day 1 F |
| 44 | ENSG00000163517 | HDAC11 | Influenza Day 1 F |
| 45 | ENSG00000221957 | KIR2DS4 | Influenza Day 1 F |
| 46 | ENSG00000186567 | CEACAM19 | Influenza Day 1 F |
| 47 | ENSG00000000971 | CFH | Influenza Day 1 F |
| 48 | ENSG00000102547 | CAB39L | Influenza Day 1 F |
| 49 | ENSG00000024526 | DEPDC1 | Influenza Day 1 F |
| 50 | ENSG00000129084 | PSMA1 | Influenza Day 1 F |
| 1 | ENSG00000187094 | CCK | Influenza Day 14 F |
| 2 | ENSG00000130766 | SESN2 | Influenza Day 14 F |
| 3 | ENSG00000136274 | NACAD | Influenza Day 14 F |
| 4 | ENSG00000169174 | PCSK9 | Influenza Day 14 F |
| 5 | ENSG00000159403 | C1R | Influenza Day 14 F |
| 6 | ENSG00000139514 | SLC7A1 | Influenza Day 14 F |
| 7 | ENSG00000143369 | ECM1 | Influenza Day 14 F |
| 8 | ENSG00000143184 | XCL1 | Influenza Day 14 F |
| 9 | ENSG00000081181 | ARG2 | Influenza Day 14 F |
| 10 | ENSG00000171621 | SPSB1 | Influenza Day 14 F |
| 11 | ENSG00000187775 | DNAH17 | Influenza Day 14 F |
| 12 | ENSG00000114854 | TNNC1 | Influenza Day 14 F |
| 13 | ENSG00000120054 | CPN1 | Influenza Day 14 F |
| 14 | ENSG00000108639 | SYNGR2 | Influenza Day 14 F |
| 15 | ENSG00000128510 | CPA4 | Influenza Day 14 F |
| 16 | ENSG00000168530 | MYL1 | Influenza Day 14 F |
| 17 | ENSG00000140279 | DUOX2 | Influenza Day 14 F |
| 18 | ENSG00000172888 | ZNF621 | Influenza Day 14 F |
| 19 | ENSG00000105679 | GAPDHS | Influenza Day 14 F |
| 20 | ENSG00000185825 | BCAP31 | Influenza Day 14 F |
| 21 | ENSG00000075711 | DLG1 | Influenza Day 14 F |
| 22 | ENSG00000056736 | IL17RB | Influenza Day 14 F |
| 23 | ENSG00000131389 | SLC6A6 | Influenza Day 14 F |
| 24 | ENSG00000129473 | BCL2L2 | Influenza Day 14 F |
| 25 | ENSG00000204388 | HSPA1B | Influenza Day 14 F |
| 26 | ENSG00000115902 | SLC1A4 | Influenza Day 14 F |
| 27 | ENSG00000215845 | TSTD1 | Influenza Day 14 F |
| 28 | ENSG00000152137 | HSPB8 | Influenza Day 14 F |
| 29 | ENSG00000178860 | MSC | Influenza Day 14 F |
| 30 | ENSG00000151849 | CENPJ | Influenza Day 14 F |
| 31 | ENSG00000143862 | ARL8A | Influenza Day 14 F |
| 32 | ENSG00000163599 | CTLA4 | Influenza Day 14 F |
| 33 | ENSG00000151892 | GFRA1 | Influenza Day 14 F |
| 34 | ENSG00000112290 | WASF1 | Influenza Day 14 F |
| 35 | ENSG00000137275 | RIPK1 | Influenza Day 14 F |
| 36 | ENSG00000108515 | ENO3 | Influenza Day 14 F |
| 37 | ENSG00000171345 | KRT19 | Influenza Day 14 F |
| 38 | ENSG00000130300 | PLVAP | Influenza Day 14 F |
| 39 | ENSG00000070950 | RAD18 | Influenza Day 14 F |
| 40 | ENSG00000087085 | ACHE | Influenza Day 14 F |
| 41 | ENSG00000140092 | FBLN5 | Influenza Day 14 F |
| 42 | ENSG00000085871 | MGST2 | Influenza Day 14 F |
| 43 | ENSG00000089053 | ANAPC5 | Influenza Day 14 F |
| 44 | ENSG00000143390 | RFX5 | Influenza Day 14 F |
| 45 | ENSG00000165806 | CASP7 | Influenza Day 14 F |
| 46 | ENSG00000159167 | STC1 | Influenza Day 14 F |
| 47 | ENSG00000071051 | NCK2 | Influenza Day 14 F |
| 48 | ENSG00000165949 | IFI27 | Influenza Day 14 F |
| 49 | ENSG00000110244 | APOA4 | Influenza Day 14 F |
| 50 | ENSG00000148450 | MSRB2 | Influenza Day 14 F |

TABLE 6
Performance of literature signatures (rows) across different datasets (columns). Shown are percentile
values obtained by comparing literature signature performance against random gene lists.

| | | | Influenza | Influenza | | | | |
|---|---|---|---|---|---|---|---|---|
| | | | pre- | pre- | | | | |
| | | | vaccine | vaccine | Influenza | Influenza | Influenza | Influenza |
| Literature Signature | Dengue | H1N1 | M | F | Day 1 M | Day 1 F | Day 14 M | Day 14 F |
| Monaco_CellRep_2019_B_Ex_signature | 69.31 | 35.64 | 38.61 | 78.22 | 59.41 | 71.29 | 74.26 | 87.13 |
| Monaco_CellRep_2019_B_NSM_signature | 34.65 | 13.86 | 98.02 | 90.1 | 46.53 | 89.11 | 41.58 | 44.55 |
| Monaco_CellRep_2019_B_Naive_signature | 47.52 | 7.92 | 98.02 | 96.04 | 11.88 | 60.4 | 23.76 | 94.06 |
| Monaco_CellRep_2019_B_SM_signature | 80.2 | 79.21 | 82.18 | 2.97 | 40.59 | 62.38 | 3.96 | 0.99 |
| Monaco_CellRep_2019_Basophils_LD_signature | 59.4 | 57.43 | 29.7 | 3.96 | 57.43 | 53.47 | 87.13 | 49.5 |
| Monaco_CellRep_2019_MAIT_signature | 80.2 | 79.21 | 13.86 | 92.08 | 77.23 | 99.01 | 44.55 | 86.14 |
| Monaco_CellRep_2019_Monocytes_C_signature | 100 | 14.85 | 52.48 | 73.27 | 15.84 | 2.97 | 20.79 | 22.77 |
| Monaco_CellRep_2019_Monocytes_I_signature | 52.48 | 34.65 | 85.15 | 44.55 | 71.29 | 53.47 | 100 | 11.88 |
| Monaco_CellRep_2019_Monocytes_NC_signature | 91.09 | 14.85 | 10.89 | 48.51 | 54.46 | 72.28 | 88.12 | 87.13 |
| Monaco_CellRep_2019_NK_signature | 73.27 | 11.88 | 83.17 | 48.51 | 2.97 | 75.25 | 88.12 | 73.27 |
| Monaco_CellRep_2019_Neutrophils_signature | 88.12 | 54.46 | 9.9 | 70.3 | 92.08 | 12.87 | 96.04 | 63.37 |
| Monaco_CellRep_2019_Plasmablasts_signature | 24.75 | 69.31 | 1.98 | 60.4 | 67.33 | 33.66 | 36.63 | 49.5 |
| Monaco_CellRep_2019_Progenitors_signature | 54.46 | 29.7 | 51.49 | 86.14 | 89.11 | 100 | 40.59 | 48.51 |
| Monaco_CellRep_2019_T_CD4_Naive_signature | 54.46 | 88.12 | 40.59 | 96.04 | 71.29 | 23.76 | 76.24 | 71.29 |
| Monaco_CellRep_2019_T_CD8_EM_signature | 46.53 | 3.96 | 79.21 | 92.08 | 55.45 | 42.57 | 70.3 | 71.29 |
| Monaco_CellRep_2019_T_CD8_Naive_signature | 58.42 | 50.5 | 39.6 | 69.31 | 60.4 | 92.08 | 94.06 | 50.5 |
| Monaco_CellRep_2019_T_CD8_TE_signature | 94.06 | 9.9 | 15.84 | 27.72 | 55.45 | 77.23 | 27.72 | 40.59 |
| Monaco_CellRep_2019_Th17_signature | 58.42 | 16.83 | 52.48 | 76.24 | 38.61 | 77.23 | 6.93 | 79.21 |
| Monaco_CellRep_2019_Th2_signature | 10.89 | 24.75 | 97.03 | 84.16 | 62.38 | 13.86 | 10.89 | 73.27 |
| Monaco_CellRep_2019_Tregs_signature | 79.21 | 6.93 | 21.78 | 74.26 | 69.31 | 36.63 | 88.12 | 97.03 |
| Monaco_CellRep_2019_mDCs_signature | 100 | 59.41 | 50.5 | 86.14 | 87.13 | 12.87 | 46.53 | 81.19 |
| Monaco_CellRep_2019_pDCs_signature | 96.04 | 41.58 | 41.58 | 45.54 | 30.69 | 25.74 | 81.19 | 84.16 |
| MSigDB_hallmark_tnfa_signaling_via_nfkb | 81.19 | 12.87 | 11.88 | 51.49 | 99.01 | 40.59 | 83.17 | 77.23 |
| MSigDB_hallmark_hypoxia | 46.53 | 57.43 | 6.93 | 9.9 | 72.28 | 55.45 | 73.27 | 90.1 |
| MSigDB_hallmark_cholesterol_homeostasis | 93.07 | 85.15 | 2.97 | 64.36 | 39.6 | 47.52 | 19.8 | 15.84 |
| MSigDB_hallmark_mitotic_spindle | 62.38 | 14.85 | 44.55 | 12.87 | 43.56 | 83.17 | 72.28 | 78.22 |
| MSigDB_hallmark_wnt_beta_catenin_signaling | 98.02 | 46.53 | 57.43 | 33.66 | 12.87 | 98.02 | 96.04 | 49.5 |
| MSigDB_hallmark_tgf_beta_signaling | 73.27 | 55.45 | 66.34 | 100 | 55.45 | 6.93 | 91.09 | 74.26 |
| MSigDB_hallmark_il6_jak_stat3_signaling | 98.02 | 78.22 | 78.22 | 85.15 | 89.11 | 53.47 | 73.27 | 85.15 |
| MSigDB_hallmark_dna_repair | 40.59 | 93.07 | 38.61 | 7.92 | 58.42 | 59.41 | 76.24 | 69.31 |
| MSigDB_hallmark_g2m_checkpoint | 9.9 | 61.39 | 31.68 | 60.4 | 48.51 | 66.34 | 53.47 | 25.74 |
| MSigDB_hallmark_apoptosis | 94.06 | 75.25 | 3.96 | 96.04 | 74.26 | 29.7 | 72.28 | 91.09 |
| MSigDB_hallmark_notch_signaling | 65.35 | 83.17 | 24.75 | 66.34 | 92.08 | 35.64 | 88.12 | 85.15 |
| MSigDB_hallmark_adipogenesis | 97.03 | 76.24 | 52.48 | 25.74 | 14.85 | 8.91 | 99.01 | 39.6 |
| MSigDB_hallmark_estrogen_response_early | 96.04 | 16.83 | 32.67 | 94.06 | 71.29 | 92.08 | 73.27 | 83.17 |
| MSigDB_hallmark_estrogen_response_late | 96.04 | 91.09 | 93.07 | 61.39 | 83.17 | 42.57 | 32.67 | 62.38 |
| MSigDB_hallmark_androgen_response | 13.86 | 34.65 | 38.61 | 42.57 | 12.87 | 71.29 | 35.64 | 22.77 |
| MSigDB_hallmark_myogenesis | 4.95 | 80.2 | 38.61 | 56.44 | 36.63 | 64.36 | 94.06 | 100 |
| MSigDB_hallmark_protein_secretion | 59.41 | 87.13 | 55.45 | 20.79 | 70.3 | 19.8 | 23.76 | 20.79 |
| MSigDB_hallmark_interferon_alpha_response | 93.07 | 32.67 | 94.06 | 85.15 | 99.01 | 68.32 | 11.88 | 32.67 |
| MSigDB_hallmark_interferon_gamma_response | 78.22 | 89.11 | 63.37 | 98.02 | 97.03 | 72.28 | 87.13 | 77.23 |
| MSigDB_hallmark_apical_junction | 92.08 | 1.98 | 17.82 | 46.53 | 45.54 | 68.32 | 51.49 | 48.51 |
| MSigDB_hallmark_apical_surface | 92.08 | 1.98 | 95.05 | 92.08 | 18.81 | 27.72 | 12.87 | 53.47 |
| MSigDB_hallmark_hedgehog_signaling | 70.3 | 56.44 | 45.54 | 42.57 | 0.99 | 98.02 | 89.11 | 91.09 |
| MSigDB_hallmark_complement | 99.01 | 72.28 | 32.67 | 66.34 | 76.24 | 43.56 | 71.29 | 91.09 |
| MSigDB_hallmark_unfolded_protein_response | 10.89 | 60.4 | 17.82 | 98.02 | 75.25 | 56.44 | 19.8 | 35.64 |
| MSigDB_hallmark_pi3k_akt_mtor_signaling | 16.83 | 82.18 | 79.21 | 44.55 | 62.38 | 28.71 | 83.17 | 25.74 |
| MSigDB_hallmark_mtorc1_signaling | 73.27 | 64.36 | 4.95 | 77.23 | 24.75 | 45.54 | 20.79 | 55.45 |
| MSigDB_hallmark_e2f_targets | 11.88 | 47.52 | 82.18 | 44.55 | 26.73 | 39.6 | 60.4 | 32.67 |
| MSigDB_hallmark_myc_targets_v1 | 1.98 | 57.43 | 34.65 | 32.67 | 42.57 | 66.34 | 73.27 | 32.67 |
| MSigDB_hallmark_myc_targets_v2 | 3.96 | 71.29 | 17.82 | 52.48 | 31.68 | 67.33 | 75.25 | 62.38 |
| MSigDB_hallmark_epithelial_mesenchymal_transition | 98.02 | 9.9 | 17.82 | 97.03 | 53.47 | 56.44 | 30.69 | 66.34 |
| MSigDB_hallmark_inflammatory_response | 69.31 | 11.88 | 11.88 | 80.2 | 94.06 | 29.7 | 90.1 | 66.34 |
| MSigDB_hallmark_xenobiotic_metabolism | 98.02 | 82.18 | 11.88 | 77.23 | 49.5 | 40.59 | 49.5 | 65.35 |
| MSigDB_hallmark_fatty_acid_metabolism | 95.05 | 60.4 | 41.58 | 65.35 | 23.76 | 12.87 | 60.4 | 60.4 |
| MSigDB_hallmark_oxidative_phosphorylation | 79.21 | 86.14 | 51.49 | 50.5 | 7.92 | 17.82 | 71.29 | 17.82 |
| MSigDB_hallmark_glycolysis | 91.09 | 92.08 | 28.71 | 93.07 | 29.7 | 28.71 | 97.03 | 65.35 |
| MSigDB_hallmark_reactive_oxygen_species_pathway | 96.04 | 80.2 | 90.1 | 89.11 | 86.14 | 2.97 | 98.02 | 60.4 |
| MSigDB_hallmark_p53_pathway | 92.08 | 99.01 | 34.65 | 56.44 | 67.33 | 11.88 | 91.09 | 36.63 |
| MSigDB_hallmark_uv_response_up | 72.28 | 21.78 | 48.51 | 67.33 | 34.65 | 0.99 | 68.32 | 97.03 |
| MSigDB_hallmark_uv_response_dn | 48.51 | 71.29 | 29.7 | 94.06 | 5.94 | 56.44 | 46.53 | 77.23 |
| MSigDB_hallmark_angiogenesis | 98.02 | 76.24 | 3.96 | 21.78 | 52.48 | 41.58 | 7.92 | 44.55 |
| MSigDB_hallmark_heme_metabolism | 6.93 | 43.56 | 52.48 | 44.55 | 90.1 | 75.25 | 99.01 | 45.54 |
| MSigDB_hallmark_coagulation | 96.04 | 15.84 | 14.85 | 82.18 | 21.78 | 59.41 | 78.22 | 71.29 |
| MSigDB_hallmark_il2_stat5_signaling | 84.16 | 64.36 | 4.95 | 88.12 | 91.09 | 20.79 | 44.55 | 80.2 |
| MSigDB_hallmark_bile_acid_metabolism | 100 | 74.26 | 66.34 | 42.57 | 12.87 | 44.55 | 78.22 | 10.89 |
| MSigDB_hallmark_peroxisome | 95.05 | 65.35 | 70.3 | 5.94 | 18.81 | 99.01 | 75.25 | 8.91 |
| MSigDB_hallmark_allograft_rejection | 96.04 | 53.47 | 57.43 | 89.11 | 91.09 | 41.58 | 86.14 | 29.7 |
| MSigDB_hallmark_spermatogenesis | 23.76 | 0.99 | 60.4 | 26.73 | 14.85 | 93.07 | 16.83 | 20.79 |
| MSigDB_hallmark_kras_signaling_up | 100 | 93.07 | 16.83 | 65.35 | 42.57 | 76.24 | 78.22 | 44.55 |
| MSigDB_hallmark_kras_signaling_dn | 18.81 | 25.74 | 29.7 | 45.54 | 47.52 | 34.65 | 73.27 | 65.35 |
| MSigDB_hallmark_pancreas_beta_cells | 82.18 | 11.88 | 24.75 | 43.56 | 5.94 | 100 | 62.38 | 32.67 |
| Ehrenberg_SciTransMed_2019 | 88.12 | 23.76 | 28.71 | 24.75 | 100 | 6.93 | 91.09 | 82.18 |
| Hansen_NatMed_2018_a | 53.47 | 22.77 | 76.24 | 86.14 | 99.01 | 36.63 | 76.24 | 18.81 |
| Hansen_NatMed_2018_b | 70.3 | 17.82 | 37.62 | 7.92 | 35.64 | 26.73 | 75.25 | 9.9 |
| Hansen_NatMed_2018_c | 93.07 | 62.38 | 47.52 | 92.08 | 80.2 | 2.97 | 81.19 | 5.94 |
| Bartholomeus_Vaccine_2018 | 86.14 | 41.58 | 13.86 | 100 | 2.97 | 35.64 | 96.04 | 67.33 |
| Franco_eLife_2013_a | 88.12 | 66.34 | 86.14 | 48.51 | 98.02 | 15.84 | 98.02 | 14.85 |
| Tsang_Cell_2014_a | 23.76 | 11.88 | 73.27 | 47.52 | 26.73 | 91.09 | 4.95 | 25.74 |
| Tsang_Cell_2014_b | 17.82 | 39.6 | 92.08 | 30.69 | 27.72 | 26.73 | 10.89 | 88.12 |
| Franco_eLife_2013_c | 77.23 | 91.09 | 91.09 | 58.42 | 87.13 | 59.41 | 86.14 | 37.62 |
| Franco_eLife_2013_d | 94.06 | 25.74 | 52.48 | 85.15 | 100 | 29.7 | 38.61 | 8.91 |
| Franco_eLife_2013_e | 83.17 | 91.09 | 78.22 | 12.87 | 10.89 | 16.83 | 34.65 | 54.46 |
| Franco_eLife_2013_f | 16.83 | 80.2 | 28.71 | 33.66 | 56.44 | 2.97 | 100 | 95.05 |
| Franco_eLife_2013_b | 35.64 | 24.75 | 87.13 | 84.16 | 84.16 | 95.05 | 98.02 | 83.17 |
| BermejoMartin_CriticCare_2010 | 43.56 | 98.02 | 92.08 | 70.3 | 96.04 | 82.18 | 96.04 | 42.57 |
| Cameron_JVirol_2007_a | 85.15 | 84.16 | 36.63 | 100 | 62.38 | 23.76 | 40.59 | 53.47 |
| Cameron_JVirol_2007_b | 91.09 | 93.07 | 19.8 | 99.01 | 94.06 | 77.23 | 7.92 | 66.34 |
| Cameron_JVirol_2007_c | 84.16 | 96.04 | 30.69 | 100 | 89.11 | 63.37 | 16.83 | 49.5 |
| Muramoto_JVirol_2014_a | 41.58 | 27.72 | 72.28 | 100 | 100 | 92.08 | 12.87 | 61.39 |
| Muramoto_JVirol_2014_b | 36.63 | 13.86 | 77.23 | 99.01 | 100 | 85.15 | 29.7 | 42.57 |
| Devignot_PLoSone_2010 | 100 | 7.92 | 80.2 | 26.73 | 61.39 | 50.5 | 29.7 | 40.59 |
| Zilliox_ClinVaccIm_2007 | 8.91 | 40.59 | 69.31 | 48.51 | 23.76 | 40.59 | 88.12 | 26.73 |
| Islam_Preprint_2020 | 94.06 | 9.9 | 40.59 | 49.5 | 91.09 | 22.77 | 85.15 | 22.77 |
| Islam_Preprint_2020_a | 55.45 | 12.87 | 81.19 | 90.1 | 100 | 50.5 | 64.36 | 90.1 |
| Islam_Preprint_2020_b | 91.09 | 21.78 | 56.44 | 91.09 | 100 | 80.2 | 52.48 | 30.69 |
| Wen_CellDiscovery_2020_a | 87.13 | 87.13 | 23.76 | 57.43 | 67.33 | 34.65 | 62.38 | 76.24 |
| Wen_CellDiscovery_2020_b | 89.11 | 93.07 | 46.53 | 97.03 | 62.38 | 2.97 | 54.46 | 60.4 |
| Wen_CellDiscovery_2020_c | 96.04 | 89.11 | 26.73 | 61.39 | 100 | 0.99 | 66.34 | 59.41 |
| Wen_CellDiscovery_2020_d | 15.84 | 52.48 | 15.84 | 67.33 | 93.07 | 0.99 | 99.01 | 47.52 |
| Wen_CellDiscovery_2020_e | 96.04 | 15.84 | 5.94 | 92.08 | 80.2 | 0.99 | 90.1 | 60.4 |
| Wen_CellDiscovery_2020_f | 96.04 | 69.31 | 6.93 | 97.03 | 96.04 | 2.97 | 86.14 | 51.49 |
| Wen_CellDiscovery_2020_g | 20.79 | 39.6 | 54.46 | 72.28 | 72.28 | 3.96 | 83.17 | 54.46 |
| Wen_CellDiscovery_2020_h | 94.06 | 79.21 | 12.87 | 91.09 | 86.14 | 2.97 | 96.04 | 51.49 |
| Hubel_NatIm_2019 | 96.04 | 67.33 | 43.56 | 86.14 | 91.09 | 93.07 | 26.73 | 79.21 |
| Mayhew_NatComm_2020 | 94.06 | 60.4 | 99.01 | 8.91 | 87.13 | 61.39 | 33.66 | 28.71 |
| Dunning_NatImm_2018_c | 96.04 | 2.97 | 94.06 | 100 | 86.14 | 3.96 | 65.35 | 13.86 |
| Dunning_NatImm_2018_b | 8.91 | 3.96 | 64.36 | 98.02 | 89.11 | 0.99 | 52.48 | 66.34 |
| Dunning_NatImm_2018_a | 100 | 1.98 | 74.26 | 48.51 | 92.08 | 42.57 | 6.93 | 45.54 |
| Liao_NatMed_2020_e | 48.51 | 16.83 | 28.71 | 61.39 | 61.39 | 35.64 | 75.25 | 42.57 |
| Liao_NatMed_2020_f | 81.19 | 60.4 | 16.83 | 22.77 | 9.9 | 10.89 | 97.03 | 1.98 |
| Liao_NatMed_2020_g | 83.17 | 38.61 | 29.7 | 67.33 | 79.21 | 71.29 | 93.07 | 99.01 |
| Liao_NatMed_2020_h | 14.85 | 39.6 | 16.83 | 35.64 | 0.99 | 56.44 | 53.47 | 19.8 |
| Liao_NatMed_2020_i | 62.38 | 13.86 | 84.16 | 59.41 | 67.33 | 39.6 | 100 | 76.24 |
| Liao_NatMed_2020_a | 100 | 44.55 | 16.83 | 35.64 | 83.17 | 21.78 | 79.21 | 81.19 |
| Liao_NatMed_2020_b | 97.03 | 5.94 | 10.89 | 17.82 | 100 | 2.97 | 60.4 | 54.46 |
| Liao_NatMed_2020_c | 96.04 | 47.52 | 35.64 | 93.07 | 72.28 | 95.05 | 75.25 | 40.59 |
| Liao_NatMed_2020_d | 100 | 86.14 | 30.69 | 60.4 | 78.22 | 50.5 | 45.54 | 37.62 |
| Liao_NatMed_2020_j | 28.71 | 60.4 | 1.98 | 42.57 | 61.39 | 23.76 | 21.78 | 28.71 |
| BlancoMelo_Cell_2020_a | 95.05 | 68.32 | 46.53 | 89.11 | 42.57 | 41.58 | 60.4 | 51.49 |
| BlancoMelo_Cell_2020_b | 4.95 | 44.55 | 95.05 | 100 | 63.37 | 12.87 | 2.97 | 85.15 |
| BlancoMelo_Cell_2020_g | 94.06 | 23.76 | 63.37 | 71.29 | 88.12 | 98.02 | 61.39 | 86.14 |
| BlancoMelo_Cell_2020_c | 40.59 | 1.98 | 78.22 | 86.14 | 85.15 | 97.03 | 38.61 | 67.33 |
| BlancoMelo_Cell_2020_d | 69.31 | 1.98 | 84.16 | 99.01 | 95.05 | 78.22 | 23.76 | 88.12 |
| BlancoMelo_Cell_2020_e | 91.09 | 3.96 | 2.97 | 19.8 | 38.61 | 31.68 | 67.33 | 82.18 |
| BlancoMelo_Cell_2020_f | 97.03 | 6.93 | 42.57 | 14.85 | 89.11 | 32.67 | 90.1 | 57.43 |
| Xiong_EmergMicrobInf_2020_a | 9.9 | 26.73 | 21.78 | 29.7 | 13.86 | 87.13 | 51.49 | 100 |
| Xiong_EmergMicrobInf_2020_b | 100 | 5.94 | 25.74 | 62.38 | 75.25 | 38.61 | 8.91 | 55.45 |
| Anderson_NEJM_2014_a | 93.07 | 65.35 | 99.01 | 56.44 | 83.17 | 23.76 | 83.17 | 45.54 |
| Anderson_NEJM_2014_b | 54.46 | 9.9 | 51.49 | 67.33 | 95.05 | 40.59 | 42.57 | 19.8 |
| Berry_Nature_2010_a | 86.14 | 14.85 | 13.86 | 89.11 | 97.03 | 9.9 | 77.23 | 11.88 |
| Berry_Nature_2010_b | 94.06 | 69.31 | 25.74 | 54.46 | 99.01 | 68.32 | 34.65 | 16.83 |
| Bloom_PLoSone_2013 | 98.02 | 68.32 | 26.73 | 82.18 | 82.18 | 27.72 | 57.43 | 35.64 |
| Jacobsen_JMolMed_2007 | 100 | 49.5 | 94.06 | 73.27 | 96.04 | 22.77 | 42.57 | 43.56 |
| Kaforou_PLoSMed_2013_a | 68.32 | 46.53 | 36.63 | 23.76 | 91.09 | 59.41 | 93.07 | 47.52 |
| Kaforou_PLoSMed_2013_b | 96.04 | 69.31 | 72.28 | 92.08 | 89.11 | 95.05 | 39.6 | 69.31 |
| Kaforou_PLoSMed_2013_c | 100 | 91.09 | 79.21 | 69.31 | 82.18 | 97.03 | 29.7 | 68.32 |
| Leong_Tuberculosis_2018_a | 83.17 | 38.61 | 86.14 | 59.41 | 86.14 | 63.37 | 63.37 | 10.89 |
| Leong_Tuberculosis_2018_b | 87.13 | 20.79 | 18.81 | 96.04 | 100 | 4.95 | 36.63 | 66.34 |
| Maertzdorf_EMBOMolMed_2016_a | 94.06 | 33.66 | 4.95 | 78.22 | 100 | 3.96 | 29.7 | 61.39 |
| Maertzdorf_EMBOMolMed_2016_b | 62.38 | 3.96 | 81.19 | 7.92 | 64.36 | 13.86 | 30.69 | 0.99 |
| Sambarey_EBioMedicine_2017 | 85.15 | 33.66 | 95.05 | 89.11 | 99.01 | 17.82 | 100 | 60.4 |
| Suliman_AmJRespCritCareMed_2018_a | 39.6 | 67.33 | 54.46 | 56.44 | 30.69 | 75.25 | 30.69 | 51.49 |
| Suliman_AmJRespCritCareMed_2018_b | 99.01 | 92.08 | 51.49 | 92.08 | 41.58 | 20.79 | 6.93 | 95.05 |
| Sweeney_LancetRespMed_2018 | 16.83 | 72.28 | 57.43 | 52.48 | 51.49 | 63.37 | 42.57 | 24.75 |
| Verhagen_BMCGenomics_2013 | 79.21 | 93.07 | 27.72 | 42.57 | 17.82 | 8.91 | 0.99 | 81.19 |
| Zak_Lancet_2016 | 93.07 | 50.5 | 22.77 | 41.58 | 99.01 | 35.64 | 62.38 | 7.92 |
| daCosta_Tuberculosis_2015 | 75.25 | 18.81 | 54.46 | 33.66 | 96.04 | 29.7 | 17.82 | 11.88 |

| | HBV | HBV | HBV | | | |
|---|---|---|---|---|---|---|
| | pre- | Day | Day | TB pre- | TB pre- | TB post- |
| Literature Gene | vaccine | 3 | 7 | vaccine | challenge | challenge |
| Monaco_CellRep_2019_B_Ex_signature | 84.16 | 2.97 | 30.69 | 27.72 | 1.98 | 37.62 |
| Monaco_CellRep_2019_B_NSM_signature | 36.63 | 5.94 | 91.09 | 37.62 | 18.81 | 14.85 |
| Monaco_CellRep_2019_B_Naive_signature | 64.36 | 3.96 | 22.77 | 16.83 | 12.87 | 94.06 |
| Monaco_CellRep_2019_B_SM_signature | 79.21 | 51.49 | 14.85 | 8.91 | 29.7 | 48.51 |
| Monaco_CellRep_2019_Basophils_LD_signature | 28.71 | 40.59 | 28.71 | 96.04 | 31.68 | 91.09 |
| Monaco_CellRep_2019_MAIT_signature | 53.47 | 70.3 | 42.57 | 10.89 | 28.71 | 15.84 |
| Monaco_CellRep_2019_Monocytes_C_signature | 93.07 | 91.09 | 82.18 | 12.87 | 1.98 | 1.98 |
| Monaco_CellRep_2019_Monocytes_I_signature | 92.08 | 61.39 | 77.23 | 18.81 | 17.82 | 99.01 |
| Monaco_CellRep_2019_Monocytes_NC_signature | 73.27 | 11.88 | 48.51 | 9.9 | 30.69 | 94.06 |
| Monaco_CellRep_2019_NK_signature | 35.64 | 32.67 | 57.43 | 100 | 7.92 | 70.3 |
| Monaco_CellRep_2019_Neutrophils_signature | 84.16 | 93.07 | 80.2 | 73.27 | 29.7 | 46.53 |
| Monaco_CellRep_2019_Plasmablasts_signature | 52.48 | 77.23 | 42.57 | 77.23 | 49.5 | 27.72 |
| Monaco_CellRep_2019_Progenitors_signature | 61.39 | 24.75 | 18.81 | 66.34 | 0.99 | 43.56 |
| Monaco_CellRep_2019_T_CD4_Naive_signature | 56.44 | 51.49 | 84.16 | 90.1 | 62.38 | 96.04 |
| Monaco_CellRep_2019_T_CD8_EM_signature | NA | NA | NA | 84.16 | 63.37 | 17.82 |
| Monaco_CellRep_2019_T_CD8_Naive_signature | 13.86 | 96.04 | 79.21 | 16.83 | 81.19 | 3.96 |
| Monaco_CellRep_2019_T_CD8_TE_signature | NA | NA | NA | NA | NA | NA |
| Monaco_CellRep_2019_Th17_signature | 69.31 | 42.57 | 91.09 | 39.6 | 44.55 | 90.1 |
| Monaco_CellRep_2019_Th2_signature | 95.05 | 48.51 | 83.17 | 19.8 | 15.84 | 50.5 |
| Monaco_CellRep_2019_Tregs_signature | 0.99 | 65.35 | 52.48 | 20.79 | 94.06 | 12.87 |
| Monaco_CellRep_2019_mDCs_signature | 62.38 | 96.04 | 55.45 | 95.05 | 11.88 | 26.73 |
| Monaco_CellRep_2019_pDCs_signature | 37.62 | 97.03 | 53.47 | 67.33 | 47.52 | 66.34 |
| MSigDB_hallmark_tnfa_signaling_via_nfkb | 61.39 | 96.04 | 66.34 | 23.76 | 7.92 | 85.15 |
| MSigDB_hallmark_hypoxia | 100 | 95.05 | 66.34 | 19.8 | 22.77 | 84.16 |
| MSigDB_hallmark_cholesterol_homeostasis | 12.87 | 77.23 | 93.07 | 32.67 | 98.02 | 100 |
| MSigDB_hallmark_mitotic_spindle | 9.9 | 77.23 | 27.72 | 64.36 | 18.81 | 48.51 |
| MSigDB_hallmark_wnt_beta_catenin_signaling | 32.67 | 44.55 | 59.41 | 28.71 | 48.51 | 67.33 |
| MSigDB_hallmark_tgf_beta_signaling | 31.68 | 24.75 | 7.92 | 41.58 | 61.39 | 16.83 |
| MSigDB_hallmark_il6_jak_stat3_signaling | 81.19 | 92.08 | 77.23 | 21.78 | 35.64 | 100 |
| MSigDB_hallmark_dna_repair | 67.33 | 25.74 | 72.28 | 95.05 | 88.12 | 37.62 |
| MSigDB_hallmark_g2m_checkpoint | 1.98 | 99.01 | 51.49 | 70.3 | 3.96 | 12.87 |
| MSigDB_hallmark_apoptosis | 64.36 | 65.35 | 41.58 | 43.56 | 0.99 | 100 |
| MSigDB_hallmark_notch_signaling | 80.2 | 39.6 | 38.61 | 0.99 | 40.59 | 21.78 |
| MSigDB_hallmark_adipogenesis | 62.38 | 96.04 | 86.14 | 93.07 | 23.76 | 61.39 |
| MSigDB_hallmark_estrogen_response_early | 60.4 | 14.85 | 0.99 | 43.56 | 6.93 | 62.38 |
| MSigDB_hallmark_estrogen_response_late | 93.07 | 50.5 | 71.29 | 99.01 | 8.91 | 84.16 |
| MSigDB_hallmark_androgen_response | 29.7 | 76.24 | 31.68 | 0.99 | 54.46 | 76.24 |
| MSigDB_hallmark_myogenesis | 51.49 | 37.62 | 58.42 | 49.5 | 59.41 | 4.95 |
| MSigDB_hallmark_protein_secretion | 21.78 | 90.1 | 43.56 | 32.67 | 16.83 | 89.11 |
| MSigDB_hallmark_interferon_alpha_response | 83.17 | 98.02 | 98.02 | 57.43 | 42.57 | 100 |
| MSigDB_hallmark_interferon_gamma_response | 79.21 | 90.1 | 87.13 | 79.21 | 6.93 | 100 |
| MSigDB_hallmark_apical_junction | 52.48 | 60.4 | 60.4 | 49.5 | 89.11 | 73.27 |
| MSigDB_hallmark_apical_surface | 11.88 | 53.47 | 99.01 | 77.23 | 32.67 | 20.79 |
| MSigDB_hallmark_hedgehog_signaling | 78.22 | 83.17 | 71.29 | 23.76 | 1.98 | 5.94 |
| MSigDB_hallmark_complement | 100 | 87.13 | 90.1 | 25.74 | 6.93 | 100 |
| MSigDB_hallmark_unfolded_protein_response | 36.63 | 91.09 | 77.23 | 42.57 | 81.19 | 89.11 |
| MSigDB_hallmark_pi3k_akt_mtor_signaling | 51.49 | 47.52 | 27.72 | 74.26 | 36.63 | 83.17 |
| MSigDB_hallmark_mtorc1_signaling | 73.27 | 42.57 | 59.41 | 24.75 | 73.27 | 80.2 |
| MSigDB_hallmark_e2f_targets | 50.5 | 83.17 | 30.69 | 96.04 | 24.75 | 17.82 |
| MSigDB_hallmark_myc_targets_v1 | 56.44 | 63.37 | 94.06 | 95.05 | 9.9 | 5.94 |
| MSigDB_hallmark_myc_targets_v2 | 70.3 | 24.75 | 46.53 | 80.2 | 87.13 | 39.6 |
| MSigDB_hallmark_epithelial_mesenchymal_transition | 32.67 | 93.07 | 72.28 | 96.04 | 46.53 | 64.36 |
| MSigDB_hallmark_inflammatory_response | 81.19 | 77.23 | 84.16 | 37.62 | 33.66 | 86.14 |
| MSigDB_hallmark_xenobiotic_metabolism | 82.18 | 92.08 | 87.13 | 36.63 | 65.35 | 65.35 |
| MSigDB_hallmark_fatty_acid_metabolism | 61.39 | 42.57 | 57.43 | 95.05 | 100 | 59.41 |
| MSigDB_hallmark_oxidative_phosphorylation | 98.02 | 91.09 | 84.16 | 96.04 | 59.41 | 20.79 |
| MSigDB_hallmark_glycolysis | 99.01 | 100 | 87.13 | 98.02 | 92.08 | 74.26 |
| MSigDB_hallmark_reactive_oxygen_species_pathway | 91.09 | 96.04 | 67.33 | 97.03 | 22.77 | 48.51 |
| MSigDB_hallmark_p53_pathway | 76.24 | 76.24 | 51.49 | 64.36 | 59.41 | 99.01 |
| MSigDB_hallmark_uv_response_up | 96.04 | 20.79 | 44.55 | 77.23 | 100 | 96.04 |
| MSigDB_hallmark_uv_response_dn | 25.74 | 19.8 | 38.61 | 28.71 | 8.91 | 34.65 |
| MSigDB_hallmark_angiogenesis | 48.51 | 70.3 | 94.06 | 26.73 | 5.94 | 94.06 |
| MSigDB_hallmark_heme_metabolism | 10.89 | 40.59 | 18.81 | 36.63 | 27.72 | 91.09 |
| MSigDB_hallmark_coagulation | 61.39 | 78.22 | 28.71 | 99.01 | 51.49 | 83.17 |
| MSigDB_hallmark_il2_stat5_signaling | 62.38 | 19.8 | 64.36 | 25.74 | 44.55 | 57.43 |
| MSigDB_hallmark_bile_acid_metabolism | 5.94 | 87.13 | 61.39 | 80.2 | 31.68 | 25.74 |
| MSigDB_hallmark_peroxisome | 34.65 | 21.78 | 63.37 | 62.38 | 11.88 | 42.57 |
| MSigDB_hallmark_allograft_rejection | 31.68 | 29.7 | 82.18 | 36.63 | 44.55 | 100 |
| MSigDB_hallmark_spermatogenesis | 15.84 | 71.29 | 92.08 | 58.42 | 0.99 | 89.11 |
| MSigDB_hallmark_kras_signaling_up | 35.64 | 92.08 | 76.24 | 71.29 | 5.94 | 99.01 |
| MSigDB_hallmark_kras_signaling_dn | 19.8 | 1.98 | 32.67 | 34.65 | 34.65 | 1.98 |
| MSigDB_hallmark_pancreas_beta_cells | 61.39 | 65.35 | 41.58 | 0.99 | 1.98 | 65.35 |
| Ehrenberg_SciTransMed_2019 | 65.35 | 98.02 | 52.48 | 71.29 | 47.52 | 25.74 |
| Hansen_NatMed_2018_a | 69.31 | 96.04 | 89.11 | 46.53 | 32.67 | 100 |
| Hansen_NatMed_2018_b | 11.88 | 86.14 | 2.97 | 23.76 | 100 | 72.28 |
| Hansen_NatMed_2018_c | 36.63 | 92.08 | 6.93 | 46.53 | 91.09 | 97.03 |
| Bartholomeus_Vaccine_2018 | 100 | 60.4 | 55.45 | 92.08 | 19.8 | 7.92 |
| Franco_eLife_2013_a | 57.43 | 88.12 | 81.19 | 12.87 | 13.86 | 100 |
| Tsang_Cell_2014_a | 17.82 | 10.89 | 67.33 | 40.59 | 74.26 | 35.64 |
| Tsang_Cell_2014_b | 10.89 | 94.06 | 56.44 | 84.16 | 38.61 | 66.34 |
| Franco_eLife_2013_c | 70.3 | 33.66 | 63.37 | 26.73 | 15.84 | 73.27 |
| Franco_eLife_2013_d | 75.25 | 89.11 | 93.07 | 47.52 | 7.92 | 100 |
| Franco_eLife_2013_e | 44.55 | 83.17 | 26.73 | 55.45 | 35.64 | 28.71 |
| Franco_eLife_2013_f | 70.3 | 14.85 | 8.91 | 14.85 | 90.1 | 17.82 |
| Franco_eLife_2013_b | 74.26 | 97.03 | 82.18 | 35.64 | 10.89 | 79.21 |
| BermejoMartin_CriticCare_2010 | 43.56 | 34.65 | 86.14 | 69.31 | 51.49 | 95.05 |
| Cameron_JVirol_2007_a | 25.74 | 80.2 | 43.56 | 77.23 | 2.97 | 96.04 |
| Cameron_JVirol_2007_b | 51.49 | 91.09 | 87.13 | 100 | 4.95 | 19.8 |
| Cameron_JVirol_2007_c | 49.5 | 95.05 | 79.21 | 85.15 | 4.95 | 43.56 |
| Muramoto_JVirol_2014_a | 82.18 | 81.19 | 99.01 | 74.26 | 31.68 | 100 |
| Muramoto_JVirol_2014_b | 81.19 | 88.12 | 94.06 | 91.09 | 19.8 | 100 |
| Devignot_PLoSone_2010 | 44.55 | 88.12 | 88.12 | 8.91 | 35.64 | 3.96 |
| Zilliox_ClinVacclm_2007 | 24.75 | 62.38 | 18.81 | 34.65 | 47.52 | 0.99 |
| Islam_Preprint_2020 | 95.05 | 58.42 | 97.03 | 80.2 | 36.63 | 98.02 |
| Islam_Preprint_2020_a | 0.99 | 38.61 | 1.98 | 60.4 | 11.88 | 98.02 |
| Islam_Preprint_2020_b | 85.15 | 87.13 | 45.54 | 1.98 | 6.93 | 100 |
| Wen_CellDiscovery_2020_a | 99.01 | 89.11 | 59.41 | 50.5 | 31.68 | 81.19 |
| Wen_CellDiscovery_2020_b | 53.47 | 86.14 | 71.29 | 59.41 | 53.47 | 45.54 |
| Wen_CellDiscovery_2020_c | 50.5 | 81.19 | 78.22 | 48.51 | 59.41 | 89.11 |
| Wen_CellDiscovery_2020_d | 16.83 | 60.4 | 42.57 | 14.85 | 66.34 | 84.16 |
| Wen_CellDiscovery_2020_e | 83.17 | 83.17 | 74.26 | 62.38 | 49.5 | 99.01 |
| Wen_CellDiscovery_2020_f | 49.5 | 87.13 | 55.45 | 54.46 | 75.25 | 99.01 |
| Wen_CellDiscovery_2020_g | 35.64 | 75.25 | 67.33 | 11.88 | 85.15 | 74.26 |
| Wen_CellDiscovery_2020_h | 82.18 | 81.19 | 76.24 | 49.5 | 62.38 | 97.03 |
| Hubel_Natlm_2019 | 95.05 | 95.05 | 94.06 | 2.97 | 56.44 | 100 |
| Mayhew_NatComm_2020 | 89.11 | 67.33 | 39.6 | 9.9 | 67.33 | 56.44 |
| Dunning_NatImm_2018_c | 60.4 | 20.79 | 38.61 | 1.98 | 78.22 | 0.99 |
| Dunning_NatImm_2018_b | 93.07 | 96.04 | 96.04 | 48.51 | 55.45 | 94.06 |
| Dunning_NatImm_2018_a | 100 | 68.32 | 57.43 | 30.69 | 56.44 | 21.78 |
| Liao_NatMed_2020_e | 67.33 | 98.02 | 52.48 | 14.85 | 2.97 | 25.74 |
| Liao_NatMed_2020_f | 82.18 | 4.95 | 86.14 | 65.35 | 64.36 | 41.58 |
| Liao_NatMed_2020_g | 40.59 | 66.34 | 96.04 | 59.41 | 42.57 | 71.29 |
| Liao_NatMed_2020_h | 15.84 | 77.23 | 87.13 | 65.35 | 48.51 | 63.37 |
| Liao_NatMed_2020_i | 84.16 | 34.65 | 70.3 | 31.68 | 89.11 | 97.03 |
| Liao_NatMed_2020_a | 85.15 | 90.1 | 96.04 | 72.28 | 1.98 | 60.4 |
| Liao_NatMed_2020_b | 99.01 | 75.25 | 71.29 | 52.48 | 0.99 | 100 |
| Liao_NatMed_2020_c | 25.74 | 70.3 | 48.51 | 28.71 | 19.8 | 83.17 |
| Liao_NatMed_2020_d | 77.23 | 19.8 | 33.66 | 28.71 | 23.76 | 61.39 |
| Liao_NatMed_2020_j | 22.77 | 91.09 | 43.56 | 80.2 | 32.67 | 46.53 |
| BlancoMelo_Cell_2020_a | 54.46 | 52.48 | 92.08 | 23.76 | 57.43 | 63.37 |
| BlancoMelo_Cell_2020_b | 86.14 | 97.03 | 27.72 | 95.05 | 71.29 | 4.95 |
| BlancoMelo_Cell_2020_g | 67.33 | 71.29 | 97.03 | 97.03 | 0.99 | 100 |
| BlancoMelo_Cell_2020_c | 51.49 | 93.07 | 78.22 | 31.68 | 0.99 | 99.01 |
| BlancoMelo_Cell_2020_d | 44.55 | 80.2 | 6.93 | 62.38 | 41.58 | 44.55 |
| BlancoMelo_Cell_2020_e | 63.37 | 60.4 | 45.54 | 14.85 | 77.23 | 98.02 |
| BlancoMelo_Cell_2020_f | 79.21 | 80.2 | 98.02 | 60.4 | 6.93 | 100 |
| Xiong_EmergMicrobInf_2020_a | 56.44 | 88.12 | 95.05 | 94.06 | 16.83 | 89.11 |
| Xiong_EmergMicrobInf_2020_b | 63.37 | 97.03 | 53.47 | 25.74 | 8.91 | 79.21 |
| Anderson_NEJM_2014_a | 6.93 | 37.62 | 20.79 | 62.38 | 23.76 | 91.09 |
| Anderson_NEJM_2014_b | 83.17 | 71.29 | 84.16 | 72.28 | 38.61 | 100 |
| Berry_Nature_2010_a | 94.06 | 85.15 | 96.04 | 42.57 | 0.99 | 100 |
| Berry_Nature_2010_b | 34.65 | 66.34 | 77.23 | 40.59 | 35.64 | 81.19 |
| Bloom_PLoSone_2013 | 89.11 | 27.72 | 98.02 | 53.47 | 4.95 | 92.08 |
| Jacobsen_JMolMed_2007 | NA | NA | NA | NA | NA | NA |
| Kaforou_PLoSMed_2013_a | 94.06 | 80.2 | 19.8 | 47.52 | 0.99 | 97.03 |
| Kaforou_PLoSMed_2013_b | 39.6 | 19.8 | 25.74 | 48.51 | 1.98 | 65.35 |
| Kaforou_PLoSMed_2013_c | 26.73 | 96.04 | 26.73 | 99.01 | 6.93 | 60.4 |
| Leong_Tuberculosis_2018_a | 40.59 | 61.39 | 28.71 | 13.86 | 16.83 | 75.25 |
| Leong_Tuberculosis_2018_b | 91.09 | 99.01 | 63.37 | 9.9 | 16.83 | 100 |
| Maertzdorf_EMBOMolMed_2016_a | 94.06 | 25.74 | 90.1 | 17.82 | 68.32 | 95.05 |
| Maertzdorf_EMBOMolMed_2016_b | NA | NA | NA | 70.3 | 33.66 | 97.03 |
| Sambarey_EBioMedicine_2017 | 94.06 | 0.99 | 95.05 | 99.01 | 63.37 | 40.59 |
| Suliman_AmJRespCritCareMed_2018_a | 48.51 | 19.8 | 77.23 | 13.86 | 27.72 | 61.39 |
| Suliman_AmJRespCritCareMed_2018_b | 77.23 | 88.12 | 39.6 | 50.5 | 79.21 | 23.76 |
| Sweeney_LancetRespMed_2018 | NA | NA | NA | NA | NA | NA |
| Verhagen_BMCGenomics_2013 | 58.42 | 33.66 | 81.19 | 63.37 | 48.51 | 73.27 |
| Zak_Lancet_2016 | 66.34 | 92.08 | 38.61 | 17.82 | 46.53 | 99.01 |
| daCosta_Tuberculosis_2015 | NA | NA | NA | NA | NA | NA |

TABLE 7A
Training and test datasets of related pairs based
on apparent biological relationships - F1 score

| | | | SARS CoV2 | H1N1 | TB |
|---|---|---|---|---|---|
| | | Training | Liao | Dunning | Zak |
| Training | Dengue | Devignot | 1 | 0.7143 | 0.28 |
| | H1N1 | BermejoMartin | NA | 0.548 | 0.4242 |
| | IAV | Franco_Male_Day 0 | NA | 0.029 | 0.3111 |
| | Vaccine | Franco_Female_Day 0 | 0.8571 | 0.0779 | 0.3809 |
| | | Franco_Male_Day 1 | 1 | 0.08 | 0.4536 |
| | | Franco_Female_Day 1 | NA | 0.0702 | 0.3164 |
| | | Franco_Male_Day 14 | NA | NA | 0.069 |
| | | Franco_Female_Day 14 | NA | NA | 0.2524 |
| | HBV | Bartholomeus_Day 0 | NA | 0.6182 | 0.1076 |
| | vaccine | Bartholomeus_Day 3 | NA | 0.0303 | 0.2667 |
| | | Bartholomeus_Day 7 | NA | 0.1429 | 0.3724 |
| | TB | Hansen_pre_Vaccine | NA | 0.0476 | 0.4299 |
| | vaccine | Hansen_preChallenge | NA | 0.7547 | 0.4386 |
| | | Hansen_postChallenge | NA | 0.4 | 0.6 |
| | | Rank 1 ( F1 score) | 1 | 0.75 | 0.6 |

TABLE 7B
Training and test datasets on presumed unrelated pairs - F1 score

| | | Asthma | | Rheumatoid Arth. | | NCI TARGET project | | | | |
|---|---|---|---|---|---|---|---|---|---|---|
| | Training | Bjornsdottir | Altman | Teixeira | Bienkowska | ALLP2 | ALLP3 | AML | OS | WT |
| Dengue | Devignot | 0.34 | 0.13 | 0.97 | 0.35 | 0.07 | 0.54 | 0.38 | 0.29 | 0.48 |
| H1N1 | BermejoMartin | 0.37 | 0.27 | 0.56 | 0.38 | 0.17 | NA | 0.33 | 0.34 | 0.42 |
| IAV | Franco_Male_Day 0 | 0.34 | 0.50 | NA | 0.41 | 0.18 | 0.47 | 0.07 | NA | 0.19 |
| Vaccine | Franco_Female_Day 0 | 0.41 | 0.29 | 0.65 | 0.30 | 0.16 | 0.42 | 0.34 | 0.36 | 0.44 |
| | Franco_Male_Day 1 | NA | 0.43 | NA | 0.40 | 0.25 | 0.24 | 0.46 | 0.18 | 0.17 |
| | Franco_Female_Day 1 | 0.32 | 0.55 | NA | 0.48 | 0.18 | 0.44 | 0.29 | 0.12 | 0.30 |
| | Franco_Male_Day 14 | 0.23 | 0.55 | NA | 0.38 | 0.26 | 0.30 | 0.35 | NA | 0.24 |
| | Franco_Female_Day 14 | 0.31 | 0.44 | 0.57 | 0.41 | 0.09 | 0.27 | 0.43 | 0.22 | 0.29 |
| HBV | Bartholomeus_Day 0 | 0.31 | 0.46 | 0.23 | 0.39 | 0.15 | 0.40 | 0.21 | 0.34 | 0.25 |
| vaccine | Bartholomeus_Day 3 | 0.29 | 0.23 | 0.56 | 0.31 | 0.17 | 0.36 | 0.38 | 0.40 | 0.10 |
| | Bartholomeus_Day 7 | 0.16 | 0.41 | 0.70 | 0.34 | 0.16 | 0.33 | 0.51 | NA | 0.10 |
| TB | Hansen_pre_Vaccine | 0.38 | 0.39 | 0.89 | 0.41 | 0.15 | 0.52 | 0.30 | 0.21 | 0.10 |
| vaccine | Hansen_preChallenge | 0.18 | 0.39 | 0.62 | 0.41 | 0.11 | 0.63 | 0.41 | 0.15 | 0.37 |
| | Hansen_postChallenge | 0.17 | 0.34 | 0.42 | 0.38 | 0.23 | 0.1 | 0.45 | 0.20 | 0.39 |
| | Rank 1 (F1 score) | 0.41 | 0.55 | 0.97 | 0.48 | 0.26 | 0.63 | 0.51 | 0.40 | 0.48 |

| | | | TCGA project | | | | | | | | |
|---|---|---|---|---|---|---|---|---|---|---|---|
| | | Training | BLCA | BRCA | CESC | CHOL | COAD | ESCA | GBM | HNSC | KIRC |
| Training | Dengue | Devignot | 0.54 | 0.27 | 0.46 | 0.40 | 0.46 | 0.58 | 0.57 | 0.52 | 0.41 |
| | H1N1 | BermejoMartin | 0.33 | 0.04 | 0.32 | 0.48 | 0.62 | 0.39 | 0.52 | 0.38 | 0.12 |
| | IAV | Franco_Male_Day 0 | 0.34 | 0.10 | 0.48 | 0.13 | 0.61 | 0.41 | 0.55 | 0.49 | 0.28 |
| | Vaccine | Franco_Female_Day 0 | 0.49 | 0.07 | 0.52 | 0.38 | 0.49 | 0.38 | 0.63 | 0.32 | 0.20 |
| | | Franco_Male_Day 1 | 0.58 | 0.24 | 0.11 | 0.47 | 0.61 | 0.41 | 0.11 | 0.53 | 0.41 |
| | | Franco_Female_Day 1 | 0.54 | 0.24 | 0.44 | NA | 0.50 | 0.41 | 0.20 | 0.50 | 0.13 |
| | | Franco_Male_Day 14 | 0.19 | 0.21 | 0.41 | 0.33 | 0.19 | 0.50 | 0.32 | 0.19 | 0.45 |
| | | Franco_Female_Day 14 | 0.28 | 0.21 | 0.33 | 0.18 | 0.36 | 0.33 | 0.29 | 0.24 | 0.19 |
| | HBV | Bartholomeus_Day 0 | 0.43 | 0.09 | 0.27 | 0.57 | 0.58 | 0.54 | 0.45 | 0.44 | 0.12 |
| | vaccine | Bartholomeus_Day 3 | 0.43 | 0.22 | 0.11 | 0.59 | 0.30 | 0.38 | 0.28 | 0.29 | 0.14 |
| | | Bartholomeus_Day 7 | 0.23 | 0.26 | 0.41 | 0.20 | 0.16 | 0.35 | 0.31 | 0.29 | 0.13 |
| | TB | Hansen_pre_Vaccine | 0.25 | 0.05 | 0.28 | 0.53 | 0.43 | 0.42 | 0.53 | 0.31 | 0.45 |
| | vaccine | Hansen_preChallenge | 0.41 | 0.04 | 0.31 | 0.60 | 0.13 | 0.45 | 0.58 | 0.54 | 0.12 |
| | | Hansen_postChallenge | 0.55 | 0.04 | 0.28 | NA | 0.49 | 0.23 | 0.22 | 0.49 | 0.12 |

| | | | | | | | | | |
|---|---|---|---|---|---|---|---|---|---|
| Rank 1 (F1 score) | 0.58 | 0.27 | 0.52 | 0.60 | 0.62 | 0.58 | 0.63 | 0.54 | 0.45 |

| | | | TCGA project | | | | | | | | |
|---|---|---|---|---|---|---|---|---|---|---|---|
| | | Training | KIRP | LAML | LGG | LIHC | LUAD | LUSC | MESO | OV | PAAD |
| Training | Dengue | Devignot | 0.47 | 0.30 | 0.48 | 0.53 | 0.26 | 0.09 | 0.16 | 0.20 | 0.34 |
| | H1N1 | BermejoMartin | NA | 0.50 | 0.52 | 0.47 | 0.31 | 0.09 | 0.44 | 0.24 | 0.26 |
| | IAV | Franco_Male_Day 0 | 0.26 | 0.71 | 0.33 | 0.48 | 0.22 | 0.51 | 0.58 | 0.24 | 0.21 |
| | Vaccine | Franco_Female_Day 0 | 0.40 | 0.73 | 0.16 | 0.52 | 0.21 | 0.53 | 0.22 | 0.16 | 0.28 |
| | | Franco_Male_Day 1 | 0.09 | 0.67 | 0.14 | 0.51 | 0.45 | 0.09 | 0.45 | 0.21 | 0.41 |
| | | Franco_Female_Day 1 | 0.33 | 0.42 | 0.32 | 0.22 | 0.27 | 0.46 | 0.38 | 0.25 | 0.22 |
| | | Franco_Male_Day 14 | NA | 0.25 | 0.15 | 0.13 | 0.26 | 0.11 | 0.33 | NA | 0.35 |
| | | Franco_Female_Day 14 | 0.17 | 0.31 | 0.18 | 0.59 | 0.23 | 0.30 | 0.36 | 0.12 | 0.30 |
| | HBV | Bartholomeus_Day 0 | 0.38 | 0.62 | 0.10 | 0.17 | 0.19 | 0.09 | 0.34 | 0.11 | 0.29 |
| | vaccine | Bartholomeus_Day 3 | NA | 0.25 | 0.16 | 0.09 | 0.30 | 0.24 | NA | 0.15 | 0.46 |
| | | Bartholomeus_Day 7 | NA | 0.74 | 0.06 | 0.27 | 0.11 | 0.52 | 0.36 | 0.22 | 0.13 |
| | TB | Hansen_pre_Vaccine | 0.09 | 0.72 | 0.12 | 0.09 | 0.17 | 0.21 | 0.41 | 0.22 | 0.57 |
| | vaccine | Hansen_preChallenge | NA | 0.34 | 0.10 | 0.14 | 0.42 | 0.09 | 0.29 | 0.16 | 0.20 |
| | | Hansen_postChallenge | NA | 0.41 | 0.26 | 0.24 | 0.41 | 0.16 | 0.13 | 0.21 | 0.23 |
| | | Rank 1 (F1 score) | 0.47 | 0.74 | 0.52 | 0.59 | 0.45 | 0.53 | 0.58 | 0.25 | 0.57 |

| | | | TCGA project | | | | | | |
|---|---|---|---|---|---|---|---|---|---|
| | | Training | READ | SARC | SKCM | STAD | UCEC | UCS | UVM |
| Training | Dengue | Devignot | 0.43 | 0.35 | 0.19 | 0.50 | 0.20 | 0.58 | 0.18 |
| | H1N1 | BermejoMartin | 0.43 | 0.25 | 0.11 | 0.51 | 0.32 | 0.29 | 0.18 |
| | IAV | Franco_Male_Day 0 | 0.45 | 0.35 | 0.21 | 0.07 | 0.37 | 0.58 | 0.40 |
| | Vaccine | Franco_Female_Day 0 | 0.43 | 0.34 | 0.07 | 0.58 | 0.12 | 0.40 | 0.29 |
| | | Franco_Male_Day 1 | 0.43 | 0.39 | 0.23 | 0.66 | 0.36 | 0.45 | 0.36 |
| | | Franco_Female_Day 1 | 0.29 | 0.17 | 0.13 | 0.44 | 0.31 | 0.24 | 0.44 |
| | | Franco_Male_Day 14 | 0.36 | 0.49 | 0.12 | 0.59 | 0.30 | 0.49 | NA |
| | | Franco_Female_Day 14 | NA | 0.42 | 0.16 | 0.30 | 0.28 | 0.27 | 0.40 |
| | HBV | Bartholomeus_Day 0 | 0.36 | 0.38 | 0.22 | 0.61 | 0.30 | 0.27 | NA |
| | vaccine | Bartholomeus_Day 3 | 0.25 | 0.42 | 0.23 | 0.22 | 0.20 | 0.40 | 0.17 |
| | | Bartholomeus_Day 7 | NA | 0.32 | 0.16 | 0.22 | 0.29 | 0.45 | NA |
| | TB | Hansen_pre_Vaccine | 0.29 | 0.08 | 0.22 | 0.16 | 0.33 | 0.24 | 0.14 |
| | vaccine | Hansen_preChallenge | NA | 0.09 | 0.20 | 0.16 | 0.33 | 0.38 | 0.46 |
| | | Hansen_postChallenge | 0.32 | 0.42 | 0.21 | 0.67 | 0.05 | 0.45 | 0.35 |
| | | Rank 1 (F1 score) | 0.45 | 0.49 | 0.23 | 0.67 | 0.37 | 0.58 | 0.46 |

TABLE 7C
Training and test datasets of related pairs based on apparent biological
relationships - log2 enrichment score. A value of >=3 indicates
that there were no true cases present in the assigned control cluster

| | | | SARS CoV2 | H1N1 | TB |
|---|---|---|---|---|---|
| | | Training | Liao | Dunning | Zak |
| Training | Dengue | Devignot | 1 | 1 | 7 |
| | H1N1 | BermejoMartin | 4 | 1 | 5 |
| | IAV | Franco_Male_Day 0 | 4 | 9 | 10 |
| | Vaccine | Franco_Female_Day 0 | 1 | 9 | 8 |
| | | Franco_Male_Day 1 | 1 | 9 | 3 |
| | | Franco_Female_Day 1 | 4 | 8 | 12 |
| | | Franco_Male_Day 14 | 4 | 9 | 13 |
| | | Franco_Female_Day 14 | 4 | 9 | 11 |
| | HBV | Bartholomeus_Day 0 | 4 | 4 | 14 |
| | vaccine | Bartholomeus_Day 3 | 4 | 9 | 9 |
| | | Bartholomeus_Day 7 | 4 | 6 | 6 |
| | TB | Hansen_—pre_Vaccine | 4 | 7 | 4 |
| | vaccine | Hansen_preChallenge | 4 | 1 | 2 |
| | | Hansen_postChallenge | 4 | 5 | 1 |
| | | Rank 1 (log2 enrichment) | >=3 | >=3 | 2.5 |

TABLE 7D
Training and test datasets on presumed unrelated pairs- log2 enrichment score. A value
of >=3 indicates that there were no true cases present in the assigned control cluster

| | | Asthma | | Rheumatoid Arth. | | NCI TARGET project | | | | |
|---|---|---|---|---|---|---|---|---|---|---|
| | Training | Bjornsdottir | Altman | Teixeira | Bienkowska | ALLP2 | ALLP3 | AML | OS | WT |
| Dengue | Devignot | 1 | 3 | 1 | 5 | 14 | 4 | 7 | 3 | 1 |
| H1N1 | BermejoMartin | 5 | 13 | 7 | 3 | 5 | NA | 8 | 4 | 4 |
| IAV | Franco_Male_Day 0 | 6 | 4 | NA | 3 | 8 | 12 | 14 | 12 | 8 |
| Vaccine | Franco_Female_Day 0 | 2 | 10 | 4 | 6 | 9 | 2 | 9 | 2 | 4 |
| | Franco_Male_Day 1 | 14 | 12 | NA | 2 | 2 | 5 | 4 | 6 | 11 |
| | Franco_Female_Day 1 | 4 | 2 | NA | 1 | 6 | 7 | 11 | 11 | 2 |
| | Franco_Male_Day 14 | 8 | 1 | NA | 13 | 1 | 9 | 10 | 12 | 10 |
| | Franco_Female_Day 14 | 7 | 5 | 10 | 7 | 13 | 7 | 5 | 7 | 7 |
| HBV | Bartholomeus_Day 0 | 10 | 8 | 9 | 11 | 7 | 3 | 12 | 4 | 6 |
| vaccine | Bartholomeus_Day 3 | 9 | 14 | 7 | 10 | 4 | 10 | 6 | 1 | 12 |
| | Bartholomeus_Day 7 | 11 | 6 | 6 | 14 | 9 | 13 | 2 | 12 | 2 |
| TB | Hansen_{——}pre_Vaccine | 3 | 7 | 2 | 7 | 9 | 6 | 12 | 9 | 14 |
| vaccine | Hansen_preChallenge | 13 | 8 | 3 | 7 | 12 | 1 | 1 | 8 | 3 |
| | Hansen_postChallenge | 12 | 11 | 5 | 12 | 3 | 11 | 3 | 10 | 9 |
| | Rank 1 (log2 enrichment) | 1.3 | 0.8 | >=3 | 0.8 | 2.1 | 1.8 | >=3 | 1.6 | >=3 |

| | | | TCGA project | | | | | | | | |
|---|---|---|---|---|---|---|---|---|---|---|---|
| | | Training | BLCA | BRCA | CESC | CHOL | COAD | ESCA | GBM | HNSC | KIRC |
| Training | Dengue | Devignot | 2 | 1 | 4 | 7 | 3 | 1 | 3 | 10 | 11 |
| | H1N1 | BermejoMartin | 4 | 13 | 12 | 9 | 14 | 8 | 10 | 9 | 6 |
| | IAV | Franco_Male_Day 0 | 11 | 9 | 1 | 12 | 8 | 9 | 4 | 11 | 7 |
| | Vaccine | Franco_Female_Day 0 | 7 | 14 | 1 | 11 | 10 | 3 | 1 | 12 | 8 |
| | | Franco_Male_Day 1 | 1 | 6 | 14 | 5 | 8 | 5 | 13 | 13 | 11 |
| | | Franco_Female_Day 1 | 10 | 3 | 6 | 13 | 12 | 14 | 5 | 3 | 14 |
| | | Franco_Male_{——}Day 14 | 13 | 7 | 3 | 6 | 5 | 6 | 14 | 2 | 3 |
| | | Franco_Female_Day 14 | 9 | 5 | 5 | 10 | 4 | 13 | 8 | 6 | 2 |
| | HBV | Bartholomeus_Day 0 | 14 | 8 | 11 | 2 | 2 | 4 | 10 | 5 | 9 |
| | vaccine | Bartholomeus_Day 3 | 2 | 4 | 13 | 3 | 7 | 11 | 12 | 7 | 5 |
| | | Bartholomeus_Day 7 | 12 | 2 | 8 | 8 | 13 | 12 | 9 | 7 | 4 |
| | TB | Hansen_pre_Vaccine | 4 | 10 | 7 | 4 | 6 | 7 | 6 | 4 | 1 |
| | vaccine | Hansen_preChallenge | 8 | 11 | 9 | 1 | 11 | 2 | 2 | 1 | 9 |
| | | Hansen_postChallenge | 6 | 12 | 10 | 13 | 1 | 10 | 7 | 14 | 13 |

| | | | | | | | | | |
|---|---|---|---|---|---|---|---|---|---|
| Rank 1 (log2 enrichment) | 0.5 | 1.6 | 1.5 | 1.9 | 0.3 | 0.5 | 1.0 | 0.6 | 0.3 |

| | | | TCGA project | | | | | | | | |
|---|---|---|---|---|---|---|---|---|---|---|---|
| | | Training | KIRP | LAML | LGG | LIHC | LUAD | LUSC | MESO | OV | PAAD |
| Training | Dengue | Devignot | 1 | 7 | 2 | 2 | 7 | 11 | 9 | 3 | 12 |
| | H1N1 | BermejoMartin | 9 | 1 | 1 | 5 | 11 | 9 | 3 | 4 | 14 |
| | IAV | Franco_Male_Day 0 | 4 | 4 | 3 | 3 | 1 | 3 | 1 | 2 | 1 |
| | Vaccine | Franco_Female_Day 0 | 2 | 3 | 7 | 6 | 7 | 1 | 11 | 5 | 6 |
| | | Franco_Male_Day 1 | 7 | 11 | 6 | 3 | 10 | 11 | 10 | 11 | 11 |
| | | Franco_Female_Day 1 | 3 | 10 | 4 | 8 | 13 | 6 | 5 | 1 | 7 |
| | | Franco_Male_Day 14 | 9 | 8 | 12 | 11 | 4 | 11 | 12 | 14 | 2 |
| | | Franco_Female_Day 14 | 6 | 2 | 10 | 1 | 9 | 8 | 8 | 12 | 9 |
| | HBV | Bartholomeus_Day 0 | 5 | 4 | 14 | 10 | 3 | 14 | 4 | 13 | 4 |
| | vaccine | Bartholomeus_Day 3 | 9 | 14 | 11 | 11 | 5 | 5 | 14 | 10 | 3 |
| | | Bartholomeus_Day 7 | 9 | 12 | 9 | 14 | 14 | 2 | 7 | 6 | 9 |
| | TB | Hansen_pre_Vaccine | 8 | 8 | 13 | 11 | 2 | 4 | 2 | 7 | 8 |
| | vaccine | Hansen_preChallenge | 9 | 13 | 8 | 9 | 6 | 9 | 13 | 9 | 13 |
| | | Hansen_postChallenge | 9 | 6 | 5 | 7 | 11 | 7 | 6 | 8 | 5 |
| | | Rank 1 (log2 enrichment) | 1.5 | 0.5 | 2.3 | 0.4 | 0.8 | 1.3 | 1.9 | 0.8 | 0.6 |

| | | | TCGA project | | | | | | |
|---|---|---|---|---|---|---|---|---|---|
| | | Training | READ | SARC | SKCM | STAD | UCEC | UCS | UVM |
| Training | Dengue | Devignot | 6 | 5 | 10 | 8 | 2 | 1 | 8 |
| | H1N1 | BermejoMartin | 1 | 10 | 14 | 8 | 12 | 14 | 8 |
| | IAV | Franco_Male_Day 0 | 4 | 7 | 5 | 14 | 9 | 1 | 4 |
| | Vaccine | Franco_Female_Day 0 | 3 | 8 | 13 | 5 | 9 | 9 | 7 |
| | | Franco_Male_Day 1 | 1 | 9 | 3 | 2 | 3 | 11 | 5 |
| | | Franco_Female_Day 1 | 9 | 12 | 8 | 10 | 4 | 7 | 1 |
| | | Franco_Male_Day 14 | 10 | 1 | 11 | 12 | 1 | 5 | 12 |
| | | Franco_Female_Day 14 | 12 | 2 | 12 | 13 | 11 | 11 | 3 |
| | HBV | Bartholomeus_Day 0 | 10 | 11 | 2 | 11 | 13 | 3 | 12 |
| | vaccine | Bartholomeus_Day 3 | 8 | 3 | 1 | 5 | 6 | 9 | 10 |
| | | Bartholomeus_Day 7 | 12 | 4 | 8 | 5 | 4 | 6 | 12 |
| | TB | Hansen_pre_Vaccine | 5 | 14 | 4 | 3 | 7 | 7 | 11 |
| | vaccine | Hansen_preChallenge | 12 | 13 | 7 | 3 | 7 | 4 | 2 |
| | | Hansen_postChallenge | 7 | 6 | 5 | 1 | 14 | 11 | 6 |
| | | Rank 1 (log2 enrichment) | 1.1 | 1.2 | 0.8 | 0.5 | 0.0 | >=3 | 1.6 |

TABLE 8
Gene Enrichment for Dengue Universal Signatures

| #term ID | Term Description | Labels |
|---|---|---|
| GO:0002376 | immune system | HMOX1, CTSG, OLFM4, LTA4H, LTF, MMP8, INHBA, |
| | process | LGALS3, KYNU, IFNGR2, PTX3, RNF31, ARG1, CD1D, |
| | | S100A8, S100A12, MAFB, KLF4, VSIG4, NOTCH4, IDH1, |
| | | TRIM26 |
| GO:0006950 | response to stress | PDK4, HMOX1, CTSG, LTF, INHBA, LGALS3, KYNU, |
| | | DUSP6, IFNGR2, PTX3, ARG1, MCRS1, MYOF, CD1D, |
| | | S100A8, S100A12, KLF4, VSIG4, NOTCH4, IDH1, PAPSS2, |
| | | TRIM26, CYP1B1 |
| GO:0043312 | neutrophil | CTSG, OLFM4, LTA4H, LTF, MMP8, LGALS3, PTX3, |
| | degranulation | ARG1, S100A8, S100A12, IDH1 |
| GO:0045055 | regulated exocytosis | CTSG, OLFM4, LTA4H, LTF, MMP8, LGALS3, PTX3, |
| | | ARG1, STX11, S100A8, S100A12, IDH1 |
| GO:0045321 | leukocyte activation | CTSG, OLFM4, LTA4H, LTF, MMP8, LGALS3, PTX3, |
| | | ARG1, CD1D, S100A8, S100A12, MAFB, IDH1 |
| GO:0006955 | immune response | CTSG, OLFM4, LTA4H, LTF, MMP8, LGALS3, KYNU, |
| | | IFNGR2, PTX3, ARG1, CD1D, S100A8, S100A12, VSIG4, |
| | | IDH1, TRIM26 |
| GO:0032940 | secretion by cell | CTSG, OLFM4, LTA4H, LTF, MMP8, INHBA, LGALS3, |
| | | PTX3, ARG1, STX11, S100A8, S100A12, IDH1 |
| GO:0006952 | defense response | HMOX1, CTSG, LTF, INHBA, LGALS3, KYNU, IFNGR2, |
| | | PTX3, ARG1, CD1D, S100A8, S100A12, VSIG4, TRIM26 |
| GO:0045087 | innate immune | LTF, LGALS3, KYNU, IFNGR2, PTX3, ARG1, CD1D, |
| | response | S100A8, S100A12, VSIG4, TRIM26 |
| GO:0098542 | defense response to | CTSG, LTF, LGALS3, KYNU, IFNGR2, PTX3, ARG1, |
| | other organism | CD1D, S100A8, S100A12, VSIG4, TRIM26 |
| GO:0050776 | regulation of immune | HMOX1, CTSG, LTF, LGALS3, CD81, IFNGR2, RNF31, |
| | response | COL17A1, ARG1, CD1D, S100A8, VSIG4 |
| GO:0002252 | immune effector | CTSG, OLFM4, LTA4H, LTF, MMP8, LGALS3, PTX3, |
| | process | ARG1, S100A8, S100A12, VSIG4, IDH1 |
| GO:0009620 | response to fungus | CTSG, LTF, PTX3, S100A8, S100A12 |
| GO:0002682 | regulation of immune | HMOX1, CTSG, LTF, INHBA, LGALS3, CD81, IFNGR2, |
| | system process | RNF31, COL17A1, ARG1, CD1D, S100A8, MAFB, VSIG4 |
| GO:0002684 | positive regulation of | HMOX1, CTSG, LTF, INHBA, LGALS3, CD81, RNF31, |
| | immune system | ARG1, CD1D, S100A8, VSIG4 |
| | process | |
| GO:0051090 | regulation of DNA- | HMOX1, LTF, RNF31, S100A8, S100A12, KLF4, TRIM26, |
| | binding transcription | CYP1B1 |
| | factor activity | |
| GO:0050832 | defense response to | CTSG, LTF, S100A8, S100A12 |
| | fungus | |
| GO:0043900 | regulation of multi- | CTSG, LTF, INHBA, IFNGR2, PTX3, ARG1, CD1D, |
| | organism process | S100A8, TRIM26 |
| GO:0019730 | antimicrobial | CTSG, LTF, LGALS3, S100A8, S100A12 |
| | humoral response | |
| GO:0006959 | humoral immune | CTSG, LTF, LGALS3, S100A8, S100A12, VSIG4 |
| | response | |
| GO:0016192 | vesicle-mediated | CTSG, OLFM4, LTA4H, LTF, MMP8, LGALS3, CD81, |
| | transport | PTX3, ARG1, STX11, S100A8, S100A12, IDH1 |
| GO:0050896 | response to stimulus | PDK4, HMOX1, CTSG, OLFM4, LTA4H, LTF, MMP8, |
| | | INHBA, LGALS3, CD81, KYNU, DUSP6, IFNGR2, PTX3, |
| | | RNF31, ARG1, MCRS1, MYOF, CD1D, S100A8, S100A12, |
| | | KLF4, VSIG4, NOTCH4, IDH1, PAPSS2, TRIM26, GSTK1, |
| | | CYP1B1 |
| GO:0031640 | killing of cells of | CTSG, LTF, LGALS3, S100A12 |
| | other organism | |
| GO:0035821 | modification of | CTSG, LTF, LGALS3, PTX3, S100A12 |
| | morphology or | |
| | physiology of other | |
| | organism | |
| GO:0044364 | disruption of cells of | CTSG, LTF, LGALS3, S100A12 |
| | other organism | |
| GO:0009605 | response to external | PDK4, HMOX1, CTSG, LTF, LGALS3, KYNU, IFNGR2, |
| | stimulus | PTX3, ARG1, CD1D, S100A8, S100A12, VSIG4, TRIM26 |
| GO:0097237 | cellular response to | HMOX1, ARG1, KLF4, GSTK1, CYP1B1 |
| | toxic substance | |
| GO:0031347 | regulation of defense | LTF, CD81, IFNGR2, ARG1, CD1D, S100A8, S100A12, |
| | response | KLF4 |
| GO:0043903 | regulation of | CTSG, LTF, PTX3, ARG1, TRIM26 |
| | symbiosis, | |
| | encompassing | |
| | mutualism through | |
| | parasitism | |
| GO:0043901 | negative regulation of | CTSG, LTF, PTX3, ARG1, TRIM26 |
| | multi-organism | |
| | process | |
| GO:0042542 | response to hydrogen | HMOX1, ARG1, KLF4, CYP1B1 |
| | peroxide | |
| GO:0001818 | negative regulation of | HMOX1, LTF, INHBA, ARG1, KLF4 |
| | cytokine production | |
| GO:0002762 | negative regulation of | LTF, INHBA, MAFB |
| | myeloid leukocyte | |
| | differentiation | |
| GO:0051091 | positive regulation of | LTF, RNF31, S100A8, S100A12, TRIM26 |
| | DNA-binding | |
| | transcription factor | |
| | activity | |
| GO:0002683 | negative regulation of | HMOX1, LTF, INHBA, LGALS3, ARG1, MAFB |
| | immune system | |
| | process | |
| GO:0044793 | negative regulation | LTF, PTX3 |
| | by host of viral | |
| | process | |
| GO:0051092 | positive regulation of | LTF, RNF31, S100A8, S100A12 |
| | NF-kappaB | |
| | transcription factor | |
| | activity | |
| GO:0048646 | anatomical structure | HMOX1, MMP8, INHBA, MYOF, MAFB, KLF4, |
| | formation involved in | NOTCH4, CYP1B1 |
| | morphogenesis | |
| GO:0030155 | regulation of cell | OLFM4, LGALS3, ARG1, CD1D, KLF4, FRMD5, CYP1B1 |
| | adhesion | |
| GO:0022610 | biological adhesion | OLFM4, CD81, CSTA, VCAN, COL17A1, CD1D, S100A8, |
| | | CYP1B1 |
| GO:0030593 | neutrophil | LGALS3, S100A8, S100A12 |
| | chemotaxis | |
| GO:0048518 | positive regulation of | HMOX1, CTSG, OLFM4, LTF, INHBA, LGALS3, CD81, |
| | biological process | DUSP6, PTX3, RNF31, ARG1, MCRS1, CD1D, S100A8, |
| | | S100A12, MAFB, KLF4, VSIG4, NOTCH4, FRMD5, |
| | | TRIM26, CYP1B1 |
| GO:0048583 | regulation of | HMOX1, CTSG, LTF, INHBA, LGALS3, CD81, DUSP6, |
| | response to stimulus | IFNGR2, RNF31, COL17A1, ARG1, MYOF, CD1D, |
| | | S100A8, S100A12, KLF4, VSIG4, CYP1B1 |
| GO:0040013 | negative regulation of | HMOX1, KLF4, FRMD5, TRIM26, CYP1B1 |
| | locomotion | |
| GO:0002695 | negative regulation of | HMOX1, INHBA, LGALS3, ARG1 |
| | leukocyte activation | |
| GO:0048856 | anatomical structure | HMOX1, LTF, MMP8, INHBA, LGALS3, CSTA, VCAN, |
| | development | DUSP6, B3GNT5, COL17A1, ARG1, MYOF, CD1D, |
| | | S100A8, MAFB, KLF4, NOTCH4, IDH1, PAPSS2, GSTK1, |
| | | CYP1B1 |
| GO:0070301 | cellular response to | ARG1, KLF4, CYP1B1 |
| | hydrogen peroxide | |
| GO:0060759 | regulation of | IFNGR2, RNF31, ARG1, KLF4 |
| | response to cytokine | |
| | stimulus | |
| GO:0002694 | regulation of | HMOX1, INHBA, LGALS3, CD81, ARG1, CD1D |
| | leukocyte activation | |
| GO:0009636 | response to toxic | HMOX1, ARG1, S100A8, KLF4, GSTK1, CYP1B1 |
| | substance | |
| GO:0046677 | response to antibiotic | HMOX1, ARG1, S100A8, KLF4, CYP1B1 |
| GO:0042493 | response to drug | HMOX1, INHBA, KYNU, DUSP6, ARG1, S100A8, KLF4, |
| | | CYP1B1 |
| GO:0051851 | modification by host | CTSG, LTF, PTX3 |
| | of symbiont | |
| | morphology or | |
| | physiology | |
| GO:1903725 | regulation of | CD81, KLF4, IDH1 |
| | phospholipid | |
| | metabolic process | |
| GO:1903901 | negative regulation of | LTF, PTX3, TRIM26 |
| | viral life cycle | |
| GO:0048584 | positive regulation of | CTSG, LTF, INHBA, CD81, DUSP6, RNF31, ARG1, |
| | response to stimulus | CD1D, S100A8, S100A12, VSIG4, CYP1B1 |
| GO:0032101 | regulation of | LTF, CD81, IFNGR2, ARG1, CD1D, S100A8, S100A12, |
| | response to external | KLF4 |
| | stimulus | |
| GO:0044419 | interspecies | CTSG, LTF, LGALS3, CD81, PTX3, CD1D, S100A12 |
| | interaction between | |
| | organisms | |
| GO:0006790 | sulfur compound | KYNU, VCAN, IDH1, PAPSS2, GSTK1 |
| | metabolic process | |
| GO:0046597 | negative regulation of | PTX3, TRIM26 |
| | viral entry into host | |
| | cell | |
| GO:0009611 | response to wounding | HMOX1, ARG1, MYOF, S100A8, NOTCH4, PAPSS2 |
| GO:0045088 | regulation of innate | LTF, IFNGR2, ARG1, CD1D, S100A8 |
| | immune response | |
| GO:0050670 | regulation of | LGALS3, CD81, ARG1, CD1D |
| | lymphocyte | |
| | proliferation | |
| GO:0009617 | response to bacterium | CTSG, LTF, ARG1, CD1D, S100A8, S100A12 |
| GO:0031349 | positive regulation of | LTF, ARG1, CD1D, S100A8, S100A12 |
| | defense response | |
| GO:0010033 | response to organic | PDK4, HMOX1, CTSG, INHBA, CD81, KYNU, DUSP6, |
| | substance | IFNGR2, ARG1, S100A8, KLF4, IDH1, TRIM26, CYP1B1 |
| GO:0006979 | response to oxidative | HMOX1, ARG1, KLF4, IDH1, CYP1B1 |
| | stress | |
| GO:0042035 | regulation of cytokine | HMOX1, INHBA, KLF4 |
| | biosynthetic process | |
| GO:0051704 | multi-organism | CTSG, LTF, LGALS3, CD81, KYNU, IFNGR2, PTX3, |
| | process | ARG1, CD1D, S100A8, S100A12, VSIG4, TRIM26 |
| GO:0034599 | cellular response to | HMOX1, ARG1, KLF4, CYP1B1 |
| | oxidative stress | |
| GO:0046916 | cellular transition | HMOX1, LTF, S100A8 |
| | metal ion | |
| | homeostasis | |
| GO:0050778 | positive regulation of | CTSG, LTF, RNF31, CD1D, S100A8, VSIG4 |
| | immune response | |
| GO:0043902 | positive regulation of | LTF, INHBA, ARG1, CD1D, S100A8 |
| | multi-organism | |
| | process | |
| GO:0002719 | negative regulation of | HMOX1, ARG1 |
| | cytokine production | |
| | involved in immune | |
| | response | |
| GO:0033993 | response to lipid | PDK4, CTSG, INHBA, ARG1, S100A8, KLF4, IDH1 |
| GO:0051249 | regulation of | INHBA, LGALS3, CD81, ARG1, CD1D |
| | lymphocyte | |
| | activation | |
| GO:0001817 | regulation of cytokine | HMOX1, LTF, INHBA, ARG1, KLF4, CYP1B1 |
| | production | |
| GO:0007155 | cell adhesion | OLFM4, CSTA, VCAN, COL17A1, CD1D, S100A8, |
| | | CYP1B1 |
| GO:0048333 | mesodermal cell | INHBA, KLF4 |
| | differentiation | |
| GO:0060334 | regulation of | IFNGR2, ARG1 |
| | interferon-gamma- | |
| | mediated signaling | |
| | pathway | |
| GO:0061844 | antimicrobial | LTF, LGALS3, S100A12 |
| | humoral immune | |
| | response mediated by | |
| | antimicrobial peptide | |
| GO:0065009 | regulation of | HMOX1, LTF, INHBA, LGALS3, CD81, CSTA, DUSP6, |
| | molecular function | PTX3, RNF31, MCRS1, S100A8, S100A12, KLF4, TRIM26, |
| | | CYP1B1 |
| GO:0007162 | negative regulation of | LGALS3, ARG1, KLF4, CYP1B1 |
| | cell adhesion | |
| GO:0071236 | cellular response to | ARG1, KLF4, CYP1B1 |
| | antibiotic | |
| GO:1901564 | organonitrogen | PDK4, HMOX1, CTSG, LTA4H, LTF, MMP8, INHBA, |
| | compound metabolic | KYNU, CSTA, VCAN, DUSP6, RNF31, B3GNT5, ARG1, |
| | process | MCRS1, S100A8, VSIG4, IDH1, PAPSS2, GSTK1 |
| GO:1903038 | negative regulation of | LGALS3, ARG1, KLF4 |
| | leukocyte cell-cell | |
| | adhesion | |
| GO:0001704 | formation of primary | MMP8, INHBA, KLF4 |
| | germ layer | |
| GO:0002698 | negative regulation of | HMOX1, LGALS3, ARG1 |
| | immune effector | |
| | process | |
| GO:0042742 | defense response to | CTSG, LTF, S100A8, S100A12 |
| | bacterium | |
| GO:0044092 | negative regulation of | HMOX1, LTF, CSTA, DUSP6, PTX3, MCRS1, KLF4, |
| | molecular function | CYP1B1 |
| GO:0045637 | regulation of myeloid | LTF, INHBA, LGALS3, MAFB |
| | cell differentiation | |
| GO:0045671 | negative regulation of | LTF, MAFB |
| | osteoclast | |
| | differentiation | |
| GO:0014070 | response to organic | INHBA, KYNU, DUSP6, ARG1, KLF4, IDH1, CYP1B1 |
| | cyclic compound | |
| GO:0042036 | negative regulation of | INHBA, KLF4 |
| | cytokine biosynthetic | |
| | process | |
| GO:2000146 | negative regulation of | HMOX1, KLF4, FRMD5, CYP1B1 |
| | cell motility | |
| GO:0070887 | cellular response to | PDK4, HMOX1, CTSG, INHBA, LGALS3, IFNGR2, ARG1, |
| | chemical stimulus | S100A8, S100A12, KLF4, TRIM26, GSTK1, CYP1B1 |
| GO:0040012 | regulation of | HMOX1, LGALS3, CD81, KLF4, FRMD5, TRIM26, |
| | locomotion | CYP1B1 |
| GO:0009966 | regulation of signal | HMOX1, LTF, INHBA, LGALS3, CD81, DUSP6, IFNGR2, |
| | transduction | RNF31, ARG1, MYOF, S100A8, S100A12, KLF4, |
| | | CYP1B1 |
| GO:0042221 | response to chemical | PDK4, HMOX1, CTSG, INHBA, LGALS3, CD81, KYNU, |
| | | DUSP6, IFNGR2, ARG1, S100A8, S100A12, KLF4, IDH1, |
| | | TRIM26, GSTK1, CYP1B1 |
| GO:0043123 | positive regulation of | LTF, RNF31, S100A12 |
| | I-kappaB kinase/NF- | |
| | kappaB signaling | |
| GO:0042060 | wound healing | HMOX1, MYOF, S100A8, NOTCH4, PAPSS2 |
| GO:0002833 | positive regulation of | LTF, ARG1, CD1D, S100A8 |
| | response to biotic | |
| | stimulus | |
| GO:1903037 | regulation of | LGALS3, ARG1, CD1D, KLF4 |
| | leukocyte cell-cell | |
| | adhesion | |
| GO:0043436 | oxoacid metabolic | LTA4H, KYNU, VCAN, ARG1, IDH1, PAPSS2, CYP1B1 |
| | process | |
| GO:0051250 | negative regulation of | INHBA, LGALS3, ARG1 |
| | lymphocyte | |
| | activation | |
| GO:0032787 | monocarboxylic acid | LTA4H, KYNU, VCAN, IDH1, CYP1B1 |
| | metabolic process | |
| GO:0042981 | regulation of | PDK4, HMOX1, LTF, INHBA, LGALS3, DUSP6, S100A8, |
| | apoptotic process | KLF4, CYP1B1 |
| GO:0050777 | negative regulation of | HMOX1, LGALS3, ARG1 |
| | immune response | |
| GO:0090049 | regulation of cell | HMOX1, KLF4 |
| | migration involved in | |
| | sprouting | |
| | angiogenesis | |
| GO:0010470 | regulation of | DUSP6, KLF4 |
| | gastrulation | |
| GO:1903672 | positive regulation of | HMOX1, KLF4 |
| | sprouting | |
| | angiogenesis | |
| GO:0001505 | regulation of | PTX3, STX11, KLF4, CYP1B1 |
| | neurotransmitter | |
| | levels | |
| GO:0071396 | cellular response to | PDK4, CTSG, INHBA, ARG1, KLF4 |
| | lipid | |
| GO:1902533 | positive regulation of | LTF, CD81, DUSP6, RNF31, S100A8, S100A12, CYP1B1 |
| | intracellular signal | |
| | transduction | |
| GO:0030198 | extracellular matrix | CTSG, MMP8, VCAN, CYP1B1 |
| | organization | |
| GO:0010035 | response to inorganic | HMOX1, ARG1, S100A8, KLF4, CYP1B1 |
| | substance | |
| GO:0032103 | positive regulation of | LTF, ARG1, CD1D, S100A8, S100A12 |
| | response to external | |
| | stimulus | |
| GO:0002548 | monocyte chemotaxis | LGALS3, S100A12 |
| GO:0035987 | endodermal cell | MMP8, INHBA |
| | differentiation | |
| GO:0043603 | cellular amide | CTSG, LTA4H, KYNU, ARG1, IDH1, GSTK1 |
| | metabolic process | |
| GO:0045429 | positive regulation of | PTX3, KLF4 |
| | nitric oxide | |
| | biosynthetic process | |
| GO:0035690 | cellular response to | HMOX1, ARG1, KLF4, CYP1B1 |
| | drug | |
| GO:0001709 | cell fate | KLF4, NOTCH4 |
| | determination | |
| GO:0001959 | regulation of | IFNGR2, RNF31, ARG1 |
| | cytokine-mediated | |
| | signaling pathway | |
| GO:0042129 | regulation of T cell | LGALS3, ARG1, CD1D |
| | proliferation | |
| GO:0048662 | negative regulation of | HMOX1, KLF4 |
| | smooth muscle cell | |
| | proliferation | |
| GO:0002886 | regulation of myeloid | HMOX1, ARG1 |
| | leukocyte mediated | |
| | immunity | |
| GO:0034605 | cellular response to | HMOX1, MYOF |
| | heat | |
| GO:0030097 | hemopoiesis | INHBA, CD1D, MAFB, KLF4, NOTCH4 |
| GO:0042127 | regulation of cell | HMOX1, LTF, INHBA, LGALS3, CD81, ARG1, CD1D, |
| | population | KLF4, CYP1B1 |
| | proliferation | |
| GO:0043433 | negative regulation of | HMOX1, KLF4, CYP1B1 |
| | DNA-binding | |
| | transcription factor | |
| | activity | |
| GO:0045646 | regulation of | INHBA, MAFB |
| | erythrocyte | |
| | differentiation | |
| GO:0048513 | animal organ | HMOX1, LTF, INHBA, CSTA, B3GNT5, ARG1, CD1D, |
| | development | MAFB, KLF4, NOTCH4, IDH1, PAPSS2, CYP1B1 |
| GO:0071466 | cellular response to | ARG1, S100A12, CYP1B1 |
| | xenobiotic stimulus | |
| GO:2001236 | regulation of extrinsic | HMOX1, INHBA, LGALS3 |
| | apoptotic signaling | |
| | pathway | |
| GO:0019731 | antibacterial humoral | CTSG, LTF |
| | response | |
| GO:0050886 | endocrine process | CTSG, INHBA |
| GO:0045766 | positive regulation of | HMOX1, KLF4, CYP1B1 |
| | angiogenesis | |
| GO:0002704 | negative regulation of | HMOX1, ARG1 |
| | leukocyte mediated | |
| | immunity | |
| GO:0009888 | tissue development | MMP8, INHBA, LGALS3, CSTA, COL17A1, KLF4, |
| | | NOTCH4, GSTK1, CYP1B1 |
| GO:0051972 | regulation of | MCRS1, KLF4 |
| | telomerase activity | |
| GO:0050727 | regulation of | CD81, S100A8, S100A12, KLF4 |
| | inflammatory | |
| | response | |
| GO:0071902 | positive regulation of | LTF, CD81, DUSP6, S100A12 |
| | protein | |
| | serine/threonine | |
| | kinase activity | |
| GO:2000377 | regulation of reactive | PTX3, KLF4, CYP1B1 |
| | oxygen species | |
| | metabolic process | |
| GO:0006749 | glutathione metabolic | IDH1, GSTK1 |
| | process | |
| GO:0010043 | response to zinc ion | ARG1, S100A8 |
| GO:0044272 | sulfur compound | VCAN, PAPSS2, GSTK1 |
| | biosynthetic process | |
| GO:0008152 | metabolic process | PDK4, HMOX1, CTSG, LTA4H, LTF, MMP8, INHBA, |
| | | LGALS3, ALDH2, CD81, KYNU, CSTA, VCAN, DUSP6, |
| | | RNF31, B3GNT5, ARG1, MCRS1, S100A8, S100A12, MAFB, |
| | | KLF4, VSIG4, NOTCH4, IDH1, PAPSS2, GSTK1, |
| | | CYP1B1 |
| GO:0034341 | response to | KYNU, IFNGR2, TRIM26 |
| | interferon-gamma | |
| GO:2000145 | regulation of cell | HMOX1, LGALS3, CD81, KLF4, FRMD5, CYP1B1 |
| | motility | |
| GO:0009653 | anatomical structure | HMOX1, LTF, MMP8, INHBA, ARG1, MYOF, MAFB, |
| | morphogenesis | KLF4, NOTCH4, CYP1B1 |
| GO:0032963 | collagen metabolic | MMP8, ARG1 |
| | process | |
| GO:0043086 | negative regulation of | LTF, CSTA, DUSP6, PTX3, MCRS1, KLF4 |
| | catalytic activity | |
| GO:0043550 | regulation of lipid | CD81, KLF4 |
| | kinase activity | |

TABLE 9
Gene Enrichment for Tuberculosis Universal Signatures

| #Term ID | Term Description | Labels |
|---|---|---|
| GO:0010033 | response to organic | CD4, PSME2, EHD4, EPOR, NAMPT, IGFBP2, SEC61A1, |
| | substance | FOSB, TRIM21, TRAFD1, RIPK1, MRPL15, CCNE1, CPT1A, |
| | | SORD, TP53, FEZ1, KCNMA1, AIFM1, HMGCR, ITGA2, |
| | | FASN, CXCL10, MCM7, STAT2, SHMT1, CALR, ANKZF1, |
| | | PDIA5, FBN1, PSEN1, TP53INP1, ATF3, FAS, STAT1, |
| | | DUSP10, GCLM, FMR1, CXCR3, PSMB8, FBXO6, |
| | | CD274, JAK2, ETS1, SLC26A6, IRF7, PPARA, SNX10, DDOST, |
| | | GCH1, CASP1, NR4A1, NUB1, EPHX1 |
| GO:0034097 | response to cytokine | CD4, PSME2, EPOR, SEC61A1, TRIM21, TRAFD1, RIPK1, |
| | | MRPL15, TP53, FASN, CXCL10, STAT2, SHMT1, FAS, |
| | | STAT1, GCLM, CXCR3, PSMB8, CD274, JAK2, ETS1, |
| | | SLC26A6, IRF7, SNX10, DDOST, GCH1, CASP1, NUB1 |
| GO:0008152 | metabolic process | B4GALT7, AAAS, PSME2, MPG, NAMPT, LAP3, RRP9, |
| | | IGFBP2, DDX39A, FOSB, IDUA, ACLY, TRIM21, RIPK1, |
| | | RNF144B, MRPL15, MOCOS, LPCAT2, CCNE1, LCT, |
| | | PSMD3, CREM, POLA2, CPT1A, EIF4H, SORD, TP53, |
| | | BCKDHA, CTSK, PRSS23, PTS, UCHL1, UBE2L6, AIFM1, |
| | | HMGCR, DDB1, FASN, BMP1, MCM7, GMPPB, NUP93, |
| | | C1QB, PRPF3, STAT2, GYS1, SHMT1, CALR, ANKZF1, |
| | | PDIA5, FBN1, PSEN1, NOC4L, MXI1, IDH2, STARD3, |
| | | ETV7, PPM1G, TP53INP1, ATF3, GPAA1, WARS, VAT1, |
| | | GMPPA, EDC4, BAZ1A, STAT1, PJA1, DUSP10, NDUFS2, |
| | | DNASE1L1, GCLM, FMR1, AKR1A1, YRDC, LDLRAP1, |
| | | C1QA, PSMB8, FOXP3, FBXO6, PDHA1, RDH11, JAK2, |
| | | DCP2, ETS1, DHRS7B, TYMP, IRF7, LSS, ATG4B, |
| | | NOLC1, PPARA, CDC7, DDOST, MGAT1, GCH1, DAPP1, |
| | | CASP1, CHI3L2, LDHC, NR4A1, NUB1, ENGASE, |
| | | PLA2G4C, EPHX1 |
| GO:0042221 | response to chemical | CD4, PSME2, EHD4, EPOR, NAMPT, IGFBP2, SEC61A1, |
| | | FOSB, TRIM21, TRAFD1, RIPK1, MRPL15, CCNE1, |
| | | CPT1A, SORD, TP53, FEZ1, SLC7A11, KCNMA1, AIFM1, |
| | | HMGCR, ITGA2, FASN, CXCL10, MCM7, STAT2, SHMT1, |
| | | CALR, ANKZF1, PDIA5, FBN1, PSEN1, RASGRP2, |
| | | TP53INP1, ATF3, FAS, STAT1, DUSP10, S100A10, VAV3, |
| | | GCLM, FMR1, CXCR3, C1QA, PSMB8, FBXO6, CD274, |
| | | JAK2, ETS1, SLC26A6, TYMP, IRF7, PPARA, SNX10, |
| | | DDOST, GCH1, CASP1, NR4A1, NUB1, EPHX1 |
| GO:0071704 | organic substance | B4GALT7, AAAS, PSME2, MPG, NAMPT, LAP3, RRP9, |
| | metabolic process | IGFBP2, DDX39A, FOSB, IDUA, ACLY, TRIM21, RIPK1, |
| | | RNF144B, MRPL15, MOCOS, LPCAT2, CCNE1, LCT, |
| | | PSMD3, CREM, POLA2, CPT1A, EIF4H, SORD, TP53, |
| | | BCKDHA, CTSK, PRSS23, PTS, UCHL1, UBE2L6, HMGCR, |
| | | DDB1, FASN, BMP1, MCM7, GMPPB, NUP93, C1QB, |
| | | PRPF3, STAT2, GYS1, SHMT1, CALR, ANKZF1, FBN1, |
| | | PSEN1, NOC4L, MXI1, IDH2, STARD3, ETV7, PPM1G, |
| | | TP53INP1, ATF3, GPAA1, WARS, EDC4, BAZ1A, STAT1, |
| | | PJA1, DUSP10, NDUFS2, DNASE1L1, GCLM, FMR1, |
| | | AKR1A1, YRDC, LDLRAP1, C1QA, PSMB8, FOXP3, FBXO6, |
| | | PDHA1, RDH11, JAK2, DCP2, ETS1, DHRS7B, TYMP, |
| | | IRF7, LSS, ATG4B, NOLC1, PPARA, CDC7, DDOST, |
| | | MGAT1, GCH1, DAPP1, CASP1, CHI3L2, LDHC, NR4A1, |
| | | NUB1, ENGASE, PLA2G4C, EPHX1 |
| GO:0070887 | cellular response to | CD4, PSME2, EHD4, EPOR, IGFBP2, FOSB, TRIM21, |
| | chemical stimulus | RIPK1, MRPL15, CCNE1, CPT1A, TP53, FEZ1, AIFM1, |
| | | ITGA2, FASN, CXCL10, MCM7, STAT2, SHMT1, CALR, |
| | | ANKZF1, PDIA5, FBN1, PSEN1, RASGRP2, TP53INP1, |
| | | ATF3, FAS, STAT1, VAV3, GCLM, FMR1, CXCR3, PSMB8, |
| | | JAK2, ETS1, SLC26A6, IRF7, PPARA, SNX10, CASP1, |
| | | NR4A1, EPHX1 |
| GO:0009605 | response to external | CD4, CLEC4A, IGFBP2, SEC61A1, FOSB, TRIM21, |
| | stimulus | SORD, TP53, FEZ1, AIFM1, HMGCR, ITGA2, CXCL10, |
| | | BANF1, C1QB, STAT2, ATF3, FAS, STAT1, DUSP10, VAV3, |
| | | GCLM, FMR1, CXCR3, C1QA, PSMB8, FOXP3, RDH11, |
| | | JAK2, ETS1, SLC26A6, TYMP, IRF7, PPARA, GCH1, |
| | | CASP1, NR4A1, NUB1 |
| GO:0042493 | response to drug | IGFBP2, FOSB, CPT1A, SORD, TP53, SLC7A11, KCNMA1, |
| | | AIFM1, HMGCR, ITGA2, MCM7, CALR, ANKZF1, |
| | | TP53INP1, STAT1, S100A10, VAV3, GCLM, FMR1, ETS1, |
| | | SLC26A6, PPARA, CASP1 |
| GO:0044238 | primary metabolic | B4GALT7, PSME2, MPG, NAMPT, LAP3, RRP9, IGFBP2, |
| | process | DDX39A, FOSB, IDUA, ACLY, TRIM21, RIPK1, |
| | | RNF144B, MRPL15, MOCOS, LPCAT2, CCNE1, LCT, PSMD3, |
| | | CREM, POLA2, CPT1A, EIF4H, SORD, TP53, BCKDHA, |
| | | CTSK, PRSS23, PTS, UCHL1, UBE2L6, HMGCR, DDB1, |
| | | FASN, BMP1, MCM7, GMPPB, C1QB, PRPF3, STAT2, |
| | | GYS1, SHMT1, CALR, ANKZF1, FBN1, PSEN1, NOC4L, |
| | | MXI1, IDH2, STARD3, ETV7, PPM1G, TP53INP1, ATF3, |
| | | GPAA1, WARS, EDC4, BAZ1A, STAT1, PJA1, DUSP10, |
| | | NDUFS2, DNASE1L1, GCLM, FMR1, AKR1A1, YRDC, |
| | | LDLRAP1, C1QA, PSMB8, FOXP3, FBXO6, PDHA1, |
| | | RDH11, JAK2, DCP2, ETS1, DHRS7B, TYMP, IRF7, LSS, |
| | | ATG4B, NOLC1, PPARA, CDC7, DDOST, MGAT1, DAPP1, |
| | | CASP1, CHI3L2, LDHC, NR4A1, NUB1, ENGASE, |
| | | PLA2G4C |
| GO:0071310 | cellular response to | CD4, PSME2, EHD4, EPOR, IGFBP2, FOSB, TRIM21, |
| | organic substance | RIPK1, MRPL15, CCNE1, CPT1A, TP53, FEZ1, AIFM1, |
| | | ITGA2, FASN, CXCL10, MCM7, STAT2, SHMT1, CALR, |
| | | ANKZF1, PDIA5, FBN1, PSEN1, TP53INP1, ATF3, FAS, |
| | | STAT1, GCLM, CXCR3, PSMB8, JAK2, SLC26A6, IRF7, |
| | | PPARA, SNX10, CASP1, NR4A1 |
| GO:0006950 | response to stress | CD4, MPG, CLEC4A, DDX39A, SEC61A1, TRIM21, |
| | | RIPK1, SORD, TP53, SLC7A11, UCHL1, KCNMA1, UBE2L6, |
| | | AIFM1, ITGA2, DDB1, CXCL10, MCM7, C1QB, STAT2, |
| | | CALR, ANKZF1, PDIA5, PSEN1, SFN, TP53INP1, ATF3, |
| | | FAS, STAT1, NDUFS2, VAV3, GCLM, FMR1, CXCR3, |
| | | C1QA, PSMB8, FBXO6, JAK2, ETS1, SLC26A6, IRF7, |
| | | IFRD1, NOLC1, PPARA, CDC7, GCH1, CASP1, NUB1, |
| | | PLA2G4C |
| GO:0044281 | small molecule | NAMPT, IDUA, ACLY, MOCOS, CREM, CPT1A, SORD, |
| | metabolic process | BCKDHA, PTS, HMGCR, FASN, GMPPB, SHMT1, |
| | | FBN1, IDH2, STARD3, ATF3, WARS, NDUFS2, GCLM, |
| | | AKR1A1, LDLRAP1, PDHA1, RDH11, DHRS7B, TYMP, LSS, |
| | | PPARA, MGAT1, GCH1, LDHC, PLA2G4C, EPHX1 |
| GO:0002376 | immune system | CD4, CLEC4A, SEC61A1, RRAS, ACLY, TRIM21, RIPK1, |
| | process | PSMD3, SEC24D, SLC7A11, FASN, CXCL10, C1QB, |
| | | STAT2, CALR, PSEN1, VAT1, FAS, STAT1, DNASE1L1, |
| | | VAV3, CXCR3, C1QA, PSMB8, FOXP3, CD274, JAK2, |
| | | ETS1, DHRS7B, SLC26A6, IRF7, PDCD1LG2, KIF2A, |
| | | BCAP31, SNX10, DDOST, GCH1, CASP1, NUB1 |
| GO:0005975 | carbohydrate | B4GALT7, IDUA, LCT, CREM, CPT1A, SORD, GYS1, |
| | metabolic process | FBN1, IDH2, ATF3, AKR1A1, PDHA1, MGAT1, CHI3L2, |
| | | LDHC |
| GO:0050896 | response to stimulus | CD4, PSME2, MPG, EHD4, EPOR, NAMPT, CLEC4A, |
| | | IGFBP2, DDX39A, SEC61A1, FOSB, RRAS, ACLY, TRIM21, |
| | | TRAFD1, RIPK1, MRPL15, CCNE1, PSMD3, CREM, |
| | | CPT1A, SORD, TP53, FEZ1, SLC7A11, UCHL1, KCNMA1, |
| | | UBE2L6, AIFM1, HMGCR, ITGA2, DDB1, FASN, |
| | | CXCL10, MCM7, BANF1, NUP93, C1QB, STAT2, SHMT1, |
| | | CALR, ANKZF1, PDIA5, FBN1, PSEN1, RASGRP2, SFN, |
| | | TP53INP1, ATF3, VAT1, FAS, STAT1, DUSP10, NDUFS2, |
| | | S100A10, DNASE1L1, VAV3, GCLM, FMR1, CXCR3, |
| | | C1QA, PSMB8, FOXP3, FBXO6, RDH11, CD274, JAK2, |
| | | ETS1, SLC26A6, TYMP, IRF7, PDCD1LG2, IFRD1, NOLC1, |
| | | PPARA, BCAP31, CDC7, SNX10, DDOST, GCH1, DAPP1, |
| | | CASP1, NR4A1, NUB1, PLA2G4C, EPHX1 |
| GO:0043065 | positive regulation of | RIPK1, TP53, KCNMA1, AIFM1, HMGCR, BCL2L14, |
| | apoptotic process | PSEN1, SFN, TP53INP1, ATF3, FAS, VAV3, CXCR3, |
| | | CD274, JAK2, BCAP31, CASP1 |
| GO:0006807 | nitrogen compound | B4GALT7, PSME2, MPG, NAMPT, LAP3, RRP9, IGFBP2, |
| | metabolic process | DDX39A, FOSB, IDUA, ACLY, TRIM21, RIPK1, |
| | | RNF144B, MRPL15, MOCOS, LPCAT2, CCNE1, PSMD3, |
| | | CREM, POLA2, CPT1A, EIF4H, TP53, BCKDHA, CTSK, |
| | | PRSS23, PTS, UCHL1, UBE2L6, HMGCR, DDB1, FASN, |
| | | BMP1, MCM7, GMPPB, C1QB, PRPF3, STAT2, SHMT1, |
| | | CALR, ANKZF1, FBN1, PSEN1, NOC4L, MXI1, IDH2, |
| | | ETV7, PPM1G, TP53INP1, ATF3, GPAA1, WARS, EDC4, |
| | | BAZ1A, STAT1, PJA1, DUSP10, NDUFS2, DNASE1L1, |
| | | GCLM, FMR1, AKR1A1, YRDC, LDLRAP1, C1QA, PSMB8, |
| | | FOXP3, FBXO6, PDHA1, JAK2, DCP2, ETS1, TYMP, |
| | | IRF7, ATG4B, NOLC1, PPARA, CDC7, DDOST, MGAT1, |
| | | GCH1, DAPP1, CASP1, LDHC, NR4A1, NUB1, ENGASE, |
| | | PLA2G4C |
| GO:0009108 | coenzyme | NAMPT, ACLY, MOCOS, PTS, FASN, IDH2, AKR1A1, |
| | biosynthetic process | PDHA1, GCH1 |
| GO:0051188 | cofactor biosynthetic | NAMPT, ACLY, MOCOS, PTS, FASN, IDH2, GCLM, |
| | process | AKR1A1, PDHA1, GCH1 |
| GO:1901564 | organonitrogen | B4GALT7, PSME2, NAMPT, LAP3, IGFBP2, IDUA, |
| | compound metabolic | ACLY, TRIM21, RIPK1, RNF144B, MRPL15, MOCOS, |
| | process | LPCAT2, CCNE1, PSMD3, CREM, CPT1A, EIF4H, TP53, |
| | | BCKDHA, CTSK, PRSS23, PTS, UCHL1, UBE2L6, HMGCR, |
| | | DDB1, FASN, BMP1, C1QB, SHMT1, CALR, ANKZF1, |
| | | FBN1, PSEN1, IDH2, PPM1G, GPAA1, WARS, PJA1, |
| | | DUSP10, NDUFS2, GCLM, AKR1A1, LDLRAP1, C1QA, |
| | | PSMB8, FBXO6, PDHA1, JAK2, TYMP, IRF7, ATG4B, |
| | | PPARA, CDC7, DDOST, MGAT1, GCH1, DAPP1, CASP1, |
| | | LDHC, NUB1, ENGASE, PLA2G4C |
| GO:1901700 | response to oxygen- | CD4, IGFBP2, FOSB, CPT1A, TP53, KCNMA1, AIFM1, |
| | containing compound | HMGCR, ITGA2, CXCL10, SHMT1, CALR, ANKZF1, |
| | | FBN1, PSEN1, TP53INP1, FAS, STAT1, DUSP10, GCLM, |
| | | JAK2, ETS1, SLC26A6, PPARA, GCH1, CASP1, NR4A1 |
| GO:2001235 | positive regulation of | RIPK1, TP53, BCL2L14, SFN, TP53INP1, ATF3, FAS, |
| | apoptotic signaling | JAK2, BCAP31 |
| | pathway | |
| GO:0014070 | response to organic | CD4, NAMPT, IGFBP2, FOSB, CCNE1, CPT1A, AIFM1, |
| | cyclic compound | ITGA2, CXCL10, SHMT1, CALR, STAT1, GCLM, JAK2, |
| | | ETS1, SLC26A6, PPARA, CASP1, NR4A1, EPHX1 |
| GO:0031667 | response to nutrient | CD4, IGFBP2, SORD, TP53, AIFM1, HMGCR, ITGA2, |
| | levels | CXCL10, ATF3, FAS, STAT1, GCLM, PPARA, CASP1 |
| GO:0034341 | response to | SEC61A1, TRIM21, STAT1, JAK2, SLC26A6, IRF7, |
| | interferon-gamma | GCH1, CASP1, NUB1 |
| GO:0071345 | cellular response to | CD4, PSME2, EPOR, TRIM21, RIPK1, MRPL15, TP53, |
| | cytokine stimulus | FASN, CXCL10, STAT2, SHMT1, FAS, STAT1, GCLM, |
| | | CXCR3, PSMB8, JAK2, SLC26A6, IRF7, SNX10, CASP1 |
| GO:0051704 | multi-organism | CD4, AAAS, EPOR, NAMPT, CLEC4A, IGFBP2, |
| | process | SEC61A1, FOSB, TRIM21, RIPK1, CREM, EIF4H, TP53, ITGA2, |
| | | DDB1, CXCL10, BANF1, NUP93, C1QB, STAT2, CALR, |
| | | FAS, STAT1, DUSP10, FMR1, SPAG4, C1QA, PSMB8, |
| | | FOXP3, JAK2, ETS1, SLC26A6, IRF7, BCAP31, GCH1, |
| | | CASP1, NUB1, PLA2G4C |
| GO:0006732 | coenzyme metabolic | NAMPT, ACLY, MOCOS, PTS, HMGCR, FASN, |
| | process | SHMT1, IDH2, AKR1A1, PDHA1, GCH1 |
| GO:0009893 | positive regulation of | CD4, PSME2, EHD4, NAMPT, FOSB, ACLY, TRIM21, |
| | metabolic process | RIPK1, RNF144B, CCNE1, CREM, CPT1A, TP53, AIFM1, |
| | | HMGCR, FYCO1, ITGA2, DDB1, FASN, CXCL10, CALR, |
| | | FBN1, PSEN1, TP53INP1, ATF3, WARS, FAS, STAT1, |
| | | VAV3, FMR1, CXCR3, LDLRAP1, FOXP3, JAK2, ETS1, |
| | | IRF7, ATG4B, NOLC1, PPARA, BCAP31, CDC7, GCH1, |
| | | CASP1, NR4A1, NUB1 |
| GO:0009894 | regulation of | PSME2, TRIM21, RNF144B, PSMD3, CPT1A, FEZ1, |
| | catabolic process | UCHL1, AIFM1, FYCO1, DDB1, PSEN1, TP53INP1, FMR1, |
| | | DCP2, ATG4B, PPARA, BCAP31, CASP1, NUB1 |
| GO:0042127 | regulation of cell | CD4, B4GALT7, NAMPT, IGFBP2, TP53, HMGCR, |
| | population | ITGA2, CXCL10, CALR, MXI1, IDH2, SFN, TP53INP1, |
| | proliferation | ATF3, WARS, FAS, STAT1, DUSP10, VAV3, CXCR3, FOXP3, |
| | | CD274, JAK2, ETS1, PDCD1LG2, NOLC1, CDC7, NR4A1 |
| GO:1901135 | carbohydrate | B4GALT7, IDUA, ACLY, MOCOS, LCT, CREM, SORD, |
| | derivative metabolic | HMGCR, FASN, GMPPB, SHMT1, PSEN1, GPAA1, |
| | process | NDUFS2, AKR1A1, FBXO6, PDHA1, TYMP, DDOST, |
| | | MGAT1, LDHC, ENGASE |
| GO:0006006 | glucose metabolic | CREM, CPT1A, SORD, FBN1, ATF3, AKR1A1, PDHA1 |
| | process | |
| GO:0044248 | cellular catabolic | IDUA, RIPK1, RNF144B, PSMD3, CPT1A, SORD, TP53, |
| | process | BCKDHA, CTSK, UCHL1, UBE2L6, DDB1, SHMT1, |
| | | ANKZF1, PSEN1, TP53INP1, EDC4, DNASE1L1, AKR1A1, |
| | | PSMB8, FBXO6, DCP2, TYMP, ATG4B, MGAT1, NUB1, |
| | | PLA2G4C, EPHX1 |
| GO:0045785 | positive regulation of | CD4, IGFBP2, ITGA2, CALR, DUSP10, S100A10, VAV3, |
| | cell adhesion | FOXP3, CD274, JAK2, ETS1, PDCD1LG2 |
| GO:0006955 | immune response | CD4, CLEC4A, SEC61A1, ACLY, TRIM21, PSMD3, |
| | | CXCL10, C1QB, STAT2, PSEN1, VAT1, FAS, STAT1, |
| | | DNASE1L1, C1QA, PSMB8, FOXP3, CD274, JAK2, ETS1, |
| | | SLC26A6, IRF7, PDCD1LG2, DDOST, GCH1, CASP1, NUB1 |
| GO:0007584 | response to nutrient | CD4, IGFBP2, AIFM1, HMGCR, ITGA2, CXCL10, STAT1, |
| | | GCLM, CASP1 |
| GO:0008284 | positive regulation of | CD4, NAMPT, IGFBP2, HMGCR, ITGA2, CXCL10, CALR, |
| | cell population | ATF3, STAT1, VAV3, CXCR3, FOXP3, CD274, JAK2, |
| | proliferation | ETS1, PDCD1LG2, NOLC1, CDC7, NR4A1 |
| GO:0051246 | regulation of protein | CD4, PSME2, EHD4, RRAS, TRIM21, RIPK1, RNF144B, |
| | metabolic process | CCNE1, PSMD3, EIF4H, TP53, UCHL1, AIFM1, HMGCR, |
| | | ITGA2, DDB1, CXCL10, C1QB, STAT2, SHMT1, CALR, |
| | | FBN1, PSEN1, SFN, ATF3, WARS, FAS, DUSP10, FMR1, |
| | | C1QA, PSMB8, FOXP3, JAK2, ATG4B, NOLC1, |
| | | BCAP31, CASP1, NUB1 |
| GO:0009896 | positive regulation of | TRIM21, RNF144B, CPT1A, FYCO1, DDB1, PSEN1, |
| | catabolic process | TP53INP1, FMR1, ATG4B, PPARA, BCAP31, NUB1 |
| GO:0009987 | cellular process | CD4, B4GALT7, PSME2, MPG, EHD4, EPOR, NAMPT, |
| | | CLEC4A, RRP9, IGFBP2, DDX39A, SEC61A1, FOSB, |
| | | RRAS, IDUA, ACLY, TRIM21, RIPK1, RNF144B, MRPL15, |
| | | MOCOS, LPCAT2, CCNE1, PSMD3, CREM, POLA2, |
| | | CPT1A, EIF4H, SORD, TP53, BCKDHA, CTSK, FEZ1, |
| | | PRSS23, PTS, SEC24D, SLC7A11, UCHL1, KCNMA1, UBE2L6, |
| | | AIFM1, HMGCR, FYCO1, ITGA2, DDB1, FASN, |
| | | CXCL10, BMP1, MCM7, GMPPB, BCL2L14, BANF1, NUP93, |
| | | PRPF3, STAT2, GYS1, SHMT1, CALR, ANKZF1, PDIA5, |
| | | FBN1, PSEN1, NOC4L, MXI1, IDH2, STARD3, RASGRP2, |
| | | SFN, ETV7, ICAM4, PPM1G, TP53INP1, ATF3, GPAA1, |
| | | WARS, VAT1, FAS, CRB3, EDC4, BAZ1A, STAT1, PJA1, |
| | | DUSP10, NDUFS2, S100A10, DNASE1L1, VAV3, GCLM, |
| | | FMR1, AKR1A1, YRDC, CXCR3, SPAG4, LDLRAP1, |
| | | C1QA, PSMB8, FOXP3, FBXO6, PDHA1, RDH11, CD274, |
| | | JAK2, DCP2, ETS1, DHRS7B, SLC26A6, TYMP, IRF7, |
| | | ATG4B, IFRD1, KIF2A, NOLC1, PPARA, SEPT9, BCAP31, |
| | | CDC7, SNX10, DDOST, MGAT1, GCH1, DAPP1, CASP1, |
| | | LDHC, NR4A1, NUB1, ENGASE, PLA2G4C, EPHX1 |
| GO:0044237 | cellular metabolic | B4GALT7, PSME2, MPG, NAMPT, RRP9, IGFBP2, DDX39A, |
| | process | FOSB, IDUA, ACLY, TRIM21, RIPK1, RNF144B, |
| | | MRPL15, MOCOS, LPCAT2, CCNE1, PSMD3, CREM, |
| | | POLA2, CPT1A, EIF4H, SORD, TP53, BCKDHA, CTSK, |
| | | PRSS23, PTS, UCHL1, UBE2L6, HMGCR, DDB1, FASN, |
| | | MCM7, GMPPB, PRPF3, STAT2, GYS1, SHMT1, ANKZF1, |
| | | FBN1, PSEN1, NOC4L, MXI1, IDH2, STARD3, ETV7, PPM1G, |
| | | TP53INP1, ATF3, GPAA1, WARS, EDC4, BAZ1A, |
| | | STAT1, PJA1, DUSP10, NDUFS2, DNASE1L1, GCLM, |
| | | FMR1, AKR1A1, YRDC, LDLRAP1, PSMB8, FOXP3, FBXO6, |
| | | PDHA1, RDH11, JAK2, DCP2, ETS1, DHRS7B, TYMP, |
| | | IRF7, ATG4B, NOLC1, PPARA, CDC7, DDOST, MGAT1, |
| | | GCH1, DAPP1, LDHC, NR4A1, NUB1, ENGASE, |
| | | PLA2G4C, EPHX1 |
| GO:0045862 | positive regulation of | PSME2, RIPK1, RNF144B, AIFM1, PSEN1, FAS, FMR1, |
| | proteolysis | JAK2, BCAP31, CASP1, NUB1 |
| GO:0019752 | carboxylic acid | IDUA, ACLY, CREM, CPT1A, SORD, BCKDHA, PTS, |
| | metabolic process | FASN, SHMT1, IDH2, WARS, GCLM, AKR1A1, PDHA1, |
| | | PPARA, GCH1, LDHC, PLA2G4C |
| GO:0006066 | alcohol metabolic | ACLY, SORD, PTS, HMGCR, IDH2, STARD3, LDLRAP1, |
| | process | RDH11, LSS, GCH1 |
| GO:00 | response to biotic | CD4, CLEC4A, SEC61A1, TRIM21, TP53, CXCL10, |
| 09607 | stimulus | BANF1, C1QB, STAT2, FAS, STAT1, DUSP10, FMR1, C1QA, |
| | | PSMB8, FOXP3, JAK2, SLC26A6, IRF7, GCH1, CASP1, |
| | | NUB1 |
| GO:0048518 | positive regulation of | CD4, PSME2, EHD4, NAMPT, CLEC4A, IGFBP2, FOSB, |
| | biological process | RRAS, ACLY, TRIM21, RIPK1, RNF144B, CCNE1, CREM, |
| | | CPT1A, TP53, FEZ1, KCNMA1, AIFM1, HMGCR, |
| | | FYCO1, ITGA2, DDB1, FASN, CXCL10, BMP1, BCL2L14, |
| | | NUP93, C1QB, CALR, FBN1, PSEN1, SFN, TP53INP1, |
| | | ATF3, WARS, FAS, STAT1, DUSP10, S100A10, VAV3, |
| | | FMR1, CXCR3, LDLRAP1, C1QA, FOXP3, CD274, JAK2, |
| | | ETS1, SLC26A6, IRF7, PDCD1LG2, ATG4B, NOLC1, |
| | | PPARA, SEPT9, BCAP31, CDC7, GCH1, CASP1, NR4A1, |
| | | NUB1 |
| GO:0009056 | catabolic process | IDUA, RIPK1, RNF144B, PSMD3, CPT1A, SORD, TP53, |
| | | BCKDHA, CTSK, UCHL1, UBE2L6, DDB1, SHMT1, |
| | | ANKZF1, PSEN1, TP53INP1, EDC4, PJA1, DNASE1L1, |
| | | AKR1A1, PSMB8, FBXO6, DCP2, TYMP, ATG4B, MGAT1, |
| | | NUB1, PLA2G4C, EPHX1 |
| GO:0016032 | viral process | CD4, AAAS, RIPK1, EIF4H, TP53, ITGA2, DDB1, |
| | | BANF1, NUP93, STAT2, STAT1, FMR1, PSMB8, IRF7 |
| GO:0002684 | positive regulation of | CD4, CLEC4A, IGFBP2, RIPK1, ITGA2, CXCL10, C1QB, |
| | immune system | CALR, PSEN1, STAT1, DUSP10, VAV3, C1QA, FOXP3, |
| | process | CD274, ETS1, IRF7, PDCD1LG2 |
| GO:0006270 | DNA replication | CCNE1, POLA2, MCM7, CDC7 |
| | initiation | |
| GO:0019221 | cytokine-mediated | CD4, PSME2, EPOR, TRIM21, RIPK1, TP53, CXCL10, |
| | signaling pathway | STAT2, FAS, STAT1, CXCR3, PSMB8, JAK2, IRF7, CASP1 |
| GO:0006979 | response to oxidative | TP53, SLC7A11, AIFM1, ANKZF1, PSEN1, TP53INP1, |
| | stress | STAT1, NDUFS2, GCLM, JAK2, ETS1 |
| GO:0046007 | negative regulation of | FOXP3, CD274, PDCD1LG2 |
| | activated T cell | |
| | proliferation | |
| GO:0030162 | regulation of | PSME2, TRIM21, RIPK1, RNF144B, AIFM1, C1QB, |
| | proteolysis | PSEN1, SFN, FAS, FMR1, C1QA, PSMB8, JAK2, BCAP31, |
| | | CASP1, NUB1 |
| GO:0031329 | regulation of cellular | PSME2, TRIM21, RNF144B, CPT1A, FEZ1, UCHL1, |
| | catabolic process | AIFM1, FYCO1, PSEN1, TP53INP1, FMR1, DCP2, PPARA, |
| | | BCAP31, CASP1, NUB1 |
| GO:0033993 | response to lipid | CD4, IGFBP2, FOSB, CCNE1, CPT1A, AIFM1, ITGA2, |
| | | CXCL10, CALR, FAS, DUSP10, JAK2, ETS1, PPARA, |
| | | GCH1, CASP1, NR4A1 |
| GO:0008285 | negative regulation of | B4GALT7, TP53, MXI1, IDH2, SFN, TP53INP1, WARS, |
| | cell population | STAT1, DUSP10, CXCR3, FOXP3, CD274, JAK2, ETS1, |
| | proliferation | PDCD1LG2 |
| GO:0051707 | response to other | CD4, CLEC4A, SEC61A1, TRIM21, CXCL10, BANF1, |
| | organism | C1QB, STAT2, FAS, STAT1, DUSP10, FMR1, C1QA, PSMB8, |
| | | FOXP3, JAK2, SLC26A6, IRF7, GCH1, CASP1, NUB1 |
| GO:2001233 | regulation of | RIPK1, TP53, BCL2L14, PSEN1, SFN, TP53INP1, ATF3, |
| | apoptotic signaling | FAS, GCLM, JAK2, BCAP31 |
| | pathway | |
| GO:0010941 | regulation of cell | RIPK1, RNF144B, TP53, KCNMA1, AIFM1, HMGCR, |
| | death | DDB1, BCL2L14, NUP93, CALR, PSEN1, SFN, TP53INP1, |
| | | ATF3, FAS, STAT1, VAV3, GCLM, CXCR3, CD274, |
| | | JAK2, ETS1, IRF7, PPARA, BCAP31, CASP1 |
| GO:0051049 | regulation of | CD4, AAAS, EHD4, RIPK1, CPT1A, TP53, FEZ1, |
| | transport | KCNMA1, HMGCR, ITGA2, CXCL10, CALR, PSEN1, IDH2, |
| | | SFN, FMR1, YRDC, CXCR3, LDLRAP1, FOXP3, CD274, |
| | | JAK2, SLC26A6, NOLC1, PPARA, BCAP31, CASP1 |
| GO:0009612 | response to | IGFBP2, FOSB, ITGA2, CXCL10, FAS, STAT1, ETS1, |
| | mechanical stimulus | CASP1 |
| GO:1901566 | organonitrogen | B4GALT7, NAMPT, ACLY, MRPL15, MOCOS, |
| | compound | LPCAT2, EIF4H, PTS, FASN, SHMT1, PSEN1, IDH2, GPAA1, |
| | biosynthetic process | WARS, GCLM, AKR1A1, PDHA1, TYMP, ATG4B, DDOST, |
| | | MGAT1, GCH1, LDHC |
| GO:0051186 | cofactor metabolic | NAMPT, ACLY, MOCOS, PTS, HMGCR, FASN, SHMT1, |
| | process | IDH2, GCLM, AKR1A1, PDHA1, GCH1 |
| GO:0010950 | positive regulation of | PSME2, RIPK1, AIFM1, FAS, JAK2, BCAP31, CASP1 |
| | endopeptidase | |
| | activity | |
| GO:0046006 | regulation of | IGFBP2, FOXP3, CD274, PDCD1LG2 |
| | activated T cell | |
| | proliferation | |
| GO:0032386 | regulation of | AAAS, TP53, FEZ1, PSEN1, SFN, FMR1, LDLRAP1, |
| | intracellular transport | JAK2, NOLC1, BCAP31 |
| GO:0006508 | proteolysis | PSME2, LAP3, RIPK1, RNF144B, PSMD3, TP53, CTSK, |
| | | PRSS23, UCHL1, UBE2L6, DDB1, BMP1, C1QB, ANKZF1, |
| | | PSEN1, C1QA, PSMB8, FBXO6, ATG4B, CASP1, NUB1 |
| GO:0046822 | regulation of | AAAS, TP53, PSEN1, SFN, JAK2, NOLC1 |
| | nucleocytoplasmic | |
| | transport | |
| GO:0002682 | regulation of immune | CD4, CLEC4A, IGFBP2, TRAFD1, RIPK1, ITGA2, CXCL10, |
| | system process | C1QB, CALR, FBN1, PSEN1, ICAM4, STAT1, DUSP10, |
| | | VAV3, CXCR3, C1QA, FOXP3, CD274, JAK2, ETS1, |
| | | IRF7, PDCD1LG2 |
| GO:0032787 | monocarboxylic acid | IDUA, ACLY, CREM, CPT1A, SORD, FASN, IDH2, |
| | metabolic process | AKR1A1, PDHA1, PPARA, LDHC, PLA2G4C |
| GO:1901137 | carbohydrate | B4GALT7, ACLY, SORD, FASN, GMPPB, SHMT1, |
| | derivative | PSEN1, GPAA1, AKR1A1, PDHA1, TYMP, DDOST, |
| | biosynthetic process | MGAT1, LDHC |
| GO:0065008 | regulation of | CD4, TRIM21, CCNE1, POLA2, CPT1A, TP53, CTSK, |
| | biological quality | SLC7A11, KCNMA1, HMGCR, ITGA2, DDB1, CXCL10, |
| | | SHMT1, CALR, PDIA5, FBN1, PSEN1, MXI1, STARD3, |
| | | SFN, GPAA1, STAT1, VAV3, GCLM, FMR1, YRDC, CXCR3, |
| | | SPAG4, LDLRAP1, FOXP3, RDH11, JAK2, DCP2, ETS1, |
| | | SLC26A6, LSS, IFRD1, PPARA, BCAP31, CDC7, SNX10, |
| | | GCH1, CASP1 |
| GO:0031331 | positive regulation of | TRIM21, RNF144B, CPT1A, FYCO1, PSEN1, TP53INP1, |
| | cellular catabolic | FMR1, PPARA, BCAP31, NUB1 |
| | process | |
| GO:0032101 | regulation of | CLEC4A, TRAFD1, RIPK1, HMGCR, ITGA2, CXCL10, |
| | response to external | C1QB, CALR, STAT1, DUSP10, CXCR3, C1QA, FOXP3, |
| | stimulus | JAK2, ETS1, IRF7, PPARA, CASP1 |
| GO:0042981 | regulation of | RIPK1, RNF144B, TP53, KCNMA1, AIFM1, HMGCR, |
| | apoptotic process | DDB1, BCL2L14, CALR, PSEN1, SFN, TP53INP1, ATF3, |
| | | FAS, STAT1, VAV3, GCLM, CXCR3, CD274, JAK2, ETS1, |
| | | IRF7, BCAP31, CASP1 |
| GO:0002660 | positive regulation of | FOXP3, CD274 |
| | peripheral tolerance | |
| | induction | |
| GO:0009628 | response to abiotic | IGFBP2, FOSB, SORD, TP53, KCNMA1, AIFM1, |
| | stimulus | HMGCR, ITGA2, DDB1, CXCL10, TP53INP1, FAS, STAT1, |
| | | FMR1, RDH11, ETS1, NOLC1, PPARA, CASP1 |
| GO:1902652 | secondary alcohol | ACLY, HMGCR, IDH2, STARD3, LDLRAP1, LSS |
| | metabolic process | |
| GO:0010035 | response to inorganic | IGFBP2, FOSB, SORD, KCNMA1, AIFM1, CALR, |
| | substance | ANKZF1, RASGRP2, STAT1, FMR1, C1QA, ETS1 |
| GO:0051770 | positive regulation of | NAMPT, STAT1, JAK2 |
| | nitric-oxide synthase | |
| | biosynthetic process | |
| GO:0051969 | regulation of | ITGA2, FMR1, TYMP |
| | transmission of nerve | |
| | impulse | |
| GO:0044419 | interspecies | CD4, AAAS, RIPK1, EIF4H, TP53, ITGA2, DDB1, |
| | interaction between | CXCL10, BANF1, NUP93, STAT2, STAT1, FMR1, PSMB8, |
| | organisms | IRF7 |
| GO:0030522 | intracellular receptor | CCNE1, CREM, CALR, JAK2, IRF7, PPARA, NR4A1 |
| | signaling pathway | |
| GO:0032879 | regulation of | CD4, AAAS, EHD4, RRAS, RIPK1, CCNE1, CPT1A, |
| | localization | TP53, FEZ1, KCNMA1, HMGCR, ITGA2, CXCL10, CALR, |
| | | PSEN1, IDH2, SFN, TP53INP1, DUSP10, FMR1, YRDC, |
| | | CXCR3, LDLRAP1, FOXP3, CD274, JAK2, DCP2, ETS1, |
| | | SLC26A6, KIF2A, NOLC1, PPARA, BCAP31, CASP1 |
| GO:0044283 | small molecule | ACLY, SORD, PTS, HMGCR, FASN, SHMT1, STARD3, |
| | biosynthetic process | ATF3, AKR1A1, TYMP, LSS, GCH1, LDHC |
| GO:0002474 | antigen processing | CLEC4A, SEC24D, CALR, BCAP31 |
| | and presentation of | |
| | peptide antigen via | |
| | MHC class I | |
| GO:0031325 | positive regulation of | CD4, PSME2, EHD4, NAMPT, FOSB, ACLY, TRIM21, |
| | cellular metabolic | RIPK1, RNF144B, CCNE1, CREM, CPT1A, TP53, AIFM1, |
| | process | HMGCR, FYCO1, ITGA2, FASN, CXCL10, FBN1, PSEN1, |
| | | TP53INP1, ATF3, FAS, STAT1, VAV3, FMR1, CXCR3, |
| | | FOXP3, JAK2, ETS1, IRF7, NOLC1, PPARA, BCAP31, |
| | | CDC7, CASP1, NR4A1, NUB1 |
| GO:0032388 | positive regulation of | TP53, FEZ1, PSEN1, SFN, LDLRAP1, JAK2, BCAP31 |
| | intracellular transport | |
| GO:0032693 | negative regulation of | FOXP3, CD274, PDCD1LG2 |
| | interleukin-10 | |
| | production | |
| GO:0043280 | positive regulation of | RIPK1, AIFM1, FAS, JAK2, BCAP31, CASP1 |
| | cysteine-type | |
| | endopeptidase | |
| | activity involved in | |
| | apoptotic process | |
| GO:0048661 | positive regulation of | NAMPT, HMGCR, ITGA2, STAT1, JAK2 |
| | smooth muscle cell | |
| | proliferation | |
| GO:1901615 | organic hydroxy | ACLY, SORD, PTS, HMGCR, IDH2, STARD3, |
| | compound metabolic | LDLRAP1, RDH11, LSS, GCH1, LDHC |
| | process | |
| GO:1901701 | cellular response to | CPT1A, TP53, AIFM1, ITGA2, CXCL10, SHMT1, |
| | oxygen-containing | ANKZF1, FBN1, PSEN1, TP53INP1, STAT1, GCLM, JAK2, |
| | compound | ETS1, SLC26A6, CASP1, NR4A1 |
| GO:0090407 | organophosphate | NAMPT, ACLY, MOCOS, LPCAT2, SORD, FASN, SHMT1, |
| | biosynthetic process | IDH2, GPAA1, AKR1A1, PDHA1, GCH1, LDHC |
| GO:0032355 | response to estradiol | CD4, IGFBP2, AIFM1, ITGA2, CALR, ETS1 |
| GO:0018904 | ether metabolic | FASN, DHRS7B, EPHX1 |
| | process | |
| GO:0032870 | cellular response to | IGFBP2, FOSB, CCNE1, AIFM1, ITGA2, CALR, FBN1, |
| | hormone stimulus | STAT1, GCLM, JAK2, SLC26A6, PPARA, NR4A1 |
| GO:0033554 | cellular response to | MPG, DDX39A, RIPK1, TP53, UBE2L6, AIFM1, DDB1, |
| | stress | CXCL10, MCM7, CALR, ANKZF1, PDIA5, PSEN1, SFN, |
| | | TP53INP1, ATF3, FAS, VAV3, FMR1, FBXO6, JAK2, |
| | | ETS1, IRF7, CDC7 |
| GO:0050671 | positive regulation of | CD4, IGFBP2, VAV3, FOXP3, CD274, PDCD1LG2 |
| | lymphocyte | |
| | proliferation | |
| GO:0006919 | activation of | RIPK1, AIFM1, FAS, JAK2, CASP1 |
| | cysteine-type | |
| | endopeptidase | |
| | activity involved in | |
| | apoptotic process | |
| GO:0031347 | regulation of defense | CLEC4A, TRAFD1, RIPK1, ITGA2, C1QB, STAT1, |
| | response | DUSP10, C1QA, FOXP3, JAK2, ETS1, IRF7, PPARA, CASP1 |
| GO:0045087 | innate immune | CLEC4A, SEC61A1, TRIM21, C1QB, STAT2, STAT1, |
| | response | C1QA, PSMB8, JAK2, SLC26A6, IRF7, GCH1, CASP1, |
| | | NUB1 |
| GO:0060341 | regulation of cellular | CD4, AAAS, CCNE1, TP53, FEZ1, HMGCR, CXCL10, |
| | localization | PSEN1, SFN, FMR1, CXCR3, LDLRAP1, JAK2, NOLC1, |
| | | BCAP31 |
| GO:0071840 | cellular component | CD4, B4GALT7, EHD4, RRP9, SEC61A1, TRIM21, |
| | organization or | RIPK1, MRPL15, LPCAT2, CCNE1, POLA2, CPT1A, EIF4H, |
| | biogenesis | TP53, CTSK, FEZ1, SEC24D, UCHL1, KCNMA1, AIFM1, |
| | | HMGCR, ITGA2, DDB1, BMP1, MCM7, BANF1, NUP93, |
| | | PRPF3, SHMT1, CALR, FBN1, PSEN1, NOC4L, |
| | | STARD3, SFN, ICAM4, TP53INP1, GPAA1, FAS, CRB3, BAZ1A, |
| | | NDUFS2, S100A10, VAV3, GCLM, SPAG4, LDLRAP1, |
| | | FOXP3, JAK2, ETS1, TYMP, ATG4B, IFRD1, KIF2A, |
| | | NOLC1, SEPT9, SNX10, GCH1 |
| GO:0009725 | response to hormone | CD4, IGFBP2, FOSB, CCNE1, SORD, AIFM1, ITGA2, |
| | | CALR, FBN1, STAT1, GCLM, JAK2, ETS1, SLC26A6, |
| | | PPARA, NR4A1 |
| GO:0046165 | alcohol biosynthetic | ACLY, PTS, HMGCR, LSS, GCH1 |
| | process | |
| GO:0098542 | defense response to | CD4, CLEC4A, SEC61A1, TRIM21, CXCL10, C1QB, |
| | other organism | STAT2, STAT1, C1QA, PSMB8, JAK2, SLC26A6, IRF7, |
| | | GCH1, CASP1, NUB1 |
| GO:0042102 | positive regulation of | CD4, IGFBP2, FOXP3, CD274, PDCD1LG2 |
| | T cell proliferation | |
| GO:0048522 | positive regulation of | CD4, PSME2, EHD4, NAMPT, IGFBP2, FOSB, ACLY, |
| | cellular process | TRIM21, RIPK1, RNF144B, CCNE1, CREM, CPT1A, TP53, |
| | | FEZ1, KCNMA1, AIFM1, HMGCR, FYCO1, ITGA2, |
| | | DDB1, FASN, CXCL10, BCL2L14, NUP93, CALR, FBN1, |
| | | PSEN1, SFN, TP53INP1, ATF3, WARS, FAS, STAT1, |
| | | DUSP10, S100A10, VAV3, FMR1, CXCR3, LDLRAP1, FOXP3, |
| | | CD274, JAK2, ETS1, IRF7, PDCD1LG2, NOLC1, PPARA, |
| | | SEPT9, BCAP31, CDC7, CASP1, NR4A1, NUB1 |
| GO:1901575 | organic substance | IDUA, RIPK1, RNF144B, PSMD3, CPT1A, SORD, |
| | catabolic process | BCKDHA, CTSK, UCHL1, UBE2L6, DDB1, SHMT1, ANKZF1, |
| | | EDC4, PJA1, DNASE1L1, AKR1A1, PSMB8, FBXO6, |
| | | DCP2, TYMP, MGAT1, NUB1, PLA2G4C |
| GO:0030155 | regulation of cell | CD4, IGFBP2, ITGA2, CALR, DUSP10, S100A10, VAV3, |
| | adhesion | FOXP3, CD274, JAK2, ETS1, PDCD1LG2, PPARA |
| GO:0006952 | defense response | CD4, CLEC4A, SEC61A1, TRIM21, CXCL10, C1QB, |
| | | STAT2, PSEN1, FAS, STAT1, CXCR3, C1QA, PSMB8, |
| | | JAK2, SLC26A6, IRF7, GCH1, CASP1, NUB1, PLA2G4C |
| GO:0010243 | response to | FOSB, TP53, AIFM1, ITGA2, SHMT1, ANKZF1, FBN1, |
| | organonitrogen | PSEN1, STAT1, GCLM, FBXO6, JAK2, SLC26A6, |
| | compound | PPARA, CASP1, NR4A1 |
| GO:0016043 | cellular component | CD4, B4GALT7, EHD4, SEC61A1, TRIM21, RIPK1, |
| | organization | MRPL15, LPCAT2, CCNE1, POLA2, CPT1A, EIF4H, TP53, |
| | | CTSK, FEZ1, SEC24D, UCHL1, KCNMA1, AIFM1, |
| | | HMGCR, ITGA2, DDB1, BMP1, MCM7, BANF1, NUP93, |
| | | PRPF3, SHMT1, CALR, FBN1, PSEN1, STARD3, SFN, ICAM4, |
| | | TP53INP1, GPAA1, FAS, CRB3, BAZ1A, NDUFS2, |
| | | S100A10, VAV3, GCLM, SPAG4, LDLRAP1, FOXP3, JAK2, |
| | | ETS1, TYMP, ATG4B, IFRD1, KIF2A, NOLC1, SEPT9, |
| | | SNX10, GCH1 |
| GO:0045185 | maintenance of | CD4, FBN1, MXI1, GPAA1, SPAG4 |
| | protein location | |
| GO:0090181 | regulation of | HMGCR, FASN, LDLRAP1, LSS |
| | cholesterol metabolic | |
| | process | |
| GO:1903039 | positive regulation of | CD4, IGFBP2, DUSP10, FOXP3, CD274, ETS1, |
| | leukocyte cell-cell | PDCD1LG2 |
| | adhesion | |
| GO:0071482 | cellular response to | TP53, DDB1, TP53INP1, FMR1, RDH11 |
| | light stimulus | |
| GO:0044085 | cellular component | EHD4, RRP9, TRIM21, RIPK1, CPT1A, EIF4H, TP53, |
| | biogenesis | SEC24D, AIFM1, HMGCR, ITGA2, DDB1, BMP1, NUP93, |
| | | PRPF3, SHMT1, CALR, PSEN1, NOC4L, TP53INP1, |
| | | GPAA1, FAS, CRB3, NDUFS2, S100A10, VAV3, JAK2, |
| | | ATG4B, KIF2A, NOLC1, SEPT9, SNX10, GCH1 |
| GO:0051235 | maintenance of | CD4, CALR, FBN1, MXI1, GPAA1, SPAG4 |
| | location | |
| GO:0051050 | positive regulation of | CD4, TP53, FEZ1, ITGA2, CXCL10, CALR, PSEN1, SFN, |
| | transport | FMR1, CXCR3, LDLRAP1, CD274, JAK2, SLC26A6, |
| | | BCAP31, CASP1 |
| GO:0050727 | regulation of | ITGA2, C1QB, DUSP10, C1QA, FOXP3, JAK2, ETS1, |
| | inflammatory | PPARA, CASP1 |
| | response | |
| GO:0019640 | glucuronate catabolic | SORD, AKR1A1 |
| | process to xylulose 5- | |
| | phosphate | |
| GO:0043281 | regulation of | RIPK1, AIFM1, SFN, FAS, JAK2, BCAP31, CASP1 |
| | cysteine-type | |
| | endopeptidase | |
| | activity involved in | |
| | apoptotic process | |
| GO:1900117 | regulation of | TP53, AIFM1, CXCR3 |
| | execution phase of | |
| | apoptosis | |
| GO:0044706 | multi-multicellular | EPOR, NAMPT, IGFBP2, FOSB, ITGA2, ETS1, |
| | organism process | PLA2G4C |
| GO:0048584 | positive regulation of | CD4, CLEC4A, RIPK1, TP53, HMGCR, ITGA2, CXCL10, |
| | response to stimulus | BCL2L14, NUP93, C1QB, CALR, PSEN1, SFN, TP53INP1, |
| | | ATF3, FAS, VAV3, FMR1, CXCR3, LDLRAP1, C1QA, |
| | | FOXP3, CD274, JAK2, ETS1, IRF7, BCAP31, CASP1 |
| GO:1901698 | response to nitrogen | FOSB, TP53, AIFM1, ITGA2, SHMT1, ANKZF1, FBN1, |
| | compound | PSEN1, STAT1, GCLM, FMR1, FBXO6, JAK2, SLC26A6, |
| | | PPARA, CASP1, NR4A1 |
| GO:1903902 | positive regulation of | CD4, TRIM21, DDB1, FMR1 |
| | viral life cycle | |
| GO:0071346 | cellular response to | TRIM21, STAT1, JAK2, SLC26A6, IRF7, CASP1 |
| | interferon-gamma | |
| GO:0097300 | programmed necrotic | RIPK1, FAS, CASP1 |
| | cell death | |
| GO:0032268 | regulation of cellular | CD4, PSME2, EHD4, RRAS, TRIM21, RIPK1, RNF144B, |
| | protein metabolic | CCNE1, EIF4H, TP53, UCHL1, AIFM1, HMGCR, ITGA2, |
| | process | CXCL10, STAT2, SHMT1, CALR, FBN1, PSEN1, SFN, |
| | | ATF3, WARS, FAS, DUSP10, FMR1, FOXP3, JAK2, |
| | | NOLC1, BCAP31, CASP1, NUB1 |
| GO:0042325 | regulation of | CD4, EHD4, RRAS, RIPK1, CCNE1, TP53, UCHL1, |
| | phosphorylation | HMGCR, CXCL10, MCM7, STAT2, FBN1, PSEN1, SFN, ATF3, |
| | | WARS, FAS, DUSP10, VAV3, FMR1, JAK2, PPARA |
| GO:0043900 | regulation of multi- | CD4, CLEC4A, TRIM21, TRAFD1, RIPK1, DDB1, BANF1, |
| | organism process | CALR, STAT1, DUSP10, FMR1, JAK2, IRF7 |
| GO:0065007 | biological regulation | CD4, B4GALT7, AAAS, PSME2, EHD4, EPOR, NAMPT, |
| | | CLEC4A, IGFBP2, DDX39A, FOSB, RRAS, ACLY, |
| | | TRIM21, TRAFD1, RIPK1, RNF144B, CCNE1, PSMD3, |
| | | CREM, POLA2, CPT1A, EIF4H, TP53, CTSK, FEZ1, SLC7A11, |
| | | UCHL1, KCNMA1, UBE2L6, AIFM1, HMGCR, FYCO1, |
| | | ITGA2, DDB1, FASN, CXCL10, BMP1, MCM7, BCL2L14, |
| | | BANF1, NUP93, C1QB, STAT2, SHMT1, CALR, PDIA5, |
| | | FBN1, PSEN1, NOC4L, MXI1, IDH2, STARD3, RASGRP2, |
| | | SFN, ETV7, ICAM4, PPM1G, TP53INP1, ATF3, GPAA1, |
| | | WARS, VAT1, FAS, EDC4, BAZ1A, STAT1, DUSP10, |
| | | S100A10, VAV3, GCLM, FMR1, YRDC, CXCR3, SPAG4, |
| | | LDLRAP1, C1QA, PSMB8, FOXP3, FBXO6, RDH11, |
| | | CD274, JAK2, DCP2, ETS1, SLC26A6, TYMP, IRF7, LSS, |
| | | PDCD1LG2, ATG4B, IFRD1, KIF2A, NOLC1, PPARA, |
| | | SEPT9, BCAP31, CDC7, SNX10, GCH1, DAPP1, CASP1, |
| | | NR4A1, NUB1, PLA2G4C |
| GO:0090087 | regulation of peptide | CPT1A, TP53, HMGCR, PSEN1, IDH2, SFN, FOXP3, |
| | transport | CD274, JAK2, SLC26A6, NOLC1, BCAP31, CASP1 |
| GO:1903037 | regulation of | CD4, IGFBP2, DUSP10, FOXP3, CD274, ETS1, |
| | leukocyte cell-cell | PDCD1LG2, PPARA |
| | adhesion | |
| GO:0006084 | acetyl-CoA metabolic | ACLY, FASN, PDHA1 |
| | process | |
| GO:0019882 | antigen processing | CLEC4A, SEC24D, CALR, PSMB8, KIF2A, BCAP31 |
| | and presentation | |
| GO:0045732 | positive regulation of | RNF144B, DDB1, PSEN1, FMR1, ATG4B, BCAP31, |
| | protein catabolic | NUB1 |
| | process | |
| GO:0071214 | cellular response to | TP53, ITGA2, DDB1, TP53INP1, FAS, FMR1, RDH11, |
| | abiotic stimulus | CASP1 |
| GO:0008611 | ether lipid | FASN, DHRS7B |
| | biosynthetic process | |
| GO:0030223 | neutrophil | FASN, DHRS7B |
| | differentiation | |
| GO:0055086 | nucleobase- | NAMPT, ACLY, MOCOS, HMGCR, FASN, GMPPB, |
| | containing small | SHMT1, IDH2, NDUFS2, PDHA1, TYMP, MGAT1, LDHC |
| | molecule metabolic | |
| | process | |
| GO:0097527 | necroptotic signaling | RIPK1, FAS |
| | pathway | |
| GO:1901617 | organic hydroxy | ACLY, PTS, HMGCR, LSS, GCH1, LDHC |
| | compound | |
| | biosynthetic process | |
| GO:0008203 | cholesterol metabolic | ACLY, HMGCR, STARD3, LDLRAP1, LSS |
| | process | |
| GO:0019222 | regulation of | CD4, PSME2, EHD4, NAMPT, DDX39A, FOSB, RRAS, |
| | metabolic process | ACLY, TRIM21, RIPK1, RNF144B, CCNE1, PSMD3, |
| | | CREM, CPT1A, EIF4H, TP53, FEZ1, UCHL1, AIFM1, |
| | | HMGCR, FYCO1, ITGA2, DDB1, FASN, CXCL10, MCM7, |
| | | C1QB, STAT2, SHMT1, CALR, FBN1, PSEN1, NOC4L, MXI1, |
| | | SFN, ETV7, TP53INP1, ATF3, WARS, FAS, EDC4, |
| | | BAZ1A, STAT1, DUSP10, VAV3, FMR1, CXCR3, LDLRAP1, |
| | | C1QA, PSMB8, FOXP3, JAK2, DCP2, ETS1, IRF7, LSS, |
| | | ATG4B, NOLC1, PPARA, BCAP31, CDC7, GCH1, CASP1, |
| | | NR4A1, NUB1 |
| GO:0071407 | cellular response to | CCNE1, AIFM1, ITGA2, SHMT1, CALR, STAT1, GCLM, |
| | organic cyclic | JAK2, SLC26A6, PPARA, NR4A1 |
| | compound | |
| GO:0050793 | regulation of | CD4, RRAS, RIPK1, CTSK, FEZ1, HMGCR, CXCL10, |
| | developmental | BMP1, STAT2, CALR, FBN1, PSEN1, IDH2, SFN, |
| | process | TP53INP1, WARS, VAT1, STAT1, DUSP10, S100A10, FMR1, |
| | | CXCR3, FOXP3, CD274, JAK2, ETS1, TYMP, IRF7, IFRD1, |
| | | PPARA, CDC7 |
| GO:0080134 | regulation of | CLEC4A, TRAFD1, RIPK1, HMGCR, ITGA2, NUP93, |
| | response to stress | C1QB, FAS, STAT1, DUSP10, FMR1, C1QA, FOXP3, JAK2, |
| | | ETS1, IRF7, PPARA, BCAP31, GCH1, CASP1 |
| GO:0048147 | negative regulation of | B4GALT7, TP53, TP53INP1 |
| | fibroblast | |
| | proliferation | |
| GO:0046824 | positive regulation of | TP53, PSEN1, SFN, JAK2 |
| | nucleocytoplasmic | |
| | transport | |
| GO:0055114 | oxidation-reduction | CPT1A, SORD, BCKDHA, AIFM1, HMGCR, FASN, |
| | process | GYS1, PDIA5, IDH2, VAT1, NDUFS2, AKR1A1, PDHA1, |
| | | RDH11, DHRS7B, LDHC |
| GO:0060337 | type I interferon | STAT2, STAT1, PSMB8, IRF7 |
| | signaling pathway | |
| GO:0010604 | positive regulation of | CD4, PSME2, EHD4, NAMPT, FOSB, RIPK1, RNF144B, |
| | macromolecule | CCNE1, CREM, TP53, AIFM1, HMGCR, ITGA2, DDB1, |
| | metabolic process | CXCL10, CALR, FBN1, PSEN1, TP53INP1, ATF3, |
| | | WARS, FAS, STAT1, FMR1, CXCR3, FOXP3, JAK2, ETS1, |
| | | IRF7, ATG4B, NOLC1, PPARA, BCAP31, CDC7, CASP1, |
| | | NR4A1, NUB1 |
| GO:0071236 | cellular response to | TP53, AIFM1, ANKZF1, TP53INP1, ETS1 |
| | antibiotic | |
| GO:1901800 | positive regulation of | RNF144B, PSEN1, FMR1, BCAP31, NUB1 |
| | proteasomal protein | |
| | catabolic process | |
| GO:0043687 | post-translational | PSME2, PSMD3, PRSS23, DDB1, FBN1, PSMB8, FBXO6, |
| | protein modification | ATG4B, NUB1 |
| GO:0006261 | DNA-dependent | CCNE1, POLA2, MCM7, BAZ1A, CDC7 |
| | DNA replication | |
| GO:0006729 | tetrahydrobiopterin | PTS, GCH1 |
| | biosynthetic process | |
| GO:0009058 | biosynthetic process | B4GALT7, NAMPT, FOSB, ACLY, MRPL15, MOCOS, |
| | | LPCAT2, CCNE1, CREM, POLA2, EIF4H, SORD, TP53, |
| | | PTS, UBE2L6, HMGCR, FASN, MCM7, GMPPB, STAT2, |
| | | GYS1, SHMT1, PSEN1, MXI1, IDH2, STARD3, ETV7, |
| | | TP53INP1, ATF3, GPAA1, WARS, GMPPA, BAZ1A, STAT1, |
| | | GCLM, AKR1A1, FOXP3, PDHA1, ETS1, DHRS7B, |
| | | TYMP, IRF7, LSS, ATG4B, PPARA, CDC7, DDOST, |
| | | MGAT1, GCH1, LDHC, NR4A1 |
| GO:0022407 | regulation of cell-cell | CD4, IGFBP2, DUSP10, FOXP3, CD274, JAK2, ETS1, |
| | adhesion | PDCD1LG2, PPARA |
| GO:0043170 | macromolecule | B4GALT7, AAAS, PSME2, MPG, LAP3, RRP9, IGFBP2, |
| | metabolic process | DDX39A, FOSB, IDUA, TRIM21, RIPK1, RNF144B, |
| | | MRPL15, MOCOS, CCNE1, PSMD3, CREM, POLA2, EIF4H, |
| | | TP53, CTSK, PRSS23, UCHL1, UBE2L6, DDB1, BMP1, |
| | | MCM7, NUP93, C1QB, PRPF3, STAT2, GYS1, CALR, |
| | | ANKZF1, FBN1, PSEN1, NOC4L, MXI1, ETV7, PPM1G, |
| | | TP53INP1, ATF3, GPAA1, WARS, EDC4, BAZ1A, STAT1, |
| | | PJA1, DUSP10, DNASE1L1, FMR1, YRDC, LDLRAP1, |
| | | C1QA, PSMB8, FOXP3, FBXO6, JAK2, DCP2, ETS1, IRF7, |
| | | ATG4B, NOLC1, PPARA, CDC7, DDOST, MGAT1, |
| | | DAPP1, CASP1, NR4A1, NUB1, ENGASE |
| GO:0048519 | negative regulation of | B4GALT7, CLEC4A, IGFBP2, FOSB, RRAS, TRIM21, |
| | biological process | TRAFD1, RIPK1, RNF144B, CCNE1, CREM, TP53, FEZ1, |
| | | UCHL1, UBE2L6, HMGCR, DDB1, CXCL10, BANF1, |
| | | NUP93, SHMT1, CALR, FBN1, PSEN1, MXI1, IDH2, SFN, |
| | | ETV7, PPM1G, TP53INP1, ATF3, WARS, VAT1, FAS, |
| | | EDC4, STAT1, DUSP10, GCLM, FMR1, YRDC, CXCR3, |
| | | FOXP3, FBXO6, CD274, JAK2, DCP2, ETS1, IRF7, |
| | | PDCD1LG2, IFRD1, PPARA, CDC7, NR4A1 |
| GO:0006970 | response to osmotic | SORD, KCNMA1, ITGA2, NOLC1 |
| | stress | |
| GO:0042176 | regulation of protein | PSME2, RNF144B, PSMD3, DDB1, PSEN1, FMR1, |
| | catabolic process | ATG4B, BCAP31, NUB1 |
| GO:0065003 | protein-containing | EHD4, TRIM21, RIPK1, CPT1A, EIF4H, TP53, SEC24D, |
| | complex assembly | AIFM1, HMGCR, DDB1, BMP1, NUP93, PRPF3, |
| | | SHMT1, CALR, GPAA1, FAS, NDUFS2, S100A10, JAK2, SEPT9, |
| | | GCH1 |
| GO:1901360 | organic cyclic | MPG, NAMPT, RRP9, DDX39A, FOSB, ACLY, MOCOS, |
| | compound metabolic | CCNE1, CREM, POLA2, TP53, PTS, UBE2L6, HMGCR, |
| | process | DDB1, FASN, MCM7, GMPPB, PRPF3, STAT2, SHMT1, |
| | | NOC4L, MXI1, IDH2, STARD3, ETV7, TP53INP1, ATF3, |
| | | WARS, EDC4, BAZ1A, STAT1, NDUFS2, DNASE1L1, |
| | | FMR1, YRDC, LDLRAP1, FOXP3, FBXO6, PDHA1, |
| | | DCP2, ETS1, TYMP, IRF7, LSS, NOLC1, PPARA, CDC7, |
| | | MGAT1, GCH1, LDHC, NR4A1, EPHX1 |
| GO:0022607 | cellular component | EHD4, TRIM21, RIPK1, CPT1A, EIF4H, TP53, SEC24D, |
| | assembly | AIFM1, HMGCR, ITGA2, DDB1, BMP1, NUP93, PRPF3, |
| | | SHMT1, CALR, PSEN1, TP53INP1, GPAA1, FAS, CRB3, |
| | | NDUFS2, S100A10, VAV3, JAK2, ATG4B, KIF2A, |
| | | SEPT9, SNX10, GCH1 |
| GO:0060333 | interferon-gamma- | TRIM21, STAT1, JAK2, IRF7 |
| | mediated signaling | |
| | pathway | |
| GO:0032689 | negative regulation of | FOXP3, CD274, PDCD1LG2 |
| | interferon-gamma | |
| | production | |
| GO:0050792 | regulation of viral | CD4, TRIM21, DDB1, BANF1, STAT1, FMR1 |
| | process | |
| GO:1901565 | organonitrogen | IDUA, RIPK1, RNF144B, PSMD3, BCKDHA, CTSK, |
| | compound catabolic | UCHL1, UBE2L6, DDB1, SHMT1, ANKZF1, PJA1, PSMB8, |
| | process | FBXO6, TYMP, NUB1 |
| GO:0046677 | response to antibiotic | TP53, AIFM1, HMGCR, ANKZF1, TP53INP1, STAT1, |
| | | JAK2, ETS1 |
| GO:1903555 | regulation of tumor | CLEC4A, RIPK1, FOXP3, CD274, JAK2 |
| | necrosis factor | |
| | superfamily cytokine | |
| | production | |
| GO:0001817 | regulation of cytokine | CD4, CLEC4A, TRIM21, RIPK1, UBE2L6, STAT1, |
| | production | FOXP3, CD274, JAK2, IRF7, PDCD1LG2, CASP1 |
| GO:0030163 | protein catabolic | RIPK1, RNF144B, PSMD3, CTSK, UCHL1, UBE2L6, |
| | process | DDB1, ANKZF1, PJA1, PSMB8, FBXO6, NUB1 |
| GO:0034641 | cellular nitrogen | MPG, NAMPT, RRP9, DDX39A, FOSB, ACLY, MRPL15, |
| | compound metabolic | MOCOS, CCNE1, CREM, POLA2, CPT1A, EIF4H, TP53, |
| | process | PTS, UBE2L6, HMGCR, DDB1, FASN, MCM7, GMPPB, |
| | | PRPF3, STAT2, SHMT1, PSEN1, NOC4L, MXI1, IDH2, |
| | | ETV7, TP53INP1, ATF3, WARS, EDC4, BAZ1A, STAT1, |
| | | NDUFS2, DNASE1L1, GCLM, FMR1, YRDC, FOXP3, |
| | | FBXO6, PDHA1, DCP2, ETS1, TYMP, IRF7, NOLC1, |
| | | PPARA, CDC7, MGAT1, GCH1, LDHC, NR4A1 |
| GO:0034976 | response to | TP53, AIFM1, CALR, ANKZF1, PDIA5, ATF3, FBXO6 |
| | endoplasmic | |
| | reticulum stress | |
| GO:0042558 | pteridine-containing | PTS, SHMT1, GCH1 |
| | compound metabolic | |
| | process | |
| GO:0046719 | regulation by virus of | DDB1, STAT1 |
| | viral protein levels in | |
| | host cell | |
| GO:0050776 | regulation of immune | CD4, CLEC4A, TRAFD1, RIPK1, C1QB, PSEN1, ICAM4, |
| | response | STAT1, DUSP10, VAV3, C1QA, FOXP3, CD274, JAK2, |
| | | IRF7 |
| GO:0050867 | positive regulation of | CD4, IGFBP2, DUSP10, VAV3, FOXP3, CD274, JAK2, |
| | cell activation | PDCD1LG2 |
| GO:1903708 | positive regulation of | CD4, RIPK1, STAT1, DUSP10, FOXP3, ETS1 |
| | hemopoiesis | |
| GO:0009057 | macromolecule | IDUA, RIPK1, RNF144B, PSMD3, CTSK, UCHL1, |
| | catabolic process | UBE2L6, DDB1, ANKZF1, EDC4, PJA1, DNASE1L1, PSMB8, |
| | | FBXO6, DCP2, NUB1 |
| GO:1901576 | organic substance | B4GALT7, NAMPT, FOSB, ACLY, MRPL15, MOCOS, |
| | biosynthetic process | LPCAT2, CCNE1, CREM, POLA2, EIF4H, SORD, TP53, |
| | | PTS, UBE2L6, HMGCR, FASN, MCM7, GMPPB, STAT2, |
| | | GYS1, SHMT1, PSEN1, MXI1, IDH2, STARD3, ETV7, |
| | | TP53INP1, ATF3, GPAA1, WARS, BAZ1A, STAT1, GCLM, |
| | | AKR1A1, FOXP3, PDHA1, ETS1, DHRS7B, TYMP, IRF7, |
| | | LSS, ATG4B, PPARA, CDC7, DDOST, MGAT1, GCH1, |
| | | LDHC, NR4A1 |
| GO:0006984 | ER-nucleus signaling | TP53, CALR, ATF3 |
| | pathway | |
| GO:0007565 | female pregnancy | EPOR, NAMPT, IGFBP2, FOSB, ITGA2, ETS1 |
| GO:0009719 | response to | CD4, IGFBP2, FOSB, CCNE1, SORD, TP53, AIFM1, |
| | endogenous stimulus | ITGA2, MCM7, SHMT1, CALR, FBN1, PSEN1, STAT1, |
| | | GCLM, JAK2, ETS1, SLC26A6, PPARA, NR4A1 |
| GO:0051223 | regulation of protein | CPT1A, TP53, HMGCR, PSEN1, IDH2, SFN, FOXP3, |
| | transport | CD274, JAK2, NOLC1, BCAP31, CASP1 |
| GO:0006997 | nucleus organization | BANF1, NUP93, SPAG4, ETS1, NOLC1 |
| GO:0019220 | regulation of | CD4, EHD4, RRAS, RIPK1, CCNE1, TP53, UCHL1, |
| | phosphate metabolic | HMGCR, ITGA2, CXCL10, MCM7, STAT2, FBN1, PSEN1, |
| | process | SFN, ATF3, WARS, FAS, DUSP10, VAV3, FMR1, JAK2, |
| | | PPARA |
| GO:0002253 | activation of immune | CD4, CLEC4A, RIPK1, C1QB, PSEN1, VAV3, C1QA, |
| | response | FOXP3, IRF7 |
| GO:0006101 | citrate metabolic | ACLY, IDH2, PDHA1 |
| | process | |
| GO:0009636 | response to toxic | SLC7A11, KCNMA1, AIFM1, HMGCR, ANKZF1, |
| | substance | TP53INP1, STAT1, ETS1, PPARA, EPHX1 |
| GO:0031958 | corticosteroid | CALR, JAK2 |
| | receptor signaling | |
| | pathway | |
| GO:0032000 | positive regulation of | CPT1A, PPARA |
| | fatty acid beta- | |
| | oxidation | |
| GO:0043589 | skin morphogenesis | ITGA2, PSEN1 |
| GO:0043933 | protein-containing | EHD4, TRIM21, RIPK1, MRPL15, CPT1A, EIF4H, TP53, |
| | complex subunit | SEC24D, AIFM1, HMGCR, DDB1, BMP1, NUP93, |
| | organization | PRPF3, SHMT1, CALR, GPAA1, FAS, NDUFS2, S100A10, |
| | | JAK2, KIF2A, SEPT9, GCH1 |
| GO:0051173 | positive regulation of | CD4, PSME2, EHD4, NAMPT, FOSB, RIPK1, RNF144B, |
| | nitrogen compound | CCNE1, CREM, TP53, AIFM1, HMGCR, ITGA2, DDB1, |
| | metabolic process | CXCL10, FBN1, PSEN1, TP53INP1, ATF3, FAS, STAT1, |
| | | FMR1, CXCR3, FOXP3, JAK2, ETS1, IRF7, ATG4B, |
| | | NOLC1, PPARA, BCAP31, CDC7, CASP1, NR4A1, NUB1 |
| GO:0052548 | regulation of | PSME2, RIPK1, AIFM1, SFN, FAS, PSMB8, JAK2, |
| | endopeptidase | BCAP31, CASP1 |
| | activity | |
| GO:0061136 | regulation of | PSME2, RNF144B, PSEN1, FMR1, BCAP31, NUB1 |
| | proteasomal protein | |
| | catabolic process | |
| GO:0090316 | positive regulation of | TP53, PSEN1, SFN, JAK2, BCAP31 |
| | intracellular protein | |
| | transport | |
| GO:1901031 | regulation of | RIPK1, NUP93, GCH1 |
| | response to reactive | |
| | oxygen species | |
| GO:0070482 | response to oxygen | TP53, KCNMA1, AIFM1, ITGA2, FAS, ETS1, PPARA, |
| | levels | CASP1 |
| GO:0034644 | cellular response to | TP53, DDB1, TP53INP1, FMR1 |
| | UV | |
| GO:0048878 | chemical homeostasis | CD4, KCNMA1, DDB1, CXCL10, CALR, FBN1, PSEN1, |
| | | SFN, STAT1, GCLM, CXCR3, LDLRAP1, JAK2, |
| | | SLC26A6, BCAP31, SNX10 |
| GO:0050789 | regulation of | CD4, B4GALT7, AAAS, PSME2, EHD4, EPOR, NAMPT, |
| | biological process | CLEC4A, IGFBP2, DDX39A, FOSB, RRAS, ACLY, |
| | | TRIM21, TRAFD1, RIPK1, RNF144B, CCNE1, PSMD3, |
| | | CREM, CPT1A, EIF4H, TP53, CTSK, FEZ1, UCHL1, |
| | | KCNMA1, UBE2L6, AIFM1, HMGCR, FYCO1, ITGA2, DDB1, |
| | | FASN, CXCL10, BMP1, MCM7, BCL2L14, BANF1, NUP93, |
| | | C1QB, STAT2, SHMT1, CALR, PDIA5, FBN1, PSEN1, |
| | | NOC4L, MXI1, IDH2, RASGRP2, SFN, ETV7, ICAM4, |
| | | PPM1G, TP53INP1, ATF3, WARS, VAT1, FAS, EDC4, |
| | | BAZ1A, STAT1, DUSP10, S100A10, VAV3, GCLM, FMR1, |
| | | YRDC, CXCR3, LDLRAP1, C1QA, PSMB8, FOXP3, |
| | | FBXO6, RDH11, CD274, JAK2, DCP2, ETS1, SLC26A6, TYMP, |
| | | IRF7, LSS, PDCD1LG2, ATG4B, IFRD1, KIF2A, NOLC1, |
| | | PPARA, SEPT9, BCAP31, CDC7, GCH1, DAPP1, CASP1, |
| | | NR4A1, NUB1, PLA2G4C |
| GO:0048583 | regulation of | CD4, NAMPT, CLEC4A, IGFBP2, RRAS, TRAFD1, |
| | response to stimulus | RIPK1, TP53, UCHL1, HMGCR, ITGA2, CXCL10, BMP1, |
| | | BCL2L14, NUP93, C1QB, CALR, FBN1, PSEN1, SFN, ICAM4, |
| | | TP53INP1, ATF3, FAS, STAT1, DUSP10, VAV3, GCLM, |
| | | FMR1, CXCR3, LDLRAP1, C1QA, FOXP3, RDH11, |
| | | CD274, JAK2, ETS1, TYMP, IRF7, PPARA, BCAP31, GCH1, |
| | | CASP1 |
| GO:0048545 | response to steroid | IGFBP2, FOSB, CCNE1, AIFM1, CALR, JAK2, PPARA, |
| | hormone | NR4A1 |
| G0:0046483 | heterocycle metabolic | MPG, NAMPT, RRP9, DDX39A, FOSB, ACLY, MOCOS, |
| | process | CCNE1, CREM, POLA2, TP53, PTS, UBE2L6, HMGCR, |
| | | DDB1, FASN, MCM7, GMPPB, PRPF3, STAT2, SHMT1, |
| | | NOC4L, MXI1, IDH2, ETV7, TP53INP1, ATF3, WARS, |
| | | EDC4, BAZ1A, STAT1, NDUFS2, DNASE1L1, FMR1, |
| | | YRDC, FOXP3, FBXO6, PDHA1, DCP2, ETS1, TYMP, IRF7, |
| | | NOLC1, PPARA, CDC7, MGAT1, GCH1, LDHC, NR4A1, |
| | | EPHX1 |
| GO:0043401 | steroid hormone | CCNE1, CALR, JAK2, PPARA, NR4A1 |
| | mediated signaling | |
| | pathway | |
| GO:0019043 | establishment of viral | BANF1, IRF7 |
| | latency | |
| GO:0046598 | positive regulation of | CD4, TRIM21 |
| | viral entry into host | |
| | cell | |
| GO:2001269 | positive regulation of | FAS, JAK2 |
| | cysteine-type | |
| | endopeptidase | |
| | activity involved in | |
| | apoptotic signaling | |
| | pathway | |
| GO:0044257 | cellular protein | RIPK1, RNF144B, PSMD3, CTSK, UCHL1, UBE2L6, |
| | catabolic process | DDB1, ANKZF1, PSMB8, FBXO6, NUB1 |
| GO:0048002 | antigen processing | CLEC4A, SEC24D, CALR, KIF2A, BCAP31 |
| | and presentation of | |
| | peptide antigen | |
| GO:0042592 | homeostatic process | CD4, CCNE1, POLA2, CTSK, KCNMA1, DDB1, |
| | | CXCL10, CALR, PDIA5, FBN1, PSEN1, SFN, STAT1, GCLM, |
| | | CXCR3, LDLRAP1, FOXP3, JAK2, SLC26A6, BCAP31, |
| | | SNX10 |
| GO:0033209 | tumor necrosis factor- | RIPK1, FAS, STAT1, JAK2 |
| | mediated signaling | |
| | pathway | |
| GO:0050870 | positive regulation of | CD4, IGFBP2, DUSP10, FOXP3, CD274, PDCD1LG2 |
| | T cell activation | |
| GO:0051716 | cellular response to | CD4, PSME2, MPG, EHD4, EPOR, NAMPT, CLEC4A, |
| | stimulus | IGFBP2, DDX39A, FOSB, RRAS, TRIM21, RIPK1, |
| | | MRPL15, CCNE1, CREM, CPT1A, TP53, FEZ1, UBE2L6, AIFM1, |
| | | ITGA2, DDB1, FASN, CXCL10, MCM7, NUP93, STAT2, |
| | | SHMT1, CALR, ANKZF1, PDIA5, FBN1, PSEN1, |
| | | RASGRP2, SFN, TP53INP1, ATF3, FAS, STAT1, VAV3, GCLM, |
| | | FMR1, CXCR3, PSMB8, FOXP3, FBXO6, RDH11, |
| | | CD274, JAK2, ETS1, SLC26A6, IRF7, PPARA, BCAP31, CDC7, |
| | | SNX10, DAPP1, CASP1, NR4A1, PLA2G4C, EPHX1 |
| GO:0051251 | positive regulation of | CD4, IGFBP2, DUSP10, VAV3, FOXP3, CD274, |
| | lymphocyte | PDCD1LG2 |
| | activation | |
| GO:0019637 | organophosphate | NAMPT, ACLY, MOCOS, LPCAT2, SORD, HMGCR, |
| | metabolic process | FASN, SHMT1, IDH2, GPAA1, NDUFS2, AKR1A1, PDHA1, |
| | | GCH1, LDHC, PLA2G4C |
| GO:0071396 | cellular response to | CCNE1, CPT1A, AIFM1, ITGA2, CXCL10, CALR, JAK2, |
| | lipid | PPARA, CASP1, NR4A1 |
| GO:0071495 | cellular response to | IGFBP2, FOSB, CCNE1, TP53, AIFM1, ITGA2, MCM7, |
| | endogenous stimulus | SHMT1, CALR, FBN1, PSEN1, STAT1, GCLM, JAK2, |
| | | SLC26A6, PPARA, NR4A1 |
| GO:1901699 | cellular response to | TP53, AIFM1, SHMT1, FBN1, PSEN1, STAT1, GCLM, |
| | nitrogen compound | FMR1, JAK2, SLC26A6, NR4A1 |
| GO:1903900 | regulation of viral life | CD4, TRIM21, DDB1, BANF1, FMR1 |
| | cycle | |
| GO:0006725 | cellular aromatic | MPG, NAMPT, RRP9, DDX39A, FOSB, ACLY, MOCOS, |
| | compound metabolic | CCNE1, CREM, POLA2, TP53, PTS, UBE2L6, HMGCR, |
| | process | DDB1, FASN, MCM7, GMPPB, PRPF3, STAT2, SHMT1, |
| | | NOC4L, MXI1, IDH2, ETV7, TP53INP1, ATF3, WARS, |
| | | EDC4, BAZ1A, STAT1, NDUFS2, DNASE1L1, FMR1, |
| | | YRDC, FOXP3, FBXO6, PDHA1, DCP2, ETS1, TYMP, IRF7, |
| | | NOLC1, PPARA, CDC7, MGAT1, GCH1, LDHC, NR4A1, |
| | | EPHX1 |
| GO:0071383 | cellular response to | CCNE1, AIFM1, CALR, JAK2, PPARA, NR4A1 |
| | steroid hormone | |
| | stimulus | |

TABLE 11
Gene Enrichment for Tuberculosis Pre-vaccine Universal Signatures

| #Term ID | Term Description | Labels |
|---|---|---|
| GO:0071383 | cellular response to | CCNE1, AIFM1, CALR, JAK2, PPARA, NR4A1 |
| | steroid hormone | |
| | stimulus | |

TABLE 12
Gene Enrichment for Tuberculosis Pre-Challenge Universal Signatures

| #Term ID | Term Description | Labels |
|---|---|---|
| GO:0042493 | response to drug | IGFBP2, CPT1A, SORD, TP53, SLC7A11, HMGCR, |
| | | CALR, ANKZF1, TP53INP1, S100A10, SLC26A6 |
| GO:0090181 | regulation of | HMGCR, FASN, LDLRAP1, LSS |
| | cholesterol | |
| | metabolic process | |
| GO:0048147 | negative regulation | B4GALT7, TP53, TP53INP1 |
| | of fibroblast | |
| | proliferation | |
| GO:0006066 | alcohol metabolic | SORD, PTS, HMGCR, STARD3, LDLRAP1, LSS |
| | process | |

TABLE 13
Gene Enrichment for Tuberculosis Pre-Challenge Universal Signatures

| #Term ID | Term description | Labels |
|---|---|---|
| GO:0034097 | response to cytokine | PSME2, EPOR, TRIM21, TRAFD1, RIPK1, MRPL15, |
| | | CXCL10, STAT2, FAS, STAT1, PSMB8, CD274, JAK2, IRF7, |
| | | SNX10, GCH1, CASP1, NUB1 |
| GO:0010033 | response to organic | PSME2, EPOR, FOSB, TRIM21, TRAFD1, RIPK1, |
| | substance | MRPL15, FEZ1, KCNMA1, ITGA2, CXCL10, STAT2, PSEN1, |
| | | ATF3, FAS, STAT1, DUSP10, PSMB8, FBXO6, CD274, |
| | | JAK2, IRF7, SNX10, GCH1, CASP1, NUB1 |
| GO:0009605 | response to external | FOSB, TRIM21, FEZ1, ITGA2, CXCL10, BANF1, C1QB, |
| | stimulus | STAT2, ATF3, FAS, STAT1, DUSP10, C1QA, PSMB8, |
| | | JAK2, TYMP, IRF7, GCH1, CASP1, NUB1 |
| GO:0019221 | cytokine-mediated | PSME2, EPOR, TRIM21, RIPK1, CXCL10, STAT2, FAS, |
| | signaling pathway | STAT1, PSMB8, JAK2, IRF7, CASP1 |
| GO:0042221 | response to chemical | PSME2, EPOR, FOSB, TRIM21, TRAFD1, RIPK1, |
| | | MRPL15, FEZ1, KCNMA1, ITGA2, CXCL10, STAT2, PSEN1, |
| | | ATF3, FAS, STAT1, DUSP10, C1QA, PSMB8, FBXO6, |
| | | CD274, JAK2, TYMP, IRF7, SNX10, GCH1, CASP1, NUB1 |
| GO:0051707 | response to other | TRIM21, CXCL10, BANF1, C1QB, STAT2, FAS, STAT1, |
| | organism | DUSP10, C1QA, PSMB8, JAK2, IRF7, GCH1, CASP1, |
| | | NUB1 |
| GO:0071345 | cellular response to | PSME2, EPOR, TRIM21, RIPK1, MRPL15, CXCL10, |
| | cytokine stimulus | STAT2, FAS, STAT1, PSMB8, JAK2, IRF7, SNX10, CASP1 |
| GO:0006952 | defense response | TRIM21, CXCL10, C1QB, STAT2, PSEN1, FAS, STAT1, |
| | | C1QA, PSMB8, JAK2, IRF7, GCH1, CASP1, NUB1, |
| | | PLA2G4C |
| GO:0030162 | regulation of | PSME2, TRIM21, RIPK1, RNF144B, C1QB, PSEN1, FAS, |
| | proteolysis | C1QA, PSMB8, JAK2, CASP1, NUB1 |
| GO:0051704 | multi-organism | EPOR, FOSB, TRIM21, RIPK1, CREM, ITGA2, CXCL10, |
| | process | BANF1, C1QB, STAT2, FAS, STAT1, DUSP10, C1QA, |
| | | PSMB8, JAK2, IRF7, GCH1, CASP1, NUB1, PLA2G4C |
| GO:0034341 | response to | TRIM21, STAT1, JAK2, IRF7, GCH1, CASP1, NUB1 |
| | interferon-gamma | |
| GO:0002376 | immune system | TRIM21, RIPK1, SEC24D, CXCL10, C1QB, STAT2, |
| | process | PSEN1, FAS, STAT1, C1QA, PSMB8, CD274, JAK2, IRF7, |
| | | PDCD1LG2, KIF2A, SNX10, GCH1, CASP1, NUB1 |
| GO:0006955 | immune response | TRIM21, CXCL10, C1QB, STAT2, PSEN1, FAS, STAT1, |
| | | C1QA, PSMB8, CD274, JAK2, IRF7, PDCD1LG2, GCH1, |
| | | CASP1, NUB1 |
| GO:0045087 | innate immune | TRIM21, C1QB, STAT2, STAT1, C1QA, PSMB8, JAK2, |
| | response | IRF7, GCH1, CASP1, NUB1 |
| GO:0071310 | cellular response to | PSME2, EPOR, FOSB, TRIM21, RIPK1, MRPL15, FEZ1, |
| | organic substance | ITGA2, CXCL10, STAT2, PSEN1, ATF3, FAS, STAT1, |
| | | PSMB8, JAK2, IRF7, SNX10, CASP1 |
| GO:0098542 | defense response to | TRIM21, CXCL10, C1QB, STAT2, STAT1, C1QA, |
| | other organism | PSMB8, JAK2, IRF7, GCH1, CASP1, NUB1 |
| GO:0045862 | positive regulation of | PSME2, RIPK1, RNF144B, PSEN1, FAS, JAK2, CASP1, |
| | proteolysis | NUB1 |
| GO:0002682 | regulation of immune | TRAFD1, RIPK1, ITGA2, CXCL10, C1QB, PSEN1, |
| | system process | ICAM4, STAT1, DUSP10, C1QA, CD274, JAK2, IRF7, |
| | | PDCD1LG2 |
| GO:0006508 | proteolysis | PSME2, LAP3, RIPK1, RNF144B, CTSK, UBE2L6, |
| | | C1QB, PSEN1, C1QA, PSMB8, FBXO6, CASP1, NUB1 |
| GO:0031347 | regulation of defense | TRAFD1, RIPK1, ITGA2, C1QB, STAT1, DUSP10, |
| | response | C1QA, JAK2, IRF7, CASP1 |
| GO:0050776 | regulation of immune | TRAFD1, RIPK1, C1QB, PSEN1, ICAM4, STAT1, |
| | response | DUSP10, C1QA, CD274, JAK2, IRF7 |
| GO:0002684 | positive regulation of | RIPK1, ITGA2, CXCL10, C1QB, PSEN1, STAT1, |
| | immune system | DUSP10, C1QA, CD274, IRF7, PDCD1LG2 |
| | process | |
| GO:0009612 | response to | FOSB, ITGA2, CXCL10, FAS, STAT1, CASP1 |
| | mechanical stimulus | |
| GO:0050896 | response to stimulus | PSME2, EPOR, FOSB, TRIM21, TRAFD1, RIPK1, |
| | | MRPL15, CREM, FEZ1, KCNMA1, UBE2L6, ITGA2, CXCL10, |
| | | BANF1, C1QB, STAT2, PSEN1, ATF3, FAS, STAT1, |
| | | DUSP10, C1QA, PSMB8, FBXO6, CD274, JAK2, TYMP, IRF7, |
| | | PDCD1LG2, SNX10, GCH1, DAPP1, CASP1, NUB1, |
| | | PLA2G4C |
| GO:0001817 | regulation of cytokine | TRIM21, RIPK1, UBE2L6, STAT1, CD274, JAK2, IRF7, |
| | production | PDCD1LG2, CASP1 |
| GO:0006950 | response to stress | TRIM21, RIPK1, KCNMA1, UBE2L6, ITGA2, CXCL10, |
| | | C1QB, STAT2, PSEN1, ATF3, FAS, STAT1, C1QA, |
| | | PSMB8, FBXO6, JAK2, IRF7, GCH1, CASP1, NUB1, |
| | | PLA2G4C |
| GO:0032101 | regulation of | TRAFD1, RIPK1, ITGA2, CXCL10, C1QB, STAT1, |
| | response to external | DUSP10, C1QA, JAK2, IRF7, CASP1 |
| | stimulus | |
| GO:0034612 | response to tumor | RIPK1, FAS, STAT1, JAK2, GCH1, NUB1 |
| | necrosis factor | |
| GO:0043065 | positive regulation of | RIPK1, KCNMA1, BCL2L14, PSEN1, ATF3, FAS, |
| | apoptotic process | CD274, JAK2, CASP1 |
| GO:0060337 | type I interferon | STAT2, STAT1, PSMB8, IRF7 |
| | signaling pathway | |
| GO:0060333 | interferon-gamma- | TRIM21, STAT1, JAK2, IRF7 |
| | mediated signaling | |
| | pathway | |
| GO:0050789 | regulation of | PSME2, EPOR, FOSB, TRIM21, TRAFD1, RIPK1, |
| | biological process | RNF144B, CREM, CTSK, FEZ1, KCNMA1, UBE2L6, ITGA2, |
| | | CXCL10, BCL2L14, BANF1, C1QB, STAT2, PSEN1, MXI1, |
| | | ETV7, ICAM4, ATF3, WARS, FAS, BAZ1A, STAT1, |
| | | DUSP10, C1QA, PSMB8, FBXO6, CD274, JAK2, TYMP, IRF7, |
| | | PDCD1LG2, KIF2A, GCH1, DAPP1, CASP1, NUB1, |
| | | PLA2G4C |
| GO:0001959 | regulation of | RIPK1, STAT1, JAK2, IRF7, CASP1 |
| | cytokine-mediated | |
| | signaling pathway | |
| GO:1901564 | organonitrogen | PSME2, LAP3, TRIM21, RIPK1, RNF144B, MRPL15, |
| | compound metabolic | MOCOS, LPCAT2, CREM, CTSK, UBE2L6, C1QB, PSEN1, |
| | process | WARS, DUSP10, C1QA, PSMB8, FBXO6, JAK2, TYMP, |
| | | IRF7, GCH1, DAPP1, CASP1, LDHC, NUB1, PLA2G4C |
| GO:0071346 | cellular response to | TRIM21, STAT1, JAK2, IRF7, CASP1 |
| | interferon-gamma | |
| GO:0097300 | programmed necrotic | RIPK1, FAS, CASP1 |
| | cell death | |
| GO:0010950 | positive regulation of | PSME2, RIPK1, FAS, JAK2, CASP1 |
| | endopeptidase | |
| | activity | |
| GO:0033209 | tumor necrosis factor- | RIPK1, FAS, STAT1, JAK2 |
| | mediated signaling | |
| | pathway | |
| GO:0051246 | regulation of protein | PSME2, TRIM21, RIPK1, RNF144B, ITGA2, CXCL10, |
| | metabolic process | C1QB, STAT2, PSEN1, ATF3, WARS, FAS, DUSP10, |
| | | C1QA, PSMB8, JAK2, CASP1, NUB1 |
| GO:0065007 | biological regulation | PSME2, EPOR, FOSB, TRIM21, TRAFD1, RIPK1, |
| | | RNF144B, CREM, CTSK, FEZ1, KCNMA1, UBE2L6, ITGA2, |
| | | CXCL10, BCL2L14, BANF1, C1QB, STAT2, PSEN1, MXI1, |
| | | ETV7, ICAM4, ATF3, WARS, FAS, BAZ1A, STAT1, |
| | | DUSP10, C1QA, PSMB8, FBXO6, CD274, JAK2, TYMP, IRF7, |
| | | PDCD1LG2, KIF2A, SNX10, GCH1, DAPP1, CASP1, |
| | | NUB1, PLA2G4C |
| GO:0006919 | activation of | RIPK1, FAS, JAK2, CASP1 |
| | cysteine-type | |
| | endopeptidase | |
| | activity involved in | |
| | apoptotic process | |
| GO:0080134 | regulation of | TRAFD1, RIPK1, ITGA2, C1QB, FAS, STAT1, DUSP10, |
| | response to stress | C1QA, JAK2, IRF7, GCH1, CASP1 |
| GO:2001235 | positive regulation of | RIPK1, BCL2L14, ATF3, FAS, JAK2 |
| | apoptotic signaling | |
| | pathway | |
| GO:0002831 | regulation of | TRAFD1, RIPK1, STAT1, DUSP10, CD274, JAK2, IRF7 |
| | response to biotic | |
| | stimulus | |
| GO:0051239 | regulation of | TRIM21, RIPK1, CTSK, FEZ1, UBE2L6, ITGA2, |
| | multicellular | CXCL10, PSEN1, WARS, STAT1, DUSP10, CD274, JAK2, |
| | organismal process | TYMP, IRF7, PDCD1LG2, GCH1, CASP1 |
| GO:0032496 | response to | CXCL10, FAS, DUSP10, JAK2, GCH1, CASP1 |
| | lipopolysaccharide | |
| GO:0097527 | necroptotic signaling | RIPK1, FAS |
| | pathway | |
| GO:0007259 | receptor signaling | STAT2, STAT1, JAK2 |
| | pathway via JAK- | |
| | STAT | |
| GO:0032479 | regulation of type I | TRIM21, UBE2L6, STAT1, IRF7 |
| | interferon production | |
| GO:0043901 | negative regulation of | TRIM21, TRAFD1, BANF1, STAT1, DUSP10 |
| | multi-organism | |
| | process | |
| GO:0050727 | regulation of | ITGA2, C1QB, DUSP10, C1QA, JAK2, CASP1 |
| | inflammatory | |
| | response | |
| GO:0043900 | regulation of multi- | TRIM21, TRAFD1, RIPK1, BANF1, STAT1, DUSP10, |
| | organism process | JAK2, IRF7 |
| GO:0006807 | nitrogen compound | PSME2, LAP3, FOSB, TRIM21, RIPK1, RNF144B, |
| | metabolic process | MRPL15, MOCOS, LPCAT2, CREM, CTSK, UBE2L6, C1QB, |
| | | STAT2, PSEN1, MXI1, ETV7, ATF3, WARS, BAZ1A, |
| | | STAT1, DUSP10, C1QA, PSMB8, FBXO6, JAK2, TYMP, IRF7, |
| | | GCH1, DAPP1, CASP1, LDHC, NUB1, PLA2G4C |
| GO:0007166 | cell surface receptor | PSME2, EPOR, TRIM21, RIPK1, ITGA2, CXCL10, |
| | signaling pathway | STAT2, PSEN1, FAS, STAT1, PSMB8, CD274, JAK2, IRF7, |
| | | CASP1 |
| GO:0048518 | positive regulation of | PSME2, FOSB, TRIM21, RIPK1, RNF144B, CREM, |
| | biological process | FEZ1, KCNMA1, ITGA2, CXCL10, BCL2L14, C1QB, PSEN1, |
| | | ATF3, WARS, FAS, STAT1, DUSP10, C1QA, CD274, |
| | | JAK2, IRF7, PDCD1LG2, GCH1, CASP1, NUB1 |
| GO:0042981 | regulation of | RIPK1, RNF144B, KCNMA1, BCL2L14, PSEN1, ATF3, |
| | apoptotic process | FAS, STAT1, CD274, JAK2, IRF7, CASP1 |
| GO:0045088 | regulation of innate | TRAFD1, RIPK1, STAT1, DUSP10, JAK2, IRF7 |
| | immune response | |
| GO:0043589 | skin morphogenesis | ITGA2, PSEN1 |
| GO:2001238 | positive regulation of | RIPK1, BCL2L14, ATF3 |
| | extrinsic apoptotic | |
| | signaling pathway | |
| GO:0019043 | establishment of viral | BANF1, IRF7 |
| | latency | |
| GO:2001269 | positive regulation of | FAS, JAK2 |
| | cysteine-type | |
| | endopeptidase | |
| | activity involved in | |
| | apoptotic signaling | |
| | pathway | |
| GO:0001819 | positive regulation of | RIPK1, STAT1, CD274, JAK2, IRF7, CASP1 |
| | cytokine production | |
| GO:0044419 | interspecies | RIPK1, ITGA2, CXCL10, BANF1, STAT2, STAT1, |
| | interaction between | PSMB8, IRF7 |
| | organisms | |
| GO:0046007 | negative regulation of | CD274, PDCD1LG2 |
| | activated T cell | |
| | proliferation | |
| GO:0052548 | regulation of | PSME2, RIPK1, FAS, PSMB8, JAK2, CASP1 |
| | endopeptidase | |
| | activity | |
| GO:0070106 | interleukin-27- | STAT1, JAK2 |
| | mediated signaling | |
| | pathway | |
| GO:0070757 | interleukin-35- | STAT1, JAK2 |
| | mediated signaling | |
| | pathway | |
| GO:1902041 | regulation of extrinsic | RIPK1, ATF3, FAS |
| | apoptotic signaling | |
| | pathway via death | |
| | domain receptors | |
| GO:2001233 | regulation of | RIPK1, BCL2L14, PSEN1, ATF3, FAS, JAK2 |
| | apoptotic signaling | |
| | pathway | |
| GO:0044257 | cellular protein | RIPK1, RNF144B, CTSK, UBE2L6, PSMB8, FBXO6, |
| | catabolic process | NUB1 |
| GO:0016032 | viral process | RIPK1, ITGA2, BANF1, STAT2, STAT1, PSMB8, IRF7 |
| GO:0009615 | response to virus | CXCL10, BANF1, STAT2, STAT1, IRF7 |
| GO:0070102 | interleukin-6- | STAT1, JAK2 |
| | mediated signaling | |
| | pathway | |
| GO:2001236 | regulation of extrinsic | RIPK1, BCL2L14, ATF3, FAS |
| | apoptotic signaling | |
| | pathway | |
| GO:1901700 | response to oxygen- | FOSB, KCNMA1, ITGA2, CXCL10, PSEN1, FAS, STAT1, |
| | containing compound | DUSP10, JAK2, GCH1, CASP1 |
| GO:0009893 | positive regulation of | PSME2, FOSB, TRIM21, RIPK1, RNF144B, CREM, ITGA2, |
| | metabolic process | CXCL10, PSEN1, ATF3, WARS, FAS, STAT1, JAK2, |
| | | IRF7, GCH1, CASP1, NUB1 |
| GO:0019538 | protein metabolic | PSME2, LAP3, TRIM21, RIPK1, RNF144B, MRPL15, |
| | process | MOCOS, CTSK, UBE2L6, C1QB, PSEN1, WARS, DUSP10, |
| | | C1QA, PSMB8, FBXO6, JAK2, IRF7, DAPP1, CASP1, |
| | | NUB1 |
| GO:0016064 | immunoglobulin | C1QB, C1QA, IRF7 |
| | mediated immune | |
| | response | |
| GO:0051770 | positive regulation of | STAT1, JAK2 |
| | nitric-oxide synthase | |
| | biosynthetic process | |
| GO:0051969 | regulation of | ITGA2, TYMP |
| | transmission of nerve | |
| | impulse | |
| GO:0000122 | negative regulation of | FOSB, CREM, PSEN1, MXI1, ETV7, ATF3, STAT1, IRF7 |
| | transcription by RNA | |
| | polymerase II | |
| GO:0032268 | regulation of cellular | PSME2, TRIM21, RIPK1, RNF144B, ITGA2, CXCL10, |
| | protein metabolic | STAT2, PSEN1, ATF3, WARS, FAS, DUSP10, JAK2, CASP1, |
| | process | NUB1 |
| GO:0032693 | negative regulation of | CD274, PDCD1LG2 |
| | interleukin-10 | |
| | production | |
| GO:0048522 | positive regulation of | PSME2, FOSB, TRIM21, RIPK1, RNF144B, CREM, FEZ1, |
| | cellular process | KCNMA1, ITGA2, CXCL10, BCL2L14, PSEN1, ATF3, |
| | | WARS, FAS, STAT1, DUSP10, CD274, JAK2, IRF7, |
| | | PDCD1LG2, CASP1, NUB1 |
| GO:0031667 | response to nutrient | ITGA2, CXCL10, ATF3, FAS, STAT1, CASP1 |
| | levels | |
| GO:0033993 | response to lipid | FOSB, ITGA2, CXCL10, FAS, DUSP10, JAK2, GCH1, |
| | | CASP1 |
| GO:0071260 | cellular response to | ITGA2, FAS, CASP1 |
| | mechanical stimulus | |
| GO:0048519 | negative regulation of | FOSB, TRIM21, TRAFD1, RIPK1, RNF144B, CREM, |
| | biological process | FEZ1, UBE2L6, CXCL10, BANF1, PSEN1, MXI1, ETV7, |
| | | ATF3, WARS, FAS, STAT1, DUSP10, FBXO6, CD274, JAK2, |
| | | IRF7, PDCD1LG2 |
| GO:0048661 | positive regulation of | ITGA2, STAT1, JAK2 |
| | smooth muscle cell | |
| | proliferation | |
| GO:0051607 | defense response to | CXCL10, STAT2, STAT1, IRF7 |
| | virus | |
| GO:0061136 | regulation of | PSME2, RNF144B, PSEN1, NUB1 |
| | proteasomal protein | |
| | catabolic process | |
| GO:0008285 | negative regulation of | MXI1, WARS, STAT1, DUSP10, CD274, JAK2, |
| | cell population | PDCD1LG2 |
| | proliferation | |
| GO:0048584 | positive regulation of | RIPK1, ITGA2, CXCL10, BCL2L14, C1QB, PSEN1, |
| | response to stimulus | ATF3, FAS, C1QA, CD274, JAK2, IRF7, CASP1 |
| GO:0051240 | positive regulation of | RIPK1, FEZ1, ITGA2, PSEN1, STAT1, DUSP10, CD274, |
| | multicellular | JAK2, IRF7, GCH1, CASP1 |
| | organismal process | |
| GO:0032436 | positive regulation of | RNF144B, PSEN1, NUB1 |
| | proteasomal | |
| | ubiquitin-dependent | |
| | protein catabolic | |
| | process | |
| GO:0032727 | positive regulation of | STAT1, IRF7 |
| | interferon-alpha | |
| | production | |
| GO:0045453 | bone resorption | CTSK, SNX10 |
| GO:0048525 | negative regulation of | TRIM21, BANF1, STAT1 |
| | viral process | |
| GO:0097191 | extrinsic apoptotic | RIPK1, FAS, JAK2 |
| | signaling pathway | |
| GO:0071704 | organic substance | PSME2, LAP3, FOSB, TRIM21, RIPK1, RNF144B, MRPL15, |
| | metabolic process | MOCOS, LPCAT2, CREM, CTSK, UBE2L6, C1QB, |
| | | STAT2, PSEN1, MXI1, ETV7, ATF3, WARS, BAZ1A, STAT1, |
| | | DUSP10, C1QA, PSMB8, FBXO6, JAK2, TYMP, IRF7, |
| | | GCH1, DAPP1, CASP1, LDHC, NUB1, PLA2G4C |
| GO:0035666 | TRIF-dependent toll- | RIPK1, IRF7 |
| | like receptor | |
| | signaling pathway | |
| GO:0042127 | regulation of cell | ITGA2, CXCL10, MXI1, ATF3, WARS, FAS, STAT1, |
| | population | DUSP10, CD274, JAK2, PDCD1LG2 |
| | proliferation | |
| GO:0007584 | response to nutrient | ITGA2, CXCL10, STAT1, CASP1 |
| GO:0019222 | regulation of | PSME2, FOSB, TRIM21, RIPK1, RNF144B, CREM, FEZ1, |
| | metabolic process | ITGA2, CXCL10, C1QB, STAT2, PSEN1, MXI1, ETV7, |
| | | ATF3, WARS, FAS, BAZ1A, STAT1, DUSP10, C1QA, |
| | | PSMB8, JAK2, IRF7, GCH1, CASP1, NUB1 |
| GO:0051171 | regulation of nitrogen | PSME2, FOSB, TRIM21, RIPK1, RNF144B, CREM, |
| | compound metabolic | ITGA2, CXCL10, C1QB, STAT2, PSEN1, MXI1, ETV7, ATF3, |
| | process | WARS, FAS, BAZ1A, STAT1, DUSP10, C1QA, PSMB8, |
| | | JAK2, IRF7, CASP1, NUB1 |
| GO:0044706 | multi-multicellular | EPOR, FOSB, ITGA2, PLA2G4C |
| | organism process | |
| GO:0044238 | primary metabolic | PSME2, LAP3, FOSB, TRIM21, RIPK1, RNF144B, |
| | process | MRPL15, MOCOS, LPCAT2, CREM, CTSK, UBE2L6, C1QB, |
| | | STAT2, PSEN1, MXI1, ETV7, ATF3, WARS, BAZ1A, STAT1, |
| | | DUSP10, C1QA, PSMB8, FBXO6, JAK2, TYMP, IRF7, |
| | | DAPP1, CASP1, LDHC, NUB1, PLA2G4C |
| GO:0048583 | regulation of | TRAFD1, RIPK1, ITGA2, CXCL10, BCL2L14, C1QB, |
| | response to stimulus | PSEN1, ICAM4, ATF3, FAS, STAT1, DUSP10, C1QA, CD274, |
| | | JAK2, TYMP, IRF7, GCH1, CASP1 |
| GO:0051603 | proteolysis involved | RNF144B, CTSK, UBE2L6, PSMB8, FBXO6, NUB1 |
| | in cellular protein | |
| | catabolic process | |
| GO:0060334 | regulation of | STAT1, JAK2 |
| | interferon-gamma- | |
| | mediated signaling | |
| | pathway | |
| GO:1903959 | regulation of anion | RIPK1, PSEN1 |
| | transmembrane | |
| | transport | |
| GO:2001025 | positive regulation of | RIPK1, PSEN1 |
| | response to drug | |
| GO:0036151 | phosphatidylcholine | LPCAT2, PLA2G4C |
| | acyl-chain | |
| | remodeling | |
| GO:0070647 | protein modification | PSME2, TRIM21, RIPK1, RNF144B, UBE2L6, PSMB8, |
| | by small protein | FBXO6, NUB1 |
| | conjugation or | |
| | removal | |
| GO:0031329 | regulation of cellular | PSME2, TRIM21, RNF144B, FEZ1, PSEN1, CASP1, NUB1 |
| | catabolic process | |
| GO:0045785 | positive regulation of | ITGA2, DUSP10, CD274, JAK2, PDCD1LG2 |
| | cell adhesion | |
| GO:1901565 | organonitrogen | RIPK1, RNF144B, CTSK, UBE2L6, PSMB8, FBXO6, |
| | compound catabolic | TYMP, NUB1 |
| | process | |
| GO:0031325 | positive regulation of | PSME2, FOSB, TRIM21, RIPK1, RNF144B, CREM, |
| | cellular metabolic | ITGA2, CXCL10, PSEN1, ATF3, FAS, STAT1, JAK2, IRF7, |
| | process | CASP1, NUB1 |
| GO:0010922 | positive regulation of | ITGA2, JAK2 |
| | phosphatase activity | |
| GO:0080090 | regulation of primary | PSME2, FOSB, TRIM21, RIPK1, RNF144B, CREM, |
| | metabolic process | ITGA2, CXCL10, C1QB, STAT2, PSEN1, MXI1, ETV7, ATF3, |
| | | WARS, FAS, BAZ1A, STAT1, DUSP10, C1QA, PSMB8, |
| | | JAK2, IRF7, CASP1, NUB1 |
| GO:0010604 | positive regulation of | PSME2, FOSB, RIPK1, RNF144B, CREM, ITGA2, CXCL10, |
| | macromolecule | PSEN1, ATF3, WARS, FAS, STAT1, JAK2, IRF7, CASP1, |
| | metabolic process | NUB1 |
| GO:0002253 | activation of immune | RIPK1, C1QB, PSEN1, C1QA, IRF7 |
| | response | |
| GO:0032689 | negative regulation of | CD274, PDCD1LG2 |
| | interferon-gamma | |
| | production | |
| GO:0043170 | macromolecule | PSME2, LAP3, FOSB, TRIM21, RIPK1, RNF144B, |
| | metabolic process | MRPL15, MOCOS, CREM, CTSK, UBE2L6, C1QB, STAT2, |
| | | PSEN1, MXI1, ETV7, ATF3, WARS, BAZ1A, STAT1, DUSP10, |
| | | C1QA, PSMB8, FBXO6, JAK2, IRF7, DAPP1, CASP1, NUB1 |
| GO:0051101 | regulation of DNA | ITGA2, PSEN1, JAK2 |
| | binding | |
| GO:1903555 | regulation of tumor | RIPK1, CD274, JAK2 |
| | necrosis factor | |
| | superfamily cytokine | |
| | production | |
| GO:0006958 | complement | C1QB, C1QA |
| | activation, classical | |
| | pathway | |
| GO:0032731 | positive regulation of | JAK2, CASP1 |
| | interleukin-1 beta | |
| | production | |
| GO:0050778 | positive regulation of | RIPK1, C1QB, PSEN1, C1QA, CD274, IRF7 |
| | immune response | |
| GO:0060255 | regulation of | PSME2, FOSB, TRIM21, RIPK1, RNF144B, CREM, ITGA2, |
| | macromolecule | CXCL10, C1QB, STAT2, PSEN1, MXI1, ETV7, ATF3, |
| | metabolic process | WARS, FAS, BAZ1A, STAT1, DUSP10, C1QA, PSMB8, |
| | | JAK2, IRF7, CASP1, NUB1 |
| GO:1901031 | regulation of | RIPK1, GCH1 |
| | response to reactive | |
| | oxygen species | |
| GO:1902042 | negative regulation of | RIPK1, FAS |
| | extrinsic apoptotic | |
| | signaling pathway via | |
| | death domain | |
| | receptors | |

1. A method for identifying one or more universal signatures useful for evaluating disease activity of two or more diseases, the method comprising:
obtaining or having obtained expressions of a plurality of markers across individuals for a first disease indication;
analyzing the expressions of the plurality of markers using a machine-learned analysis to identify one or more universal signatures from the first disease indication,
wherein the one or more universal signatures are features that are predictive for a second disease indication,
wherein each of the first disease indication and the second disease indication is characterized by a common condition.

2. A method for generating a prediction of a second disease indication for a patient, the method comprising:
obtaining or having obtained expressions of one or more universal signatures from the subject, the one or more universal signatures derived from a machine-learned analysis of a plurality of markers across individuals associated with a first disease indication, wherein each of the first disease indication and the second disease indication is characterized by a common condition; and
based on the expressions for the one or more universal signatures, generating the prediction of the second disease indication.

3. The method of claim 1 or 2, wherein the one or more universal signatures comprise one or more of genes, nucleic acids, metabolites, or protein biomarkers.

4. The method of any one of claims 1-3, wherein the common condition is any one of a precursor to a disease, a sub phenotype of a disease, progression from latent to acute infection, progression from acute to chronic infection, response to an intervention, susceptibility to disease or infection, presence of acute inflammation, presence of chronic inflammation, a dysregulated pathway expression, a cellular phenotype, or a clinical phenotype.

5. The method of claim 4, wherein the clinical phenotype is any one of high blood pressure, fever, loss of blood, loss of consciousness, increased heart rate, or need for mechanical ventilation.

6. The method of any one of claims 1-5, wherein the first disease indication describes a disease activity of a first disease, and wherein the second disease indication describes a disease activity of a second disease, and wherein the first disease indication differs from the second disease indication by any of a different disease activity of a disease, a disease activity of different diseases, different disease activity of different diseases.

7. The method of any one of claims 1-6, wherein each of the first disease indication or second disease indication is any one of activity of an inflammatory disease, activity of a disease observed in an animal model, activity of a bacterial infectious disease, a progression from latent to acute infection, and wherein the disease activity of the second disease is any one of disease of a cancer, activity of a human disease that represents an equivalent phenotype of a disease in an animal, activity of an infectious disease from a non-bacterial infectious agent, protection after vaccination, estimated time to death due to disease, or a diseased condition.

8. The method of claim 6, wherein the first disease is an inflammatory disease and the second disease is a cancer.

9. The method of claim 6, wherein the first disease is observed in an animal model and wherein the second disease is an equivalent disease phenotype in humans.

10. The method of claim 6, wherein the first disease is a bacterial infectious disease and wherein the second disease is a disease from a non-bacterial infectious agent.

11. The method of claim 6, wherein the disease activity of the first disease is a progression from latent to acute infection and wherein the disease activity of the second disease is protection after vaccination.

12. The method of any one of claims 1-11, wherein the machine-learned analysis is random forest or gradient boosting for identifying the one or more universal signatures.

13. The method of any one of claims 4-12, wherein the intervention is any one of a small molecule therapeutic, a biologic, a vaccine, or a gene therapy.

14. The method of any one of claims 1-13, wherein individuals with the second disease have encountered or are likely to encounter the common condition.

15. The method of claim 2, wherein generating a prediction of the second disease indication for the patient comprises performing an unsupervised clustering of the expressions of the one or more universal signatures to classify the patient.

16. The method of claim 2 or 15, wherein generating the prediction of the second disease indication for a patient comprises performing a dimensionality reduction analysis of the expressions of the one or more universal signatures.

17. The method of any one of claim 2 or 15-16, further comprising:
determining whether to include the subject in a clinical trial study according to the predicted disease activity of the disease in the subject.

18. The method of any one of claims 1-17, wherein the one or more universal signatures comprise one or more genes selected from NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, BCAP31, ATG4B, CSRP1, RDH11, GCLM, CDC7, GLOD5, IDH2, FMR1, PPARA, CCNE1, DDB1, BMP1, EHD4, VAV3, MPG, SPAG4, PSMD3, BCKDHA, GRAMD1B, and SEC61A1.

19. The method of any one of claims 1-17, wherein the one or more universal signatures comprise one or more genes selected from CRB3, BCAP31, GMPPB, CD4, STARD3, CALR, CSRP1, CPT1A, LDLRAP1, RRAS, HMGCR, RASGRP2, PTS, SORDSLC26A6, VAT1, GPAA1, CXCR3, NAMPT, EPHX1, SEPT9, GMPPA, B4GALT7, AAAS, TP53INP1, GYS1, FASN, NOC4L, RRP9, MXI1, TP53, SLC7A11, FOXP3, DNASE1L1, MGAT1, SEC61A1, FYCO1, S100A10, LSS, IFRD1, DCP2, EDC4, ANKZF1, IDUA, IGFBP2, DDX39A, UCHL1, NR4A1, PDIA5, and ENGASE.

20. The method of any one of claims 1-17, wherein the one or more universal signatures comprise one or more genes selected from NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, ITGA2, MPZL2, FEZ1, JAK2, BAZ1A, ICAM4, DAPP1, RIPK1, RNF144B, LAP3, C1QA, TYMP, GCH1, C1QB, CREM, ETV7, FOSB, MRPL15, PSEN1, MXI1, and TRAFD1.

21. The method of any one of claims 1-17, wherein the one or more universal signatures comprise one or more genes selected from DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, RTP4, CTSD, HIST1H2BG, IL27, TNFRSF1B, SORBS1, NOP2, TNFSF13B, HLA-DRB5, RHOG, PSMB9, HSPA6, CD63, SLC2A8, IFITM1, CKB, ALDOA, MSRB1, OSMR, DRAP1, and PLA2G4A.

22. The method of any one of claims 1-17, wherein the one or more universal signatures comprise one or more genes selected from LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, KIAA1324, CENPN, IKBKE, SERPINF2, FAM162A, SNX2, SERPING1, CLCA2, DPEP3, TNFAIP2, FSTL4, CTSD, BCAR1, MKX, RGS2, SAMD9, GCLM, BST1, IRS2, RNASE6, and ELOVL3.

23. The method of any one of claims 1-17, wherein the one or more universal signatures comprise one or more genes selected from GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, TNFRSF21, PSMB9, GNAI1, TSPAN13, ATP6V0B, SLC4A4, ILF2, AKAP12, HLA-DRB5, PGR, AGTRAP, P3H1, CDADC1, TRIM5, PTGER3, ADCY6, ERBB2, NFYA, STATE, MMD, and RPL10A.

24. The method of any one of claims 1-17, wherein the one or more universal signatures comprise one or more genes selected from MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, IFNGR2, B3GNT5, KYNU, LPGAT1, SLC9A3R1, HP, PADI4, PSME1, MGST2, NR4A1, SPP1, DEFA3, ME1, RBP7, DUSP6, and MCRS1.

25. The method of any one of claims 1-17, wherein the one or more universal signatures comprise one or more genes selected from POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, RFC2, CAMK1G, CBX8, HM13, PSMB10, GCLM, SLC25A3, MYD88, IL33, ITGAM, PPIA, SEC22B, CXCR3, SCRN1, RXRA, SDHA, GLDC, FGF6, PRKG2, TFPI, and IMMT.

26. The method of any one of claims 1-17, wherein the one or more universal signatures comprise one or more genes selected from CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, PICALM, KRT6A, FMR1, PCSK9, DNASE1L3, ENDOG, TPD52L1, PEX6, MPO, CHRNA7, SLFN5, TNFRSF1A, CD24, CASC1, LLGL2, DLG5, MYO5C, PGR, PFKFB2, AK2, and COL19A1.

27. The method of any one of claims 1-17, wherein the one or more universal signatures comprise one or more genes selected from HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, ROCK1, CD83, AK7, MSR1, LCN2, SPN, ASS1, HDGF, CXCL16, POLR3D, GK, OLFM4, STK3, RCBTB1, FOLR3, FBXO32, TMEM98, PRDX2, CKB, UHRF1BP1L and CTSG.

28. The method of any one of claims 1-17, wherein the one or more universal signatures comprise one or more genes selected from AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMPI, HSD11B2, SLC25A25, RAB32, CXCL9, KCNE2, FCAR, CFP, IGF1, PEX16, RNF214, PIM1, JUNB, MDM2, PFKFB4, SIAH2, EGR2, KCNK10, EHMT2, FPR1, CD27, CETN2, and TGM1.

29. The method of any one of claims 1-17, wherein the one or more universal signatures comprise one or more genes selected from SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, MT2A, COL4A2, PLCB4, GYS1, PRKCG, RXFP2, PLA2G4C, ALDH1A2, ILIA, IBTK, SPARC, OAS3, EPHA4, HLA-B, MICB, CCL18, SLC39A6, GLCE, TUBB2B, FBXO8, and SNX6.

30. The method of any one of claims 1-17, wherein the one or more universal signatures comprise one or more genes selected from NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, SLC25A19, MAP7, XCL1, ACSL6, TFRC, CAT, NKD1, CNBP, ALDH1L1, CCL7, SLC20A1, KRAS, CSF1, CASP2, HDAC11, KIR2DS4, CEACAM19, CFH, CAB39L, DEPDC1, and PSMA1.

31. The method of any one of claims 1-17, wherein the one or more universal signatures comprise one or more genes selected from CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, CENPJ, ARL8A, CTLA4, GFRA1, WASF1, RIPK1, ENO3, KRT19, PLVAP, RAD18, ACHE, FBLN5, MGST2, ANAPC5, RFX5, CASP7, STC1, NCK2, IFI27, APOA4, and MSRB2.

32. A non-transitory computer-readable medium for identifying one or more universal signatures useful for evaluating two or more disease indications, the computer-readable medium comprising instructions that, when executed by a processor, cause the processor to perform the steps comprising:
obtaining or having obtained expressions of a plurality of markers across individuals for a first disease indication;
analyzing the expressions of the plurality of markers using a machine-learned analysis to identify one or more universal signatures from the first disease indication,
wherein the one or more universal signatures are features that are predictive for a second disease indication,
wherein each of the first disease indication and the second disease indication is characterized by a common condition.

33. A non-transitory computer-readable medium for generating a prediction of a second disease indication for a patient, the computer-readable medium comprising instructions that, when executed by a processor, cause the processor to perform the steps comprising:
obtaining or having obtained expressions of one or more universal signatures from the subject, the one or more universal signatures derived from a machine-learned analysis of a plurality of markers across individuals associated with a first disease indication, wherein each of the first disease indication and the second disease indication is characterized by a common condition; and
based on the expressions for the one or more universal signatures, generating the prediction of the second disease indication.

34. The non-transitory computer-readable medium of claim 32 or 33, wherein the one or more universal signatures comprise one or more of genes, nucleic acids, metabolites, or protein biomarkers.

35. The non-transitory computer-readable medium of any one of claims 32-34, wherein the common condition is any one of a precursor to a disease, a sub phenotype of a disease, progression from latent to acute infection, progression from acute to chronic infection, response to an intervention, susceptibility to disease or infection, presence of acute inflammation, presence of chronic inflammation, a dysregulated pathway expression, a cellular phenotype, or a clinical phenotype (e.g., high blood pressure, fever, loss of blood, loss of consciousness, or increased heart rate).

36. The non-transitory computer-readable medium of claim 35, wherein the clinical phenotype is any one of high blood pressure, fever, loss of blood, loss of consciousness, increased heart rate, or need for mechanical ventilation.

37. The non-transitory computer-readable medium of any one of claims 32-36, wherein the first disease indication describes a disease activity of a first disease, and wherein the second disease indication describes a disease activity of a second disease, and wherein the first disease indication differs from the second disease indication by any of a different disease activity of a disease, a disease activity of different diseases, different disease activity of different diseases.

38. The non-transitory computer-readable medium of any one of claims 32-37, wherein each of the first disease indication or second disease indication is any one of activity of an inflammatory disease, activity of a disease observed in an animal model, activity of a bacterial infectious disease, a progression from latent to acute infection, a dysregulated blood cell population makeup, or a dysregulated pathway expression, and wherein the disease activity of the second disease is any one of disease of a cancer, activity of a human disease that represents an equivalent phenotype of a disease in an animal, activity of an infectious disease from a non-bacterial infectious agent, protection after vaccination, estimated time to death due to disease, or a diseased condition.

39. The non-transitory computer-readable medium of claim 37, wherein the first disease is an inflammatory disease and the second disease is a cancer.

40. The non-transitory computer-readable medium of claim 37, wherein the first disease is observed in an animal model and wherein the second disease is an equivalent disease phenotype in humans.

41. The non-transitory computer-readable medium of claim 37, wherein the first disease is a bacterial infectious disease and wherein the second disease is a disease from a non-bacterial infectious agent.

42. The non-transitory computer-readable medium of claim 37, wherein the disease activity of the first disease is a progression from latent to acute infection and wherein the disease activity of the second disease is protection after vaccination.

43. The non-transitory computer-readable medium of any one of claims 32-42, wherein the machine-learned analysis is random forest or gradient boosting for identifying the one or more universal signatures.

44. The non-transitory computer-readable medium of any one of claims 35-43, wherein the intervention is any one of a small molecule therapeutic, a biologic, a vaccine, or a gene therapy.

45. The non-transitory computer-readable medium of any one of claims 32-44, wherein individuals with the second disease have encountered or are likely to encounter the common condition.

46. The non-transitory computer-readable medium of claim 33, wherein generating the prediction of the second disease indication for the patient comprises performing an unsupervised clustering of the expressions of the one or more universal signatures to classify the subject.

47. The non-transitory computer-readable medium of claim 33 or 46, wherein generating the prediction of the second disease indication for the patient comprises performing a dimensionality reduction analysis of the expressions of the one or more universal signatures.

48. The non-transitory computer-readable medium of any one of claim 33 or 46-47, further comprising instructions that, when executed by the processor, cause the processor to perform the steps comprising:
determining whether to include the subject in a clinical trial study according to the prediction of the disease indication for the patient.

49. The non-transitory computer-readable medium of any one of claims 33-48, wherein the one or more universal signatures comprise one or more genes selected from NUP93, PPM1G, C6orf62, PJA1, MEST, NDUFS2, DDOST, DHRS7B, NOLC1, POLA2, PRSS23, SHMT1, RIPK1, AKR1A1, PRPF3, ETS1, MANSC1, PDHA1, ACLY, CHI3L2, MCMI, DNAJC18, LCT, YRDC, AIFM1, SFN, FBN1, EIF4H, CLEC4A, BCAP31, ATG4B, CSRP1, RDH11, GCLM, CDC7, GLOD5, IDH2, FMR1, PPARA, CCNE1, DDB1, BMP1, EHD4, VAV3, MPG, SPAG4, PSMD3, BCKDHA, GRAMD1B, and SEC61A1.

50. The non-transitory computer-readable medium of any one of claims 33-48, wherein the one or more universal signatures comprise one or more genes selected from CRB3, BCAP31, GMPPB, CD4, STARD3, CALR, CSRP1, CPT1A, LDLRAP1, RRAS, RASGRP2, PTS, SORDSLC26A6, VAT1, GPAA1, CXCR3, NAMPT, EPHX1, SEPT9, GMPPA, B4GALT7, AAAS, TP53INP1, GYS1, FASN, NOC4L, RRP9, MXI1, TP53, SLC7A11, FOXP3, DNASE1L1, MGAT1, SEC61A1, FYCO1, S100A10, LSS, IFRD1, DCP2, EDC4, ANKZF1, IDUA, IGFBP2, DDX39A, UCHL1, NR4A1, PDIA5, and ENGASE.

51. The non-transitory computer-readable medium of any one of claims 33-48, wherein the one or more universal signatures comprise one or more genes selected from NUB1, CASP1, WARS, TRIM21, STAT1, MOCOS, BCL2L14, ATF3, KIF2A, PDCD1LG2, SNX10, SEC24D, UBE2L6, LDHC, FAS, CXCL10, STAT2, IRF7, CD274, PSME2, LPCAT2, PSMB8, FBXO6, DUSP10, PLA2G4C, BANF1, EPOR, KCNMA1, CTSK, ITGA2, MPZL2, FEZ1, JAK2, BAZ1A, ICAM4, DAPP1, RIPK1, RNF144B, LAP3, C1QA, TYMP, GCH1, C1QB, CREM, ETV7, FOSB, MRPL15, PSEN1, MXI1, and TRAFD1.

52. The non-transitory computer-readable medium of any one of claims 33-48, wherein the one or more universal signatures comprise one or more genes selected from DNAAF1, UQCRC2, XPNPEP1, ACSM1, DDX60, TPI1, EFNA3, ZDHHC19, DDIT3, DNAJC12, RET, IL20RB, TNFSF10, DLG4, CKAP4, NDST1, GAPDH, ARL3, PLG, MDH2, GSTP1, S100A9, B4GALT7, H2AFJ, LTB4R, TAGLN2, IRF7, NDUFV1, CD300LB, RTP4, CTSD, HIST1H2BG, IL27, TNFRSF1B, SORBS1, NOP2, TNFSF13B, HLA-DRB5, RHOG, PSMB9, HSPA6, CD63, SLC2A8, IFITM1, CKB, ALDOA, MSRB1, OSMR, DRAP1, and PLA2G4A.

53. The non-transitory computer-readable medium of any one of claims 33-48, wherein the one or more universal signatures comprise one or more genes selected from LRRC28, E2F4, MRPL15, CCL22, OTUD1, NSUN7, CHEK1, ADGRA2, ZFPM2, GYS2, CD151, RAD51C, ARHGEF2, PFN1, AP4B1, IGFBP4, OASL, PDGFC, MIEN1, BEST3, SH3RF1, RACGAP1, FMO3, HNRNPA2B1, F2RL1, CAMKK2, ITGB5, FLVCR2, ZNF462, KIAA1324, CENPN, IKBKE, SERPINF2, FAM162A, SNX2, SERPING1, CLCA2, DPEP3, TNFAIP2, FSTL4, CTSD, BCAR1, MKX, RGS2, SAMD9, GCLM, BST1, IRS2, RNASE6, and ELOVL3.

54. The non-transitory computer-readable medium of any one of claims 33-48, wherein the one or more universal signatures comprise one or more genes selected from GSTM3, GYG1, CCL22, MOCS2, LY6E, CD151, S100A12, HEBP2, EIF3B, BAAT, MRPL11, OAS1, RFX5, PSMD7, ALDH2, STAP1, GYS2, GMFB, CCL3, PSMA4, CTHRC1, CMTM2, CD36, B4GALT2, EDF1, CDK5R1, TREML3P, PML, HEPHL1, TNFRSF21, PSMB9, GNAI1, TSPAN13, ATP6V0B, SLC4A4, ILF2, AKAP12, HLA-DRB5, PGR, AGTRAP, P3H1, CDADC1, TRIM5, PTGER3, ADCY6, ERBB2, NFYA, STATE, MMD, and RPL10A.

55. The non-transitory computer-readable medium of any one of claims 33-48, wherein the one or more universal signatures comprise one or more genes selected from MAFB, LGALS3, VCAN, PDK4, CD81, OLFM4, MMP8, CD1D, KLF4, CSTA, IDH1, ITPRIPL2, HMOX1, VSIG4, FRMD5, INHBA, ALDH2, PAPSS2, LTF, S100A12, MS4A6A, GSTK1, RNF31, NOTCH4, COL17A1, S100A8, CTSG, STX11, PTX3, MYOF, LTA4H, TRIM26, CYP1B1, ARG1, IFNGR2, B3GNT5, KYNU, LPGAT1, SLC9A3R1, HP, PADI4, PSME1, MGST2, NR4A1, SPP1, DEFA3, ME1, RBP7, DUSP6, and MCRS1.

56. The non-transitory computer-readable medium of any one of claims 33-48, wherein the one or more universal signatures comprise one or more genes selected from POLH, PTGER3, RUNX1, CASP6, CHPT1, APOBEC3F, USP14, PEX16, HLA-DQA1, IRF4, TNNC2, RIT1, ALG1, PDCD4, CYP2E1, GABARAPL2, B4GALT7, IFNAR1, MEF2C, TLR8, TSPYL2, M6PR, IKZF1, CNDP2, SLCO2A1, RBM4, FH, MRTO4, DTX4, RFC2, CAMK1G, CBX8, HM13, PSMB10, GCLM, SLC25A3, MYD88, IL33, ITGAM, PPIA, SEC22B, CXCR3, SCRN1, RXRA, SDHA, GLDC, FGF6, PRKG2, TFPI, and IMMT.

57. The non-transitory computer-readable medium of any one of claims 33-48, wherein the one or more universal signatures comprise one or more genes selected from CPEB4, CDKN3, TRIM14, ANXA9, CRYAB, CHST11, ANAPC11, RNASE3, FN1, ARNTL2, KRT82, PRIM2, MOCS2, IL21R, MAPK8, NMNAT1, ZNF107, CTSG, IL7, ANKRD34B, TMF1, HPS3, CIT, TRAP1, MSH2, PDGFC, TMLHE, MVP, TBX21, PICALM, KRT6A, FMR1, PCSK9, DNASE1L3, ENDOG, TPD52L1, PEX6, MPO, CHRNA7, SLFN5, TNFRSF1A, CD24, CASC1, LLGL2, DLG5, MYO5C, PGR, PFKFB2, AK2, and COL19A1.

58. The non-transitory computer-readable medium of any one of claims 33-48, wherein the one or more universal signatures comprise one or more genes selected from HUWE1, KCNK5, STX11, MORC3, NETO2, BATF2, CCL3L1, SAMD9, CCL2, PPFIA4, RPH3A, CXCL11, ERMAP, GBP2, CASP1, TLR7, EPX, ANKH, ARFGAP3, BAZ1A, COL5A1, COP1, BIRC2, SLC7A5, TRO, CXCL6, TNFSF10, GYPE, COL17A1, ROCK1, CD83, AK7, MSR1, LCN2, SPN, ASS1, HDGF, CXCL16, POLR3D, GK, OLFM4, STK3, RCBTB1, FOLR3, FBXO32, TMEM98, PRDX2, CKB, UHRF1BP1L and CTSG.

59. The non-transitory computer-readable medium of any one of claims 33-48, wherein the one or more universal signatures comprise one or more genes selected from AKR1A1, NDST1, RNF144B, HDAC9, PSMB3, PFKP, MB, MYC, PEX14, TAF13, BMX, PRKAA2, PTGER3, C3, SPTAN1, PROCR, AARS2, RHOT2, PHEX, THOP1, TIMM10, TBL1X, HNF4A, SLC6A9, FECH, CLCN3, CEACAM4, MMPI, HSD11B2, SLC25A25, RAB32, CXCL9, KCNE2, FCAR, CFP, IGF1, PEX16, RNF214, PIM1, JUNB, MDM2, PFKFB4, SIAH2, EGR2, KCNK10, EHMT2, FPR1, CD27, CETN2, and TGM1.

60. The non-transitory computer-readable medium of any one of claims 33-48, wherein the one or more universal signatures comprise one or more genes selected from SPOCK3, PVR, CHTF8, SLC20A1, PARP8, FGG, ZFAND2A, CCL25, CALR, TM7SF2, FUS, DDAH2, SPAG4, FBXL14, LGALS8, GNE, HAS2, IGSF6, B4GALT1, POLK, PLK4, NDUFB4, GNG8, MUC1, AGGF1, PPIB, SLC1A4, HLA-DQB1, SEMA4G, MT2A, COL4A2, PLCB4, GYS1, PRKCG, RXFP2, PLA2G4C, ALDH1A2, IL1A, IBTK, SPARC, OAS3, EPHA4, HLA-B, MICB, CCL18, SLC39A6, GLCE, TUBB2B, FBXO8, and SNX6.

61. The non-transitory computer-readable medium of any one of claims 33-48, wherein the one or more universal signatures comprise one or more genes selected from NLRC5, CACNB2, CELSR1, PARP8, ECT2, HTATIP2, NRP1, NCK2, TMEM100, CLCA2, BAALC, PTPN14, IRF9, SAA2, HR, IRGQ, AKT3, SYNGR1, NKX2-2, MT1H, SERPINA6, CAMK2N1, CCT6B, WDHD1, NKX3-1, LDHC, MALT1, CD9, CLGN, SLC25A19, MAP7, XCL1, ACSL6, TFRC, CAT, NKD1, CNBP, ALDH1L1, CCL7, SLC20A1, KRAS, CSF1, CASP2, HDAC11, KIR2DS4, CEACAM19, CFH, CAB39L, DEPDC1, and PSMA1.

62. The non-transitory computer-readable medium of any one of claims 33-48, wherein the one or more universal signatures comprise one or more genes selected from CCK, SESN2, NACAD, PCSK9, C1R, SLC7A1, ECM1, XCL1, ARG2, SPSB1, DNAH17, TNNC1, CPN1, SYNGR2, CPA4, MYL1, DUOX2, ZNF621, GAPDHS, BCAP31, DLG1, IL17RB, SLC6A6, BCL2L2, HSPA1B, SLC1A4, TSTD1, HSPB8, MSC, CENPJ, ARL8A, CTLA4, GFRA1, WASF1, RIPK1, ENO3, KRT19, PLVAP, RAD18, ACHE, FBLN5, MGST2, ANAPC5, RFX5, CASP7, STC1, NCK2, IFI27, APOA4, and MSRB2.
